(19) United States
(12) Patent Application Publication
(10) Pub. No.: US 2024/0269271 A1
(43) Pub. Date: Aug. 15, 2024

(54) GLYCOPROTEIN D VARIANTS AS VACCINE ADJUVANTS

(71) Applicants: VIRION THERAPEUTICS, LLC, Newark, DE (US); THE WISTAR INSTITUTE, Philadelphia, PA (US)

(72) Inventors: Hildegund CJ Ertl, Villanova, PA (US); Sue L. Currie, Newark, DE (US); Andrew D. Luber, Newark, DE (US)

(21) Appl. No.: 18/407,567

(22) Filed: Jan. 9, 2024

Related U.S. Application Data
(60) Provisional application No. 63/479,106, filed on Jan. 9, 2023.
(60) Provisional application No. 63/479,111, filed on Jan. 9, 2023.

(51) Int. Cl. A61K 39/39; A61K 39/00; A61K 39/12; C07K 14/005; C07K 14/47

(52) U.S. Cl. CPC A61K 39/39; A61K 39/12; C07K 14/005; C07K 14/47; A61K 2039/5256; A61K 2039/55516; A61K 2039/585; C07K 2319/00; C12N 2710/20022; C12N 2710/20034

(57) ABSTRACT

Disclosed herein are compositions for increasing the immunogenicity of a vaccine antigen and methods of inducing an immune response in a subject using the compositions described herein. Disclosed herein are compositions for a therapeutic vaccine to HPV-associated cancers and methods of inducing an immune response to HPV in a subject using the compositions described herein.

### CROSS-REFERENCE TO RELATED APPLICATIONS

This application claims priority to United States Provisional Application Ser. No. 63/479,106, filed on Jan. 9, 2023, and United States Provisional Application Ser. No. 63/479,111, filed on Jan. 9, 2023, the disclosures of each of which are hereby incorporated by reference in their entireties.

### SEQUENCE LISTING

The contents of the electronic sequence listing (111876.000084_Sequence_Listing.xml; Size: 254,947 bytes; and Date of Creation: Apr. 3, 2024) is herein incorporated by reference in its entirety.

### TECHNICAL FIELD

Disclosed herein are compositions and methods of increasing the immunogenicity of an antigen, and compositions and methods for a therapeutic vaccine to HPV-associated cancers.

### BACKGROUND

Generating an efficient immune response to an antigen is important to the success of vaccines. One strategy to increase the immunogenicity of an antigen is to include a protein adjuvant within the vaccine. Vaccines against viruses, bacteria, or cancer could benefit from a protein adjuvant, which can enhance the T-cell response and the generation of neutralizing antibodies. Proteins adjuvants with improved performance have the potential to increase vaccine success and better prevent disease.

Several viruses, including oncogenic types of the human papilloma virus (HPV), have been linked to cancer in humans. HPVs have been shown to be causal agents in cervical cancer as well as cancers of the penis, anus, vagina, vulva, mouth, and throat. HPV infections can be cleared by HPV-specific CD8+ T cells. Most HPV infected individuals mount such a CD8+ T cell response and eliminate the infected cells. Others, however, fail to develop an effective CD8+ T cell response and instead maintain a persistent infection that may eventually progress to cancer. Vaccines that induce CD8+ T cells to the oncoproteins of HPV that are expressed by persistently infected or transformed cells may be able to prevent or treat HPV-associated malignancies.

### SUMMARY

Disclosed herein are nucleic acid molecules encoding a polypeptide comprising the amino acid sequence of SEQ ID NO: 11.

Also disclosed herein are gDM5 proteins comprising the amino acid sequence of SEQ ID NO: 11.

Also disclosed herein are fusion proteins comprising an N-terminal gDM5 sequence comprising the amino acid sequence of SEQ ID NO: 15, an antigen, and a C-terminal gDM5 sequence comprising the amino acid sequence of SEQ ID NO: 17.

Also disclosed herein are nucleic acid molecules encoding a mutant human papilloma virus 16 (HPV 16) E7 protein comprising the amino acid sequence of SEQ ID NO: 34, a mutant HPV 16 E6 protein comprising the amino acid sequence of SEQ ID NO: 36, a mutant HPV 16 E5 protein comprising the amino acid sequence of SEQ ID NO: 38, a mutant HPV 16 E2 protein comprising the amino acid sequence of SEQ ID NO: 40, a C-terminal mutant HPV 16 E2 protein comprising the amino acid sequence of SEQ ID NO: 56, a N-terminal mutant HPV 16 E2 protein comprising the amino acid sequence of SEQ ID NO: 58, a mutant HPV 16 E7 protein v2 comprising the amino acid sequence of SEQ ID NO: 64, a mutant HPV 16 E6 protein v2 comprising the amino acid sequence of SEQ ID NO: 66, or a mutant HPV 16 E5 protein v2 comprising the amino acid sequence of SEQ ID NO: 68.

Also disclosed herein are nucleic acid molecules encoding an HPV 16 fusion protein, wherein the HPV 16 fusion protein comprises any one of the HPV 16 E7 proteins provided in Table 10, any one of the HPV 16 E6 proteins provided in Table 10, and any one of the HPV 16 E5 proteins provided in Table 10.

Also disclosed herein are nucleic acid molecules encoding an HPV 16 E2-antigen fusion protein, wherein the HPV 16 E2-antigen fusion protein comprises any one of the HPV 16 E2 proteins provided in Table 14, and any one of the antigens provided in Table 14.

Also disclosed herein are proteins comprising any one of the amino acid sequences provided in Tables 7, 8, 10, 12, 14, 16, 17, or 19.

### BRIEF DESCRIPTION OF THE DRAWINGS

The summary, as well as the following detailed description, is further understood when read in conjunction with the appended drawings. For the purpose of illustrating the disclosed compositions and methods, the drawings show exemplary embodiments of the compositions and methods; however, the compositions and methods are not limited to the specific embodiments disclosed. In the drawings:

FIG. 1 illustrates a flow cytometry analysis of gD expression upon transduction of HEK293 cells with adenovirus (Ad) vectors encoding wild-type glycoprotein D (gD) and various gD variants (gDM1, gDM2, or gDM3).

FIG. 2 shows a Western blot analysis of lysates from HEK293 cells that had been transduced with 1000 virus particles (vp) of the indicated vector per cell using a monoclonal anti-gD antibody.

FIG. 3 shows the induction of CD8⁺ T cell responses to vectors expressing fusion proteins of the indicated gDs and E765dt1 at day 14 (d14) (left) and day 28 (d28) (right).

FIG. 4A illustrates the frequencies of IFN-γ+CD44+CD8+ cells over CD44+CD8+ cells from C57Bl/6 mice immunized with 5×10¹⁰ vp of vectors encoding the indicated fusion proteins. FIG. 4B shows the frequencies of IFN-γ+CD44+CD8+ cells over CD44+CD8+ cells in HLA-A02 transgenic mice immunized with 1×10⁹ vp of vectors encoding the indicated fusion proteins. Splenocytes were tested 6 weeks later against the E7 peptide pool.

FIG. 5 shows a Western blot analysis of protein expression upon transfection of HEK293 cells with Ad vectors encoding the indicated proteins using a monoclonal anti-gD antibody.

FIG. 6 shows the induction of CD8⁺ T cell responses from the indicated vectors containing a Melapoly #2 insert fused into wild type gD or gDM5.

FIG. 7 illustrates the CD8+ T cell response upon vaccination with Ad vectors encoding the indicated fusion proteins. Cells were tested using peptide pools representing the PolN sequence or the most immunodominant peptides thereof.

FIG. 8 shows the induction of CD8⁺ T cell responses upon vaccination with Ad vectors encoding the indicated gDs and the mutant E765 insert. Significant differences indicated by line and stars above (p-value >0.0001) were calculated by t-test.

FIG. 9A, FIG. 9B, FIG. 9C, and FIG. 9D show responses to individual peptides from C57Bl/6 mice immunized with 5×10¹⁰ vp of vectors encoding the indicated inserts or as a control naïve mice.

FIG. 10A and FIG. 10B show frequencies of IFN-γ+CD44+CD8+ cells over CD44+CD8+ cells from spleens from HLA-A2 mice immunized with 1×10⁹ vp of Ad vectors encoding the indicated inserts.

FIG. 11 depicts the tumor volume over time in individual C57Bl/6 mice that were vaccinated with 1×10¹⁰ vp of an Ad vector encoding the gDM5-E7652 insert or a control vector encoding HIV-1 gag genetically fused into gDM5. Mice were challenged 4 weeks later with 5×10⁵ TC1 cells given subcutaneously (lines indicate means).

FIG. 12A and FIG. 12B show the results for groups of C57Bl/6 mice that were challenged with 5×10⁴ TC1 cells and vaccinated three days later with 1×10¹⁰ vp of the Ad vectors encoding gDM5-E7652 or a control vector encoding HIV-1 gag genetically fused into gDM5. FIG. 12A depicts the tumor volume over time. FIG. 12B illustrates the percentage of tumor-free mice over time.

FIG. 13A and FIG. 13B show the results from mice that were vaccinated with modified versions of E7, E6, and E5 fused into wild type gD by the chimpanzee adenovirus vector AdC68 and challenged 3 days earlier with 5×10⁴ TC1 cells. FIG. 13A depicts the tumor volume over time. FIG. 13B illustrates the percentage of tumor free mice over time.

FIG. 14A, FIG. 14B, FIG. 14C, and FIG. 14D show the results from mice that were challenged with a higher dose of 2×10⁵ TC1 cells prior to vaccination with 10¹⁰ vp of AdC6-gDM5-E7652, AdC6-gDM5-E765dt3, or the control vector AdC6-gDM5-gag. FIG. 14A, FIG. 14B, and FIG. 14C depict the tumor volume over time from mice immunized with 1×10¹⁰ virus particles of the indicated vectors. FIG. 14D illustrates the percentage of tumor-free mice over time.

FIG. 15 shows the results of Western blots analyses. HEK293 cells were transduced with a vector encoding SgD-PA2-E7652 which contains a FLAG sequence as the C terminus of E7652 (SgD-PA2-FLAG-E7652) or a vector encoding a fusion protein of gD from which the transmembrane domain had been removed and wild-type E765 (gD(-TM)-E765). Both cell lysate and cell supernatant were probed with an antibody against gD for presence of the inserts.

FIG. 16 shows the CD8+ T cell response to the immunodominant E7 epitope in mice immunized 2 weeks before with Ad vectors expressing the indicated proteins.

FIG. 17 shows the CD8+ T cell response to the immunodominant E7 epitope in mice immunized 2 weeks before with Ad vectors expressing the indicated proteins. Results were analyzed by t-test and found to be significantly different.

FIG. 18A shows a Western blot analysis of lysates from HEK293 cells transduced with 1000 virus particles (vp) of the indicated vector per cell (left two blots) or that had been transduced with plasmid vectors (right 2 blots). To determine the size of the N-terminal fragment, a plasmid vector termed pHis-gDE7652 was developed that contained a His-tag followed by a cleavage site and then the gDE7652 sequence. Western Blot analyses of cells transduced with this plasmid showed upon staining with an antibody to gD a fragment of ˜45 kDa, which was also detected in cells transfected with AdC6-gDE7652. The anti-His-tag antibody detected a larger protein of ˜60 kDa. FIG. 18B shows the results of an ELISA assay testing whether the larger protein could bind HVEM with plates coated with BTLA. Plates were then treated with either a commercially available gD protein or lysates of cells, which had been transfected with pHis-gDE7652 or a control plasmid that expresses a sequence of the V2 loop of HIV envelope linked to a His-tag. Before testing, both lysates were purified over Ni++columns. Other wells were treated with the proteins' diluent. Wells were then tested for binding of HVEM, which has a site that binds both BTLA and with higher affinity gD. A commercially available gD inhibited HVEM binding to BTLA by ˜90%. The was some non-specific inhibition of ˜45% by the V2 protein while the gD-E7652 N-terminal fragment caused complete inhibition confirming that even after cleavage of the C-terminus, the N-terminal part of the gD-HPV fusion protein was able to bind HVEM. Data were analyzed by 2-way Anova. FIG. 18C shows a Western Blot to detect proteins encoded by the Ad vectors expressing gD-Melapoly #2E2-Flag as compared to gD-Melalapoly. Lysate of cells infected with the gD-Melapoly #2E2-Flag vector showed upon staining with an anti-gD antibody a small band of the full-length protein which was slightly larger than the band seen in lysate of cells infected with the gD-Melapoly #2 expressing vector. A more pronounced band was smaller at about 55 kD corresponding in size to the smaller band seen in lysate of cells infected with the Ad vector encoding the mutants E7, E6, E5 and E2 protein again indicating that the E2 protein had been spliced.

FIG. 19 shows the frequencies of CD8+ cells over CD44+CD8+ cells from C57Bl/6 mice immunized with 1×10¹⁰ virus particles (vp) of the indicated vectors comparing vectors expressing gDE765 or gDE7652. One month later splenocytes were tested for production of interferon (IFN)-γ, perforin, and granzyme B (Gzmb) by intracellular cytokine staining in response to peptide pools of E5, E6 and E7.

FIG. 20 shows combinations of functions of CD8+ T cell responses of splenocytes of mice vaccinated with 1×10¹⁰ vp of AdC68-gDE765 or AdC6-gDE7652 that were tested 4 weeks after vaccination for responses to peptide pools representing the HPV sequences.

FIG. 21A and FIG. 21B show CD8+ T cell responses of splenocytes of mice vaccinated with 1×10¹⁰ vp of AdC68-gDE765 or AdC6-gDE7652 that were tested 4 weeks after vaccination for responses to peptide pools (P) or individual peptides representing the HPV sequences. Data were analyzed by 2-way Anova. FIG. 21C and FIG. 21D show the proportion of the responses to different peptides. Overall frequencies are shown below the circles.

FIG. 22A, FIG. 22B, and FIG. 22C show CD8+ T cell responses to individual peptides corresponding to the inserted HPV 16 sequences tested from splenocytes of mice immunized 4 weeks previously with 1×10¹⁰ vp of the AdC6-gDE7652 or AdC6-E7652 vectors. Naïve mice served as controls.

FIG. 23 shows a graph depicting groups of 5 C57Bl/6 mice per group vaccinated with 5×10¹⁰ vp of the indicated vectors. Four weeks later splenocytes were tested for IFN-γ production in response to peptide pools of E7, E6, E5 and E2. The graph shows percentages of responding CD44+CD8+ cells over all CD44+CD8+ cells.

FIG. 24 shows tumor development in mice challenged with 2×10⁵ TC-1 cells and then vaccinated 3 days later with 1×10¹⁰ vp of the indicated vectors given intramuscularly.

FIG. 25 shows tumor sizes as volume over time in mice challenged with 2×10⁵ TC-1 cells and then vaccinated 3 days (top graph) or 9 days (bottom graph) later with 1×10¹⁰ vp of the indicated vectors given intramuscularly.

FIG. 26 shows tumor sizes as volume over time in mice challenged with 5×10⁴ TC-1 cells and then vaccinated 9 days later with 1×10¹⁰ vp of the indicated vectors given intramuscularly (top graph) and the percentage of the same mice remaining alive and at what day after challenge had ongoing tumors above a volume of 50 mm³ (bottom graph), where 50 mm³ was a size where tumors could no longer regress.

FIG. 27 shows graphs depicting groups of 10 mice that were challenged with 2×10⁵ TC1 cells. Mice were vaccinated 3 days later with 1×10¹⁰ vp of the indicated vaccines. Tumor growth (top graph) and survival (bottom graph) were recorded.

FIG. 28 shows responses of CD8+ T cells from spleens or tumors (TILs) of mice challenged with 2×10⁵ TC-1 cells and then vaccinated 10 days later with the indicated vaccines given at 1×10¹⁰ vp. T cell responses were tested 10 days later by ICS for IFN-γ, perforin and granzyme B. The graphs show frequencies of CD8+CD44+ T cells over all CD44+CD8+ T cells that were positive for the different combinations of function.

FIG. 29 shows graphs depicting the same lymphocytes described in FIG. 28 that were tested with a dextramer to an immunodominant epitopes for E7, and were co-stained with markers for T cell activation/exhaustion. The graphs show percentages of dextramer+CD44+CD8+ T cells that expressed either individual markers or combination of markers.

FIG. 30 shows the frequencies of Dextramer+CD44+CD8+ T cells over all CD44+CD8+ T cells. Mice were injected with 5×10⁴ TC-1 cells and were vaccinated with the indicated vaccines given at 1×10¹⁰ vp. T cells were isolated from spleens and small tumors 9 days later or from large tumors roughly 30 days later. Cells were stained with T cell identifying markers and a dextramer to an immunodominant epitopes for E7. Data were analyzed by 2-way Anova.

FIG. 31A shows the percentages of dextramer+CD8+ T cells that stained for a given marker. FIG. 31B shows the percentages of dextramer+CD8+ T cells that stained for a given marker (left graph and middle graph) and a graph depicting the number of markers expressed by the different CD8+ T cell population (right graph). For both FIG. 31A and FIG. 31B, the same cells described in FIG. 30 isolated from tumors were stained with the dextramer to an immunodominant epitopes for E7 and antibodies to T cell activation/differentiation/exhaustion markers. Data were analyzed by 2-way Anova.

FIG. 32 shows CD8+ T cell responses to an immunodominant trp1 antigen present in the Melapoly sequence in mice immunized 4 weeks earlier with an AdC vector expressing gD-Melapoly #2 fused to E2 or gD-Melapoly #2 only.

### DETAILED DESCRIPTION OF ILLUSTRATIVE EMBODIMENTS

The disclosed compositions and methods may be understood more readily by reference to the following detailed description taken in connection with the accompanying figures, which form a part of this disclosure. It is to be understood that the disclosed compositions and methods are not limited to the specific compositions and methods described and/or shown herein, and that the terminology used herein is for the purpose of describing particular embodiments by way of example only and is not intended to be limiting of the claimed compositions and methods.

Unless specifically stated otherwise, any description as to a possible mechanism or mode of action or reason for improvement is meant to be illustrative only, and the disclosed compositions and methods are not to be constrained by the correctness or incorrectness of any such suggested mechanism or mode of action or reason for improvement.

Throughout this text, the descriptions refer to compositions and methods of using said compositions. Where the disclosure describes or claims a feature or embodiment associated with a composition, such a feature or embodiment is equally applicable to the methods of using said composition. Likewise, where the disclosure describes or claims a feature or embodiment associated with a method of using a composition, such a feature or embodiment is equally applicable to the composition.

Where a range of numerical values is recited or established herein, the range includes the endpoints thereof and all the individual integers and fractions within the range, and also includes each of the narrower ranges therein formed by all the various possible combinations of those endpoints and internal integers and fractions to form subgroups of the larger group of values within the stated range to the same extent as if each of those narrower ranges was explicitly recited. Where a range of numerical values is stated herein as being greater than a stated value, the range is nevertheless finite and is bounded on its upper end by a value that is operable within the context of the herein disclosure. Where a range of numerical values is stated herein as being less than a stated value, the range is nevertheless bounded on its lower end by a non-zero value. It is not intended that the scope of the compositions and methods be limited to the specific values recited when defining a range. All ranges are inclusive and combinable.

It is to be appreciated that certain features of the disclosed compositions and methods which are, for clarity, described herein in the context of separate embodiments, may also be provided in combination in a single embodiment. Conversely, various features of the disclosed compositions and methods that are, for brevity, described in the context of a single embodiment, may also be provided separately or in any subcombination.

As used herein, the singular forms “a,” “an,” and “the” include the plural.

Various terms relating to aspects of the description are used throughout the specification and claims. Such terms are to be given their ordinary meaning in the art unless otherwise indicated. Other specifically defined terms are to be construed in a manner consistent with the definitions provided herein.

The term “comprising” is intended to include examples encompassed by the terms “consisting essentially of” and “consisting of”; similarly, the term “consisting essentially of” is intended to include examples encompassed by the term “consisting of.”

As used herein, “administering to said subject,” “providing to the subject,” and similar terms indicate a procedure by which the herein disclosed nucleic acid molecules, vectors, fusion proteins, viruses, pharmaceutical compositions, and/or vaccines are provided to a subject such that target cells, tissues, or segments of the body of the subject are contacted with the herein disclosed nucleic acid molecules, vectors, fusion proteins, viruses, pharmaceutical compositions, and/or vaccines.

As used herein, the phrase “effective amount” refers to an amount of the nucleic acids, vectors, fusion proteins, vectors, pharmaceutical composition, or vaccines as described herein, effective to achieve a particular biological or therapeutic result such as, but not limited to, biological or therapeutic results disclosed, described, or exemplified herein. The therapeutically effective amount may vary according to factors such as the disease state, age, sex, and weight of the subject, and the ability of the composition to cause a desired response in a subject. Exemplary indicators of a therapeutically effective amount include, for example, activation of the immune response, improved well-being of the subject, reduction of a tumor burden, arrested or slowed growth of a cancer, and/or absence of metastasis of cancer cells to other locations in the body.

The term “subject” as used herein is intended to mean any animal, in particular, mammals. Although the induction of an immune response in mice and treatment and/or vaccination in mice is exemplified herein, any type of mammal can be treated using the disclosed methods. Thus, the methods are applicable to human and nonhuman animals, although preferably used with mice and humans, and most preferably with humans. “Subject,” “individual,” and “patient” are used interchangeably herein.

### Glycoprotein D Variants

Described herein are nucleic acid molecules encoding mutant herpes simplex virus (HSV) glycoprotein D (gD) proteins (referred to herein as gDM1 (gD1), gDM2 (gD2), gDM3 (gD3), gDM4 (gD4), and gDM5 (gD5)). The nucleic acid molecules can encode the gDM1 protein, gDM2 protein, gDM3 protein, gDM4 protein, or gDM5 protein having the sequence provided in Table 23.

The nucleic acid molecule can encode a gDM1 protein. In some embodiments, the nucleic acid molecule encodes a gDM1 polypeptide comprising the amino acid sequence of SEQ ID NO: 1. In some embodiments, the nucleic acid molecule comprises the nucleotide sequence of SEQ ID NO: 2.

The nucleic acid molecule can encode a gDM2 protein. In some embodiments, the nucleic acid molecule encodes a gDM2 polypeptide comprising the amino acid sequence of SEQ ID NO: 3. In some embodiments, the nucleic acid molecule comprises the nucleotide sequence of SEQ ID NO: 4.

The nucleic acid molecule can encode a gDM3 protein. In some embodiments, the nucleic acid molecule encodes a gDM3 polypeptide comprising the amino acid sequence of SEQ ID NO: 5. In some embodiments, the nucleic acid molecule comprises the nucleotide sequence of SEQ ID NO: 6.

The nucleic acid molecule can encode a gDM4 protein. In some embodiments, the nucleic acid molecule encodes a gDM4 polypeptide comprising the amino acid sequence of SEQ ID NO: 7. In some embodiments, the nucleic acid molecule comprises the nucleotide sequence of SEQ ID NO: 8.

The nucleic acid molecule can encode a gDM5 protein. In some embodiments, the nucleic acid molecule encodes a gDM5 polypeptide comprising the amino acid sequence of SEQ ID NO: 11. In some embodiments, the nucleic acid molecule comprises the nucleotide sequence of SEQ ID NO: 12. In some embodiments, the nucleic acid molecule encodes a gDM5 polypeptide comprising the amino acid sequence of SEQ ID NO: 9. In some embodiments, the nucleic acid molecule comprises the nucleotide sequence of SEQ ID NO: 10.

The nucleic acid molecule encoding a mutant gD protein can be a fragment or portion thereof. Suitable mutant gD fragments or mutant gD portions that the nucleic acid molecule can encode include N-terminal gD polypeptides and C-terminal gD polypeptides. The nucleic acid molecule can encode an N-terminal polypeptide of gDM5. In some embodiments, the nucleic acid molecule encodes a polypeptide comprising the amino acid sequence of SEQ ID NO: 15. In some embodiments, the nucleic acid molecule comprises the nucleotide sequence of SEQ ID NO: 16. In some embodiments, the nucleic acid molecule encodes a polypeptide comprising the amino acid sequence of SEQ ID NO: 13. In some embodiments, the nucleic acid molecule comprises the nucleotide sequence of SEQ ID NO: 14. The nucleic acid molecule can encode a C-terminal polypeptide of gDM5. In some embodiments, the nucleic acid molecule encodes a polypeptide comprising the amino acid sequence of SEQ ID NO: 17. In some embodiments, the nucleic acid molecule comprises the nucleotide sequence of SEQ ID NO: 18.

The nucleic acid molecules encoding a mutant gD protein can further encode an antigen. In some embodiments, the nucleic acid molecules can encode a fusion protein comprising the mutant gD and the antigen. The nucleic acid molecules can encode a gDM5 protein and an antigen. In some embodiments, the nucleic acid molecule encodes a fusion protein comprising a gDM5 sequence and an antigen. The nucleic acid molecule encoding a fusion protein comprising a gDM5 sequence and an antigen can encode one or more of the amino acid sequences of SEQ ID NO: 9, SEQ ID NO: 11, SEQ ID NO: 15, SEQ ID NO: 13, or SEQ ID NO: 17. The nucleic acid molecule encoding a fusion protein comprising a gDM5 sequence and an antigen can comprise one or more of the nucleotide sequences of SEQ ID NO: 10, SEQ ID NO: 12, SEQ ID NO: 16, SEQ ID NO: 14, or SEQ ID NO: 18.

The nucleic acid molecule can encode a fusion protein comprising an N-terminal gDM5 polypeptide and an antigen. The nucleic acid molecule can encode a fusion protein comprising a C-terminal gDM5 polypeptide and an antigen. The nucleic acid molecule can encode a fusion protein comprising both an N-terminal gDM5 polypeptide and a nucleotide sequence encoding a C-terminal gDM5 polypeptide and an antigen. In some embodiments, the nucleic acid molecule encodes a fusion protein comprising an N-terminal gDM5 polypeptide, an antigen, and a C-terminal gDM5 polypeptide.

The nucleic acid molecule that encodes an N-terminal gDM5 protein can encode the amino acid sequence of SEQ ID NO: 15. The nucleic acid molecule that encodes an N-terminal gDM5 protein can comprise the nucleotide sequence of SEQ ID NO: 16. The nucleic acid molecule that encodes an N-terminal gDM5 protein can encode the amino acid sequence of SEQ ID NO: 13. The nucleic acid molecule that encodes an N-terminal gDM5 protein can comprise the nucleotide sequence of SEQ ID NO: 14.

The nucleic acid molecule that encodes a C-terminal gDM5 protein can encode the amino acid sequence of SEQ ID NO: 17. The nucleic acid molecule that encodes a C-terminal gDM5 protein can comprise the nucleotide sequence of SEQ ID NO: 18.

The nucleic acid molecule can encode a fusion protein, wherein the nucleic acid molecule comprises a nucleotide sequence encoding an N-terminal gDM5 sequence comprising the amino acid sequence of SEQ ID NO: 15, a nucleotide sequence encoding an antigen, and a nucleotide sequence encoding a C-terminal gDM5 sequence comprising the amino acid sequence of SEQ ID NO: 17. The nucleic acid molecule can encode a fusion protein, wherein the nucleic acid molecule comprises an N-terminal gDM5 nucleotide sequence comprising SEQ ID NO: 16, a nucleotide sequence encoding an antigen, and a C-terminal gDM5 nucleotide sequence comprising SEQ ID NO: 18.

The nucleic acid molecule can encode a fusion protein, wherein the nucleic acid molecule comprises a nucleotide sequence encoding an N-terminal gDM5 sequence comprising the amino acid sequence of SEQ ID NO: 13, a nucleotide sequence encoding an antigen, and a nucleotide sequence encoding a C-terminal gDM5 sequence comprising the amino acid sequence of SEQ ID NO: 17. The nucleic acid molecule can encode a fusion protein, wherein the nucleic acid molecule comprises an N-terminal gDM5 nucleotide sequence comprising SEQ ID NO: 14, a nucleotide sequence encoding an antigen, and a C-terminal gDM5 nucleotide sequence comprising SEQ ID NO: 18.

The nucleic acids can encode a fusion protein comprising an antigen. Suitable antigens include, for example, a hepatitis virus antigen, an HIV antigen, a melanoma antigen, or an HPV antigen. In some embodiments, the nucleic acid molecule encodes a fusion protein comprising a hepatitis virus antigen. In some embodiments, the nucleic acid molecule encodes a fusion protein comprising an HIV antigen. In some embodiments, the nucleic acid molecule encodes a fusion protein comprising a melanoma antigen. In some embodiments, the nucleic acid molecule encodes a fusion protein comprising an HPV antigen.

In some embodiments, the nucleic acid molecule encodes a fusion protein comprising a PolN protein from HBV. In some embodiments, the nucleic acid molecule encodes a fusion protein comprising a gag protein from HIV. In some embodiments, the nucleic acid molecule encodes a fusion protein comprising an E protein of HPV. In some embodiments, the nucleic acid molecule encodes a fusion protein comprising HBV3 Protein. In some embodiments, the nucleic acid molecule encodes a fusion protein comprising Melapoly Protein. In some embodiments, the nucleic acid molecule encodes a fusion protein comprising E765-wt Protein. In some embodiments, the nucleic acid molecule encodes a fusion protein comprising Melapoly Protein #2. In some embodiments, the nucleic acid molecule encodes a fusion protein comprising Melanoma antigens with universal helper epitope Protein. The nucleic acid molecules can encode a fusion protein comprising an antigen comprising the amino acid sequence of SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 54, SEQ ID NO: 72, or SEQ ID NO: 74. The nucleic acid molecules can comprise an antigen comprising the nucleotide sequence of SEQ ID NO: 20, SEQ ID NO: 22 SEQ ID NO: 24, SEQ ID NO: 26, SEQ ID NO: 28, SEQ ID NO: 30, SEQ ID NO: 32, SEQ ID NO: 55, or SEQ ID NO: 73, SEQ ID NO: 75.

Also described herein are mutant herpes simplex virus (HSV) glycoprotein D proteins (gD). The mutant gD proteins can be gDM1 protein, gDM2 protein, gDM3 protein, gDM4 protein, or gDM5 protein. In some embodiments, the gDM1 protein comprises the amino acid sequence of SEQ ID NO: 1. In some embodiments, the gDM2 protein comprises the amino acid sequence of SEQ ID NO: 3. In some embodiments, the gDM3 protein comprises the amino acid sequence of SEQ ID NO: 5. In some embodiments, the gDM4 protein comprises the amino acid sequence of SEQ ID NO: 7. In some embodiments, the gDM5 protein comprises the amino acid sequence of SEQ ID NO: 11. In some embodiments, the gDM5 protein comprises the amino acid sequence of SEQ ID NO: 9.

The mutant gD protein can comprise a fragment or portion thereof. Suitable mutant gD fragments or mutant gD portions include N-terminal gD polypeptides and C-terminal gD polypeptides. The mutant gDM5 protein can comprise an N-terminal gDM5 polypeptide, a C-terminal gDM5 polypeptide, or both the N-terminal gDM5 polypeptide and the C-terminal gDM5 polypeptide. In some embodiments, the gDM5 protein comprises the amino acid sequence of SEQ ID NO: 15. In some embodiments, the gDM5 protein comprises the amino acid sequence of SEQ ID NO: 13. In some embodiments, the gDM5 protein comprises the amino acid sequence of SEQ ID NO: 17.

Also disclosed herein are fusion proteins comprising any of the herein described mutant gD proteins and an antigen. In some embodiments, the fusion protein comprises a gDM5 sequence and antigen. The fusion protein comprising a gDM5 sequence and an antigen can comprise the amino acid sequence of SEQ ID NO: 11 or SEQ ID NO: 9. The fusion protein can comprise a mutant gD protein fragment or mutant gD protein portion. Suitable mutant gD fragments or mutant gD portions include N-terminal gDM5 polypeptides and C-terminal gDM5 polypeptides. In some embodiments, the fusion protein comprises an N-terminal gDM5 polypeptide, a C-terminal gDM5 polypeptide, or both an N-terminal gDM5 polypeptide and a C-terminal gDM5 polypeptide. The gDM5 fragment portion of the fusion protein can comprise one or more of the amino acid sequences of SEQ ID NO: 15, SEQ ID NO: 13, or SEQ ID NO: 17.

The fusion protein can comprise an N-terminal gDM5 sequence comprising the amino acid sequence of SEQ ID NO: 15, an antigen, and a C-terminal gDM5 sequence comprising the amino acid sequence of SEQ ID NO: 17. The fusion protein can comprise an N-terminal gDM5 sequence comprising the amino acid sequence of SEQ ID NO: 13, an antigen, and a C-terminal gDM5 sequence comprising the amino acid sequence of SEQ ID NO: 17.

The fusion proteins can comprise any antigen. Suitable antigens include, for example, a hepatitis virus antigen, an HIV antigen, a melanoma antigen, or an HPV antigen. In some embodiments, the fusion protein comprises a hepatitis virus antigen. In some embodiments, the fusion protein comprises an HIV antigen. In some embodiments, the fusion protein comprises a melanoma antigen. In some embodiments, the fusion protein comprises an HPV antigen.

In some embodiments, the fusion protein comprises a PolN protein from HBV. In some embodiments, the fusion protein comprises a gag protein from HIV. In some embodiments, the fusion protein comprises an E protein of HPV. In some embodiments, the fusion protein comprises HBV3 Protein. In some embodiments, the fusion protein comprises Melapoly Protein. In some embodiments, the fusion protein comprises E765-wt Protein. In some embodiments, the fusion protein comprises Melapoly Protein #2. In some embodiments, the fusion protein comprises Melanoma antigens with universal helper epitope Protein. The fusion proteins can comprise an antigen comprising the amino acid sequence of SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 54, SEQ ID NO: 72, or SEQ ID NO: 74.

Also described herein are vectors and viruses comprising any of the herein described nucleic acid molecules. In some embodiments, the vectors comprise any of the herein described nucleic acid molecules. Disclosed herein are also host cells comprising any of the herein disclosed vectors. Suitable host cells include eukaryotic cells and prokaryotic cells. In some embodiments, the viruses comprise any of the herein described nucleic acid molecules. In some embodiments, the viruses can comprise any one of the herein described vectors.

The vectors and/or viruses can comprise one or more of any of the herein described nucleic acid molecules. For example, in some exemplary embodiments, the vectors and/or viruses comprise nucleic acid molecules encoding mutant herpes simplex virus (HSV) glycoprotein D (gD) proteins, such as mutant gDM1 protein, gDM2 protein, gDM3 protein, gDM4 protein, or gDM5 protein. In some exemplary embodiments, the vectors and/or viruses can comprise nucleic acid molecules encoding a gDM5 polypeptide of SEQ ID NO: 9 or SEQ ID NO: 11. In some exemplary embodiments, the gDM5 can be a gDM5 fragment or gDM5 portion as described herein, such as an N-terminal gDM5 polypeptide, a C-terminal gDM5 polypeptide, or both an N-terminal gDM5 polypeptide and a C-terminal gDM5 polypeptide. Suitable gDM5 fragments or gDM5 portions include, for example, the amino sequences of SEQ ID NO: 13, SEQ ID NO: 15, and SEQ ID NO: 17.

In some exemplary embodiments, the vectors and/or viruses comprise a nucleic acid molecule encoding a fusion protein comprising an antigen and a gDM5 sequence of SEQ ID NO: 9 or SEQ ID NO: 11. In some exemplary embodiments, the gDM5 can be a gDM5 fragment or gDM5 portion as described herein, such as an N-terminal gDM5 polypeptide, a C-terminal gDM5 polypeptide, or both an N-terminal gDM5 polypeptide and a C-terminal gDM5 polypeptide. Suitable gDM5 fragments or gDM5 portions include, for example, the amino sequences of SEQ ID NO: 13, SEQ ID NO: 15, and SEQ ID NO: 17. The virus comprising any of the herein described vectors or any of the herein described nucleic acid molecules can be an adenovirus. Suitable adenoviruses include, for example, an AdC6, AdC68, or AdC7.

Also described herein are vaccines comprising any of the herein described nucleic acid molecules, any one of the herein described vectors, or any of the herein described viruses. In some embodiments, the vaccine comprises one or more of the herein described nucleic acid molecules. In some embodiments, the vaccine comprises one or more of the herein described vectors. In some embodiments, the vaccine comprises one or more of the herein described viruses.

Further described herein are methods of inducing an immune response in a subject, the methods comprising providing to the subject an effective amount of any of the herein described nucleic acid molecules, any of the herein described vectors, any of the herein described fusion proteins, any of the herein described viruses, or any of the herein described vaccines to thereby induce an immune response. In some embodiments, the methods of inducing an immune response comprise providing to the subject one or more of the herein described nucleic acid molecules. In some embodiments, the methods of inducing an immune response comprise providing to the subject one or more of the herein described vectors. In some embodiments, the methods of inducing an immune response comprise providing to the subject one or more of the herein described fusion proteins. In some embodiments, the methods of inducing an immune response comprise providing to the subject one or more of the herein described viruses. In some embodiments, the methods of inducing an immune response comprise providing to the subject one or more of the herein described vaccines.

Disclosed herein are gD mutants (“gDM;” also referred to herein as gD variants) comprising any of the below amino acid sequences. Also disclosed are nucleic acid molecules encoding the gD mutants, the nucleic acid molecules comprising any of the below nucleotide sequences.

TABLE 1

| gDM Protein or Nucleotide Sequence | SEQ ID NO |
|---|---|
| gDM1 Protein | SEQ ID NO: 1 |
| gDM1 Nucleotide | SEQ ID NO: 2 |
| gDM2 Protein | SEQ ID NO: 3 |
| gDM2 Nucleotide | SEQ ID NO: 4 |
| gDM3 Protein | SEQ ID NO: 5 |
| gDM3 Nucleotide | SEQ ID NO: 6 |
| gDM4 Protein | SEQ ID NO: 7 |
| gDM4 Nucleotide | SEQ ID NO: 8 |
| gDM5 Protein | SEQ ID NO: 9 |
| gDM5 Nucleotide | SEQ ID NO: 10 |
| gDM5 No Signal Seq Protein | SEQ ID NO: 11 |
| gDM5 No Signal Seq Nucleotide | SEQ ID NO: 12 |
| gDM1 Protein #2 | SEQ ID NO: 49 |
| gD without transmembrane domain (TM) Protein | SEQ ID NO: 50 |
| gD without transmembrane domain (TM) Nucleotide | SEQ ID NO: 51 |
| Super gD with P2A sequence Protein | SEQ ID NO: 52 |
| Super gD with P2A sequence Nucleotide | SEQ ID NO: 53 |

Disclosed herein are gDM5 fragments comprising any of the below amino acid sequences. Also disclosed are nucleic acid molecules encoding the gDM5 fragments, the nucleic acid molecules comprising any of the below nucleotide sequences.

TABLE 2

| gDM5 Fragment Protein or Nucleotide Sequence | SEQ ID NO |
|---|---|
| gDM5 N-Terminal Protein | SEQ ID NO: 13 |
| gDM5 N-Terminal Nucleotide | SEQ ID NO: 14 |
| gDM5 N-Terminal No Signal Seq Protein | SEQ ID NO: 15 |
| gDM5 N-Terminal No Signal Seq Nucleotide | SEQ ID NO: 16 |
| gDM5 C-Terminal Protein | SEQ ID NO: 17 |
| gDM5 C-Terminal Nucleotide | SEQ ID NO: 18 |

Disclosed herein are gDM-antigen fusion proteins. Exemplary antigens include, but are not limited to, the following:

TABLE 3

| Antigen Protein or Nucleotide Seqeunce | SEQ ID NO |
|---|---|
| PolN Protein | SEQ ID NO: 25 |
| PolN Nucleotide | SEQ ID NO: 26 |
| HBV3 Protein | SEQ ID NO: 27 |
| HBV3 Nucleotide | SEQ ID NO: 28 |
| HIV-1 gag Protein | SEQ ID NO: 29 |
| HIV-1 gag Nucleotide | SEQ ID NO: 30 |
| Melapoly Protein | SEQ ID NO: 31 |
| Melapoly Nucleotide | SEQ ID NO: 32 |
| E765-wt Protein | SEQ ID NO: 54 |
| E765-wt Nucleotide | SEQ ID NO: 55 |
| Melapoly Protein #2 | SEQ ID NO: 72 |
| Melapoly Nucleotide #2 | SEQ ID NO: 73 |
| Melanoma antigens with universal helper epitope | SEQ ID NO: 74 |
| Protein | |
| Melanoma antigens with universal helper epitope | SEQ ID NO: 75 |
| Nucleotide | |

The gDM-antigen fusion proteins can comprise any one of the below gD proteins and any one of the below antigens:

TABLE 4

| gD Protein | | Antigen | |
|---|---|---|---|
| Name | SEQ ID NO | Name | SEQ ID NO |
| gDM1 Protein | 1 | PolN Protein | 25 |
| gDM2 Protein | 3 | HBV3 Protein | 27 |
| gDM3 Protein | 5 | HIV-1 gag Protein | 29 |
| gDM4 Protein | 7 | Melapoly Protein | 31 |
| gDM5 Protein | 9 | E765-wt Protein | 54 |
| gDM5 No Signal Seq | 11 | Melapoly Protein #2 | 72 |
| Protein | | | |
| gDM1 Protein #2 | 49 | Melanoma antigens | 74 |
| | | with universal | |
| | | helper epitope | |
| | | Protein | |
| gD without | 50 | | |
| transmembrane | | | |
| domain (TM) Protein | | | |
| Super gD with P2A | 52 | | |
| sequence Protein | | | |

The gDM-antigen fusion proteins can be encoded by any one of the below gD nucleotide sequences and any one of the below antigen nucleotide sequences:

TABLE 5

| gD Nucleotide | | Antigen | |
|---|---|---|---|
| Name | SEQ ID NO | Name | SEQ ID NO |
| gDM1 | 2 | PolN Nucleotide | 26 |
| Nucleotide | | | |
| gDM2 Nucleotide | 4 | HBV3 Nucleotide | 28 |
| gDM3 Nucleotide | 6 | HIV-1 gag Nucleotide | 30 |
| gDM4 Nucleotide | 8 | Melapoly Nucleotide | 32 |
| gDM5 Nucleotide | 10 | E765-wt Nucleotide | 55 |
| gDM5 No Signal | 12 | Melapoly | 73 |
| Seq Nucleotide | | Nucleotide #2 | |
| gD without | 51 | Melanoma antigens | 75 |
| transmembrane | | with universal | |
| domain (TM) | | helper epitope | |
| Nucleotide | | Nucleotide | |
| Super gD with P2A | 53 | | |
| sequence Nucleotide | | | |

For example, the gDM-antigen fusion proteins can comprise any of the below amino acid sequences, or be encoded by any of the below nucleotide sequences:

TABLE 6

| gD-Antigen Fusion Protein | |
|---|---|
| or Nucleotide Sequence | SEQ ID NO |
| Mutant HPV E7, E6, E5, and E2 Fusion | SEQ ID NO: 44 |
| Protein Fused to gDM5 Protein | |
| (also referred to herein as gDM5-E7652 Protein) | |
| Mutant HPV E7, E6, E5, and E2 Fusion | SEQ ID NO: 45 |
| Protein Fused to gDM5 | |
| Nucleotide (also referred to herein | |
| as gDM5-E7652 Nucleotide) | |
| Mutant HPV E7, E6, E5, and E2 | SEQ ID NO: 46 |
| Fusion Protein Fused to gDM5 | |
| No Signal Seq Protein | |
| Mutant HPV E7, E6, E5, and E2 | SEQ ID NO: 47 |
| Fusion Protein Fused to gDM5 | |
| No Signal Seq Nucleotide | |
| gDM5-E765 Protein | SEQ ID NO: 92 |
| gDM5-E765 Nucleotide | SEQ ID NO: 93 |
| Mutant E7, E6, and E5 fused into gDM5 Protein | SEQ ID NO: 110 |
| Mutant E7, E6, and E5 fused into gDM5 Nucleotide | SEQ ID NO: 111 |
| gDM5-PolN Protein | SEQ ID NO: 114 |
| gDM5-PolN Nucleotide | SEQ ID NO: 115 |
| gDM5-Melapoly#2 Protein | SEQ ID NO: 118 |
| gDM5-Melapoly#2 Nucleotide | SEQ ID NO: 119 |
| gD-E765dt1 Protein | SEQ ID NO: 82 |
| gD-E765dt1 Nucleotide | SEQ ID NO: 83 |
| gDM1-E765dt1 Protein | SEQ ID NO: 84 |
| gDM1-E765dt1 Nucleotide | SEQ ID NO: 85 |
| gDM2-E765dt1 Protein | SEQ ID NO: 86 |
| gDM2-E765dt1 Nucleotide | SEQ ID NO: 87 |
| gDM3-E765dt1 Protein | SEQ ID NO: 88 |
| gDM3-E765dt1 Nucleotide | SEQ ID NO: 89 |
| gD-E765 Protein | SEQ ID NO: 90 |
| gD-E765 Nucleotide | SEQ ID NO: 91 |
| gD-E7652 Protein | SEQ ID NO: 94 |
| gD-E7652 Nucleotide | SEQ ID NO: 95 |
| gD(-TM)-E765 Protein | SEQ ID NO: 96 |
| gD(-TM)-E765 Nucleotide | SEQ ID NO: 97 |
| SgD-PA2-E7652 Protein | SEQ ID NO: 98 |
| SgD-PA2-E7652 Nucleotide | SEQ ID NO: 99 |
| Mutant HPV E7, E6, E5, and E2 Fusion | SEQ ID NO: 100 |
| Protein Fused to gD Protein | |
| Mutant HPV E7, E6, E5, and E2 Fusion | SEQ ID NO: 101 |
| Protein Fused to gD Nucleotide | |
| gD-PolN Protein | SEQ ID NO: 112 |
| gD-PolN Nucleotide | SEQ ID NO: 113 |
| gD-Melapoly#2 Protein | SEQ ID NO: 116 |
| gD-Melapoly#2 Nucleotide | SEQ ID NO: 117 |

Disclosed herein are nucleic acid molecules encoding a gD mutant comprising the amino acid sequence of any one of SEQ ID NOs: 1, 3, 5, 7, 49, 50, or 52. The nucleic acid molecule can encode the amino acid sequence of SEQ ID NO: 1. The nucleic acid molecule can encode the amino acid sequence of SEQ ID NO: 3. The nucleic acid molecule can encode the amino acid sequence of SEQ ID NO: 5. The nucleic acid molecule can encode the amino acid sequence of SEQ ID NO: 7. The nucleic acid molecule can encode the amino acid sequence of SEQ ID NO: 49. The nucleic acid molecule can encode the amino acid sequence of SEQ ID NO: 50. The nucleic acid molecule can encode the amino acid sequence of SEQ ID NO: 52.

The nucleic acid molecules encoding a gD mutant can comprise the nucleotide sequence of any one of SEQ ID NOs: 2, 4, 6, 8, 51, or 53. The nucleic acid molecule can comprise the nucleotide sequence of SEQ ID NO: 2. The nucleic acid molecule can comprise the nucleotide sequence of SEQ ID NO: 4. The nucleic acid molecule can comprise the nucleotide sequence of SEQ ID NO: 6. The nucleic acid molecule can comprise the nucleotide sequence of SEQ ID NO: 8. The nucleic acid molecule can comprise the nucleotide sequence of SEQ ID NO: 51. The nucleic acid molecule can comprise the nucleotide sequence of SEQ ID NO: 53.

The nucleic acid molecules encoding a gD mutant can further comprise a nucleotide sequence encoding an antigen. In some embodiments, the antigen comprises any one of the amino acid sequences or is encoded by any one of the nucleotide sequences provided in Table 3. The antigen can comprise the amino acid sequence of SEQ ID NO: 25. The antigen can be encoded by the nucleotide sequence of SEQ ID NO: 26. The antigen can comprise the amino acid sequence of SEQ ID NO: 27. The antigen can be encoded by the nucleotide sequence of SEQ ID NO: 28. The antigen can comprise the amino acid sequence of SEQ ID NO: 29. The antigen can be encoded by the nucleotide sequence of SEQ ID NO: 30. The antigen can be encoded by the amino acid sequence of SEQ ID NO: 31. The antigen can be encoded by the nucleotide sequence of SEQ ID NO: 32. The antigen can comprise the amino acid sequence of SEQ ID NO: 54. The antigen can be encoded by the nucleotide sequence of SEQ ID NO: 55. The antigen can comprise the amino acid sequence of SEQ ID NO: 72. The antigen can be encoded by the nucleotide sequence of SEQ ID NO: 73. The antigen can comprise the amino acid sequence of SEQ ID NO: 74. The antigen can be encoded by the nucleotide sequence of SEQ ID NO: 75.

The nucleic acid molecules encoding a gD mutant protein can encode the amino acid sequence of any one of the amino acid sequences provided in Table 4 or Table 6. The nucleic acid molecule can encode the amino acid sequence of SEQ ID NO: 1. The nucleic acid molecule can encode the amino acid sequence of SEQ ID NO: 3. The nucleic acid molecule can encode the amino acid sequence of SEQ ID NO: 5. The nucleic acid molecule can encode the amino acid sequence of SEQ ID NO: 7. The nucleic acid molecule can encode the amino acid sequence of SEQ ID NO: 9. The nucleic acid molecule can encode the amino acid sequence of SEQ ID NO: 25. The nucleic acid molecule can encode the amino acid sequence of SEQ ID NO: 11. The nucleic acid molecule can encode the amino acid sequence of SEQ ID NO: 49. The nucleic acid molecule can encode the amino acid sequence of SEQ ID NO: 50. The nucleic acid molecule can encode the amino acid sequence of SEQ ID NO: 52. The nucleic acid molecule can encode the amino acid sequence of SEQ ID NO: 27. The nucleic acid molecule can encode the amino acid sequence of SEQ ID NO: 29. The nucleic acid molecule can encode the amino acid sequence of SEQ ID NO: 31. The nucleic acid molecule can encode the amino acid sequence of SEQ ID NO: 54. The nucleic acid molecule can encode the amino acid sequence of SEQ ID NO: 72. The nucleic acid molecule can encode the amino acid sequence of SEQ ID NO: 74. The nucleic acid molecule can encode the amino acid sequence of SEQ ID NO: 44. The nucleic acid molecule can encode the amino acid sequence of SEQ ID NO: 46. The nucleic acid molecule can encode the amino acid sequence of SEQ ID NO: 92. The nucleic acid molecule can encode the amino acid sequence of SEQ ID NO: 110. The nucleic acid molecule can encode the amino acid sequence of SEQ ID NO: 114. The nucleic acid molecule can encode the amino acid sequence of SEQ ID NO: 118. The nucleic acid molecule can encode the amino acid sequence of SEQ ID NO: 82. The nucleic acid molecule can encode the amino acid sequence of SEQ ID NO: 84. The nucleic acid molecule can encode the amino acid sequence of SEQ ID NO: 86. The nucleic acid molecule can encode the amino acid sequence of SEQ ID NO: 88. The nucleic acid molecule can encode the amino acid sequence of SEQ ID NO: 90. The nucleic acid molecule can encode the amino acid sequence of SEQ ID NO: 94. The nucleic acid molecule can encode the amino acid sequence of SEQ ID NO: 96. The nucleic acid molecule can encode the amino acid sequence of SEQ ID NO: 98. The nucleic acid molecule can encode the amino acid sequence of SEQ ID NO: 100. The nucleic acid molecule can encode the amino acid sequence of SEQ ID NO: 112. The nucleic acid molecule can encode the amino acid sequence of SEQ ID NO: 116.

The nucleic acid molecules encoding a gD mutant protein can comprise the nucleotide sequence of any one of the nucleotide sequences provided in Table 5 or Table 6. The nucleic acid molecule can comprise the nucleotide sequence of SEQ ID NO: 2. The nucleic acid molecule can comprise the nucleotide sequence of SEQ ID NO: 4. The nucleic acid molecule can comprise the nucleotide sequence of SEQ ID NO: 6. The nucleic acid molecule can comprise the nucleotide sequence of SEQ ID NO: 8. The nucleic acid molecule can comprise the nucleotide sequence of SEQ ID NO: 10. The nucleic acid molecule can comprise the nucleotide sequence of SEQ ID NO: 12. The nucleic acid molecule can comprise the nucleotide sequence of SEQ ID NO: 51. The nucleic acid molecule can comprise the nucleotide sequence of SEQ ID NO: 53. The nucleic acid molecule can comprise the nucleotide sequence of SEQ ID NO: 26. The nucleic acid molecule can comprise the nucleotide sequence of SEQ ID NO: 28. The nucleic acid molecule can comprise the nucleotide sequence of SEQ ID NO: 30. The nucleic acid molecule can comprise the nucleotide sequence of SEQ ID NO: 32. The nucleic acid molecule can comprise the nucleotide sequence of SEQ ID NO: 55. The nucleic acid molecule can comprise the nucleotide sequence of SEQ ID NO: 73. The nucleic acid molecule can comprise the nucleotide sequence of SEQ ID NO: 75. The nucleic acid molecule can comprise the nucleotide sequence of SEQ ID NO: 45. The nucleic acid molecule can comprise the nucleotide sequence of SEQ ID NO: 47. The nucleic acid molecule can comprise the nucleotide sequence of SEQ ID NO: 93. The nucleic acid molecule can comprise the nucleotide sequence of SEQ ID NO: 111. The nucleic acid molecule can comprise the nucleotide sequence of SEQ ID NO: 114. The nucleic acid molecule can comprise the nucleotide sequence of SEQ ID NO: 115. The nucleic acid molecule can comprise the nucleotide sequence of SEQ ID NO: 119. The nucleic acid molecule can comprise the nucleotide sequence of SEQ ID NO: 83. The nucleic acid molecule can comprise the nucleotide sequence of SEQ ID NO: 85. The nucleic acid molecule can comprise the nucleotide sequence of SEQ ID NO: 87. The nucleic acid molecule can comprise the nucleotide sequence of SEQ ID NO: 89. The nucleic acid molecule can comprise the nucleotide sequence of SEQ ID NO: 91. The nucleic acid molecule can comprise the nucleotide sequence of SEQ ID NO: 95. The nucleic acid molecule can comprise the nucleotide sequence of SEQ ID NO: 97. The nucleic acid molecule can comprise the nucleotide sequence of SEQ ID NO: 99. The nucleic acid molecule can comprise the nucleotide sequence of SEQ ID NO: 101. The nucleic acid molecule can comprise the nucleotide sequence of SEQ ID NO: 113. The nucleic acid molecule can comprise the nucleotide sequence of SEQ ID NO: 117.

Disclosed herein are fusion proteins comprising a gD mutant comprising the amino acid sequence of any one of SEQ ID NOs: 1, 3, 5, 7, 49, 50, or 52, and an antigen. The fusion protein can comprise the amino acid sequence of SEQ ID NO: 1 and an antigen. The fusion protein can comprise the amino acid sequence of SEQ ID NO: 3 and an antigen. The fusion protein can comprise the amino acid sequence of SEQ ID NO: 5 and an antigen. The fusion protein can comprise the amino acid sequence of SEQ ID NO: 7 and an antigen. The fusion protein can comprise the amino acid sequence of SEQ ID NO: 49 and an antigen. The fusion protein can comprise the amino acid sequence of SEQ ID NO: 50 and an antigen. The fusion protein can comprise the amino acid sequence of SEQ ID NO: 52 and an antigen.

The fusion protein can comprise an antigen comprising the amino acid sequence of any one of the amino acid sequences provided in Table 3. The antigen can comprise the amino acid sequence of SEQ ID NO: 25. The antigen can comprise the amino acid sequence of SEQ ID NO: 27. The antigen can comprise the amino acid sequence of SEQ ID NO: 29. The antigen can comprise the amino acid sequence of SEQ ID NO: 31. The antigen can comprise the amino acid sequence of SEQ ID NO: 54. The antigen can comprise the amino acid sequence of SEQ ID NO: 72. The antigen can comprise the amino acid sequence of SEQ ID NO: 74.

The fusion protein can comprise the amino acid sequence of any one of the amino acid sequences provided in Table 6. The fusion protein can comprise the amino acid sequence of SEQ ID NO: 44. The fusion protein can comprise the amino acid sequence of SEQ ID NO: 46. The fusion protein can comprise the amino acid sequence of SEQ ID NO: 92. The fusion protein can comprise the amino acid sequence of SEQ ID NO: 110. The fusion protein can comprise the amino acid sequence of SEQ ID NO: 114. The fusion protein can comprise the amino acid sequence of SEQ ID NO: 118. The fusion protein can comprise the amino acid sequence of SEQ ID NO: 82. The fusion protein can comprise the amino acid sequence of SEQ ID NO: 84. The fusion protein can comprise the amino acid sequence of SEQ ID NO: 86. The fusion protein can comprise the amino acid sequence of SEQ ID NO: 88. The fusion protein can comprise the amino acid sequence of SEQ ID NO: 90. The fusion protein can comprise the amino acid sequence of SEQ ID NO: 94. The fusion protein can comprise the amino acid sequence of SEQ ID NO: 96. The fusion protein can comprise the amino acid sequence of SEQ ID NO: 98. The fusion protein can comprise the amino acid sequence of SEQ ID NO: 100. The fusion protein can comprise the amino acid sequence of SEQ ID NO: 112. The fusion protein can comprise the amino acid sequence of SEQ ID NO: 116.

Disclosed herein are vectors comprising any of the herein disclosed nucleic acid molecules. The vector can comprise a nucleic acid molecule encoding a gD mutant comprising the amino acid sequence of any one of SEQ ID NOs: 1, 3, 5, 7, 49, 50, or 52. The vector can comprise a nucleic acid molecule comprising the nucleotide sequence of any one of SEQ ID NOs: 2, 4, 6, 8, 51, or 53. The vector can comprise a nucleic acid molecule encoding a gD mutant and an antigen, wherein the antigen comprises an amino acid sequence provided in Table 3. The vector can comprise a nucleic acid molecule encoding a protein comprising an amino acid sequence of any one of the amino acid sequences provided in Table 4 or Table 6. The vector can comprise a nucleic acid molecule comprising a nucleotide sequence of any one of the nucleotide sequences provided in Table 5 or Table 6.

Disclosed herein are host cells comprising any of the herein disclosed vectors. The host cell can be a eukaryotic cell or a prokaryotic cell. The cell can be grown under conditions suitable for replication of the vector or conditions suitable for expression of the protein encoded by the vector comprising the nucleic acid molecule.

Disclosed herein are viruses comprising any of the herein disclosed nucleic acid molecules or any of the herein disclosed vectors. The virus can comprise at least one of the herein disclosed nucleic acid molecules. The virus can comprise at least one of the herein disclosed vectors. In some embodiments, the virus is an adenovirus. The adenovirus can be AdC6, AdC68, or AdC7. The adenovirus can be AdC6. The adenovirus can be AdC68. The adenovirus can be AdC7.

Disclosed herein are vaccines comprising any of the herein disclosed nucleic acid molecules, any of the herein disclosed vectors, or any of the herein disclosed viruses. In some embodiments, the vaccine comprises at least one of the herein disclosed nucleic acid molecules. In some embodiments, the vaccine comprises at least one of the herein disclosed vectors. In some embodiments, the vaccine comprises at least one of the herein disclosed vectors. The vaccine can further comprise a pharmaceutically acceptable excipient. Suitable pharmaceutically acceptable excipients include, for example, carriers, buffers, and/or stabilizers.

Disclosed herein are pharmaceutical compositions comprising any of the herein disclosed nucleic acid molecules, any of the herein disclosed vectors, or any of the herein disclosed viruses. In some embodiments, the pharmaceutical composition comprises at least one of the herein disclosed nucleic acid molecules. In some embodiments, the pharmaceutical composition comprises at least one of the herein disclosed vectors. In some embodiments, the pharmaceutical composition comprises at least one of the herein disclosed vectors. The pharmaceutical composition can further comprise a pharmaceutically acceptable excipient. Suitable pharmaceutically acceptable excipients include, for example, carriers, buffers, and/or stabilizers.

Disclosed herein are methods of inducing an immune response in a subject, the method comprising providing to the subject an effective amount of any of the herein disclosed nucleic acid molecules, any of the herein disclosed vectors, any of the herein disclosed fusion proteins, any of the herein disclosed viruses, or any of the herein disclosed vaccines to thereby induce an immune response. In some embodiments, the subject in provided at least one of the herein disclosed nucleic acid molecules. In some embodiments, the subject in provided at least one of the herein disclosed vectors. In some embodiments, the subject in provided at least one of the herein disclosed fusion proteins. In some embodiments, the subject in provided at least one of the herein disclosed viruses. In some embodiments, the subject in provided at least one of the herein disclosed vaccines.

Disclosed herein are pharmaceutical compositions comprising any of the herein disclosed nucleic acid molecules, any of the herein disclosed vectors, any of the herein disclosed fusion proteins, any of the herein disclosed viruses, or any of the herein disclosed vaccines for use in inducing an immune response in a subject. In some embodiments, the pharmaceutical composition comprises at least one of the herein disclosed nucleic acid molecules. In some embodiments, the pharmaceutical composition comprises at least one of the herein disclosed vectors. In some embodiments, the pharmaceutical composition comprises at least one of the herein disclosed fusion proteins. In some embodiments, the pharmaceutical composition comprises at least one of the herein disclosed viruses. In some embodiments, the pharmaceutical composition comprises at least one of the herein disclosed vaccines.

Disclosed herein are any of the herein disclosed nucleic acid molecules, any of the herein disclosed vectors, any of the herein disclosed fusion proteins, any of the herein disclosed viruses, or any of the herein disclosed vaccines for use in the preparation of a medicament useful for inducing an immune response in a subject. In some embodiments, at least one of the herein disclosed nucleic acid molecules are used in the preparation of the medicament. In some embodiments, at least one of the herein disclosed vectors are used in the preparation of the medicament. In some embodiments, at least one of the herein disclosed fusion proteins are used in the preparation of the medicament. In some embodiments, at least one of the herein disclosed viruses are used in the preparation of the medicament. In some embodiments, at least one of the herein disclosed viruses are used in the preparation of the medicament. In some embodiments, at least one of the herein disclosed vaccines are used in the preparation of the medicament.

### Fusion Protein Vaccines To Human Papilloma Virus

Described herein are nucleic acid molecules encoding mutant E7, mutant E6, mutant E5, or mutant E2 proteins from human papilloma virus 16 (HPV 16). The nucleic acid molecule can encode a mutant HPV 16 E7 protein comprising the amino acid sequence of SEQ ID NO: 34. The nucleic acid molecule encoding a mutant HPV 16 E7 protein can comprise the nucleotide sequence of SEQ ID NO: 35. In some embodiments, the nucleic acid molecule can encode a mutant HPV 16 E6 protein comprising the amino acid sequence of SEQ ID NO: 36. The nucleic acid molecule encoding a mutant HPV 16 E6 protein can comprise the nucleotide sequence of SEQ ID NO: 37. In some embodiments, the nucleic acid molecule can encode a mutant HPV 16 E5 protein comprising the amino acid sequence of SEQ ID NO: 38. The nucleic acid molecule encoding a mutant HPV 16 E5 protein can comprise the nucleotide sequence of SEQ ID NO: 39. In some embodiments, the nucleic acid molecule can encode a mutant HPV 16 E2 protein comprising the amino acid sequence of SEQ ID NO: 40. The nucleic acid molecule encoding a mutant HPV 16 E2 protein can comprise the nucleotide sequence of SEQ ID NO: 41. The nucleic acid molecules can encode for mutant HPV 16 E7, mutant HPV 16 E6, mutant HPV 16 E5, and mutant HPV 16 E2 proteins.

In some embodiments, the nucleic acid molecule comprises a nucleotide sequence encoding a mutant HPV 16 E7 protein comprising the amino acid sequence of SEQ ID NO: 34, a nucleotide sequence encoding a mutant HPV 16 E6 protein comprising the amino acid sequence of SEQ ID NO: 36, a nucleotide sequence encoding a mutant HPV 16 E5 protein comprising the amino acid sequence of SEQ ID NO: 38, and a nucleotide sequence encoding a mutant HPV 16 E2 protein comprising the amino acid sequence of SEQ ID NO: 40.

In some embodiments, the nucleic acid molecule encodes a fusion protein comprising the amino acid sequence of SEQ ID NO: 42. In some embodiments, the nucleic acid molecule encoding the fusion protein comprises the nucleotide sequence of one or more of SEQ ID NO: 35, SEQ ID NO: 37, SEQ ID NO: 39, and SEQ ID NO: 41. In some embodiments, the nucleic acid molecule comprises the nucleotide sequence of SEQ ID NO: 43.

Any one of the herein described nucleic acid molecules can further comprise a nucleotide sequence encoding a mutant glycoprotein D (referred to herein as “gDM5”) protein. The nucleic acid molecules can comprise a nucleotide sequence encoding the amino acid sequence of SEQ ID NO: 9. The nucleic acid molecules can comprise a nucleotide sequence of SEQ ID NO: 10. The nucleic acid molecules can comprise a nucleotide sequence encoding the amino acid sequence of SEQ ID NO: 11. The nucleic acid molecules can comprise a nucleotide sequence of SEQ ID NO: 12.

The nucleic acid molecules can further comprise a nucleotide sequence encoding an N-terminal gDM5 sequence, a C-terminal gDM5 sequence, or both the N-terminal gDM5 sequence and the C-terminal gDM5 sequence. In some embodiments, the nucleic acid molecule comprises a nucleotide sequence encoding an N-terminal gDM5 sequence, a nucleotide sequence encoding a mutant HPV 16 E7 protein comprising the amino acid sequence of SEQ ID NO: 34, a nucleotide sequence encoding a mutant HPV 16 E6 protein comprising the amino acid sequence of SEQ ID NO: 36, a nucleotide sequence encoding a mutant HPV 16 E5 protein comprising the amino acid sequence of SEQ ID NO: 38, a nucleotide sequence encoding a mutant HPV 16 E2 protein comprising the amino acid sequence of SEQ ID NO: 40, and a nucleotide sequence encoding a C-terminal gDM5 sequence.

In some embodiments, the nucleotide sequence encodes an N-terminal gDM5 sequence comprising the amino sequence of SEQ ID NO: 15. In some embodiments, the nucleotide sequence encoding the N-terminal gDM5 sequence comprises the nucleotide sequence of SEQ ID NO: 16. In some embodiments, the nucleotide sequence encodes an N-terminal gDM5 sequence comprising the amino sequence of SEQ ID NO: 13. In some embodiments, the nucleotide sequence encoding the N-terminal gDM5 sequence comprises the nucleotide sequence of SEQ ID NO: 14.

In some embodiments, the nucleic acid molecules encode a C-terminal gDM5 sequence comprising the amino sequence of SEQ ID NO: 17. In some embodiments, the nucleic acid molecules encode a C-terminal gDM5 sequence comprising the nucleotide sequence of SEQ ID NO: 18.

In some embodiments, the nucleic acid molecules comprise a nucleotide sequence encoding an N-terminal gDM5 sequence comprising the amino sequence of SEQ ID NO: 15, a nucleotide sequence encoding a mutant HPV 16 E7 protein comprising the amino acid sequence of SEQ ID NO: 34, a nucleotide sequence encoding a mutant HPV 16 E6 protein comprising the amino acid sequence of SEQ ID NO: 36, a nucleotide sequence encoding a mutant HPV 16 E5 protein comprising the amino acid sequence of SEQ ID NO: 38, a nucleotide sequence encoding a mutant HPV 16 E2 protein comprising the amino acid sequence of SEQ ID NO: 40, and a nucleotide sequence encoding a C-terminal gDM5 sequence comprising the amino sequence of SEQ ID NO: 17.

In some embodiments, the nucleic acid molecules comprise a nucleotide sequence encoding an N-terminal gDM5 sequence comprising the amino sequence of SEQ ID NO: 13, a nucleotide sequence encoding a mutant HPV 16 E7 protein comprising the amino acid sequence of SEQ ID NO: 34, a nucleotide sequence encoding a mutant HPV 16 E6 protein comprising the amino acid sequence of SEQ ID NO: 36, a nucleotide sequence encoding a mutant HPV 16 E5 protein comprising the amino acid sequence of SEQ ID NO: 38, a nucleotide sequence encoding a mutant HPV 16 E2 protein comprising the amino acid sequence of SEQ ID NO: 40, and a nucleotide sequence encoding a C-terminal gDM5 sequence comprising the amino sequence of SEQ ID NO: 17.

In some embodiments, the nucleic acid molecules comprise a nucleotide sequence encoding an N-terminal gDM5 sequence comprising the amino sequence of SEQ ID NO: 13, a nucleotide sequence encoding a fusion protein comprising the amino acid sequence of SEQ ID NO: 42, and a nucleotide sequence encoding a C-terminal gDM5 sequence comprising the amino sequence of SEQ ID NO: 17. In some embodiments, the nucleic acid molecules encode a fusion protein comprising the nucleotide sequence of SEQ ID NO: 43.

In some embodiments, the nucleic acid molecules encode the amino sequence of SEQ ID NO: 46. In some embodiments, the nucleic acid molecules comprise the nucleotide sequence of SEQ ID NO: 47. In some embodiments, the nucleic acid molecules encode the amino sequence of SEQ ID NO: 44. In some embodiments, the nucleic acid molecules comprise the nucleotide sequence of SEQ ID NO: 45.

Further described herein are mutant E7, mutant E6, mutant E5, or mutant E2 proteins from human papilloma virus 16 (HPV 16). The mutant HPV 16 E7, mutant HPV 16 E6, mutant HPV 16 E5, or mutant HPV 16 E2 proteins can comprise the amino acid sequences of SEQ ID NO: 34, SEQ ID NO: 36, SEQ ID NO: 38, or SEQ ID NO: 40, respectively. In some embodiments, the mutant HPV 16 E7 protein comprises the amino acid sequence of SEQ ID NO: 34. In some embodiments, the mutant HPV 16 E6 protein comprises the amino acid sequence of SEQ ID NO: 36. In some embodiments, the mutant HPV 16 E5 protein comprises the amino acid sequence of SEQ ID NO: 38. In some embodiments, the mutant HPV 16 E2 protein comprises the amino acid sequence of SEQ ID NO: 40.

Also disclosed herein are fusion proteins comprising a mutant HPV 16 E7 protein comprising the amino acid sequence of SEQ ID NO: 34, a mutant HPV 16 E6 protein comprising the amino acid sequence of SEQ ID NO: 36, a mutant HPV 16 E5 protein comprising the amino acid sequence of SEQ ID NO: 38, and a mutant HPV 16 E2 protein comprising the amino acid sequence of SEQ ID NO: 40. In some embodiments, the fusion protein comprises the amino acid sequence of SEQ ID NO: 42.

The fusion proteins can further comprise a gDM5 protein. The fusion protein can comprise the amino acid sequence of SEQ ID NO: 9. The fusion protein can comprise the amino acid sequence of SEQ ID NO: 11. In some embodiments, the fusion proteins can further comprise an N-terminal gDM5 sequence, a C-terminal gDM5 sequence, or both the N-terminal gDM5 sequence and the C-terminal gDM5 sequence. The fusion protein can comprise an N-terminal gDM5 sequence of SEQ ID NO: 15. The fusion protein can comprise an N-terminal gDM5 sequence of SEQ ID NO: 13 The fusion protein can comprise a C-terminal gDM5 sequence of SEQ ID NO: 17.

In some embodiments, the fusion protein comprises an N-terminal gDM5 sequence, a mutant HPV 16 E7 protein comprising the amino acid sequence of SEQ ID NO: 34, a mutant HPV 16 E6 protein comprising the amino acid sequence of SEQ ID NO: 36, a mutant HPV 16 E5 protein comprising the amino acid sequence of SEQ ID NO: 38, a mutant HPV 16 E2 protein comprising the amino acid sequence of SEQ ID NO: 40, and a C-terminal gDM5 sequence.

In some embodiments, the fusion proteins comprise an N-terminal gDM5 sequence, wherein the N-terminal gDM5 sequence comprises the amino sequence of SEQ ID NO: 15. In some embodiments, the fusion proteins comprise an N-terminal gDM5 sequence, wherein the N-terminal gDM5 sequence comprises the amino sequence of SEQ ID NO: 13. In some embodiments, the fusion proteins comprise a C-terminal gDM5 sequence, wherein the C-terminal gDM5 sequence comprises the amino sequence of SEQ ID NO: 17.

The fusion proteins can comprise an N-terminal gDM5 sequence comprising the amino sequence of SEQ ID NO: 15, a mutant HPV 16 E7 protein comprising the amino acid sequence of SEQ ID NO: 34, a mutant HPV 16 E6 protein comprising the amino acid sequence of SEQ ID NO: 36, a mutant HPV 16 E5 protein comprising the amino acid sequence of SEQ ID NO: 38, a mutant HPV 16 E2 protein comprising the amino acid sequence of SEQ ID NO: 40, and a C-terminal gDM5 sequence comprising the amino sequence of SEQ ID NO: 17.

The fusion proteins can comprise an N-terminal gDM5 sequence comprising the amino sequence of SEQ ID NO: 13, a mutant HPV 16 E7 protein comprising the amino acid sequence of SEQ ID NO: 34, a mutant HPV 16 E6 protein comprising the amino acid sequence of SEQ ID NO: 36, a mutant HPV 16 E5 protein comprising the amino acid sequence of SEQ ID NO: 38, a mutant HPV 16 E2 protein comprising the amino acid sequence of SEQ ID NO: 40, and a C-terminal gDM5 sequence comprising the amino sequence of SEQ ID NO: 17.

The fusion proteins can comprise the amino acid sequence of SEQ ID NO: 42. The fusion protein comprising the amino acid sequence of SEQ ID NO: 42 can further comprise an N-terminal gDM5 sequence, a C-terminal gDM5 sequence, or both the N-terminal gDM5 sequence and the C-terminal gDM5 sequence.

The fusion proteins can comprise an N-terminal gDM5 sequence comprising the amino sequence of SEQ ID NO: 15, the amino acid sequence of SEQ ID NO: 42, and a C-terminal gDM5 sequence comprising the amino sequence of SEQ ID NO: 17.

The fusion proteins can comprise an N-terminal gDM5 sequence comprising the amino sequence of SEQ ID NO: 13, the amino acid sequence of SEQ ID NO: 42, and a C-terminal gDM5 sequence comprising the amino sequence of SEQ ID NO: 17.

The fusion proteins can comprise the amino acid sequence of SEQ ID NO: 46. In some embodiments, the fusion proteins can comprise the amino acid sequence of SEQ ID NO: 44.

Also described herein are vectors and viruses comprising any of the herein disclosed nucleic acid molecules. In some embodiments, the vectors comprise any of the herein disclosed nucleic acid molecules. Disclosed herein are host cells comprising any of the herein disclosed vectors. Suitable host cells include eukaryotic cells and prokaryotic cells. The viruses can comprise any of the herein disclosed nucleic acid molecules. In some embodiments, the viruses can comprise any one of the herein described vectors.

Disclosed herein is a vector comprising a nucleic acid molecule that encodes a fusion protein comprising an N-terminal gDM5 sequence comprising the amino sequence of SEQ ID NO: 15, a mutant HPV 16 E7 protein comprising the amino acid sequence of SEQ ID NO: 34, a mutant HPV 16 E6 protein comprising the amino acid sequence of SEQ ID NO: 36, a mutant HPV 16 E5 protein comprising the amino acid sequence of SEQ ID NO: 38, a mutant HPV 16 E2 protein comprising the amino acid sequence of SEQ ID NO: 40, and a C-terminal gDM5 sequence comprising the amino sequence of SEQ ID NO: 17.

Disclosed herein is a vector comprising a nucleic acid molecule that encodes a fusion protein comprising an N-terminal gDM5 sequence comprising the amino sequence of SEQ ID NO: 13, a mutant HPV 16 E7 protein comprising the amino acid sequence of SEQ ID NO: 34, a mutant HPV 16 E6 protein comprising the amino acid sequence of SEQ ID NO: 36, a mutant HPV 16 E5 protein comprising the amino acid sequence of SEQ ID NO: 38, a mutant HPV 16 E2 protein comprising the amino acid sequence of SEQ ID NO: 40, and a C-terminal gDM5 sequence comprising the amino sequence of SEQ ID NO: 17.

Disclosed herein is a vector comprising a nucleic acid molecule that encodes a fusion protein comprising the amino acid sequence of SEQ ID NO: 46. In some embodiments, the vector comprises a nucleic acid molecule that encodes a fusion protein comprising the amino acid sequence of SEQ ID NO: 44.

Disclosed herein is a virus comprising a nucleic acid molecule that encodes a fusion protein comprising an N-terminal gDM5 sequence comprising the amino sequence of SEQ ID NO: 15, a mutant HPV 16 E7 protein comprising the amino acid sequence of SEQ ID NO: 34, a mutant HPV 16 E6 protein comprising the amino acid sequence of SEQ ID NO: 36, a mutant HPV 16 E5 protein comprising the amino acid sequence of SEQ ID NO: 38, a mutant HPV 16 E2 protein comprising the amino acid sequence of SEQ ID NO: 40, and a C-terminal gDM5 sequence comprising the amino sequence of SEQ ID NO: 17.

Disclosed herein is a virus comprising a nucleic acid molecule that encodes a fusion protein comprising an N-terminal gDM5 sequence comprising the amino sequence of SEQ ID NO: 13, a mutant HPV 16 E7 protein comprising the amino acid sequence of SEQ ID NO: 34, a mutant HPV 16 E6 protein comprising the amino acid sequence of SEQ ID NO: 36, a mutant HPV 16 E5 protein comprising the amino acid sequence of SEQ ID NO: 38, a mutant HPV 16 E2 protein comprising the amino acid sequence of SEQ ID NO: 40, and a C-terminal gDM5 sequence comprising the amino sequence of SEQ ID NO: 17.

Disclosed herein is a virus comprising a nucleic acid molecule that encodes a fusion protein comprising the amino acid sequence of SEQ ID NO: 46. In some embodiments, the virus comprises a nucleic acid molecule that encodes a fusion protein comprising the amino acid sequence of SEQ ID NO: 44.

Suitable viruses include, for example, an adenovirus. The adenoviruses can be an AdC6, AdC68, or AdC7.

Further disclosed herein are vaccines comprising any of the herein disclosed vectors, any of the herein disclosed nucleic acid molecules, or any of the herein disclosed viruses. In some embodiments, the vaccine comprises one or more of the herein described vectors. In some embodiments, the vaccine comprises one or more of the herein described nucleic acid molecules. In some embodiments, the vaccine comprises one or more of the herein described viruses.

Also described herein are methods of inducing an immune response to HPV in a subject, the methods comprising providing to the subject an effective amount of any of the herein described vectors, any of the herein described nucleic acid molecules, any of the herein described fusion proteins, any of the herein described viruses, or any of the herein described vaccines to thereby induce an immune response to HPV.

In some embodiments, the methods of inducing an immune response comprise providing to the subject one or more of the herein described vectors.

The vector(s) provided to the subject can comprise a nucleic acid molecule that encodes a fusion protein comprising an N-terminal gDM5 sequence comprising the amino sequence of SEQ ID NO: 15, a mutant HPV 16 E7 protein comprising the amino acid sequence of SEQ ID NO: 34, a mutant HPV 16 E6 protein comprising the amino acid sequence of SEQ ID NO: 36, a mutant HPV 16 E5 protein comprising the amino acid sequence of SEQ ID NO: 38, a mutant HPV 16 E2 protein comprising the amino acid sequence of SEQ ID NO: 40, and a C-terminal gDM5 sequence comprising the amino sequence of SEQ ID NO: 17.

The vector(s) provided to the subject can comprise a nucleic acid molecule that encodes a fusion protein comprising an N-terminal gDM5 sequence comprising the amino sequence of SEQ ID NO: 13, a mutant HPV 16 E7 protein comprising the amino acid sequence of SEQ ID NO: 34, a mutant HPV 16 E6 protein comprising the amino acid sequence of SEQ ID NO: 36, a mutant HPV 16 E5 protein comprising the amino acid sequence of SEQ ID NO: 38, a mutant HPV 16 E2 protein comprising the amino acid sequence of SEQ ID NO: 40, and a C-terminal gDM5 sequence comprising the amino sequence of SEQ ID NO: 17.

The vector(s) provided to the subject can comprise a nucleic acid molecule that encodes a fusion protein comprising the amino acid sequence of SEQ ID NO: 46. The vector(s) provided to the subject can comprise a nucleic acid molecule that encodes a fusion protein comprising the amino acid sequence of SEQ ID NO: 44.

In some embodiments, the methods of inducing an immune response comprise providing to the subject one or more of the herein described nucleic acid molecules.

The nucleic acid molecule(s) provided to the subject can encode a fusion protein comprising an N-terminal gDM5 sequence comprising the amino sequence of SEQ ID NO: 15, a mutant HPV 16 E7 protein comprising the amino acid sequence of SEQ ID NO: 34, a mutant HPV 16 E6 protein comprising the amino acid sequence of SEQ ID NO: 36, a mutant HPV 16 E5 protein comprising the amino acid sequence of SEQ ID NO: 38, a mutant HPV 16 E2 protein comprising the amino acid sequence of SEQ ID NO: 40, and a C-terminal gDM5 sequence comprising the amino sequence of SEQ ID NO: 17.

The nucleic acid molecule(s) provided to the subject can encode a fusion protein comprising an N-terminal gDM5 sequence comprising the amino sequence of SEQ ID NO: 13, a mutant HPV 16 E7 protein comprising the amino acid sequence of SEQ ID NO: 34, a mutant HPV 16 E6 protein comprising the amino acid sequence of SEQ ID NO: 36, a mutant HPV 16 E5 protein comprising the amino acid sequence of SEQ ID NO: 38, a mutant HPV 16 E2 protein comprising the amino acid sequence of SEQ ID NO: 40, and a C-terminal gDM5 sequence comprising the amino sequence of SEQ ID NO: 17.

The nucleic acid molecule(s) provided to the subject can encode a fusion protein comprising the amino acid sequence of SEQ ID NO: 46. The nucleic acid molecule(s) provided to the subject can encode a fusion protein comprising the amino acid sequence of SEQ ID NO: 44.

In some embodiments, the methods of inducing an immune response comprise providing to the subject one or more of the herein described fusion proteins.

The fusion protein(s) provided to the subject can comprise an N-terminal gDM5 sequence comprising the amino sequence of SEQ ID NO: 15, a mutant HPV 16 E7 protein comprising the amino acid sequence of SEQ ID NO: 34, a mutant HPV 16 E6 protein comprising the amino acid sequence of SEQ ID NO: 36, a mutant HPV 16 E5 protein comprising the amino acid sequence of SEQ ID NO: 38, a mutant HPV 16 E2 protein comprising the amino acid sequence of SEQ ID NO: 40, and a C-terminal gDM5 sequence comprising the amino sequence of SEQ ID NO: 17.

The fusion protein(s) provided to the subject can comprise an N-terminal gDM5 sequence comprising the amino sequence of SEQ ID NO: 13, a mutant HPV 16 E7 protein comprising the amino acid sequence of SEQ ID NO: 34, a mutant HPV 16 E6 protein comprising the amino acid sequence of SEQ ID NO: 36, a mutant HPV 16 E5 protein comprising the amino acid sequence of SEQ ID NO: 38, a mutant HPV 16 E2 protein comprising the amino acid sequence of SEQ ID NO: 40, and a C-terminal gDM5 sequence comprising the amino sequence of SEQ ID NO: 17.

The fusion protein(s) provided to the subject can comprise the amino acid sequence of SEQ ID NO: 46. The fusion protein(s) provided to the subject can comprise the amino acid sequence of SEQ ID NO: 44.

In some embodiments, the methods of inducing an immune response comprise providing to the subject one or more of the herein described viruses.

The virus(es) provided to the subject can comprise a nucleic acid molecule that encodes a fusion protein comprising an N-terminal gDM5 sequence comprising the amino sequence of SEQ ID NO: 15, a mutant HPV 16 E7 protein comprising the amino acid sequence of SEQ ID NO: 34, a mutant HPV 16 E6 protein comprising the amino acid sequence of SEQ ID NO: 36, a mutant HPV 16 E5 protein comprising the amino acid sequence of SEQ ID NO: 38, a mutant HPV 16 E2 protein comprising the amino acid sequence of SEQ ID NO: 40, and a C-terminal gDM5 sequence comprising the amino sequence of SEQ ID NO: 17.

The virus(es) provided to the subject can comprise a nucleic acid molecule that encodes a fusion protein comprising an N-terminal gDM5 sequence comprising the amino sequence of SEQ ID NO: 13, a mutant HPV 16 E7 protein comprising the amino acid sequence of SEQ ID NO: 34, a mutant HPV 16 E6 protein comprising the amino acid sequence of SEQ ID NO: 36, a mutant HPV 16 E5 protein comprising the amino acid sequence of SEQ ID NO: 38, a mutant HPV 16 E2 protein comprising the amino acid sequence of SEQ ID NO: 40, and a C-terminal gDM5 sequence comprising the amino sequence of SEQ ID NO: 17.

The virus(es) provided to the subject can comprise a nucleic acid molecule that encodes a fusion protein comprising the amino acid sequence of SEQ ID NO: 46. The virus(es) provided to the subject can comprise a nucleic acid molecule that encodes a fusion protein comprising the amino acid sequence of SEQ ID NO: 44.

In some embodiments, the methods of inducing an immune response comprise providing to the subject one or more of the herein described vaccines. The vaccines can comprise any of the herein disclosed vectors, any of the herein disclosed nucleic acid molecules, or any of the herein disclosed viruses.

In some embodiments, the vaccine provided to the subject can comprise any of the herein disclosed vectors. In some embodiments, the vaccine provided to the subject can comprise any of the herein disclosed vectors. In some embodiments, the vaccine provided to the subject can comprise any of the herein disclosed nucleic acid molecules. In some embodiments, the vaccine provided to the subject can comprise any of the herein disclosed viruses.

The vaccine(s) provided to the subject can comprise a nucleic acid molecule that encodes a fusion protein comprising an N-terminal gDM5 sequence comprising the amino sequence of SEQ ID NO: 15, a mutant HPV 16 E7 protein comprising the amino acid sequence of SEQ ID NO: 34, a mutant HPV 16 E6 protein comprising the amino acid sequence of SEQ ID NO: 36, a mutant HPV 16 E5 protein comprising the amino acid sequence of SEQ ID NO: 38, a mutant HPV 16 E2 protein comprising the amino acid sequence of SEQ ID NO: 40, and a C-terminal gDM5 sequence comprising the amino sequence of SEQ ID NO: 17.

The vaccine(s) provided to the subject can comprise a nucleic acid molecule that encodes a fusion protein comprising an N-terminal gDM5 sequence comprising the amino sequence of SEQ ID NO: 13, a mutant HPV 16 E7 protein comprising the amino acid sequence of SEQ ID NO: 34, a mutant HPV 16 E6 protein comprising the amino acid sequence of SEQ ID NO: 36, a mutant HPV 16 E5 protein comprising the amino acid sequence of SEQ ID NO: 38, a mutant HPV 16 E2 protein comprising the amino acid sequence of SEQ ID NO: 40, and a C-terminal gDM5 sequence comprising the amino sequence of SEQ ID NO: 17.

The vaccine(s) provided to the subject can comprise a nucleic acid molecule that encodes a fusion protein comprising the amino acid sequence of SEQ ID NO: 46. The vaccine(s) provided to the subject can comprise a nucleic acid molecule that encodes a fusion protein comprising the amino acid sequence of SEQ ID NO: 44.

Disclosed herein are HPV 16 E7, HPV 16 E6, HPV 16 E5, and HVP 16 E2 proteins comprising any of the below amino acid sequences. Also disclosed are nucleic acid molecules encoding the HPV 16 E7, HPV 16 E6, HPV 16 E5, and HVP 16 E2 proteins, the nucleic acid molecules comprising any of the below nucleotide sequences.

TABLE 7

| HPV 16 Protein or Nucleotide Sequence | SEQ ID NO |
|---|---|
| Mutant HPV E7 Protein | SEQ ID NO: 34 |
| Mutant HPV E7 Nucleotide | SEQ ID NO: 35 |
| Mutant HPV E6 Protein | SEQ ID NO: 36 |
| Mutant HPV E6 Nucleotide | SEQ ID NO: 37 |
| Mutant HPV E5 Protein | SEQ ID NO: 38 |
| Mutant HPV E5 Nucleotide | SEQ ID NO: 39 |
| Mutant HPV E2 Protein | SEQ ID NO: 40 |
| Mutant HPV E2 Nucleotide | SEQ ID NO: 41 |
| C-terminal HPV E2 protein | SEQ ID NO: 56 |
| C-terminal HPV E2 Nucleotide | SEQ ID NO: 57 |
| N-terminal E2 protein | SEQ ID NO: 58 |
| N-terminal E2 Nucleotide | SEQ ID NO: 59 |
| Mutant HPV E7 Protein Version 2 | SEQ ID NO: 64 |
| Mutant HPV E7 Nucleotide Version 2 | SEQ ID NO: 65 |
| Mutant HPV E6 Protein Version 2 | SEQ ID NO: 66 |
| Mutant HPV E6 Nucleotide Version 2 | SEQ ID NO: 67 |
| Mutant HPV E5 Protein Version 2 | SEQ ID NO: 68 |
| Mutant HPV E5 Nucleotide Version 2 | SEQ ID NO: 69 |

Disclosed herein are HPV 16 E765 fusion proteins, which can comprise any one of the below HPV 16 E7 proteins, any one of the below HPV 16 E6 proteins, and any one of the below HPV 16 E5 proteins.

TABLE 8

| HPV E7 | SEQ ID | HPV E6 | SEQ ID | HPV E5 | SEQ ID |
|---|---|---|---|---|---|
| Protein | NO | Protein | NO | Protein | NO |
| Mutant HPV E7 | 34 | Mutant HPV E6 Protein | 36 | Mutant HPV E5 Protein | 38 |
| Protein | | | | | |
| Mutant HPV E7 | 64 | Mutant HPV E6 Protein | 66 | Mutant HPV E5 Protein | 68 |
| Protein Version 2 | | Version 2 | | Version 2 | |

The HPV 16 E765 fusion proteins can be encoded by any one of the below HPV 16 E7 nucleotide sequences, any one of the below HPV 16 E6 nucleotide sequences, and any one of the below HPV 16 E5 nucleotide sequences.

TABLE 9

| HPV E7 | SEQ ID | HPV E6 | SEQ ID | HPV E5 | SEQ ID |
|---|---|---|---|---|---|
| Nucleotide | NO | Nucleotide | NO | Nucleotide | NO |
| Mutant HPV E7 | 35 | Mutant HPV E6 | 37 | Mutant HPV E5 | 39 |
| Nucleotide | | Nucleotide | | Nucleotide | |
| Mutant HPV E7 | 65 | Mutant HPV E6 | 67 | Mutant HPV E5 | 69 |
| Nucleotide | | Nucleotide | | Nucleotide | |
| Version 2 | | Version 2 | | Version 2 | |

Disclosed herein are HPV 16 E7652 fusion proteins, which can comprise any one of the below HPV 16 E7 proteins, any one of the below HPV 16 E6 proteins, any one of the below HPV 16 E5 proteins, and any of the below HPV 16 E2 proteins.

TABLE 10

| HPV E7 | SEQ ID | HPV E6 | SEQ ID | HPV E5 | SEQ ID | HPV E2 | SEQ ID |
|---|---|---|---|---|---|---|---|
| Protein | NO | Protein | NO | Protein | NO | Protein | NO |
| Mutant HPV E7 Protein | 34 | Mutant HPV E6 | 36 | Mutant HPV E5 | 38 | Mutant HPV E2 | 40 |
| | | Protein | | Protein | | Protein | |
| Mutant HPV E7 Protein | 64 | Mutant HPV E6 | 66 | Mutant HPV E5 | 68 | C-terminal HPV | 56 |
| Version 2 | | Protein Version 2 | | Protein Version 2 | | E2 protein | |
| | | | | | | N-terminal E2 | 58 |
| | | | | | | protein | |

The HPV 16 E7652 fusion proteins can be encoded by any one of the below HPV 16 E7 nucleotide sequences, any one of the below HPV 16 E6 nucleotide sequences, any one of the below HPV 16 E5 nucleotide sequences, and any one of the below HPV 16 E2 nucleotide sequences.

TABLE 11

| HPV E7 | SEQ ID | HPV E6 | SEQ ID | HPV E5 | SEQ ID | HPV E2 | SEQ ID |
|---|---|---|---|---|---|---|---|
| Nucleotide | NO | Nucleotide | NO | Nucleotide | NO | Nucleotide | NO |
| Mutant HPV E7 | 35 | Mutant HPV E6 | 37 | Mutant HPV E5 | 39 | Mutant HPV E2 | 41 |
| Nucleotide | | Nucleotide | | Nucleotide | | Nucleotide | |
| Mutant HPV E7 | 65 | Mutant HPV E6 | 67 | Mutant HPV E5 | 69 | C-terminal HPV | 57 |
| Nucleotide Version 2 | | Nucleotide | | Nucleotide | | E2 Nucleotide | |
| | | Version 2 | | Version 2 | | | |
| | | | | | | N-terminal E2 | 59 |
| | | | | | | Nucleotide | |

For example, the HPV 16 E765 and E7652 fusion proteins can comprise any of the below amino acid sequences, or be encoded by any of the below nucleotide sequences:

TABLE 12

| HPV 16 E765/E7652 Protein or Nucleotide | SEQ ID NO |
|---|---|
| E765dt1 Protein | SEQ ID NO: 19 |
| E765dt1 Nucleotide | SEQ ID NO: 20 |
| E765dt3 Protein (also referred to herein as E765 | SEQ ID NO: 21 |
| Protein) | |
| E765dt3 Nucleotide | SEQ ID NO: 22 |
| (also referred to herein as E765 Nucleotide) | |
| Mutant HPV E7, E6, E5, and E2 Fusion Protein | SEQ ID NO: 23 |
| E7652 Protein) | |
| Mutant HPV E7, E6, E5, and E2 Fusion Nucleotide | SEQ ID NO: 24 |
| (E7652 Nucleotide) | |
| Mutant HPV E7, E6, E5, and E2 Fusion Protein | SEQ ID NO: 42 |
| (E7652 Protein)(without N-term M) | |
| Mutant HPV E7, E6, E5, and E2 Fusion Nucleotide | SEQ ID NO: 43 |
| (E7652 Nucleotide) (without N-term atg) | |
| Mutant HPV E7, E6, E5 and C-terminus E2 Fusion | SEQ ID NO: 60 |
| Protein | |
| Mutant HPV E7, E6, E5 and C-terminus E2 Fusion | SEQ ID NO: 61 |
| Nucleotide | |
| Mutant HPV E7, E6, E5 and N-terminus E2 Fusion | SEQ ID NO: 62 |
| Protein | |
| Mutant HPV E7, E6, E5 and N-terminus E2 Fusion | SEQ ID NO: 63 |
| Nucleotide | |
| Mutant E7, E6 and E5 fusion Protein | SEQ ID NO: 70 |
| Mutant E7, E6 and E5 fusion Nucleotide | SEQ ID NO: 71 |

Disclosed herein are fusion proteins comprising an HPV 16 E2 protein and an antigen. Exemplary antigens include, but are not limited to, the following:

TABLE 13

| Antigen Protein or Nucleotide Sequence | SEQ ID NO |
|---|---|
| PolN Protein | SEQ ID NO: 25 |
| PolN Nucleotide | SEQ ID NO: 26 |
| HBV3 Protein | SEQ ID NO: 27 |
| HBV3 Nucleotide | SEQ ID NO: 28 |
| HIV-1 gag Protein | SEQ ID NO: 29 |
| HIV-1 gag Nucleotide | SEQ ID NO: 30 |
| Melapoly Protein | SEQ ID NO: 31 |
| Melapoly Nucleotide | SEQ ID NO: 32 |
| E765-wt Protein | SEQ ID NO: 54 |
| E765-wt Nucleotide | SEQ ID NO: 55 |
| Melapoly Protein #2 | SEQ ID NO: 72 |
| Melapoly Nucleotide #2 | SEQ ID NO: 73 |
| Melanoma antigens with universal helper epitope | SEQ ID NO: 74 |
| Protein | |
| Melanoma antigens with universal helper epitope | SEQ ID NO: 75 |
| Nucleotide | |

The HPV 16 E2-antigen fusion proteins can comprise any one of the below HPV 16 E2 proteins and any one of the below antigens:

TABLE 14

| HPV 16 E2 Protein | | Antigen | |
|---|---|---|---|
| Name | SEQ ID NO | Name | SEQ ID NO |
| Mutant HPV E2 | 40 | PolN Protein | 25 |
| Protein | | | |
| C-terminal HPV E2 | 56 | HBV3 Protein | 27 |
| protein | | | |
| N-terminal E2 protein | 58 | HIV-1 gag Protein | 29 |
| | | Melapoly Protein | 31 |
| | | E765-wt Protein | 54 |
| | | Melapoly Protein #2 | 72 |
| | | Melanoma antigens | 74 |
| | | with universal | |
| | | helper epitope | |
| | | Protein | |

The HPV 16 E2-antigen fusion proteins can be encoded by any one of the below HPV 16 E2 nucleotide sequences and any one of the below antigen nucleotide sequences:

TABLE 15

| HPV 16 E2 Nucleotide | | Antigen | |
|---|---|---|---|
| Name | SEQ ID NO | Name | SEQ ID NO |
| Mutant HPV E2 | 41 | PolN Nucleotide | 26 |
| Nucleotide | | | |
| C-terminal HPV | 57 | HBV3 Nucleotide | 28 |
| Nucleotide | | | |
| N-terminal E2 | 59 | HIV-1 gag Nucleotide | 30 |
| Nucleotide | | | |
| | | Melapoly Nucleotide | 32 |
| | | E765-wt Nucleotide | 55 |
| | | Melapoly Nucleotide #2 | 73 |
| | | Melanoma antigens with | 75 |
| | | universal helper epitope | |
| | | Nucleotide | |

For example, the HPV 16 E2-antigen fusion proteins can comprise any of the below amino acid sequences, or be encoded by any of the below nucleotide sequences:

TABLE 16

| HPV 16 E2 Fusion Protein | SEQ ID NO |
|---|---|
| Melanoma antigens with universal | SEQ ID NO: 76 |
| helper epitope and E2 Protein | |
| Melanoma antigens with universal | SEQ ID NO: 77 |
| helper epitope and E2 Nucleotide | |
| Melanoma antigens with universal | SEQ ID NO: 78 |
| helper epitope and C-terminus E2 Protein | |
| Melanoma antigens with universal helper | SEQ ID NO: 79 |
| epitope and C-terminus E2 Nucleotide | |
| Melanoma antigens with universal helper | SEQ ID NO: 80 |
| epitope and N-terminus E2 Protein | |
| Melanoma antigens with universal helper | SEQ ID NO: 81 |
| epitope and N-terminus E2 Nucleotide | |

The HPV 16 E2-antigen fusion proteins can further comprise a gD protein. The gD-HPV 16 E2-antigen fusion proteins can comprise any one of the below gD proteins, any one of the below HPV 16 E2 proteins, and any one of the below antigens:

TABLE 17

| gD Protein | | HPV 16 E2 Protein | | Antigen | |
|---|---|---|---|---|---|
| | SEQ ID | | SEQ ID | | SEQ ID |
| Name | NO | Name | NO | Name | NO |
| gDM1 Protein | 1 | Mutant HPV E2 Protein | 40 | PolN Protein | 25 |
| gDM2 Protein | 3 | C-terminal HPV E2 | 56 | HBV3 Protein | 27 |
| | | protein | | | |
| gDM3 Protein | 5 | N-terminal E2 protein | 58 | HIV-1 gag Protein | 29 |
| gDM4 Protein | 7 | | | Melapoly Protein | 31 |
| gDM5 Protein | 9 | | | E765-wt Protein | 54 |
| gDM5 No Signal Seq | 11 | | | Melapoly Protein #2 | 72 |
| Protein | | | | | |
| gDM1 Protein #2 | 49 | | | Melanoma antigens with universal | 74 |
| | | | | helper epitope Protein | |
| gD without transmembrane | 50 | | | | |
| domain (TM) Protein | | | | | |
| Super gD with P2A | 52 | | | | |
| sequence Protein | | | | | |
| WT gD Protein | 33 | | | | |

The gD-HPV 16 E2-antigen fusion proteins can be encoded by any one of the below gD nucleotide sequences, any one of the below HPV 16 E2 nucleotide sequences, and any one of the below antigen nucleotide sequences:

TABLE 18

| gD Nucleotide | | HPV 16 E2 Nucleotide | | Antigen | |
|---|---|---|---|---|---|
| | SEQ ID | | SEQ ID | | SEQ ID |
| Name | NO | Name | NO | Name | NO |
| gDM1 Nucleotide | 2 | Mutant HPV E2 | 41 | PolN Nucleotide | 26 |
| | | Nucleotide | | | |
| gDM2 Nucleotide | 4 | C-terminal HPV | 57 | HBV3 Nucleotide | 28 |
| | | Nucleotide | | | |
| gDM3 Nucleotide | 6 | N-terminal E2 | 59 | HIV-1 gag Nucleotide | 30 |
| | | Nucleotide | | | |
| gDM4 Nucleotide | 8 | | | Melapoly Nucleotide | 32 |
| gDM5 Nucleotide | 10 | | | E765-wt Nucleotide | 55 |
| gDM5 No Signal Seq | 12 | | | Melapoly Nucleotide #2 | 73 |
| Nucleotide | | | | | |
| gD without transmembrane | 51 | | | Melanoma antigens with universal | 75 |
| domain (TM) Nucleotide | | | | helper epitope Nucleotide | |
| Super gD with P2A | 53 | | | | |
| sequence Nucleotide | | | | | |
| WT gD Nucleotide | 48 | | | | |

For example, the gD-HPV 16 E2-antigen fusion proteins can comprise any of the below amino acid sequences, or be encoded by any of the below nucleotide sequences:

TABLE 19

| gD-HPV 16 E2-Antigen Fusion Protein or Nucleotide | SEQ ID NO |
|---|---|
| Mutant HPV E7, E6, E5, and C-terminus E2 Fusion Protein Fused to | SEQ ID NO: 102 |
| gD Protein | |
| Mutant HPV E7, E6, E5, and C-terminus E2 Fusion Protein Fused to | SEQ ID NO: 103 |
| gD Nucleotide | |
| Mutant HPV E7, E6, E5, and N-terminus E2 Fusion Protein Fused to | SEQ ID NO: 104 |
| gD Protein | |
| Mutant HPV E7, E6, E5, and N-terminus E2 Fusion Protein Fused to | SEQ ID NO: 105 |
| gD Nucleotide | |
| Melanoma antigens with universal helper epitope and E2 fused into | SEQ ID NO: 120 |
| gD Protein | |
| Melanoma antigens with universal helper epitope and E2 fused into | SEQ ID NO: 121 |
| gD Nucleotide | |
| Melanoma antigens with universal helper epitope and C-terminus E2 | SEQ ID NO: 122 |
| fused into gD Protein | |
| Melanoma antigens with universal helper epitope and C-terminus E2 | SEQ ID NO: 123 |
| fused into gD Nucleotide | |
| Melanoma antigens with universal helper epitope and N-terminus E2 | SEQ ID NO: 124 |
| fused into gD Protein | |
| Melanoma antigens with universal helper epitope and N-terminus E2 | SEQ ID NO: 125 |
| fused into gD Nucleotide | |
| Melanoma antigens with universal helper epitope and E2 fused into | SEQ ID NO: 126 |
| gD with C-terminal Flag Protein | |
| Melanoma antigens with universal helper epitope and E2 fused into | SEQ ID NO: 127 |
| gD with C-terminal Flag Nucleotide | |
| Melanoma antigens with universal helper epitope and the C-terminus | SEQ ID NO: 128 |
| of E2 fused into gD with C-terminal Flag Protein | |
| Melanoma antigens with universal helper epitope and the C-terminus | SEQ ID NO: 129 |
| of E2 fused into gD with C-terminal Flag Nucleotide | |
| Melanoma antigens with universal helper epitope and the N-terminus | SEQ ID NO: 130 |
| of E2 fused into gD with C-terminal Flag Protein | |
| Melanoma antigens with universal helper epitope and the N-terminus | SEQ ID NO: 131 |
| of E2 fused into gD with C-terminal Flag Nucleotide | |
| Mutant HPV E7, E6, E5, and C-terminus E2 Fusion Protein Fused to | SEQ ID NO: 106 |
| gDM5 Protein | |
| Mutant HPV E7, E6, E5, and C-terminus E2 Fusion Protein Fused to | SEQ ID NO: 107 |
| gDM5 Nucleotide | |
| Mutant HPV E7, E6, E5, and N-terminus E2 Fusion Protein Fused to | SEQ ID NO: 108 |
| gDM5 Protein | |
| Mutant HPV E7, E6, E5, and N-terminus E2 Fusion Protein Fused to | SEQ ID NO: 109 |
| gDM5 Nucleotide | |
| Melanoma antigens with universal helper epitope and E2 fused into | SEQ ID NO: 132 |
| gDM5 Protein | |
| Melanoma antigens with universal helper epitope and E2 fused into | SEQ ID NO: 133 |
| gDM5 Nucleotide | |
| Melanoma antigens with universal helper epitope and C-terminus E2 | SEQ ID NO: 134 |
| fused into gDM5 Protein | |
| Melanoma antigens with universal helper epitope and C-terminus E2 | SEQ ID NO: 135 |
| fused into gDM5 Nucleotide | |
| Melanoma antigens with universal helper epitope and N-terminus E2 | SEQ ID NO: 136 |
| fused into gDM5 Protein | |
| Melanoma antigens with universal helper epitope and N-terminus E2 | SEQ ID NO: 137 |
| fused into gDM5 Nucleotide | |
| Melanoma antigens with universal helper epitope and E2 fused into | SEQ ID NO: 138 |
| gDM5 with C-terminal Flag Protein | |
| Melanoma antigens with universal helper epitope and E2 fused into | SEQ ID NO: 139 |
| gDM5 with C-terminal Flag Nucleotide | |
| Melanoma antigens with universal helper epitope and the C-terminus | SEQ ID NO: 140 |
| of E2 fused into gDM5 with C-terminal Flag Protein | |
| Melanoma antigens with universal helper epitope and the C-terminus | SEQ ID NO: 141 |
| of E2 fused into gDM5 with C-terminal Flag Nucleotide | |
| Melanoma antigens with universal helper epitope and the N-terminus | SEQ ID NO: 142 |
| of E2 fused into gDM5 with C-terminal Flag Protein | |
| Melanoma antigens with universal helper epitope and the N-terminus | SEQ ID NO: 143 |
| of E2 fused into gDM5 with C-terminal Flag Nucleotide | |

Disclosed herein are nucleic acid molecules encoding a mutant human papilloma virus 16 (HPV 16) E7 protein comprising the amino acid sequence of SEQ ID NO: 34, a mutant HPV 16 E6 protein comprising the amino acid sequence of SEQ ID NO: 36, a mutant HPV 16 E5 protein comprising the amino acid sequence of SEQ ID NO: 38, a mutant HPV 16 E2 protein comprising the amino acid sequence of SEQ ID NO: 40, a C-terminal mutant HPV 16 E2 protein comprising the amino acid sequence of SEQ ID NO: 56, a N-terminal mutant HPV 16 E2 protein comprising the amino acid sequence of SEQ ID NO: 58, a mutant HPV 16 E7 protein v2 comprising the amino acid sequence of SEQ ID NO: 64, a mutant HPV 16 E6 protein v2 comprising the amino acid sequence of SEQ ID NO: 66, or a mutant HPV 16 E5 protein v2 comprising the amino acid sequence of SEQ ID NO: 68.

In some embodiments, the nucleic acid molecule encodes the mutant HPV 16 E2 protein comprising the amino acid sequence of SEQ ID NO: 40. In some embodiments, the nucleic acid molecule encodes the C-terminal mutant HPV 16 E2 protein comprising the amino acid sequence of SEQ ID NO: 56. In some embodiments, the nucleic acid molecule encodes the N-terminal mutant HPV 16 E2 protein comprising the amino acid sequence of SEQ ID NO: 58.

The nucleic acid molecules can comprise the mutant HPV 16 E7 comprising the nucleotide sequence of SEQ ID NO: 35, mutant HPV 16 E6 comprising the nucleotide sequence of SEQ ID NO: 37, mutant HPV 16 E5 comprising the nucleotide sequence of SEQ ID NO: 39, mutant HPV 16 E2 comprising the nucleotide sequence of SEQ ID NO: 41. C-terminal mutant HPV 16 E2 comprising the nucleotide sequence of SEQ ID NO: 57, N-terminal mutant HPV 16 E2 comprising the nucleotide sequence of SEQ ID NO: 59, mutant HPV 16 E7 v2 comprising the nucleotide sequence of SEQ ID NO: 65, mutant HPV 16 E6 v2 comprising the nucleotide sequence of SEQ ID NO: 67, or mutant HPV 16 E5 v2 comprising the nucleotide sequence of SEQ ID NO: 69.

In some embodiments, the nucleic acid molecule comprises the mutant HPV 16 E2 comprising the nucleotide sequence of SEQ ID NO: 41. In some embodiments, the nucleic acid molecule comprises the C-terminal mutant HPV 16 E2 comprising the nucleotide sequence of SEQ ID NO: 57. In some embodiments, the nucleic acid molecule comprises the N-terminal mutant HPV 16 E2 comprising the nucleotide sequence of SEQ ID NO: 59.

Disclosed herein are nucleic acid molecules encoding an HPV 16 fusion protein, wherein the HPV 16 fusion protein comprises any one of the HPV 16 E7 proteins provided in Table 10, any one of the HPV 16 E6 proteins provided in Table 10, and any one of the HPV 16 E5 proteins provided in Table 10. The nucleic acid encoding an HPV 16 fusion protein can further comprise a nucleotide sequence encoding any of the HPV 16 E2 proteins provided in Table 10.

The nucleic acid encoding an HPV 16 fusion protein can comprise any one of the HPV 16 E7 nucleotide sequences provided in Table 11, any one of the HPV 16 E6 nucleotide sequences provided in Table 11, and any one of the HPV 16 E5 nucleotide sequences provided in Table 11. The nucleic acid can further comprise any of the HPV 16 E2 nucleotide sequences provided in Table 11.

The nucleic acid molecule encoding an HPV fusion protein can encode any one of the HPV 16 fusion proteins provided in Table 12. The nucleic acid molecule can comprise any one of the nucleotide sequences provided in Table 12.

Disclosed herein are nucleic acid molecules encoding an HPV 16 E2-antigen fusion protein, wherein the HPV 16 E2-antigen fusion protein comprises any one of the HPV 16 E2 proteins provided in Table 14, and any one of the antigens provided in Table 14. The HPV 16 E2-antigen fusion protein can comprise any one of the amino acid sequences provided in Table 16. In some embodiments, the nucleic acid molecule encodes an HPV 16 E2-melanoma antigen with a universal T helper cell epitope comprising the amino acid sequence of SEQ ID NO: 76. The nucleic acid molecule encoding an HPV 16 E2-antigen fusion protein can comprise any one of the HPV 16 E2 nucleotide sequences provided in

Table 15, and any one of the antigen nucleotide sequences provided in Table 15. The nucleic acid molecule encoding an HPV 16 E2-antigen fusion protein can comprise any one of the nucleotide sequences provided in Table 16.

The nucleic acid molecule encoding an HPV 16 E2-antigen fusion protein can comprise the HPV 16 E2-melanoma antigen with a universal T helper cell epitope comprising the nucleotide sequence of SEQ ID NO: 77.

Any of the nucleic acid molecules encoding an HPV 16 E2-antigen fusion proteins can further comprise a nucleotide sequence that encodes a gD. The gD can comprise any one of the amino acid sequences provided in Table 17. The nucleotide sequence encoding the gD can comprise any one of the nucleotide sequences provided in Table 18. The nucleic acid molecule encoding an HPV 16 E2-antigen fusion protein can encode any one of the gD-HPV 16 E2-antigen fusion proteins provided in Table 19. The nucleic acid molecule encoding an HPV 16 E2-antigen fusion protein can comprise the nucleotide sequence of any one of the nucleotide sequences provided in Table 19.

The nucleic acid molecule can encode a melanoma antigens with universal helper epitope and E2 fused into gD Protein comprising the amino acid sequence of SEQ ID NO: 120, a melanoma antigens with universal helper epitope and C-terminus E2 fused into gD Protein comprising the amino acid sequence of SEQ ID NO: 122, or a melanoma antigens with universal helper epitope and N-terminus E2 fused into gD Protein comprising the amino acid sequence of SEQ ID NO: 124. In some embodiments, the nucleic acid encodes the amino acid sequence of SEQ ID NO: 120. In some embodiments, the nucleic acid encodes the amino acid sequence of SEQ ID NO: 122. In some embodiments, the nucleic acid encodes the amino acid sequence of SEQ ID NO: 124. The nucleic acid molecule encoding a melanoma antigen with universal helper epitope and E2 fused into gD Protein can comprise the nucleotide sequence of any one of SEQ ID NOs: 121, 123, or 125. In some embodiments, the nucleic acid molecule comprises SEQ ID NO 121. In some embodiments, the nucleic acid molecule comprises SEQ ID NO 123. In some embodiments, the nucleic acid molecule comprises SEQ ID NO 125.

Disclosed herein are proteins comprising any one of the amino acid sequences provided in Tables 7, 8, 10, 12, 14, 16, 17, or 19. The protein can comprise a mutant human papilloma virus 16 (HPV 16) E7 protein comprising the amino acid sequence of SEQ ID NO: 34, a mutant HPV 16 E6 protein comprising the amino acid sequence of SEQ ID NO: 36, a mutant HPV 16 E5 protein comprising the amino acid sequence of SEQ ID NO: 38, a mutant HPV 16 E2 protein comprising the amino acid sequence of SEQ ID NO: 40, a C-terminal mutant HPV 16 E2 protein comprising the amino acid sequence of SEQ ID NO: 56, a N-terminal mutant HPV 16 E2 protein comprising the amino acid sequence of SEQ ID NO: 58, a mutant HPV 16 E7 protein v2 comprising the amino acid sequence of SEQ ID NO: 64, a mutant HPV 16 E6 protein v2 comprising the amino acid sequence of SEQ ID NO: 66, a mutant HPV 16 E5 protein v2 comprising the amino acid sequence of SEQ ID NO: 68, an HPV 16 E2-melanoma antigen with a universal T helper cell epitope comprising the amino acid sequence of SEQ ID NO: 76, a melanoma antigens with universal helper epitope and E2 fused into gD Protein comprising the amino acid sequence of SEQ ID NO: 120, a melanoma antigens with universal helper epitope and C-terminus E2 fused into gD Protein comprising the amino acid sequence of SEQ ID NO: 122, or a melanoma antigens with universal helper epitope and N-terminus E2 fused into gD Protein comprising the amino acid sequence of SEQ ID NO: 124.

Disclosed herein are vectors comprising any of the herein disclosed nucleic acid molecules.

Disclosed herein are host cells comprising any of the herein disclosed vectors.

Disclosed herein are viruses comprising any of the herein disclosed nucleic acid molecules or any of the herein disclosed vectors. In some embodiments, the virus comprises a herein disclosed nucleic acid molecule. In some embodiments, the virus comprises a herein disclosed vector. The virus can be an adenovirus. Suitable adenoviruses include an AdC6, AdC68, or AdC7.

Disclosed herein are vaccines comprising any of the herein disclosed nucleic acid molecules, any of the herein disclosed vectors, or any of the herein disclosed viruses. In some embodiments, the vaccine comprises a herein disclosed nucleic acid molecule. In some embodiments, the vaccine comprises a herein disclosed vector. In some embodiments, the vaccine comprises a herein disclosed virus. The vaccine can comprise a pharmaceutically acceptable carrier.

Disclosed herein are pharmaceutical compositions comprising any of the herein disclosed nucleic acid molecules, any of the herein disclosed vectors, or any of the herein disclosed viruses. In some embodiments, the pharmaceutical composition comprises at least one of the herein disclosed nucleic acid molecules. In some embodiments, the pharmaceutical composition comprises at least one of the herein disclosed vectors. In some embodiments, the pharmaceutical composition comprises at least one of the herein disclosed vectors. The pharmaceutical composition can further comprise a pharmaceutically acceptable carrier.

Disclosed herein are methods of inducing an immune response in a subject, the method comprising providing to the subject an effective amount of any of the herein disclosed nucleic acid molecules, any of the herein disclosed vectors, any of the herein disclosed fusion proteins, any of the herein disclosed viruses, or any of the herein disclosed vaccines to thereby induce an immune response. In some embodiments, the subject in provided at least one of the herein disclosed nucleic acid molecules. In some embodiments, the subject in provided at least one of the herein disclosed vectors. In some embodiments, the subject in provided at least one of the herein disclosed fusion proteins. In some embodiments, the subject in provided at least one of the herein disclosed viruses. In some embodiments, the subject in provided at least one of the herein disclosed vaccines.

Disclosed herein are pharmaceutical compositions comprising any of the herein disclosed nucleic acid molecules, any of the herein disclosed vectors, any of the herein disclosed fusion proteins, any of the herein disclosed viruses, or any of the herein disclosed vaccines for use in inducing an immune response in a subject. In some embodiments, the pharmaceutical composition comprises at least one of the herein disclosed nucleic acid molecules. In some embodiments, the pharmaceutical composition comprises at least one of the herein disclosed vectors. In some embodiments, the pharmaceutical composition comprises at least one of the herein disclosed fusion proteins. In some embodiments, the pharmaceutical composition comprises at least one of the herein disclosed viruses. In some embodiments, the pharmaceutical composition comprises at least one of the herein disclosed vaccines.

Disclosed herein are any of the herein disclosed nucleic acid molecules, any of the herein disclosed vectors, any of the herein disclosed fusion proteins, any of the herein disclosed viruses, or any of the herein disclosed vaccines for use in the preparation of a medicament useful for inducing an immune response in a subject. In some embodiments, at least one of the herein disclosed nucleic acid molecules are used in the preparation of the medicament. In some embodiments, at least one of the herein disclosed vectors are used in the preparation of the medicament. In some embodiments, at least one of the herein disclosed fusion proteins are used in the preparation of the medicament. In some embodiments, at least one of the herein disclosed viruses are used in the preparation of the medicament. In some embodiments, at least one of the herein disclosed viruses are used in the preparation of the medicament. In some embodiments, at least one of the herein disclosed vaccines are used in the preparation of the medicament.

### EXAMPLES

The following examples are provided to further describe some of the embodiments disclosed herein. The examples are intended to illustrate, not to limit, the disclosed embodiments.

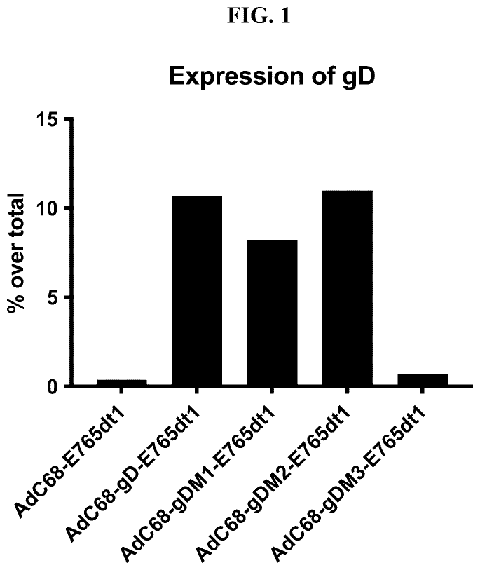

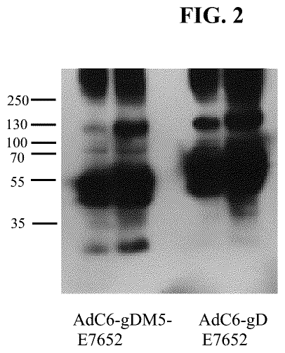

Genes encoding mutant gD proteins (gDM1, gDM2, gDM3, gDM4, gDM5, gD(-TM) and SgD-P2A) having the sequences provided in Table 23 were generated. Sequences of antigens such as those derived from HPV 16 early proteins or the other inserts described were cloned into the gD in between amino acids 277 and 278 of the gD, gDM1, gDM2, gDM3, gDM4, gDM5, or gD(-TM) which contain a site for the ApaI restriction enzyme. For the SgD-P2A vaccine, a vaccine with SgD followed by a P2A site was prepared. The sequences were then cloned into a transfer vector and from there into the viral molecular clones of AdC68, AdC7, or AdC6. The recombinant viral molecular clones were used to transfect HEK293 cells. Once virus was rescued in the transfected HEK cells it was expanded in HEK293 cells, purified and tested for genetic integrity by restriction enzyme digest of the purified viral genome and titrated for virus particle (VP) content and VP to infectious units (IU) ratios (Table 20). All vectors showed upon restriction enzyme digest the expected banding pattern. All vectors except the vector based on the gDM4 mutant gave VP yield within an acceptable range (>0.5×10e13). All except the vectors containing gDM3 and gDM4 gave VP to IU ratios within the acceptable range (<1000). Promising constructs were sequentially passaged 12 times and their genome was tested for genetic integrity by restriction enzyme digest to ensure vectors were genetically stable. All of the tested vectors except for the vectors containing the gDM3 mutant were genetically stable (Table 20). Vectors were tested for expression either by cell surface staining of transduced cells for gD followed by analysis by flow cytometry (wild-type gD, gDM1, gDM2, gDM3) or by Western blots (wild type, gDM5, gD(-TM) or gD-P2A) using lysates or in some cases supernatant of HEK293 cells that had been transduced with 1000 vp of vector per cell. Flow cytometry expression of gD was similar upon transduction with wild-type gD, gDM1 or gDM2 while gDM3 showed very low levels of gD expression (FIG. 1). Western blot levels of expression tended to be higher by the vector containing gD than gDM5. The gD(-TM) vaccine expressing a fusion protein of wild-type E7, E6 and E5 resulted in a secreted gD fusion protein as had been expected.

TABLE 20
Recovery of vectors

| | | | Stability |
|---|---|---|---|
| | Yield/20 | VP to IU | after 12 |
| Vaccine Name | flasks | ratio | passages |
| AdC68-gD-E765dt1 | 3.60E+14 | 120 | yes |
| AdC68-gDM1-E765dt1 | 1.50E+14 | 31 | nt |
| AdC68-gDM2-E765dt1 | 6.00E+13 | 214 | yes |
| AdC68-gDM3-E765dt1 | 1.90E+14 | 2400 | no |
| AdC68-gDM4-E765dt1 | 3.10E+12 | >10000 | nt |
| AdC6-gD-E7652 | 1.00E+14 | 89 | yes |
| AdC6-gDM5-E7652 | 2.00E+14 | 42 | yes |
| AdC6-E7652 | 1.10E+13 | 286 | nt |
| AdC68-gD(-TM)-E765 | 5.00E+13 | nt | nt |
| AdC6-SgD-P2A-E7652 | 3.90E+13 | 157 | nt |

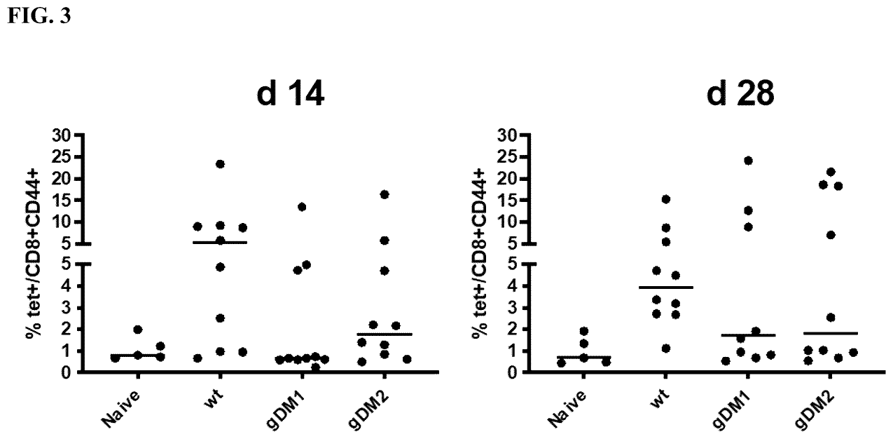

CD8+ T cell responses to the inserted antigen. Vectors that passed the initial quality control studies were tested for induction of CD8+ T cell responses in mice. gDM3 which was genetically unstable and gDM4, which grew poorly, were not tested. For testing of the gDM1 and gDM2 vectors, groups of 10 C57Bl/6 mice were injected with 10¹⁰ vp of the AdC68 vector expressing a modified version of HPV 16 E765 fusion protein (called dt1) fused either into wild-type gD or the gDM1 or gDM2 mutants. CD8+ T cell responses were tested 14 and 28 days later using peripheral blood mononuclear cells (PBMCs). Cells were isolated and purified and stained with antibodies to CD8, CD44, a tetramer to the immunodominant epitope pf E7 and a live cells stain. Cells were then analyzed by flow cytometry. As shown in FIG. 3, the mutant gD vector induced lower CD8⁺ T cell responses compared to the original gD.

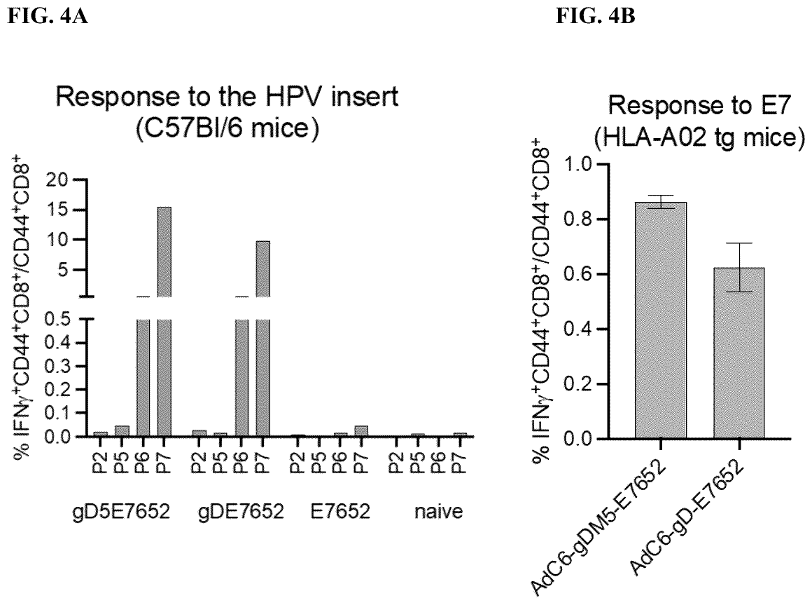

The gDM5 mutant was tested in comparison to wild-type gD or no gD using an HPV insert that contained immunogenic fragments of HPV 16 E7, E6, E5 and E2 in C57Bl/6 mice using peptide pools for the 4 inserted oncoprotein fragments or in HLA-A02 transgenic mice using peptides of E7 only. As shown in FIG. 4 the gD5 vectors tended to be slightly more immunogenic.

To test the performance of gDS with different inserts, additional vectors were constructed in which antigens of hepatitis B virus (PolN-N terminus of polymerase, HBV3—fragments of core and polymerase), gag of HIV-1, epitopes expressed by melanoma cells (Melapoly) or HPV sequences (E765dt1, mutant E765 (called E765), wild type E765, and mutant E7652) were genetically fused into gD, gDM5, gD without a transmembrane domain, or SgD-P2A and expressed by the chimpanzee adenovirus vectors of serotypes 6, 7, or 68. As shown in Table 21, vector yields were comparable. The VP to IU ratios tended to be lower with the gDM5 vectors.

TABLE 21

| | Recovery | VP to IU | | Recovery | VP to IU |
|---|---|---|---|---|---|
| Vector | (VP) | Ratio | Vector | (VP) | Ratio |
| C6-gDM5-PolN | 1.16E+14 | 435 | C6-gD-Poln | 1.55E+14 | 193 |
| C6-gDM5-HBV3 | 1.35E+14 | nt | C6-gD-HBV3 | 1.95E+14 | 2500 |
| C6-gDM5-gag | 2.11E+14 | 261 | C6-gD-gag | 2.32E+14 | 462 |
| C6-gDM5-Melapoly#2 | 2.00E+14 | 227 | C6-gD-Melapoly#2 | 1.2E+14 | 846 |
| C68-gDM5-E765dt3 | 2.00E+14 | nt | C68-gD-E765dt3 | 2.40E+14 | 250 |
| C6-gDMS-E7652 | 2.00E+14 | 42 | C6-gD-E7652 | 1.00E+14 | 89 |
| Averages | 1.77E+14 | 241 | | 1.74E+14 | 723 |

nt—not tested

Protein expression tested for by Western Blots using a monoclonal anti-gD antibody showed that under the experimental conditions (48 hour transduction of HEK293 cells with 1000 VP of vector/cell) expression levels tended to be higher with the gD vectors for Melapoly, gag, and E7652.

CD8+ T cell responses to different inserts fused into gD or gD5. T cell responses were tested in mice as follows.

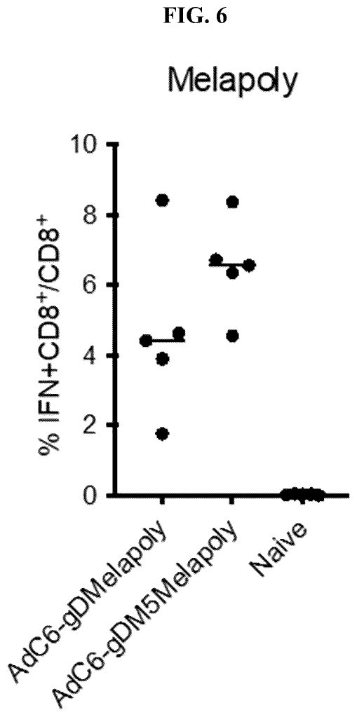

Mice were injected with 10¹⁰ vp of AdC6-gD-Melapoly #2 or AdC6-gDM5-Melapoly #2. Splenocytes were tested 17 days later for responses to the immunodominant epitope present within Melapoly for ICS for IFN-γ. As shown in FIG. 6 the AdC6-gD5-Melapoly #2 vector tended to induce a higher response than the AdC6-gD-Melapoly #2 vector (p value by 2 way Anova=0.076).

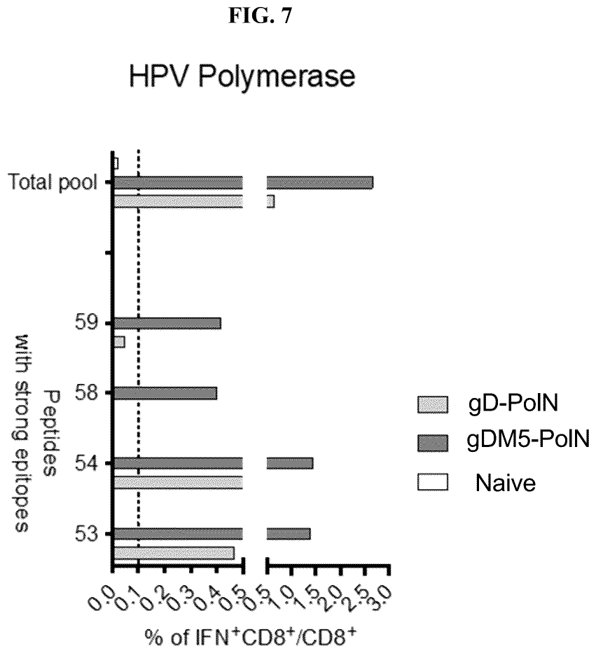

Mice (n=5) were injected with 1×10⁹ vp of the AdC6-gD-PolN or AdC6-gD5-PolN vaccines. Four weeks later splenocytes pooled from the 5 mice were tested by ICS for IFN-γ production in response to the peptide pool or individual peptides. Results in FIG. 7 show the response against the pool and the most immunodominant peptides. Responses were consistently higher in response to the AdC6-gD5-PolN vector

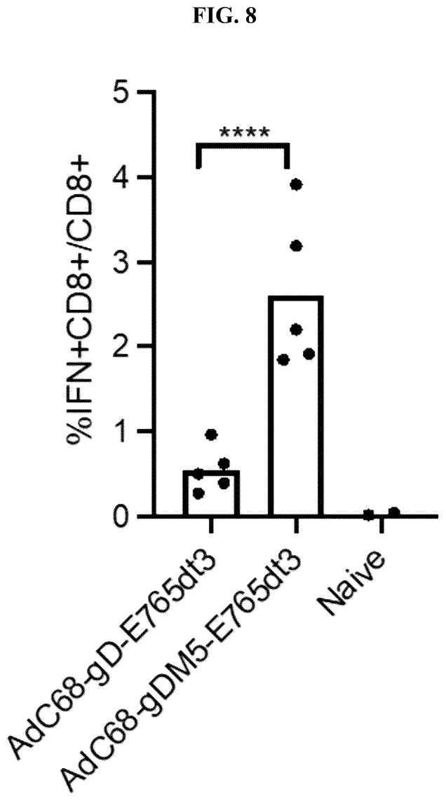

Groups of mice were injected with 2×10¹⁰ vp of the AdC67-gD-E765 or the AdC68-gDM5-E765 vectors. Splenocytes were tested 4 weeks later by ICS for CD8+ T cell responses to peptides representing the E7 sequence. As shown in FIG. 8, the AdC68-gDM5-E765 vector induced higher T cell responses compared to the AdC68-gD-E765 vector.

Sequences of HPV16 were cloned genetically into gD or gDM5 in between amino acids 277 and 278 (amino acid numbering based upon the original gD) which contains a site for the ApaI restriction enzyme. The gDM5 fusion sequences of the E7652 fusion gene or the E765 fusion gene or the helper cell epitope-Melanoma-E2 sequences without gD or gDM5 were then cloned into a transfer vector and from there into the viral molecular clones of AdC6. For the melanoma sequences a Flag sequence was added to the C-terminus of gD. The recombinant viral molecular clones were used to rescue virus upon transfection in HEK293 cells. Virus was then expanded, purified, and tested for genetic integrity by restriction enzyme digest of the purified viral genome and titrated for virus particle (VP) content and VP to infectious units (IU) ratios (Table 22). All vectors showed the expected banding pattern upon restriction enzyme digest. Vectors expressing gD or gDM5 fusion proteins had VP yield within an acceptable range (>0.5×10e13) and gave VP to IU ratios within the acceptable range (<1000) (Table 22). The gD and gDM5 containing constructs were sequentially passaged 12 times and their genome was tested for genetic integrity by restriction enzyme digest to ensure vectors were genetically stable. Both vectors were genetically stable. Vectors were tested for expression by Western blot (gD, gDM5) using lysates of HEK293 cells that had been transduced with 1000 vp of vector per cell. Cells were lysed in RIPA buffer supplemented with a 1% μl protease inhibitor (Santa Cruz Biotechnology Inc., Dallas. TX). A 15 μl of lysate was resolved by 12% SDS-PAGE gel electrophoresis (NuPage 4-12% Bis-Tris Gel. InVitrogen. Carlsbad. CA) and transferred to a polyvinylidene difluoride (PVDF) membrane (Merck Millipore, Burlington. MA). The membrane was blocked in 5% powder milk overnight in 4° C. The primary antibody to gD diluted to 1:1000 in saline (clone ABM19C9. Abenomics. San Diego, CA) was added for 1 hour at room temperature. Membranes were washed with 1X TBS-T prior to incubating with HRP-conjugated goat anti-rabbit secondary IgG (ab6721. Abcam. Cambridge UK) for 1 hour at room temperature. In parallel the membrane was probed with a mouse monoclonal IgG antibody to β-actin (Sc-47778. Santa Cruz Biotechnology. Dallas, TX) as a loading control for 1 hour at room temperature. The loading control antibody was probed with HRP-conjugated goat anti-mouse secondary IgG (SAB3701047. Sigma. St. Louis, MO) for 1 hour at room temperature. Membranes were washed 3 times with 1X TBS-T. The developing agent Super Signal West Pico Chemiluminescent (Thermo Fisher Scientific. Waltham, MA) was added. Membranes were shaken in the dark for 5 min. dried and developed. Western blot levels of expression tended to be higher by the vector containing gD than gDM5 (FIG. 2).

TABLE 22
Recovery of vectors

| | Recovery | VP to IU |
|---|---|---|
| Vector | (VP) | Ratio |
| C6-gDM5-E7652 | 2.00E+14 | 42 |
| C6-gD-E7652 | 1.00E+14 | 89 |
| C6-E7652 | 1.1E+13 | 286 |

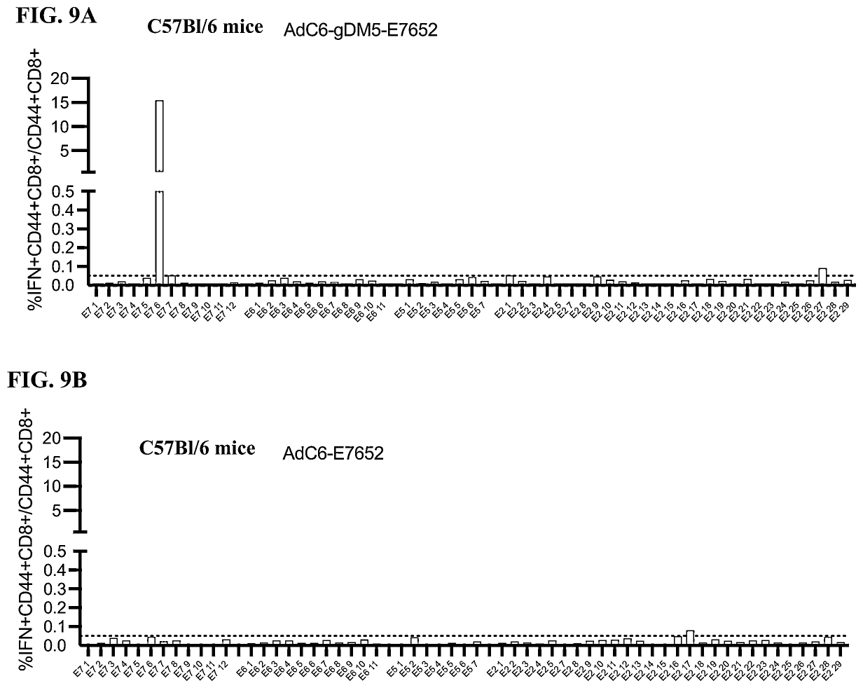

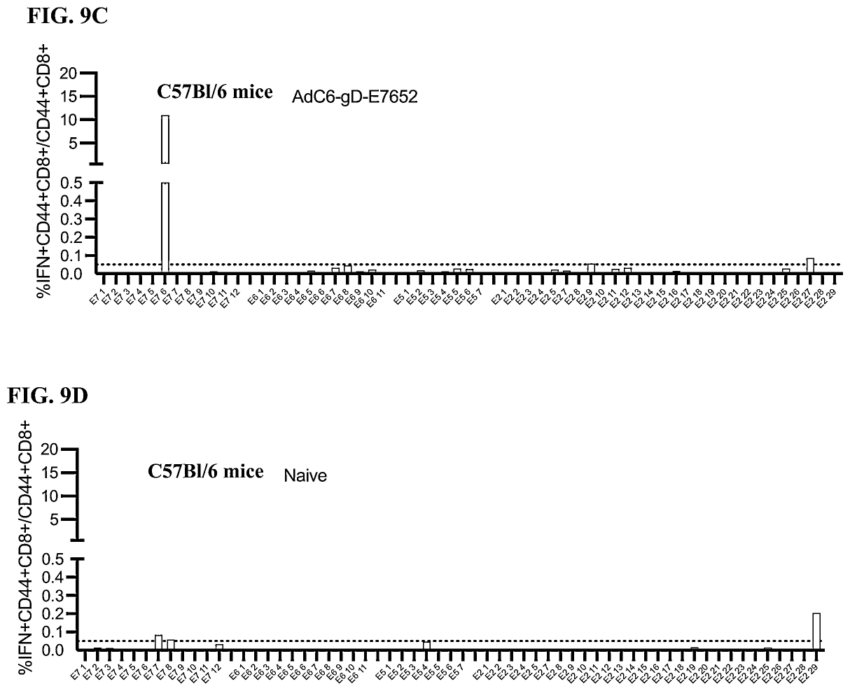

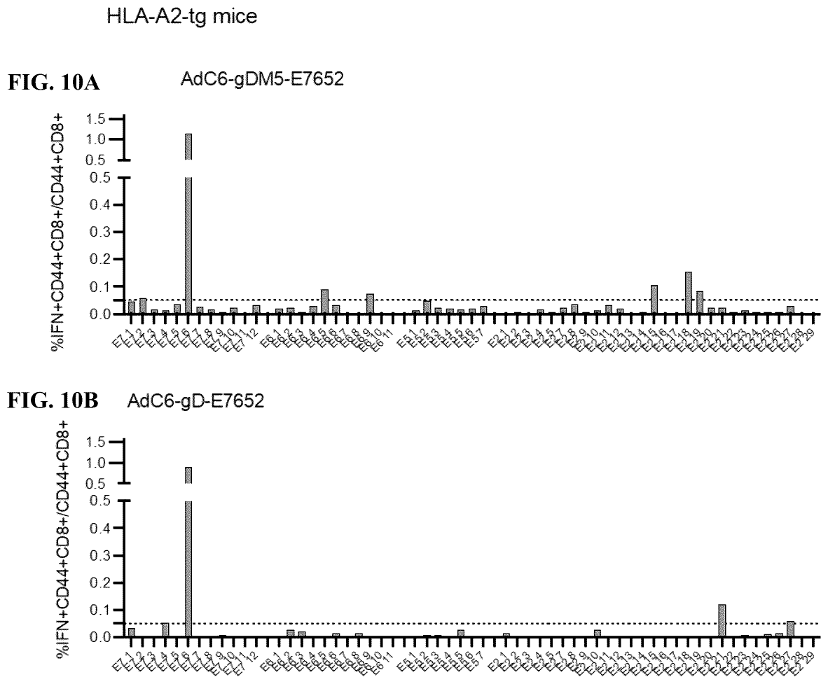

CD8+ T cell responses to the inserted antigen were tested. Vectors that passed the initial quality control studies were tested for induction of CD8+ T cell responses in C57Bl/6 mice using peptide pools for the 4 inserted oncoprotein fragments or in HLA-A02 transgenic mice using peptides of E7 only for in vitro stimulation of lymphocytes from vaccinated animals. As shown in FIG. 4A, FIG. 4B, FIG. 9A, FIG. 9B, FIG. 9C, FIG. 9D, FIG. 10A, and FIG. 10B the gDM5 vectors tended to be slightly more immunogenic than the gD vectors. gDM3 which was genetically unstable and gDM4, which grew poorly, were not tested.

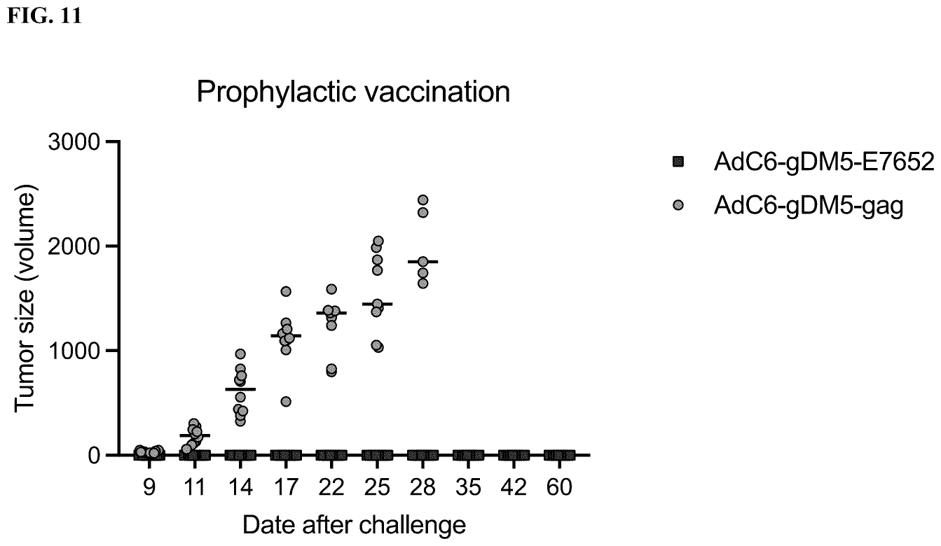

The data show protection against TC-1 tumor cell challenge in a pre-challenge vaccination model. Groups of 10 C57Bl/6 mice were vaccinated with 10¹⁰ vp of the AdC6-gDM5-E7652 vector or a control vector expressing HIV-1 gag genetically fused into gDM5. Mice were challenged 4 weeks later with 5×10⁵ TC1 cells given subcutaneously and tumor progression was recorded. As shown in FIG. 11 all the control mice rapidly developed tumors while all the AdC6-gDM5-E7652 mice remained tumor-free.

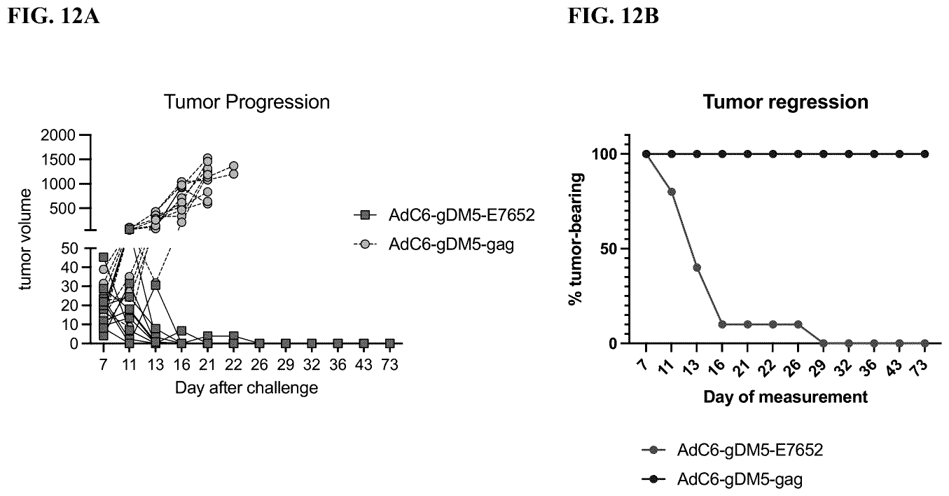

The data show protection against TC-1 tumor cell challenge in a post-challenge vaccination model. Groups of C57Bl/6 mice were challenged with 5×10⁴ TC1 cells. Three days later they were vaccinated with 10¹⁰ vp of the AdC6-gDM5-E7652 vector or a control vector expressing HIV-1 gag genetically fused into gDM5 and tumor growth was recoded. As shown in FIG. 12A and FIG. 12B all mice initially developed tumors, but while tumors continued to grow in the control mice the vaccinated mice rapidly controlled tumor growth and were tumor-free by 23 days after vaccination.

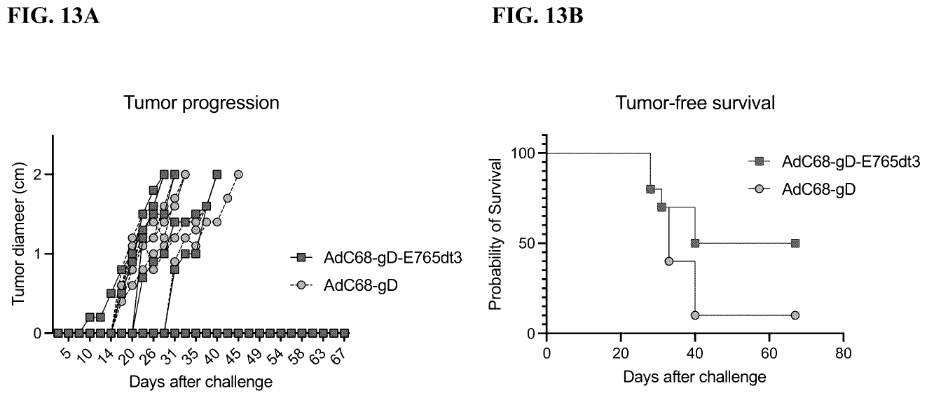

These results compare favorably to those that were generated with a previous vaccine expressing modified versions of E7, E6 and E5 fused into wild type gD and delivered by the chimpanzee adenovirus vector AdC68, which only achieved 50% protection of mice if used at the 10¹⁰ vp vector dose in mice that had been challenged 3 days earlier with 5×10⁴ TC1 cells (FIG. 13A and FIG. 13B).

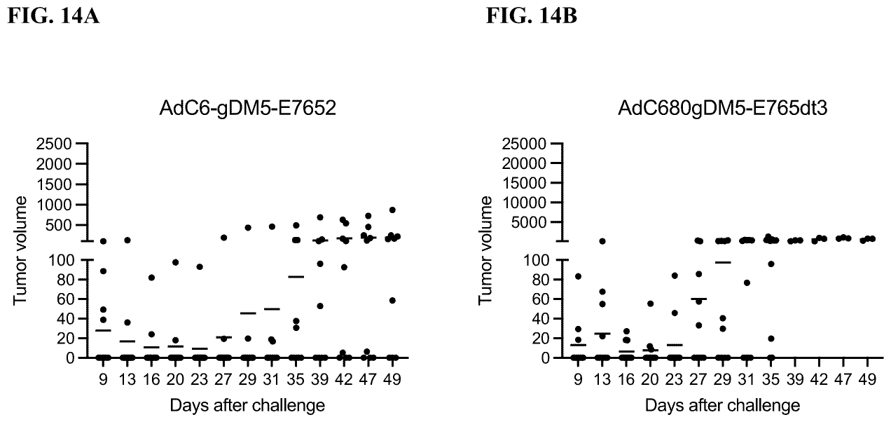

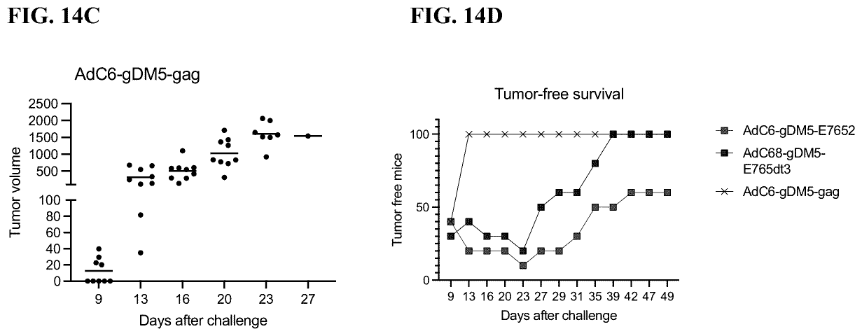

In another experiment mice were challenged with a higher dose of 2×10⁵ TC1 cells prior to vaccination with 10¹⁰ vp of AdC6-gDM5-E7652. AdC6-gD5-E765dt3 or the control vector AdC6-gDM5-gag. Control mice developed tumors that rapidly progressed. Tumor development and progression was delayed in vaccinated mice and this was more pronounced in mice vaccinated with the AdC6-gDM5-E7652 than the AdC6-gDM5-E765dt3 vaccine (FIG. 14A, FIG. 14B, FIG. 14C, and FIG. 14D).

T-cell responses were analyzed as follows. Mice were immunized with the indicated dose of vector given in 200 μl of saline i.m. into the left leg muscle. The assays were conducted with peripheral blood lymphocytes or splenocytes. Briefly, blood samples were collected by submandibular puncture and PBMCs were isolated by Histopaque (Sigma) gradient centrifugation. Single cell suspension was generated by mincing spleens and lymph nodes with mesh screens in Leibovitz's L15 medium followed by passing cells through a 70 μm filter (Fisher Scientific). Red blood cells were lysed by 1× RBC lysis buffer (eBioscience). T cell responses from spleens were analyzed at the indicated times shown in the figures. Splenocytes were purified by Percoll gradient centrifugation and stimulated with pools of peptides or individual peptides representing the HPV sequences present in the vaccines. Peptides were 15 amino acids in length and overlapped by 10 amino acids with the adjacent peptides. Individual peptides were diluted according to the manufacturer's instructions. For stimulation ˜10⁶ lymphocytes plated in medium containing 2% fetal calf serum and Golgiplug (BD Bioscience: San Jose, CA), at 1.5 μl/ml were cultured with peptides each present at a final concentration of 2 μg/ml for 5 hr at 37° C. in a 5% CO₂ incubator. Control cells were cultured without peptides.

Following stimulation, cells were incubated with anti-CD8-APC (clone 53-6.7, BioLegend, San Diego CA), anti-CD4-BV605 (clone RM4-5, BioLegend), anti-CD44-Alexa Flour 700 (clone IM7, BioLegend) and violet live/dead dye (Thermo Fisher Scientific) at 4° C. for 30 min in the dark. Cells were washed once with PBS and then fixed and permeabilized with Cytofix/Cytoperm (BD Biosciences. San Jose, CA) for 20 min. Cells were then incubated with an anti-IFN-γ-FITC antibody (Clone. XMG1.2 BioLegend), an antibody to perforin labeled with PE/Dazzle 594 clone S16009A from Biolegend. and a PE/Cyanide 7-labeled antibody to granzyme B, clone QA18428 from Biolegend at 4° C. for 30 min in the dark. Cells were washed and fixed in 1:3 dilution of BD Cytofix fixation buffer (BD Pharmingen. San Diego CA). They were analyzed by a BD FACS Celesta (BD Biosciences. San Jose. CA) and DiVa software. Post-acquisition analyses were performed with FlowJo (TreeStar. Ashland. OR). Data shown in the figures represents % of IFN-γ production by CD8⁺ or CD44⁺CD8⁺ cells upon peptide stimulation. Background values obtained for the same cells cultured without peptide(s) were subtracted. Sometimes responses were only analyzed to the immunodominant epitope of E7 using either the specific peptide for stimulation of staining cells directly ex vivo with T cell identifying antibodies or an MHC class I tetramer or dextramer able to identify the E7-specific T cell receptor. Melanoma vaccine immunized mice were probed with an MHC class I tetramer for responses to an immunodominant TRP-1 epitope.

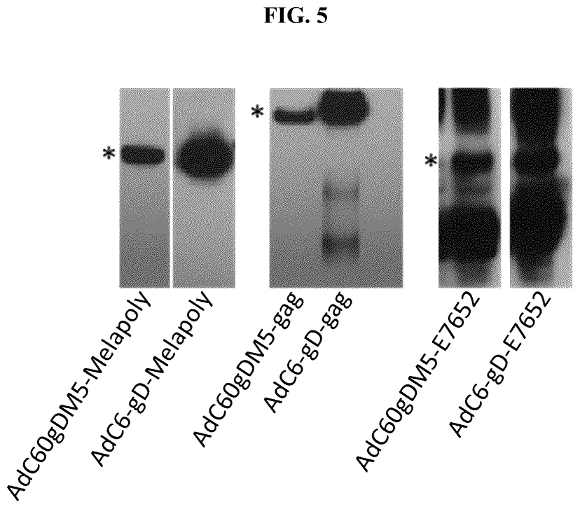

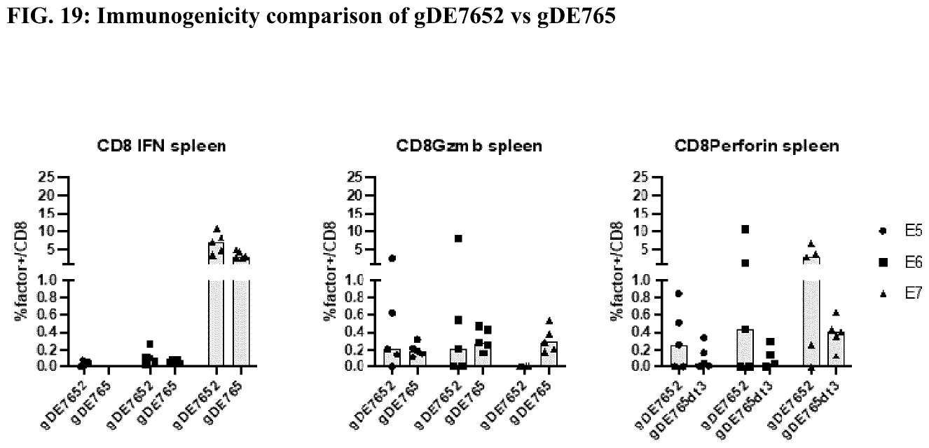

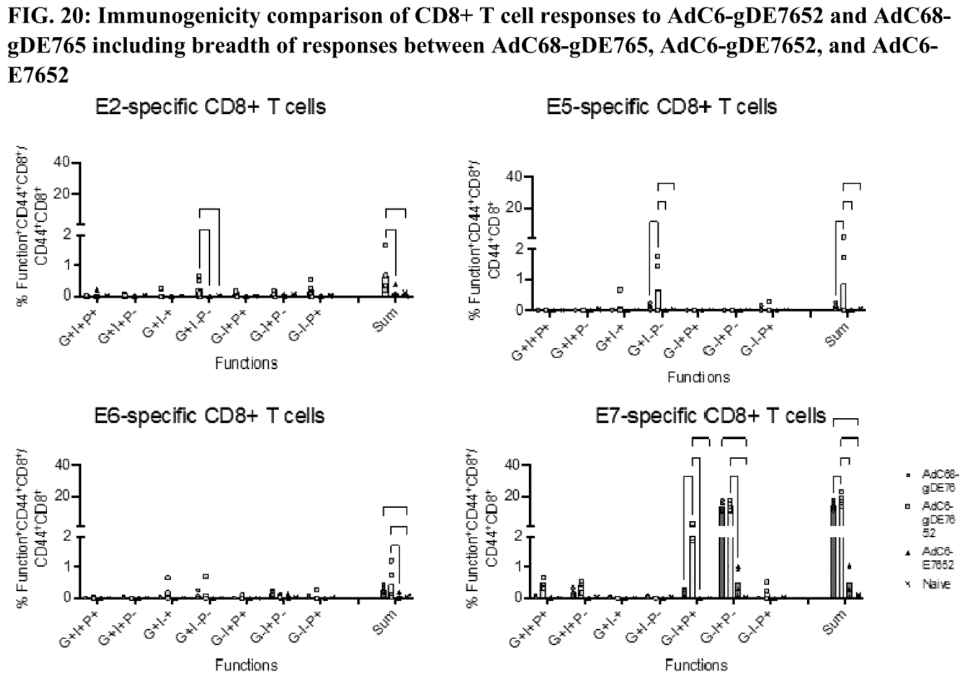

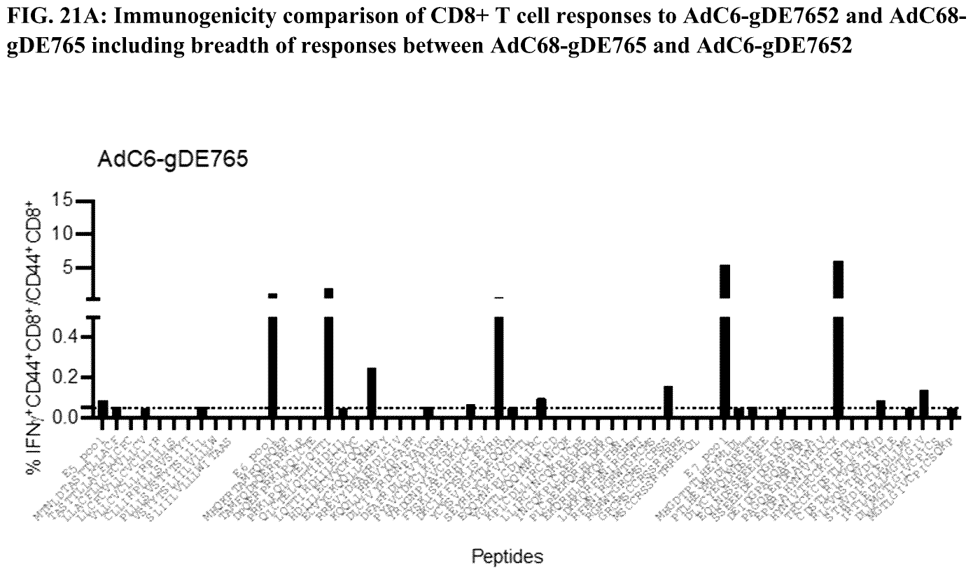

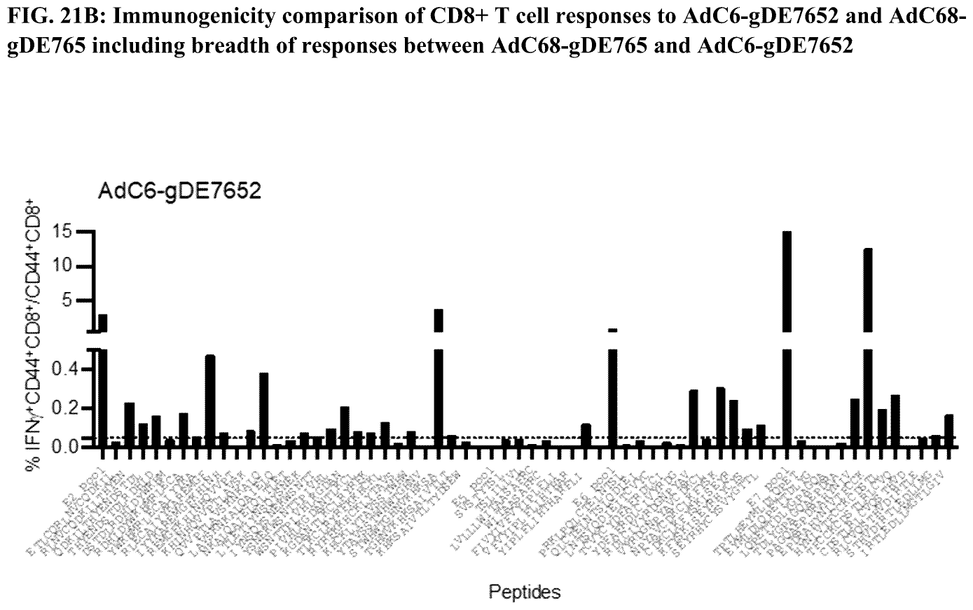

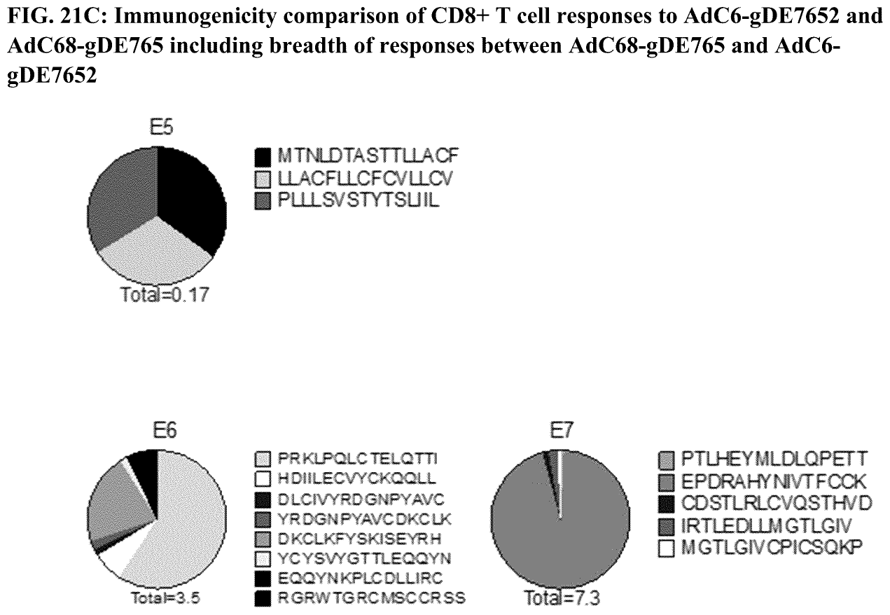

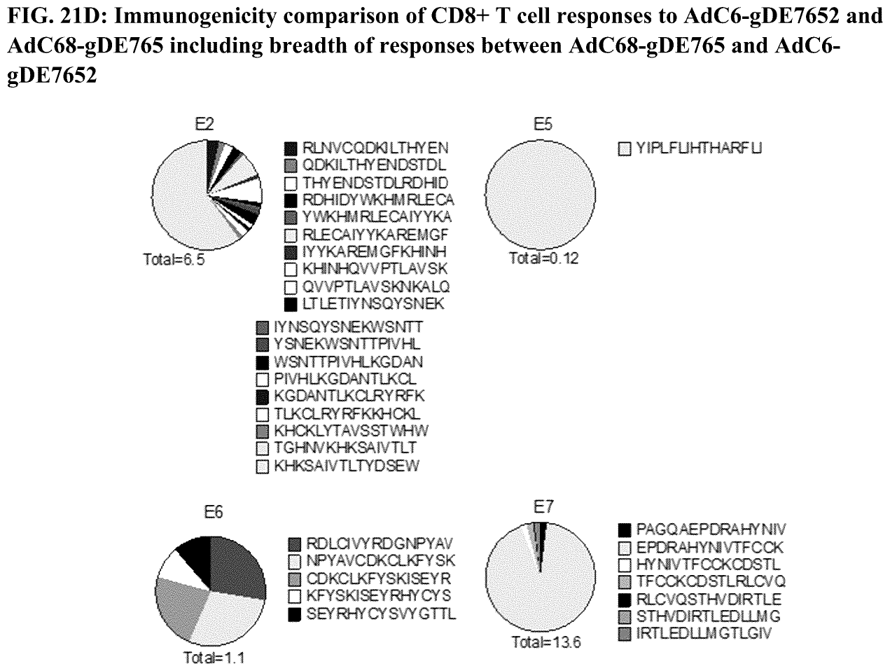

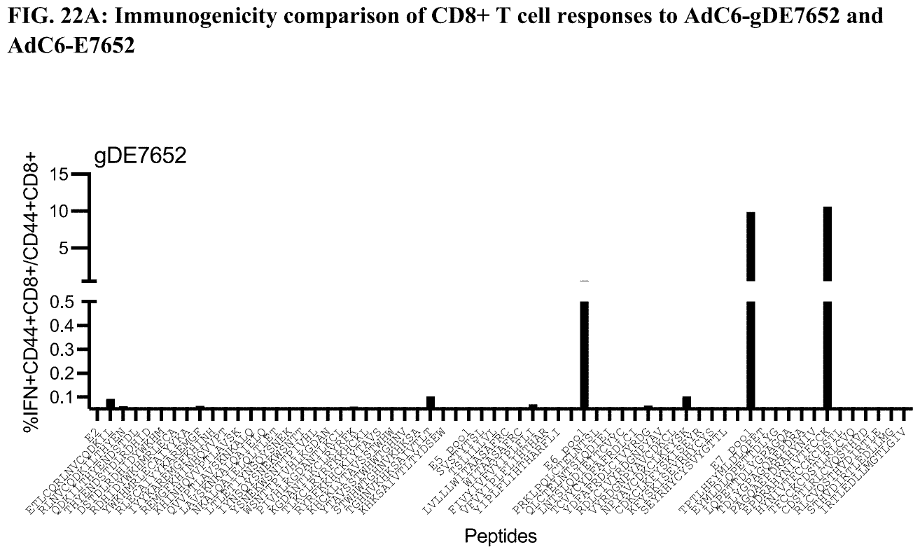

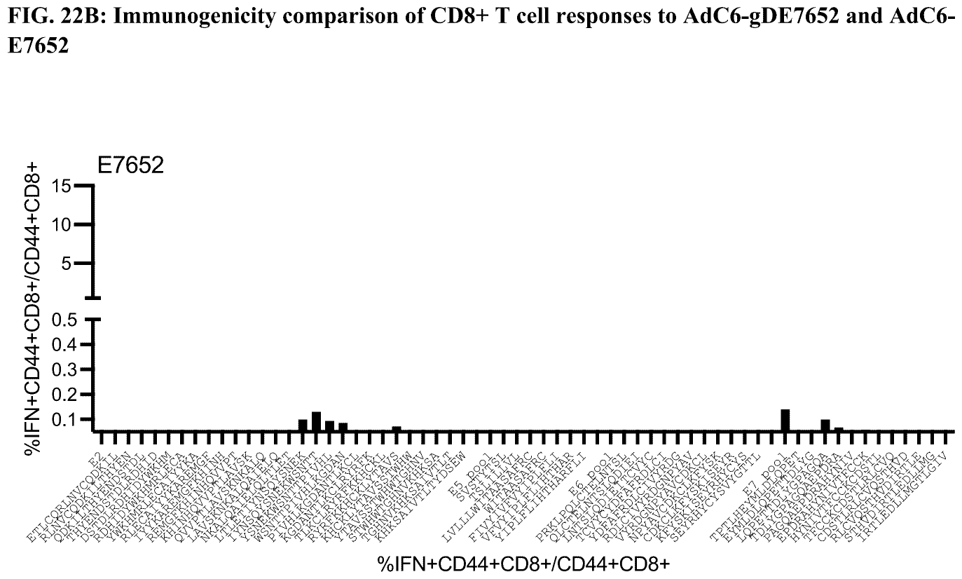

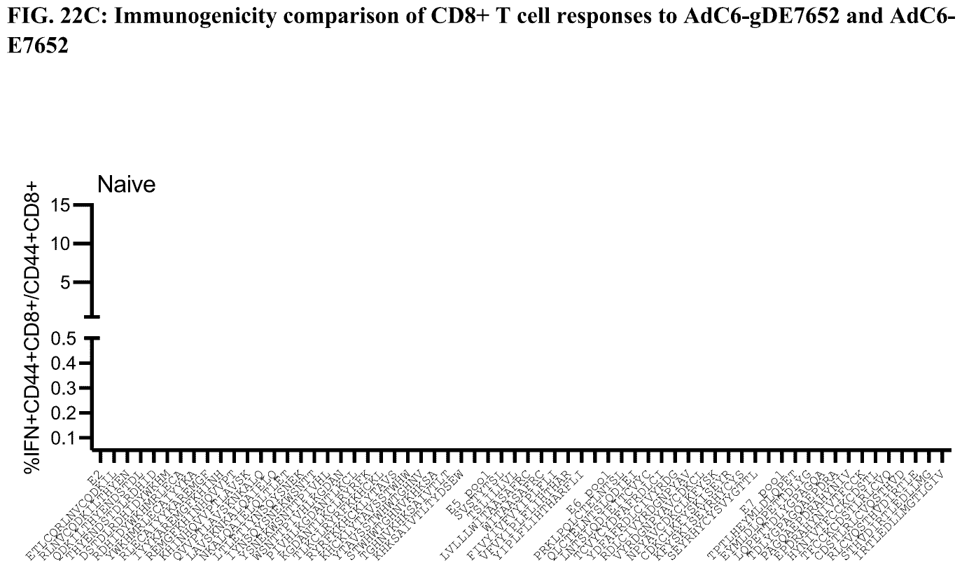

As shown in FIG. 19, FIG. 20, FIG. 21A, FIG. 21B, FIG. 21C, and FIG. 21D, HPV vectors expressing E7, E6, E5 and E2 fused into gD were slightly more immunogenic than vectors expressing E7, E6, and E5 fused into gD. As shown in FIGS. 5 and 6 vectors expressing E7, E6, E5 and E2 without gD were poorly immunogenic.

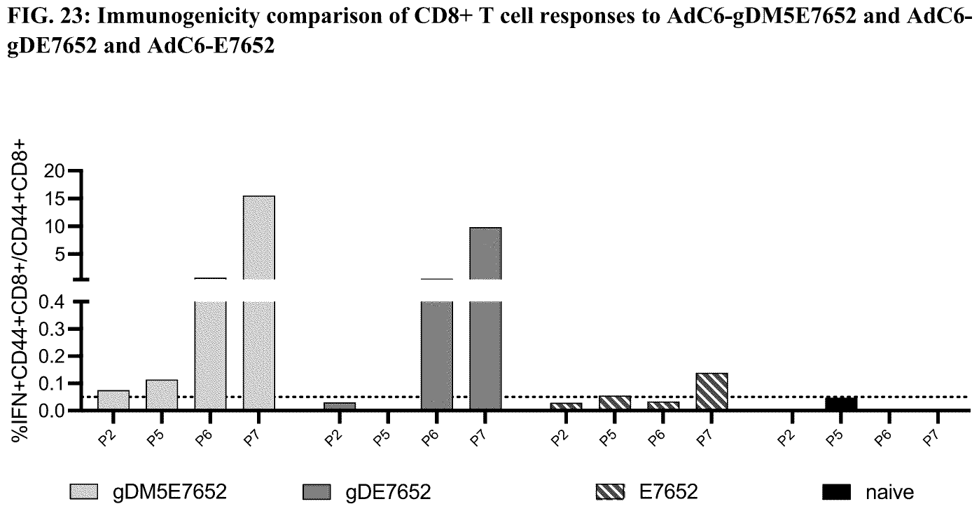

As shown in FIG. 23 vectors expressing E7, E6, E5 and E2 in HSV gDM5 were slightly more immunogenic than the vectors expressing the same inserts fused into gD.

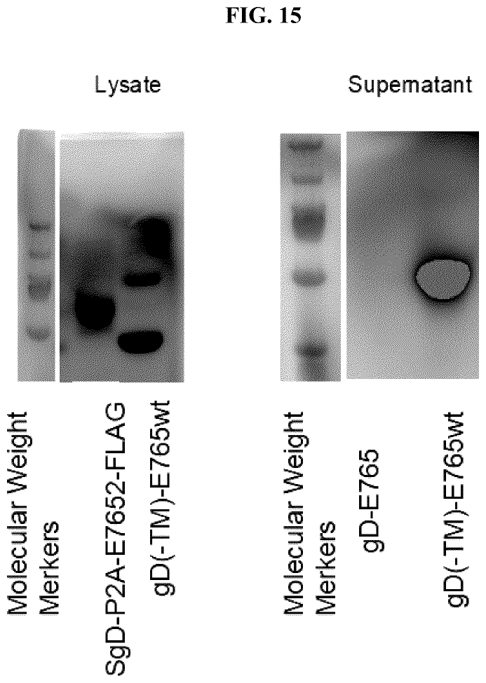

Expression of the vaccine inserts was tested for by flow cytometry as shown in FIG. 1 or Western Blots as shown in FIG. 2, FIG. 5, and FIG. 15.

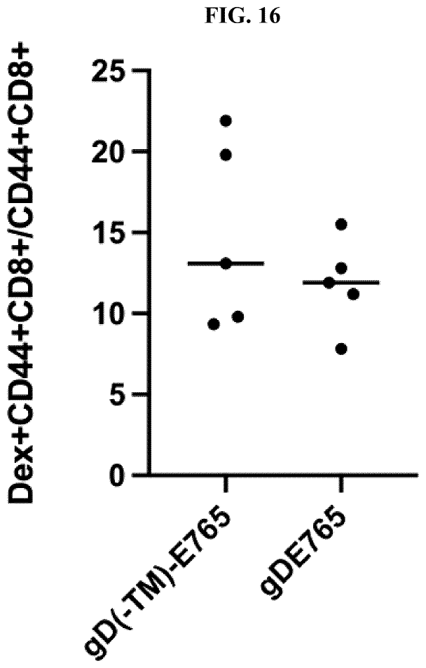

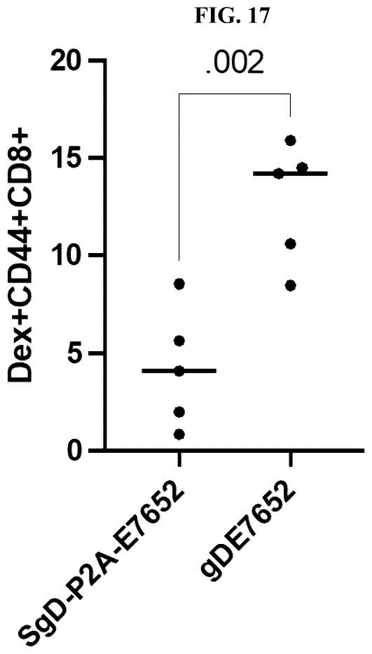

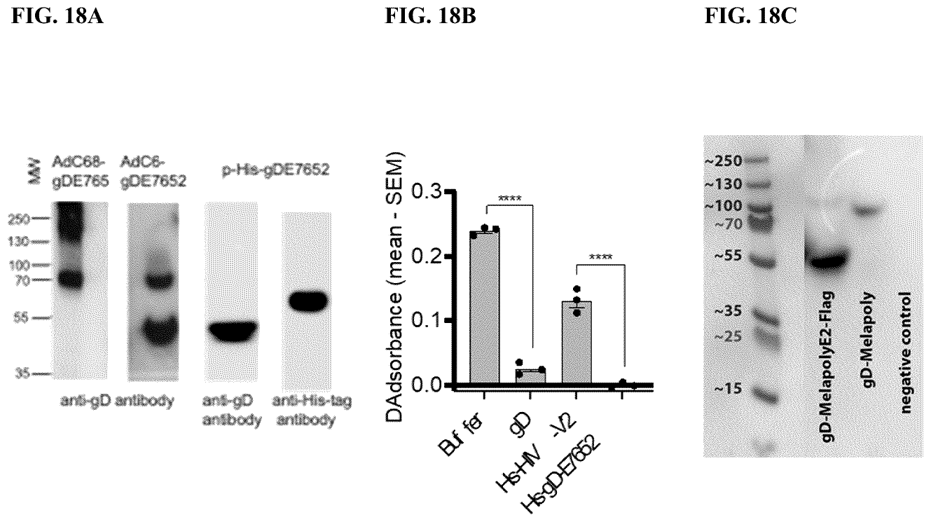

FIG. 3, FIG. 4A, FIG. 6, FIG. 7, FIG. 8 FIG. 9A, FIG. 9B, FIG. 9C, FIG. 9D, FIG. 16, and FIG. 17 show the results for C57Bl/6 mice. In FIG. 4A, pooled spleens from mice immunized with 5×10¹⁰ virus particles (vp) of the indicated vectors were tested 5 weeks later for interferon-gamma (IFN-g) production in response to peptide pools representing the inserted sequences of E7, E6, E5 or E2. FIG. 9A, FIG. 9B, FIG. 9C, and FIG. 9D show the results of splenocytes that were stimulated with individual peptides.

FIG. 4B, FIG. 10A, and FIG. 10B show results for HLA-A02 transgenic mice immunized with 1×10⁹ vp of the indicated vectors. Mice were tested 6 weeks later against the E7 peptide pool as shown in FIG. 4B. FIG. 10A and FIG. 10B shows responses of splenocytes to individual peptides of pools for E2, E5, E6 and E7.

Protection against TC-1 tumor cell challenge was tested in a pre-challenge vaccination model. Groups of 10 C57Bl/6 mice were vaccinated with 10¹⁰ vp of the AdC6-gDM5-E7652 vector or a control vector expressing HIV-1 gag genetically fused into gDM5. Mice were challenged 4 weeks later with 5×10⁵ TC1 cells given subcutaneously and tumor progression was recorded.

As shown in FIG. 11, which shows tumor volume over time for individual mice with lines indicating means, all the control mice rapidly developed tumors while all the AdC6-gDM5-E7652 mice remained tumor-free.

Protection against TC-1 tumor cell challenge was tested in a post-challenge vaccination model in FIG. 12A, FIG. 12B, FIG. 14A, FIG. 14B, FIG. 14C, and FIG. 14D. Groups of C57Bl/6 mice were challenged with 5×10⁴ TC1 cells. Three days later they were vaccinated with 10¹⁰ vp of the AdC6-gDM5-E7652 vector or a control vector expressing HIV-1 gag genetically fused into gDM5 and tumor growth was recoded. As shown in FIG. 12A, which shows tumor volumes over time, all mice initially developed tumors, but while tumors continued to grow in the control mice the vaccinated mice rapidly controlled tumor growth and were tumor-free by 23 days after vaccination. They remained tumor-free until the end of the experiment (day 73). This is shown in FIG. 12B which shows the percentage of tumor-free mice over time.

These results compare favorably to those were generated with a previous vaccine expressing modified versions of E7, E6 and E5 fused into wild type gD by the chimpanzee adenovirus vector AdC68, which only achieved 50% protection of mice if used at the 10¹⁰ vp vector dose in mice that had been challenged 3 days earlier with 5×10⁴ TC1 cells. FIG. 13A shows tumor volume of individual mice over time and FIG. 13B shows % tumor free mice over time.

In another experiment, mice were challenged with a higher dose of 2×10⁵ TC1 cells prior to vaccination with 10¹⁰ vp of AdC6-gDM5-E7652, AdC6-gDM5-E765dt3, or the control vector AdC6-gDM5-gag. All the control mice developed tumors that rapidly progressed. This is shown in FIG. 9A, FIG. 9B, and FIG. 9C which show tumor volumes for individual mice over time. FIG. 9D shows percent tumor-free mice over time.

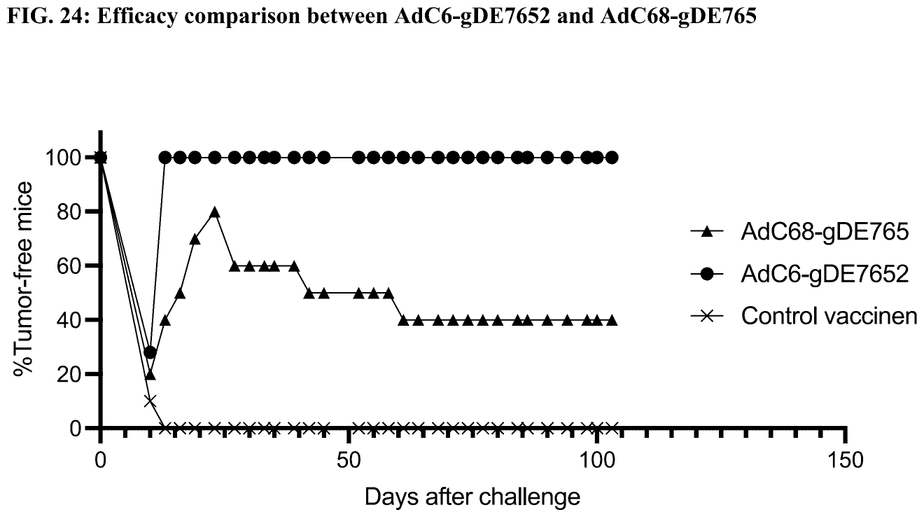

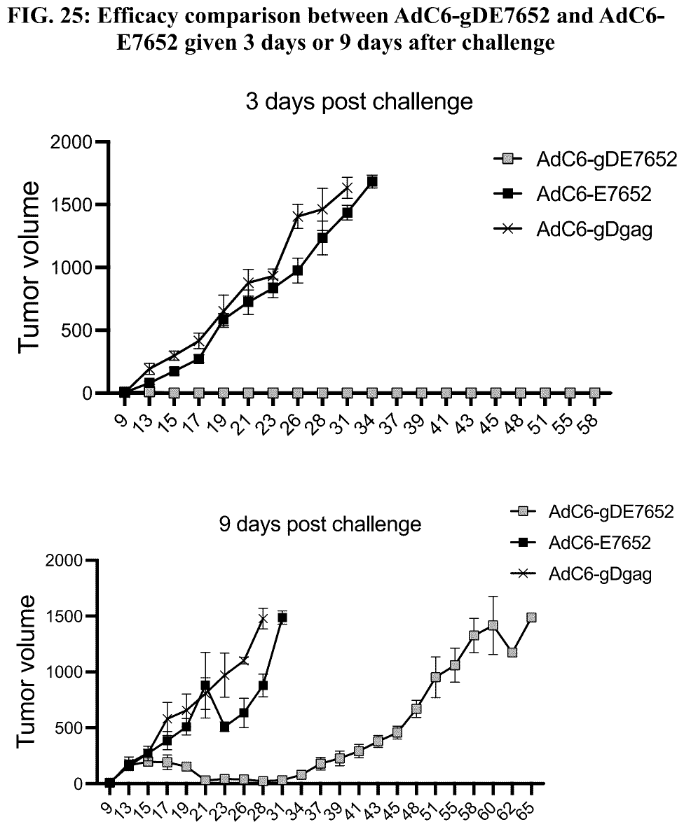

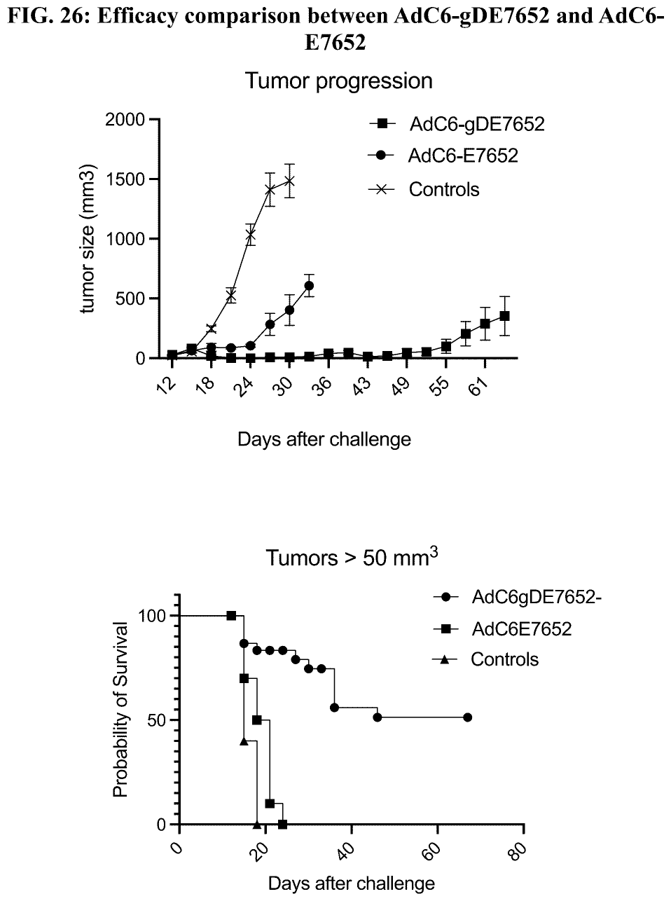

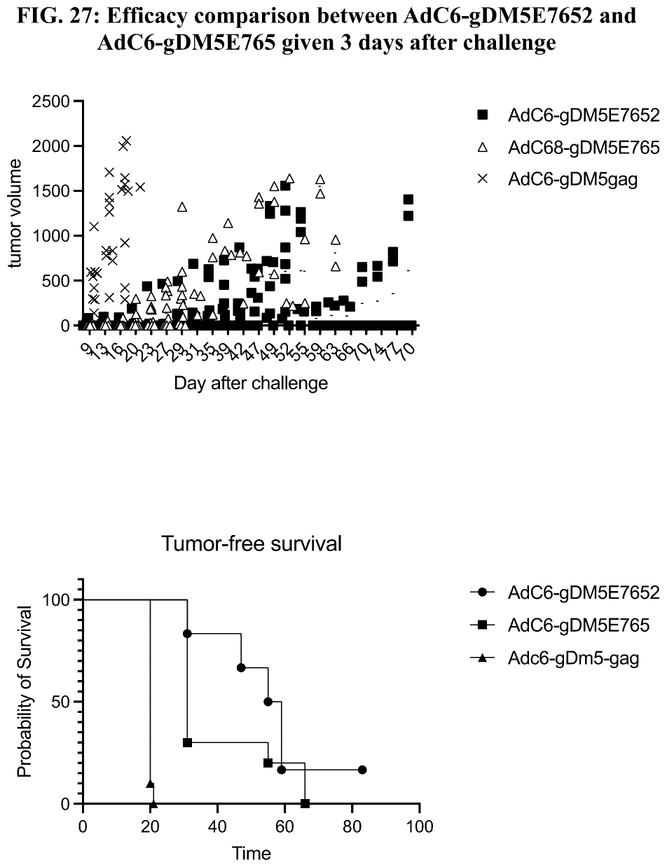

The data show protection against TC-1 tumor cell challenge. TC-1 cells express E7 and E6 of HPV 16 but they do not express E5 or E2. In most experiments, vectors were given 3 days (FIG. 24, FIG. 25, FIG. 27, FIG. 12A, FIG. 12B, FIG. 14A, FIG. 14B, FIG. 14C, and FIG. 14D) or 9 days (FIG. 25, FIG. 26) after tumor challenge. FIG. 11 shows results of a pre-challenge vaccination model.

Numbers of TC-1 cells used for subcutaneous challenge varied. 2×10⁵ cells were used for FIG. 24, FIG. 25, FIG. 27 FIG. 13; 5×10⁴ cells for FIG. 26, FIG. 12A and FIG. 12B; and 5×10⁵ cells for FIG. 11. Mice were vaccinated with 1×10¹⁰ vp of vectors. After challenge tumor sizes were measured in 2-3 day intervals. Mice were euthanized once tumors exceeded a volume of 2 cm³. The data show that all mice that received control vectors rapidly developed tumors.

FIG. 24 shows that the AdC6-gDE7652 achieved complete and sustained remission in all mice while the AdC68-gDE765 vector only achieved remission that was sustained in 40% of mice. This difference in efficacy is unlikely to reflect a difference in the vector backbone (AdC6 vs. AdC68) as these two vectors are closely related and tend to give comparable results. Rather, it is presumed that the difference is related to the presence of the E2 sequence. E2 has two domains—one is a DNA binding domain that activates hundreds of host cell genes which conceivable could influence immune response. Another domain of E2 has a splice site which causes rapid degradation of the protein. This in turn would separate the HVEM binding site of gD from the C-terminus of gD which contains a transmembrane domain. This in turn could affect intracellular trafficking of the fusion protein or fragments thereof and thereby affect antigen presentation including presentation by cross-presentation.

FIG. 25 shows vaccine efficacy against tumor progression comparing the AdC6-gDE7652 insert to an E7652 (without gD) insert after a challenge given 3 days or 9 days before vaccination. Results clearly show that the latter failed to protect while the former provided complete protection to mice challenged 3 days before vaccination and partial protection to mice vaccinated 9 days after challenge demonstrating that gD is essential to induce an immune response that prevents tumor growth.

The same comparison is made in FIG. 26 which used a lower tumor cell dose than FIG. 24. In this experiment, the E7652 vector afforded some delay of tumor cell growth but again inserting this sequence into gD markedly increased vaccine efficacy.

FIG. 27 shows vector efficacy comparing AdC6-gDM5E7652 to AdC68-gDM5E765. Again, insertion of E2 induced an immune response that more effectively delayed tumor progression.

FIG. 11 shows that the AdC6-gDM5E7652 vector given 4 weeks before tumor challenge completely prevented tumor formation.

FIG. 12A and FIG. 12B show that upon lowering the tumor cell challenge dose, the AdC6-gDM5E7652 vector could completely prevent tumor growth in all mice.

FIG. 14A, FIG. 14B, FIG. 14C, and FIG. 14D compare the efficacy of the AdC6-gDM5E7652 vector to that of the AdC68-gDM5E765 vector. Again, the presence of E2 improved vaccine efficacy.

Additional data were generated from lymphocytes of mice challenged with TC-1 cells that were than vaccinate 3 days or 9 days later with 1×10¹⁰ vp of the Ad vectors given intramuscularly.

The assays were conducted with PBMCs, splenocytes and purified tumor infiltrating lymphocytes (TILs). PBMCs and splenocytes were collected and purified as described herein. To obtain tumor-infiltrating lymphocytes, tumors were harvested, cut into small fragments and treated with 2 mg/ml Collagenase P, 1 mg/ml DNase I (all from Roche) and 2% FBS (Tissue Culture Biologicals) in Hank's balanced salt solution (HBSS, 1X, Thermo Fisher Scientific) under agitation for 1 hour. Tumor fragments were homogenized, filtrated through 70 μm strainers and lymphocytes were purified by Percoll-gradient centrifugation and washed with DMEM supplemented with 10% FBS. Following stimulation, cells were incubated with anti-CD8-APC (clone 53-6.7. BioLegend. San Diego CA), anti-CD4-BV605 (clone RM4-5. BioLegend), anti-CD44-Alexa Flour 700 (clone IM7, BioLegend) and violet live/dead dye (Thermo Fisher Scientific) at 4° C. for 30 min in the dark. Cells were washed once with PBS and then fixed and permeabilized with Cytofix/Cytoperm (BD Biosciences. San Jose, CA) for 20 min. Cells were then incubated with an anti-IFN-γ-FITC antibody (Clone, XMG1.2 BioLegend) at 4° C. for 30 min in the dark an antibody to mouse perforin and an antibody to granzyme B. Cells were washed and fixed in 1:3 dilution of BD Cytofix fixation buffer (BD Pharmingen. San Diego CA) and analyzed by a BD FACS Celesta (BD Biosciences. San Jose, CA) and DiVa software. Post-acquisition analyses were performed with FlowJo (TreeStar, Ashland, OR). Data shown in the figures represents % of IFN-γ production by CD8+ or CD44+CD8+ cells upon peptide stimulation. Background values obtained for the same cells cultured without peptide(s) were subtracted.

In some experiments, lymphocytes were stained with the above mentioned antibodies to CD8 and CD44, an APC-labeled MHC class I tetramer or MHC class I dextramer for the immunodominant epitope of E7. In some of these experiments additional stains for KLRG1 (PerCP/Cyanided 5.5 labeled, clone 2F1/KLRG1 from Biolegend), PD1 (BUV295 labeled, clone J43 from Biolegend), TIM3 (BV785 labeled, clone RMT3-23 from Biolegend), CTLA4 (PE labeled, clone UC10-4B9 from Biolegend), TIGIT (PE/Dazzle 594 labeled, 1G9 from Biolegend), and LAG3 (PE/Fire labeled, clone C9BW7 from Biolegend) were included. After staining cells were washed an analyzed. Post-acquisition analyses by FlowJo gated Dextramer positive CD8+ T cells on cells that were positive for a given marker.

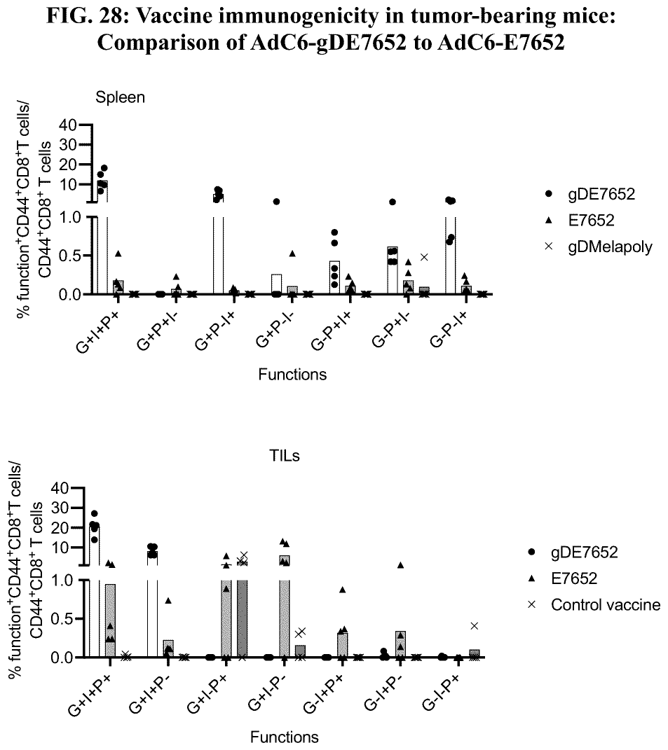

FIG. 28 shows T cell responses from spleens and tumors in response to the AdC6-gDE7652 and AdC6-E7652 or a control vector given to tumor bearing mice. The cells were analyzed by ICS for IFN-g, perforin and granzyme B. The graphs show frequencies of CD8+CD44+ T cells over all CD44+CD8+ T cells that were positive for the different combinations of function. The graphs show that both in spleen and tumors the gDE7652 vector induced higher frequencies of mainly polyfunctional CD8+ T cells that the E7652 vector.

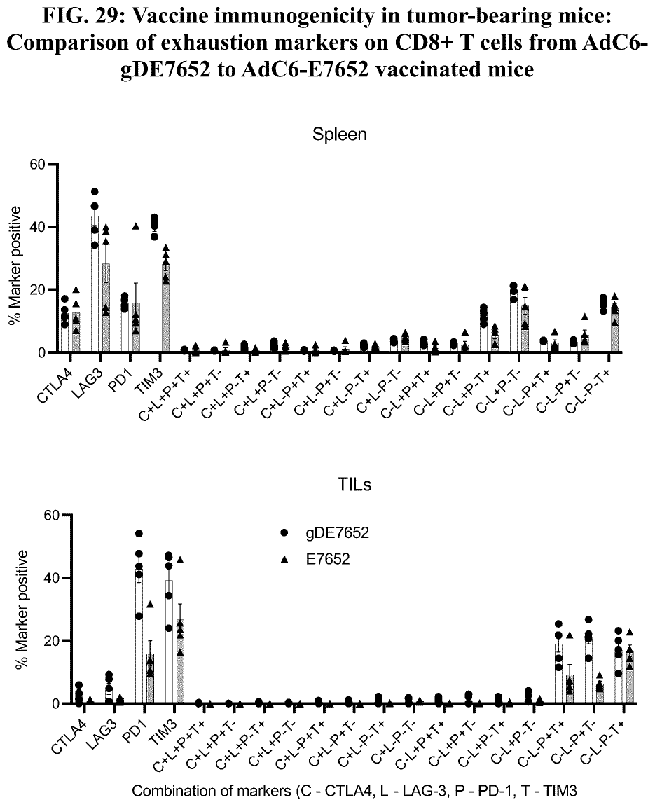

For FIG. 29, lymphocytes from the same mice described in FIG. 28 were stained with an MHC class I E7-specific dextramer and antibodies for activation/exhaustion markers. The graphs show that most markers were expressed on higher percentages of gDE7652 than E7652 induced CD8+ T cells.

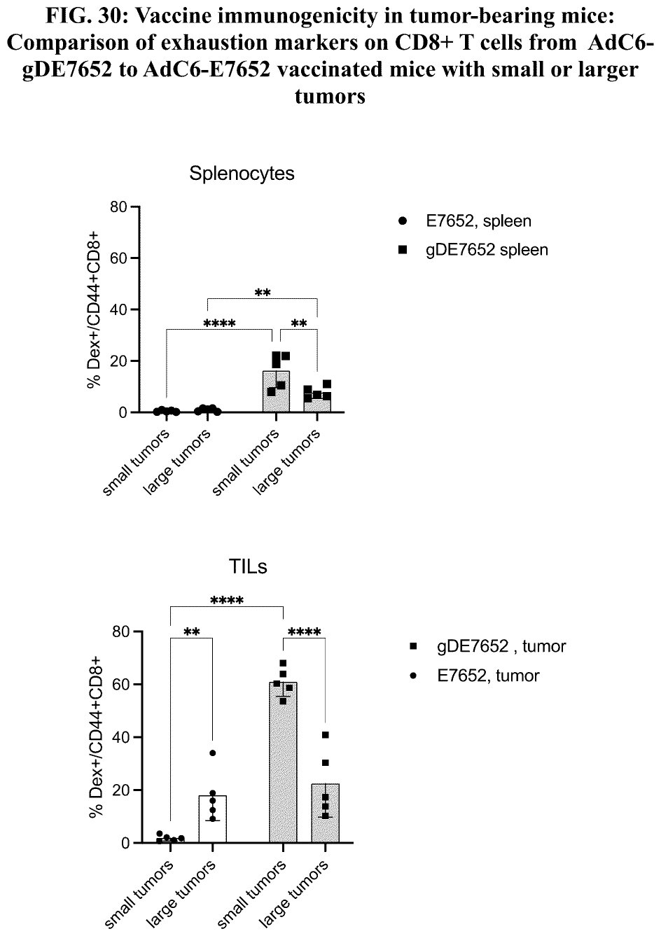

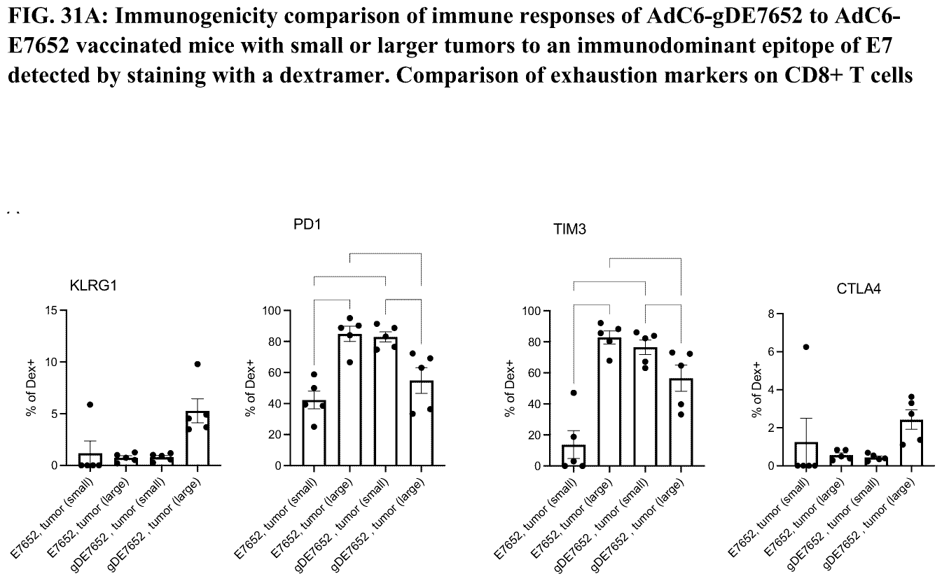

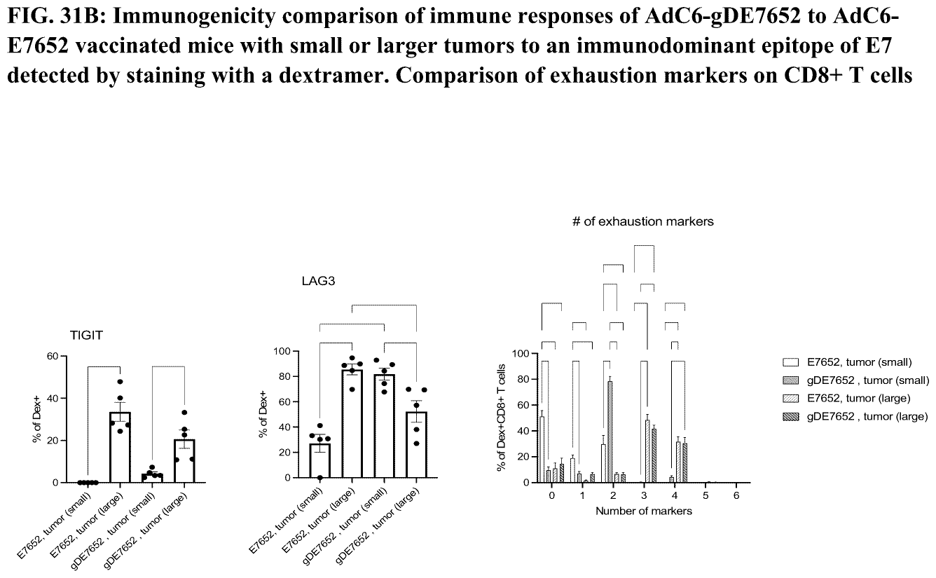

In FIG. 30, T cell frequencies to the immunodominant epitope of E7 tested for by staining for T cell markers and the MHC class I E7-specific dextramer were compared from spleens and tumors vaccinated with gDE7652 or E7652 once mice had developed small or large tumors. In mice with small tumors the gDE7652 vector achieved higher frequencies of E7-specific CD8+ T cells. This difference was no longer seen in large tumors presumable reflecting that cells were isolated earlier from the E7652 vaccinated than the gDE7652 vaccinated mice.

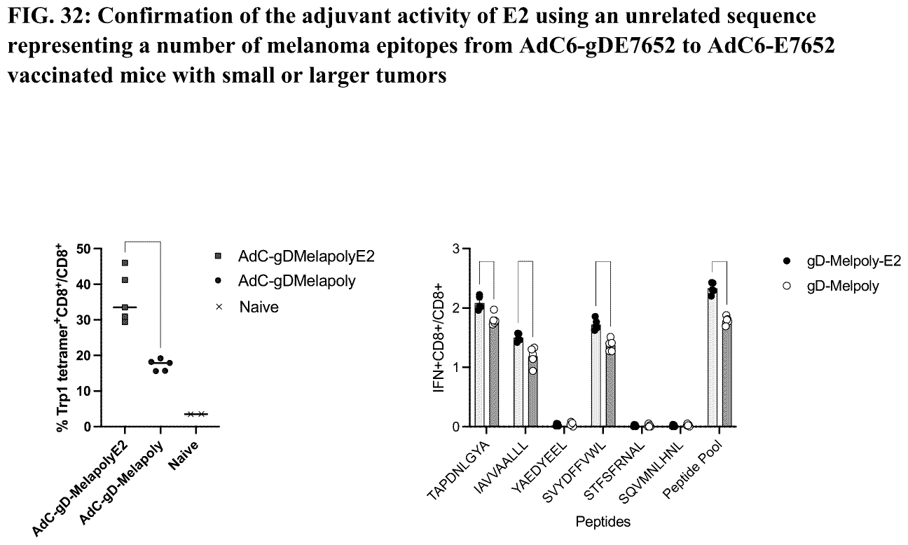

FIG. 32A and FIG. 32B show results from the same T cell populations shown in FIG. 30. T cells were stained with the dextramer and antibodies to activation/exhaustion markers. Several of the markers were higher expressed on E7-specific CD8+ T cells from large tumors of E7652 than gDE7652 vaccinated mice, while in small tumors CD8+ T cells from gDE7652 vaccinated mice tended to have higher expression levels. This presumably reflects that most of the markers are indicative not only for exhaustion but also activation and that at the earlier time point in mice with small tumor the gD adjuvanted vector achieved a higher level of activation. The percentages of CD8+ T cells expressing different numbers of the markers were subtle and mainly observed comparing small tumors between the two vector groups.

FIG. 32 shows a comparison of the magnitude of CD8+ T cell responses to a melanoma epitope vector termed Melapoly that was expressed by itself within gD or expressed as a fusion protein with E2 within gD. The study was designed to further test if E2 serves as an adjuvant of co-expressed with a different sequence. This was confirmed as the inclusion of E2 significantly enhanced the CD8+ T cell response top one of the melanoma epitopes.

Analyses of protein expression by flow cytometry in FIG. 1 using an anti-gD antibody shows comparable expression of the gD-E765dt1, gDM1-E765dt1 and gDM2-E765dt1 fusion proteins while gDM3-E765 was poorly expressed.

Analyses of protein expression by Western Blot in FIG. 2 using an anti-gD antibody shows slower expression of the gDM5-E7652 than the gD-E7652 fusion protein.

For testing of the gDM1 and gDM2 vectors, groups of 10 C57Bl/6 mice were injected with 1×10¹⁰ vp of the AdC68 vector expressing a modified version of HPV 16 E765 fusion protein (called dt1) fused either into wild-type gD or the gDM1 or gDM2 mutants. CD8+ T cell responses were tested 14 days and 28 days later using peripheral blood mononuclear cells (PBMCs). Cells were isolated and purified and stained with antibodies to CD8, CD44, a tetramer to the immunodominant epitope pf E7 and a live cells stain. Cells were then analyzed by flow cytometry. As shown in FIG. 3, the mutant gD vector induced lower CD8+ T cell responses compared to the original gD.

T cell responses to the gDM5 mutant was tested in comparison to wild-type gD or no gD using an HPV insert that contained immunogenic fragments of HPV 16 E7, E6, E5 and E2 in C57Bl/6 mice using peptide pools for the 4 inserted oncoprotein fragments or in HLA-A02 transgenic mice using peptides of E7 only. As shown in FIG. 4, the gD5 vectors tended to be slightly more immunogenic.

Lysates of HEK 293 cell transduced with Ad vectors expressing the indicated proteins were tested by Western blot for expression of the insert using an antibody to gD. As shown in FIG. 5 the gDM5 vectors expressed as expected a protein that was slightly smaller than that expressed by the corresponding gD carrying vectors. Under the experimental conditions (48-hour transduction of HEK293 cells with 1000 VP of vector/cell) expression levels tended to be higher with the gD vectors for Melapoly, gag and E7652 as shown in FIG. 5.

CD8+ T cell responses were tested using lymphocytes from mice injected with 1×10¹⁰ vp of AdC6-gD-Melapoly #2 or AdC6-gDM5- Melapoly #2. Splenocytes were tested 17 days later for responses to the immunodominant epitope present within Melapoly for ICS for IFN-γ. As shown in FIG. 6 the AdC6-gDM5-Melapoly #2 vector tended to induce a slightly higher response than the AdC6-gD-Melapoly #2 vector (p value by 2-way Anova=0.076).

Mice (n=5) were injected with 1×10⁹ vp of the AdC6-gD-PolN or AdC6-gDM5-PolN vectors. Four weeks later splenocytes pooled from the 5 mice were tested by ICS for IFN-γ production in response to the peptide pool or individual peptides. Results in FIG. 7 show the response against the pool and the most immunodominant peptides. Responses were consistently higher in response to the AdC6-gDM5-PolN vector.

Groups of mice were injected with 2×10¹⁰ vp of the AdC68-gD-E765 or the AdC68-gDM5-E765 vectors. Splenocytes were tested 4 weeks later by ICS for CD8+ T cell responses to peptide pools representing the E7 sequence. As shown in FIG. 8, the AdC68-gDM5-E765 vector induced significantly higher T cell responses compared to the AdC68-gD-E765 vector.

Groups of mice were injected with 2×10¹⁰ vp of the AdC68-gDM5-E765, AdC68-gD-E765, AdC68-E765 or nothing. Splenocytes were tested 4 weeks later by ICS for CD8+ T cell responses to individual peptides the HPV 16 oncoproteins' sequences. As shown in FIG. 9A, FIG. 9B, FIG. 9C, and FIG. 9D responses in naïve mice and mice vaccinated with the AdC68-gDM5-E765 vector were significantly high than in mice vaccinated with the E765 vector without gD which responses to the vectors expressing the E765 sequence within gD or gDM5 were largely comparable.

CD8+ T cell responses to the inserted antigen were tested for induction of CD8+ T cell responses HLA-A02 transgenic mice using individual peptides the HPV 16 oncoproteins' sequences. As shown in FIG. 10A, and FIG. 10B the gDM5 vectors tended to be slightly more immunogenic than the gD vectors.

The data show protection against TC-I tumor cell challenge in a pre-challenge vaccination model. Groups of 10 C57Bl/6 mice were vaccinated with 1×10¹⁰ vp of the AdC6-gDM5-E7652 vector or a control vector expressing HIV-1 gag genetically fused into gDM5. Mice were challenged 4 weeks later with 5×10⁵ TC1 cells given subcutaneously and tumor progression was recorded. As shown in FIG. 11, all the control mice rapidly developed tumors while all the AdC6-gDM5-E7652 mice remained tumor-free.

The data show protection against TC-1 tumor cell challenge in a post-challenge vaccination model. Groups of C57Bl/6 mice were challenged with 5×104 TC1 cells. Three days later they were vaccinated with 1×10¹⁰ vp of the AdC6-gDM5-E7652 vector or a control vector expressing HIV-1 gag genetically fused into gDM5 and tumor growth was recoded. As shown in FIG. 12A and FIG. 12B all mice initially developed tumors, but while tumors continued to grow in mice immunized with the control vector, i.e., the AdC6-gDM5-gag vector all the AdC6-gDM5-E7652 immunized mice showed remission and failed to develop tumors at later times.

The data show protection against TC-1 tumor cell challenge in a post-challenge vaccination model. Groups of C57Bl/6 mice were challenged with 2×10⁵ TC1 cells. Three days later they were vaccinated with 10¹⁰ vp of AdC6-gDM5-E7652, AdC6-gDM5-E765, or the control vector AdC6-gDM5-gag and tumor growth was recoded. As shown FIG. 14C all control mice rapidly developed tumors. Which as shown in FIG. 14A, tumor progression was delayed in mice vaccinated with Ad AdC6-gDM5-E7652, several of which completely controlled tumor growth. Tumor progression was also delayed upon vaccination with the AdC68-gDM5-E765 vector although in this group all mice developed tumors as shown in FIG. 14B. FIG. 14D shows the same data as tumor free survival over time and again shows a tumor growth delay in the 2 vector groups and complete protection in 40% of mice that received the AdC6-gDM5-E7652 vector.

FIG. 15 shows the results of Western Blot analyses. FIG. 15 shows that the Ad vector encoding the SgD-PA2-E765 wt fusion protein expresses an anti-gD antibody binding protein that corresponds to the size of SgD indication that SgD and the E7652 insert were generated from the same Ad vector as two separate polypeptide chains. FIG. 15 also shows that the Ad vector encoding gD(-T)-E765 wt induces a small amount of protein that can be detected in cell lysate but as shown in FIG. 15 most of the protein is secreted and can be detected in the supernatant.

CD8+ T Cell responses to the gD(-TM)-E765 insert vector were tested in comparison to those induced by the vector encoding the mutant E765. As shown in FIG. 16 both inserts induced CD8+ T cell responses of comparable magnitude. These results show that the transmembrane domain is not needed for the adjuvant effect of gD which can be delivered by a secreted protein. This will be useful for construction of Ad vectors that carry a lengthy antigen within gD. The packaging capacity of Ad virus vectors is limiting and shortening gD by removing the transmembrane domain will allow for insertion of longer sequences encoding the antigen.

CD8+ T Cell responses to the SgD-P2A-E7652 insert expressing vector were tested in comparison to those induced by the vector expressing the mutant gDE7652. As shown in FIG. 16 the SgD-P2A-E7652 insert induced a significantly lower CD8+ T cell response than the gD-E7652 insert vector. These results show that for gD to exert its adjuvant effect on CD8+ T cells to a co-expressed antigen both the antigen and gD must be expressed as a fusion protein. This may reflect that antigen presentation of Ad vector delivered transgene products relays primarily on cross-presentation. For gD to enhance T cell receptor signaling to an immunogenic peptide displayed by an MHC class I antigens, both the MHC-antigen complex and gD has to be expressed by the same antigen presenting cells. If the two polypeptides, i.e., gD and the antigen, are produced by the same transduced cell as two separate proteins, many of them are likely to be taken up, processed, and presented by different antigen presenting cells.

Those skilled in the art will appreciate that numerous changes and modifications can be made to the preferred embodiments disclosed herein and that such changes and modifications can be made without departing from the spirit of the invention. It is, therefore, intended that the appended claims cover all such equivalent variations as fall within the true spirit and scope of the invention.

TABLE 23
Sequences

| | | |
|---|---|---|
| gDM1 | SEQ ID | MGGAAARLGAVILFVVIVGLHGVRGKYCLADASLKMADPN |
| Protein | NO: 1 | RFRGKDLPVLDQLTDPPGVRRVYHIQAGLPDPFQPPSLPITVC |
| | | YAVLERACRSVLLNAPSEAPQIVRGASEDVRKQPYNLTIAWF |
| | | RMGGNCAIPITVMEYTECSYNKSLGACPIRTQPRWNYYDSFS |
| | | AVSEDNLGFLMHAPAFETAGTYLRLVKINDWTEITQFILEHR |
| | | AKGSCKYALPLRIPPSACLSPQAYQQGVTVDSIGMLPRFIPEN |
| | | QRTVAVYSLKIAGWHGPKAPYTSTLLPPELSETPNATQPELA |
| | | PEDPEDSALLEDPVGTVAPQIPPNWHIPSIQDAATPYHPPATP |
| | | NNMGLIAGAVGGSLLAALVICGIVYWMHRRTRKAPKRIRLP |
| | | HIREDDQPSSHQPLFY |
| gDM1 | SEQ ID | atggggggggctgccgccaggttgggggccgtgattttgtttgtcgtcatagtgggcctccatgg |
| Nucleotide | NO: 2 | ggtccgcggcaaatattgtttggcggatgcctctctcaagatggccgaccccaatcgctttcgcg |
| | | gcaaagaccttccggtcctggaccagctgaccgaccctccgggggtccggcgcgtgtaccaca |
| | | tccaggcgggcctaccggacccgttccagccccccagcctcccgatcacggtttgctacgccgt |
| | | gttggagcgcgcctgccgcagcgtgctcctaaacgcaccgtcggaggccccccagattgtccg |
| | | cggggcctccgaagacgtccggaaacaaccctacaacctgaccatcgcttggtttcggatggga |
| | | ggcaactgtgctatccccatcacggtcatggagtacaccgaatgctcctacaacaagtctctggg |
| | | ggcctgtcccatccgaacgcagccccgctggaactactatgacagcttcagcgccgtcagcga |
| | | ggataacctggggttcctgatgcacgcccccgcgtttgagaccgccggcacgtacctgcggctc |
| | | gtgaagataaacgactggacggagattacacagtttatcctggagcaccgagccaagggctcct |
| | | gtaagtacgccctcccgctgcgcatccccccgtcagcctgcctctccccccaggcctaccagca |
| | | gggggtgacggtggacagcatcgggatgctgccccgcttcatccccgagaaccagcgcaccg |
| | | tcgccgtatacagcttgaagatcgccgggtggcacgggcccaaggccccatacacgagcacc |
| | | ctgctgcccccggagctgtccgagacccccaacgccacgcagccagaactcgccccggaaga |
| | | ccccgaggattcggccctcttggaggaccccgtggggacggtggcgccgcaaatcccaccaa |
| | | actggcacatcccgtcgatccaggacgccgcgacgccttaccatcccccggccaccccgaaca |
| | | acatgggcctgatcgccggcgcggtgggcggcagtctcctggcagccctggtcatttgcggaat |
| | | tgtgtactggatgcaccgccgcactcggaaagccccaaagcgcatacgcctcccccacatccg |
| | | ggaagacgaccagccgtcctcgcaccagcccttgttttac* |
| gDM2 | SEQ ID | MGGAAARLGAVILFVVIVGLHGVRGKYALADASLKMADPN |
| Protein | NO: 3 | RFRGKDLPVLDQLTDPPGVRRVYHIQAGLPDPFQPPSLPITVY |
| | | YAVLERACRSVLLNAPSEAPQIVRGASEDVRKQPYNLTIAWF |
| | | RMGGNCAIPITVMEYTECSYNKSLGACPIRTQPRWNYYDSFS |
| | | AVSEDNLGFLMHAPAFETAGTYLRLVKINDWTEITQFILEHR |
| | | AKGSCKYALPLRIPPSACLSPQAYQQGVTVDSIGMLPRAIPEN |
| | | QRTVAVYSLKIAGWHGPKAPYTSTLLPPELSETPNATQPELA |
| | | PEDPEDSALLEDPVGTVAPQIPPNWHIPSIQDAATPYHPPATP |
| | | NNMGLIAGAVGGSLLAALVICGIVYWMHRRTRKAPKRIRLP |
| | | HIREDDQPSSHQPLFY |
| gDM2 | SEQ ID | atggggggggctgccgccaggttgggggccgtgattttgtttgtcgtcatagtgggcctccatgg |
| Nucleotide | NO: 4 | ggtccgcggcaaatatgccttggcggatgcctctctcaagatggccgaccccaatcgctttcgcg |
| | | gcaaagaccttccggtcctggaccagctgaccgaccctccgggggtccggcgcgtgtaccaca |
| | | tccaggcgggcctaccggacccgttccagccccccagcctcccgatcacggtttactacgccgt |
| | | gttggagcgcgcctgccgcagcgtgctcctaaacgcaccgtcggaggccccccagattgtccg |
| | | cggggcctccgaagacgtccggaaacaaccctacaacctgaccatcgcttggtttcggatggga |
| | | ggcaactgtgctatccccatcacggtcatggagtacaccgaatgctcctacaacaagtctctggg |
| | | ggcctgtcccatccgaacgcagccccgctggaactactatgacagcttcagcgccgtcagcga |
| | | ggataacctggggttcctgatgcacgcccccgcgtttgagaccgccggcacgtacctgcggctc |
| | | gtgaagataaacgactggacggagattacacagtttatcctggagcaccgagccaagggctcct |
| | | gtaagtacgccctcccgctgcgcatccccccgtcagcctgcctctccccccaggcctaccagca |
| | | gggggtgacggtggacagcatcgggatgctgccccgcgctatccccgagaaccagcgcacc |
| | | gtcgccgtatacagcttgaagatcgccgggtggcacgggcccaaggccccatacacgagcac |
| | | cctgctgcccccggagctgtccgagacccccaacgccacgcagccagaactcgccccggaag |
| | | accccgaggattcggccctcttggaggaccccgtggggacggtggcgccgcaaatcccacca |
| | | aactggcacatcccgtcgatccaggacgccgcgacgccttaccatcccccggccaccccgaac |
| | | aacatgggcctgatcgccggcgcggtgggcggcagtctcctggcagccctggtcatttgcgga |
| | | attgtgtactggatgcaccgccgcactcggaaagccccaaagcgcatacgcctcccccacatcc |
| | | gggaagacgaccagccgtcctcgcaccagcccttgttttac* |
| gDM3 | SEQ ID | MGGAAARLGAVILFVVIVGLHGVRGKYALADASLKMADPN |
| Protein | NO: 5 | RFRGKDLPVLDQLTDPPGVRRVYHIQAGLPDPFQPPSLPITVY |
| | | YAVLERACRSVLLNAPSEAPQIVRGASEDVRKQPYNLTIAWF |
| | | RMGGNCAIPITVMEYTECSYNKSLGACPIRTQPRWNYYDSFS |
| | | AVSEDNLGFLMHAPAFETAGTYLRLVKINDWTEITQFILEHR |
| | | AKGSCKYALPLRIPPSACLSPQAYQQGVTVDSIGMLPRFIPEN |
| | | QRTVAVYSLKIAGWHGPGLIAGAVGGSLLAALVICGIVYWM |
| | | HRRTRKA |
| gDM3 | SEQ ID | atggggggggctgccgccaggttgggggccgtgattttgtttgtcgtcatagtgggcctccatgg |
| Nucleotide | NO: 6 | ggtccgcggcaaatatgccttggcggatgcctctctcaagatggccgaccccaatcgctttcgcg |
| | | gcaaagaccttccggtcctggaccagctgaccgaccctccgggggtccggcgcgtgtaccaca |
| | | tccaggcgggcctaccggacccgttccagccccccagcctcccgatcacggtttactacgccgt |
| | | gttggagcgcgcctgccgcagcgtgctcctaaacgcaccgtcggaggccccccagattgtccg |
| | | cggggcctccgaagacgtccggaaacaaccctacaacctgaccatcgcttggtttcggatggga |
| | | ggcaactgtgctatccccatcacggtcatggagtacaccgaatgctcctacaacaagtctctggg |
| | | ggcctgtcccatccgaacgcagccccgctggaactactatgacagcttcagcgccgtcagcga |
| | | ggataacctggggttcctgatgcacgcccccgcgtttgagaccgccggcacgtacctgcggctc |
| | | gtgaagataaacgactggacggagattacacagtttatcctggagcaccgagccaagggctcct |
| | | gtaagtacgccctcccgctgcgcatccccccgtcagcctgcctctccccccaggcctaccagca |
| | | gggggtgacggtggacagcatcgggatgctgccccgcttcatccccgagaaccagcgcaccg |
| | | tcgccgtatacagcttgaagatcgccgggtggcacgggcccggcctgatcgccggcgcggtg |
| | | ggcggcagtctcctggcagccctggtcatttgcggaattgtgtactggatgcaccgccgcactcg |
| | | gaaagcc* |
| gDM4 | SEQ ID | MGGAAARLGAVILFVVIVGLHGVRGKYCLADASLKMADPN |
| Protein | NO: 7 | RFRGKDLPVLDQLTDPPGVRRVYHIQAGLPDPFQPPSLPITVC |
| | | YAVLERACRSVLLNAPSEAPQIVRGASEDVRKQPYNLTIAWF |
| | | RMGGNCAIPITVMEYTECSYNKSLGACPIRTQPRWNYYDSFS |
| | | AVSEDNLGFLMHAPAFETAGTYLRLVKINDWTEITQFILEHR |
| | | AKGSCKYALPLRIPPSACLSPQAYQQGVTVDSIGMLPRAIPEN |
| | | QRTVAVYSLKIAGWHGPKAPYTSTLLPPELSETPNATQPELA |
| | | PEDPEDSALLEDPVGTVAPQIPPNWHIPSIQDAATPYHPPATP |
| | | NNMGLIAGAVGGSLLAALVICGIVYWMHRRTRKAPKRIRLP |
| | | HIREDDQPSSHQPLFY |
| gDM4 | SEQ ID | atggggggggctgccgccaggttgggggccgtgattttgtttgtcgtcatagtgggcctccatgg |
| Nucleotide | NO: 8 | ggtccgcggcaaatattgtttggcggatgcctctctcaagatggccgaccccaatcgctttcgcg |
| | | gcaaagaccttccggtcctggaccagctgaccgaccctccgggggtccggcgcgtgtaccaca |
| | | tccaggcgggcctaccggacccgttccagccccccagcctcccgatcacggtttgctacgccgt |
| | | gttggagcgcgcctgccgcagcgtgctcctaaacgcaccgtcggaggccccccagattgtccg |
| | | cggggcctccgaagacgtccggaaacaaccctacaacctgaccatcgcttggtttcggatggga |
| | | ggcaactgtgctatccccatcacggtcatggagtacaccgaatgctcctacaacaagtctctggg |
| | | ggcctgtcccatccgaacgcagccccgctggaactactatgacagcttcagcgccgtcagcga |
| | | ggataacctggggttcctgatgcacgcccccgcgtttgagaccgccggcacgtacctgcggctc |
| | | gtgaagataaacgactggacggagattacacagtttatcctggagcaccgagccaagggctcct |
| | | gtaagtacgccctcccgctgcgaatccccccgtcagcctgcctctccccccaggcctaccagca |
| | | gggggtgacggtggacagcatcgggatgctgccccgcgctatccccgagaaccagcgcacc |
| | | gtcgccgtatacagcttgaagatcgccgggtggcacgggcccaaggccccatacacgagcac |
| | | cctgctgcccccggagctgtccgagacccccaacgccacgcagccagaactcgccccggaag |
| | | accccgaggattcggccctcttggaggaccccgtggggacggtggcgccgcaaatcccacca |
| | | aactggcacatcccgtcgatccaggacgccgcgacgccttaccatcccccggccaccccgaac |
| | | aacatgggcctgatcgccggcgcggtgggcggcagtctcctggcagccctggtcatttgcgga |
| | | attgtgtactggatgcaccgccgcactcggaaagccccaaagcgcatacgcctcccccacatcc |
| | | gggaagacgaccagccgtcctcgcaccagcccttgttttac* |
| gDM5 | SEQ ID | MGGAAARLGAVILFVVIVGLHGVRGKYCLADASLKMADPN |
| Protein | NO: 9 | RFRGKDLPVLDQLTDPPGVRRVYHIQAGLPDPFQPPSLPITVC |
| | | YAVLERACRSVLLNAPSEAPQIVRGASEDVRKQPYNLTIAWF |
| | | RMGGNCAIPITVMEYTECSYNKSLGACPIRTQPRWNYYDSFS |
| | | AVSEDNLGFLMHAPAFETAGTYLRLVKINDWTEITQFILEHR |
| | | AKGSCKYALPLRIPPSACLSPQAYQQGVTVDSIGPATQPELAP |
| | | EDPEDSALLEDPVGTVAPQIPPNWHIPSIQDAATPYHPPATPN |
| | | NMGLIAGAVGGSLLAALVICGIVYWMHRRTRKAPKRIRLPHI |
| | | REDDQPSSHQPLFY |
| gDM5 | SEQ ID | atggggggggctgccgccaggttgggggccgtgattttgtttgtcgtcatagtgggcctccatgg |
| Nucleotide | NO: 10 | ggtccgcggcaaatattgtttggcggatgcctctctcaagatggccgaccccaatcgctttcgcg |
| | | gcaaagaccttccggtcctggaccagctgaccgaccctccgggggtccggcgcgtgtaccaca |
| | | tccaggcgggcctaccggacccgttccagccccccagcctcccgatcacggtttgctacgccgt |
| | | gttggagcgcgcctgccgcagcgtgctcctaaacgcaccgtcggaggccccccagattgtccg |
| | | cggggcctccgaagacgtccggaaacaaccctacaacctgaccatcgcttggtttcggatggga |
| | | ggcaactgtgctatccccatcacggtcatggagtacaccgaatgctcctacaacaagtctctggg |
| | | ggcctgtcccatccgaacgcagccccgctggaactactatgacagcttcagcgccgtcagcga |
| | | ggataacctggggttcctgatgcacgcccccgcgtttgagaccgccggcacgtacctgcggctc |
| | | gtgaagataaacgactggacggagattacacagtttatcctggagcaccgagccaagggctcct |
| | | gtaagtacgccctcccgctgcgcatccccccgtcagcctgcctctccccccaggcctaccagca |
| | | gggggtgacggtggacagcatcgggcccgccacgcagccagaactcgccccggaagaccc |
| | | cgaggattcggccctcttggaggaccccgtggggacggtggcgccgcaaatcccaccaaact |
| | | ggcacatcccgtcgatccaggacgccgcgacgccttaccatcccccggccaccccgaacaac |
| | | atgggcctgatcgccggcgcggtgggcggcagtctcctggcagccctggtcatttgcggaattg |
| | | tgtactggatgcaccgccgcactcggaaagccccaaagcgcatacgcctcccccacatccggg |
| | | aagacgaccagccgtcctcgcaccagcccttgttttac* |
| gDM5 No | SEQ ID | HGVRGKYCLADASLKMADPNRFRGKDLPVLDQLTDPPGVR |
| Signal Seq | NO: 11 | RVYHIQAGLPDPFQPPSLPITVCYAVLERACRSVLLNAPSEAP |
| Protein | | QIVRGASEDVRKQPYNLTIAWFRMGGNCAIPITVMEYTECSY |
| | | NKSLGACPIRTQPRWNYYDSFSAVSEDNLGFLMHAPAFETA |
| | | GTYLRLVKINDWTEITQFILEHRAKGSCKYALPLRIPPSACLS |
| | | PQAYQQGVTVDSIGPATQPELAPEDPEDSALLEDPVGTVAPQ |
| | | IPPNWHIPSIQDAATPYHPPATPNNMGLIAGAVGGSLLAALVI |
| | | CGIVYWMHRRTRKAPKRIRLPHIREDDQPSSHQPLFY |
| gDM5 No | SEQ ID | catggggtccgcggcaaatattgtttggcggatgcctctctcaagatggccgaccccaatcgcttt |
| Signal Seq | NO: 12 | cgcggcaaagaccttccggtcctggaccagctgaccgaccctccgggggtccggcgcgtgta |
| Nucleotide | | ccacatccagggggcctaccggacccgttccagccccccagcctcccgatcacggtttgctac |
| | | gccgtgttggagcgcgcctgccgcagcgtgctcctaaacgcaccgtcggaggccccccagatt |
| | | gtccgcggggcctccgaagacgtccggaaacaaccctacaacctgaccatcgcttggtttcgga |
| | | tgggaggcaactgtgctatccccatcacggtcatggagtacaccgaatgctcctacaacaagtct |
| | | ctgggggcctgtcccatccgaacgcagccccgctggaactactatgacagcttcagcgccgtca |
| | | gcgaggataacctggggttcctgatgcacgcccccgcgtttgagaccgccggcacgtacctgc |
| | | ggctcgtgaagataaacgactggacggagattacacagtttatcctggagcaccgagccaagg |
| | | gctcctgtaagtacgccctcccgctgcgcatccccccgtcagcctgcctctccccccaggcctac |
| | | cagcagggggtgacggtggacagcatcgggcccgccacgcagccagaactcgccccggaa |
| | | gaccccgaggattcggccctcttggaggaccccgtggggacggtggcgccgcaaatcccacc |
| | | aaactggcacatcccgtcgatccaggacgccgcgacgccttaccatcccccggccaccccgaa |
| | | caacatgggcctgatcgccggcgcggtgggcggcagtctcctggcagccctggtcatttgcgg |
| | | aattgtgtactggatgcaccgccgcactcggaaagccccaaagcgcatacgcctcccccacatc |
| | | cgggaagacgaccagccgtcctcgcaccagcccttgttttac* |
| gDM5 N- | SEQ ID | MGGAAARLGAVILFVVIVGLHGVRGKYCLADASLKMADPN |
| Terminal | NO: 13 | RFRGKDLPVLDQLTDPPGVRRVYHIQAGLPDPFQPPSLPITVC |
| Protein | | YAVLERACRSVLLNAPSEAPQIVRGASEDVRKQPYNLTIAWF |
| | | RMGGNCAIPITVMEYTECSYNKSLGACPIRTQPRWNYYDSFS |
| | | AVSEDNLGFLMHAPAFETAGTYLRLVKINDWTEITQFILEHR |
| | | AKGSCKYALPLRIPPSACLSPQAYQQGVTVDSIGP |
| gDM5 N- | SEQ ID | atggggggggctgccgccaggttgggggccgtgattttgtttgtcgtcatagtgggcctccatgg |
| Terminal | NO: 14 | ggtccgcggcaaatattgtttggcggatgcctctctcaagatggccgaccccaatcgctttcgcg |
| Nucleotide | | gcaaagaccttccggtcctggaccagctgaccgaccctccgggggtccggcgcgtgtaccaca |
| | | tccaggcgggcctaccggacccgttccagccccccagcctcccgatcacggtttgctacgccgt |
| | | gttggagcgcgcctgccgcagcgtgctcctaaacgcaccgtcggaggccccccagattgtccg |
| | | cggggcctccgaagacgtccggaaacaaccctacaacctgaccatcgcttggtttcggatggga |
| | | ggcaactgtgctatccccatcacggtcatggagtacaccgaatgctcctacaacaagtctctggg |
| | | ggcctgtcccatccgaacgcagccccgctggaactactatgacagcttcagcgccgtcagcga |
| | | ggataacctggggttcctgatgcacgcccccgcgtttgagaccgccggcacgtacctgcggctc |
| | | gtgaagataaacgactggacggagattacacagtttatcctggagcaccgagccaagggctcct |
| | | gtaagtacgccctcccgctgcgcatccccccgtcagcctgcctctccccccaggcctaccagca |
| | | gggggtgacggtggacagcatcgggccc |
| gDM5 N- | SEQ ID | HGVRGKYCLADASLKMADPNRFRGKDLPVLDQLTDPPGVR |
| Terminal | NO: 15 | RVYHIQAGLPDPFQPPSLPITVCYAVLERACRSVLLNAPSEAP |
| No Signal | | QIVRGASEDVRKQPYNLTIAWFRMGGNCAIPITVMEYTECSY |
| Seq Protein | | NKSLGACPIRTQPRWNYYDSFSAVSEDNLGFLMHAPAFETA |
| | | GTYLRLVKINDWTEITQFILEHRAKGSCKYALPLRIPPSACLS |
| | | PQAYQQGVTVDSIGP |
| gDM5 N- | SEQ ID | catggggtccgcggcaaatattgtttggoggatgcctctctcaagatggccgaccccaatcgcttt |
| Terminal | NO: 16 | cgcggcaaagaccttccggtcctggaccagctgaccgaccctccgggggtccggcgcgtgta |
| No Signal | | ccacatccagggggcctaccggacccgttccagccccccagcctcccgatcacggtttgctac |
| Seq | | gccgtgttggagcgcgcctgccgcagcgtgctcctaaacgcaccgtcggaggccccccagatt |
| Nucleotide | | gtccgcggggcctccgaagacgtccggaaacaaccctacaacctgaccatcgcttggtttcgga |
| | | tgggaggcaactgtgctatccccatcacggtcatggagtacaccgaatgctcctacaacaagtct |
| | | ctgggggcctgtcccatccgaacgcagccccgctggaactactatgacagcttcagcgccgtca |
| | | gcgaggataacctggggttcctgatgcacgcccccgcgtttgagaccgccggcacgtacctgc |
| | | ggctcgtgaagataaacgactggacggagattacacagtttatcctggagcaccgagccaagg |
| | | gctcctgtaagtacgccctcccgctgcgcatccccccgtcagcctgcctctccccccaggcctac |
| | | cagcagggggtgacggtggacagcatcgggccc |
| gDM5 C- | SEQ ID | ATQPELAPEDPEDSALLEDPVGTVAPQIPPNWHIPSIQDAATP |
| Terminal | NO: 17 | YHPPATPNNMGLIAGAVGGSLLAALVICGIVYWMHRRTRKA |
| Protein | | PKRIRLPHIREDDQPSSHQPLFY |
| gDM5 C- | SEQ ID | gccacgcagccagaactcgccccggaagaccccgaggattcggccctcttggaggaccccgt |
| Terminal | NO: 18 | ggggacggtggcgccgcaaatcccaccaaactggcacatcccgtcgatccaggacgccgcga |
| Nucleotide | | cgccttaccatcccccggccaccccgaacaacatgggcctgatcgccggcgcggtgggcggc |
| | | agtctcctggcagccctggtcatttgcggaattgtgtactggatgcaccgccgcactcggaaagc |
| | | cccaaagcgcatacgcctcccccacatccgggaagacgaccagccgtcctcgcaccagccctt |
| | | gttttac* |
| E765dt1 | SEQ ID | MHGDTPTLHEYMLDLQPETTGLYGYGQLNDSSEEEDEIDGP |
| Protein | NO: 19 | AGQAEPDRAHYNIVTFCCKCDSTLRLCVQSTHVDIRTLEDLL |
| | | MGTLGIVCPICSQKPGAHQKRTAMFQDPQERPRKLPQLCTEL |
| | | QTTIHDIILECVYCKQQLLRREVYDYAYRHLCIVYRDGNPYA |
| | | VCDKCLKFYSKISEYRHYCYSLYGTTLEQQYNKPLCDLLIRCI |
| | | NCQKPQCPEEKQRHLDKKQRFHNIRGRWTGRCMSCCRSSRT |
| | | RRETQLAAATNLDTASTTLLACFLLCFCVLLCVCLLIRPLLLS |
| | | VSTYTSLIILVLLLWITAASAFRCFIVYIVFVYIPLFLIHTHA |
| E765dt1 | SEQ ID | atgcacggggacactcccaccctgcatgaatatatgctggatctgcagcctgagaccacaggcc |
| Nucleotide | NO: 20 | tgtacggctatggacagctgaacgacagctccgaggaagaggacgaaatcgatgggccagca |
| | | ggacaggcagagccagacagagcccactacaatattgtgaccttctgctgtaagtgcgatagta |
| | | cactgcgactgtgcgtgcagtcaacacatgtcgacatccgcactctggaggatctgctgatggga |
| | | accctggggatcgtgtgccctatttgttctcagaagccaggcgctcaccagaaaaggactgcaat |
| | | gtttcaggatccacaggaacgacccaggaagctgcctcagctgtgcaccgagctgcagactac |
| | | catccacgacatcattctggaatgcgtgtattgtaaacagcagctgctgaggagagaggtctacg |
| | | attatgcatacaggcatctgtgcatcgtgtatagagacggaaacccatacgccgtctgcgataagt |
| | | gtctgaaattctattccaagatctctgagtacaggcactattgttacagcctgtatggcacaactctg |
| | | gaacagcagtacaacaaacccctgtgcgacctgctgatcagatgcattaattgtcagaagcccca |
| | | gtgtcctgaagagaaacagcggcacctggataagaaacagaggttccataacatcagaggccg |
| | | gtggacaggaagatgcatgtcttgctgtcgctctagtcgaacccgacgagagacacagctggca |
| | | gctgcaactaatctggacaccgccagtaccacactgctggcttgtttcctgctgtgcttttgcgtgct |
| | | gctgtgcgtctgtctgctgatccggcctctgctgctgtcagtgagcacatacactagcctgatcatt |
| | | ctggtcctgctgctgtggattacagccgcttccgctttccgctgttttatcgtgtatatcgtgttcgtgt |
| | | acatccccctgtttctgattcacactcatgcc* |
| E765dt3 | SEQ ID | MHGDTPTLHEYMLDLQPETTAAAAAAQLNDSSEEEDEIDGP |
| Protein | NO: 21 | AGQAEPDRAHYNIVTFCCKCDSTLRLCVQSTHVDIRTLEDLL |
| (also | | MGTLGIVCPICSQKPGAHQKRTAMFQDPQERPRKLPQLCTEL |
| referred to | | QTTIHDIILECVYCKQQLLRREVYDAAAAALCIVYRDGNPYA |
| herein as | | VCDKCLKFYSKISEYRHYCYSLYGTTLEQQYNKPLCDLLIRCI |
| E765 | | NCQKPQCPEEKQRHLDKKQRFHNIRGRWTGRCMSCCRSSRT |
| Protein) | | RRETQLGACVLLCVCLLIRPLLLSVSTYTSLIILVLLLWITAAS |
| | | AFRCFIVYIAFVYIPLFLIHTHA |
| E765dt3 | SEQ ID | atgcacggcgacacccccaccctgcacgagtacatgctggacctgcagcccgagaccaccgc |
| Nucleotide | NO: 22 | cgccgccgccgccgcccagctgaacgacagcagcgaggaggaggacgagatcgacggccc |
| (also | | cgccggccaggccgagcccgacagggctcactacaacatcgtgaccttctgctgcaagtgcga |
| referred to | | cagcaccctgaggctgtgcgtgcagagcacccacgtggacatcaggaccctggaggacctgct |
| herein as | | gatgggcaccctgggcatcgtgtgccccatctgcagccagaagcccggcgcccaccagaaga |
| E765 | | ggaccgccatgttccaggacccccaggagaggcccaggaagctgccccagctgtgcaccga |
| Nucleotide) | | gctgcagaccaccatccacgacatcatcctggagtgcgtgtactgcaagcagcagctgctgagg |
| | | agggaggtgtacgacgccgccgccgccgccctgtgcatcgtgtacagggacggcaaccccta |
| | | cgccgtgtgcgacaagtgcctgaagttctacagcaagatcagcgagtacaggcactactgctac |
| | | agcctgtacggcaccaccctggagcagcagtacaacaagcccctgtgcgacctgctgatcagg |
| | | tgcatcaactgccagaagccccagtgccccgaggagaagcagaggcacctggacaagaagc |
| | | agaggttccacaacatcaggggcaggtggaccggcaggtgcatgagctgctgcaggagcagc |
| | | aggaccaggagggagacccagctgggcgcctgcgtgctgctgtgcgtgtgcctgctgatcagg |
| | | cccctgctgctgagcgtgagcacctacaccagcctgatcatcctggtgctgctgctgtggatcac |
| | | cgccgccagcgccttcaggtgcttcatcgtgtacatcgccttcgtgtacatccccctgttcctgatc |
| | | cacacccacgcc |
| Mutant | SEQ ID | MTPTLHEYMLDLQPETTDLYGPAGQAEPDRAHYNIVTFCCK |
| HPV E7, | NO: 23 | CDSTLRLCVQSTHVDIRTLEDLLMGTLGIVCPICAAPRKLPQL |
| E6, E5, and | | CTELNTSLQDIEITCVYCYDFAFRAACIVYRDGNPYAVCDKC |
| E2 Fusion | | LKFYSKISEYRHYCYSVYGTTLAASVSTYTSLILLVLLLWITA |
| Protein | | ASAFRCFIVYIVFVYIPLFLIHTHARFLIAAETLCQRLNVCQDK |
| (E7652 | | ILTHYENDSTDLRDHIDYWKHMRLECAIYYKAREMGFKHIN |
| Protein) | | HQVVPTLAVSKNKALQAIELQLTLETIYNSQYSNEKWSNTTP |
| | | IVHLKGDANTLKCLRYRFKKHCKLYTAVSSTWHWTGHNVK |
| | | HKSAIVTLTYDSEW |
| Mutant | SEQ ID | atgacacctacattgcatgaatatatgttagatttgcaaccagagacaactgatctctacggtccag |
| HPV E7, | NO: 24 | ctggacaagcagaaccggacagagcccattacaatattgtaaccttttgttgcaagtgtgactcta |
| E6, E5, and | | cgcttcggttgtgcgtacaaagcacacacgtagacattcgtactttggaagacctgttaatgggca |
| E2 Fusion | | cactaggaattgtgtgccccatctgtgctgctcccagaaagttaccacagttatgcacagagctga |
| Nucleotide | | acacttcactgcaagacatagaaataacctgtgtatattgctatgactttgcttttcgggctgcttgca |
| (E7652 | | tagtatatagagatgggaatccatatgctgtatgtgataaatgtttaaagttttattctaaaattagtga |
| Nucleotide) | | gtatagacattattgttatagtgtatatggaacaacattagctgcttctgtgtctacatacacatcatta |
| | | atattgttggtattactattgtggataacagcagcctctgcgtttaggtgttttattgtatatattgtattt |
| | | gtttatataccattatttttaatacatacacatgcacgctttttaattgctgctgagactctttgccaacg |
| | | tttaaatgtgtgtcaggacaaaatactaacacattatgaaaatgatagtacagacctacgtgaccat |
| | | atagactattggaaacacatgcgcctagaatgtgctatttattacaaggccagagaaatgggattta |
| | | aacatattaaccaccaggtggtgccaacactggctgtatcaaagaataaagcattacaagcaatt |
| | | gaactgcaactaacgttagaaacaatatataactcacaatatagtaatgaaaagtggagtaacact |
| | | acacccatagtacatttaaaaggtgatgctaatactttaaaatgtttaagatatagatttaaaaagcat |
| | | tgtaaattgtatactgcagtgtcgtctacatggcattggacaggacataatgtaaaacataaaagtg |
| | | caattgttacacttacatatgatagtgaatgg |
| PolN | SEQ ID | MPLSYQHFRKLLLLDEEAGPLEEELPRLADEGLNRRVAEDLN |
| Protein | NO: 25 | LGNLNVSIPWTHKVGNFTGLYSSTVPVFNPEWQTPSFPKIHL |
| | | QEDIVDRCKQFVGPLTVNEKRRLKLIMPARFYPNVTKYLPLD |
| | | KGIKPYYPEHAVNHYFQTRHYLHTLWKAGILYKRETTRSAS |
| | | FCGSPYSWEQELQHGSCWWLQFRNSKPCSEYCLTHLVNLLE |
| | | DWGPCDEHGEHHIRIPRTPARVTGGVFLVDKNPHNTAESRL |
| | | VVDFSQFSRGITRVSWPKFAVPNLQSLTNLLSSNLSWLSLDV |
| | | SAAFYHIPLHPAAMP |
| PolN | SEQ ID | atgcccctgagctaccagcacttcaggaagctgctgctgctggacgaggaggccggccccctg |
| Nucleotide | NO: 26 | gaggaggagctgcccaggctggccgacgagggcctgaacaggagggtggccgaggacctg |
| | | aacctgggcaacctgaacgtgagcatcccctggacccacaaggtgggcaacttcaccggcctg |
| | | tacagcagcaccgtgcccgtgttcaaccccgagtggcagacccccagcttccccaagatccac |
| | | ctgcaggaggacatcgtggacaggtgcaagcagttcgtgggtcccctgaccgtgaacgagaag |
| | | aggaggctgaagctgatcatgcccgccaggttctaccccaacgtgaccaagtacctgcccctgg |
| | | acaagggcatcaagccctactaccccgagcacgccgtgaaccactacttccagaccaggcact |
| | | acctgcacaccctgtggaaggccggcatcctgtacaagagggagaccaccaggagcgccagc |
| | | ttctgcggcagcccctacagctgggagcaggagctgcagcacggcagctgctggtggctgca |
| | | gttcaggaacagcaagccctgcagcgagtactgcctgacccacctggtgaacctgctggagga |
| | | ctggggtccctgcgacgagcacggcgagcaccacatcaggatccccaggacccccgccagg |
| | | gtgaccggcggcgtgttcctggtggacaagaacccccacaacaccgccgagagcaggctggt |
| | | ggtggacttcagccagttcagcaggggcatcaccagggtgagctggcccaagttcgccgtgcc |
| | | caacctgcagagcctgaccaacctgctgagcagcaacctgagctggctgagcctggacgtgag |
| | | cgccgccttctaccacatccccctgcaccccgccgccatgccc |
| HBV3 | SEQ ID | MHFRKLLLLDEEAGPLEEELPRLADEGLNRRVAEDLNLGNL |
| Protein | NO: 27 | PEWQTPSFPKIHLQEDIVDRCKQFVGPLTVNEKRRLKLIMPA |
| | | RFYPNVTKYLPLDKGIKPYYPEHAVNHYFQTRHYLHTLWKA |
| | | GILYKRETTRSASFCGSPYSWEQELQHGSCWWLQFRNSKPCS |
| | | EYCLTHLVNLLEDWGPCDEHGEHHIRIPRTPARVTQAFTFSP |
| | | TYKAFLSKQYLNLYPVARQRPGLCQVFADATPTGWGLAMG |
| | | HQRMRGTFVAPLPIHTAELLAACFARSRSGAKILGTDNSVVL |
| | | SRKYTSFPWLLGCAANWILRGTSFVYVPSALNPADDVGSNL |
| | | EDPASRELVVSYVNVNMGLKIRQLLWFHISCLTFGRETVIEY |
| | | LVSFGVWIRTPPAYRPPNAPILSTLPETTVVRRRDRGR |
| HBV3 | SEQ ID | atgcattttcgcaaactgctgctgctggatgaagaagcgggaccgctggaagaagaactgccgc |
| Nucleotide | NO: 28 | gcctggcggatgaaggcctgaaccgccgcgtggcggaagatctgaacctgggcaacctgccg |
| | | gaatggcagaccccgagctttccgaaaattcatctgcaggaagatattgtggatcgctgcaaaca |
| | | gtttgtgggaccgctgaccgtgaacgaaaaacgccgcctgaaactgattatgccggcgcgctttt |
| | | atccgaacgtgaccaaatatctgccgctggataaaggcattaaaccgtattatccggaacatgcg |
| | | gtgaaccattattttcagacccgccattatctgcataccctgtggaaagcgggcattctgtataaac |
| | | gcgaaaccacccgcagcgcgagcttttgcggcagcccgtatagctgggaacaggaactgcag |
| | | catggcagctgctggtggctgcagtttcgcaacagcaaaccgtgcagcgaatattgcctgaccc |
| | | atctggtgaacctgctggaagattggggaccgtgcgatgaacatggcgaacatcatattcgcatt |
| | | ccgcgcaccccggcgcgcgtgacccaggcgtttacctttagcccgacctataaagcgtttctga |
| | | gcaaacagtatctgaacctgtatccggtggcgcgccagcgcccgggcctgtgccaggtgtttgc |
| | | ggatgcgaccccgaccggctggggcctggcgatgggccatcagcgcatgcgcggcacctttg |
| | | tggcgccgctgccgattcataccgcggaactgctggcggcgtgctttgcgcgcagccgcagcg |
| | | gcgcgaaaattctgggcaccgataacagcgtggtgctgagccgcaaatataccagctttccgtg |
| | | gctgctgggctgcgcggcgaactggattctgcgcggcaccagctttgtgtatgtgccgagcgcg |
| | | ctgaacccggcggatgatgtgggcagcaacctggaagatccggcgagccgcgaactggtggt |
| | | gagctatgtgaacgtgaacatgggcctgaaaattcgccagctgctgtggtttcatattagctgcct |
| | | gacctttggccgcgaaaccgtgattgaatatctggtgagctttggcgtgtggattcgcaccccgcc |
| | | ggcgtatcgcccgccgaacgcgccgattctgagcaccctgccggaaaccaccgtggtgcgcc |
| | | gccgagatcgaggccgc |
| HIV-1 gag | SEQ ID | MGARASVLSGGELDRWEKIRLRPGGKKKYKLKHIVWASRE |
| Protein | NO: 29 | LERFAVNPGLLETSEGCRQILGQLQPSLQTGSEELRSLYNTVA |
| | | TLYCVHQRIEVKDTKEALEKIEEEQNKSKKKAQQAAADTGN |
| | | SSQVSQNYPIVQNLQGQMVHQAISPRTLNAWVKVVEEKAFS |
| | | PEVIPMFSALSEGATPQDLNTMLNTVGGHQAAMQMLKETIN |
| | | EEAAEWDRLHPVHAGPIAPGQMREPRGSDIAGTTSTLQEQIG |
| | | WMTNNPPIPVGEIYKRWIILGLNKIVRMYSPTSILDIRQGPKEP |
| | | FRDYVDRFYKTLRAEQASQEVKNWMTETLLVQNANPDCKT |
| | | ILKALGPAATLEEMMTACQGVGGPGHKARVLAEAMSQVTN |
| | | SATIMMQRGNFRNQRKTVKCFNCGKEGHIAKNCRAPRKKG |
| | | CWKCGKEGHQMKDCTERQANFLGKIWPSHKGRPGNFLQSR |
| | | PEPTAPPEESFRFGEETTTPSQKQEPIDKELYPLASLRSLFGND |
| | | PSSQ |
| HIV-1 gag | SEQ ID | atgggcgccagagccagcgtgctgagcgggggagagctggatcgctgggaaaagattagact |
| Nucleotide | NO: 30 | gaggcccggagggaagaaaaagtacaagctgaaacacatcgtgtgggccagcagggagctg |
| | | gagcgcttcgccgtgaaccccggactgctggagacctccgaaggctgtcggcagatcctgggg |
| | | cagctgcagccttccctgcagacaggctccgaggagctgcggtctctgtataatacagtggcca |
| | | cactgtactgtgtgcaccagcggattgaggtgaaggatacaaaagaggccctggagaagattga |
| | | ggaggagcagaacaagagcaagaagaaagcccagcaggccgccgccgacaccggcaatag |
| | | ctcccaggtgagccagaactatccaattgtgcagaatctgcagggccagatggtgcaccaggcc |
| | | atttccccacggacactgaacgcctgggtgaaggtggtggaggagaaggccttcagccccgaa |
| | | gtgatccctatgttttccgccctgtccgaaggcgccacccctcaggacctgaacaccatgctgaa |
| | | cacagtggggggacaccaggccgccatgcagatgctgaaggaaaccatcaacgaggaggcc |
| | | gccgagtgggatcggctgcaccccgtgcacgccgggccaatcgcccctggccagatgaggg |
| | | agcccagaggctctgacatcgccggcaccacatctaccctgcaggaacagatcgggtggatga |
| | | ccaataacccaccaatcccagtgggcgagatctacaagaggtggattattctgggactgaacaa |
| | | aattgtgcgcatgtattcccctacatccatcctggacatcaggcagggacctaaggaacctttccg |
| | | cgactacgtggatcggttctacaaaaccctgcgcgccgagcaggccagccaggaagtgaaga |
| | | attggatgacagagaccctgctggtgcagaacgccaatcctgactgtaaaaccatcctgaaggc |
| | | cctgggacctgccgccacactggaggaaatgatgaccgcctgccagggcgtgggcggccctg |
| | | gccacaaagccagggtgctggccgaggccatgtctcaggtgaccaactctgccacaattatgat |
| | | gcagcgggggaactttcggaaccagaggaagaccgtgaagtgcttcaactgtggcaaggagg |
| | | gacacatcgccaagaattgccgcgccccacggaagaaggggtgttggaaatgcgggaagga |
| | | gggccaccagatgaaagactgcacagagcggcaggccaactttctgggcaagatttggcccag |
| | | ccacaagggccgccccggaaactttctgcagtccaggcctgagcctaccgccccccctgagga |
| | | atccttccggttcggcgaggagacaaccaccccaagccagaagcaggaacccatcgacaaag |
| | | agctgtaccccctggccagcctgagatccctgttcgggaatgaccccagctctcag |
| Melapoly | SEQ ID | MGMQVQIQSLFLLLLWVPGSRGAKFVAAWTLKAAAAAARK |
| Protein | NO: 31 | FFHRTCKCTGNFAAAANESFALPYWNFATGRNECDVAAAD |
| | | QLGYSYAIDLPVSVAAAAAASVYDFFVWLAAAAAASTFSFR |
| | | NALAASQVMNLHNLAATAPDNLGYMAAAIAVVNALLLYA |
| | | YDYEELYAKVPRNQDWLAAAAFGLANEKSIAAAA |
| Melapoly | SEQ ID | atgggtatgcaggtccagatccagtcactcttcctcctcctcctgtgggtgcccggttcacggggt |
| Nucleotide | NO: 32 | gctaagtttgtggccgcttggacactgaaggccgctgcagccgctgcaaggaagttctttcacag |
| | | aacctgcaaatgtacaggaaacttcgccgctgcagccaatgagagcttcgccctgccctattgga |
| | | actttgctaccggccggaatgaatgcgacgtggctgcagccgatcagctcggatactcttatgcc |
| | | atcgacctgcctgtgagtgtcgctgcagccgctgcagcctcagtgtacgatttctttgtctggctgg |
| | | ctgcagccgctgcagccagcactttctcctttcgcaacgccctggctgcatctcaggtcatgaacc |
| | | tccataatctggcagctaccgcaccagacaacctcggatatatggcagccgctattgccgtggtc |
| | | aatgctctgctcctgtacgcctatgactacgaggaactgtacgctaaagtccccaggaatcagga |
| | | ttggctcgcagccgctgcatttggtctggccaacgaaaagagcattgcagcagcagca* |
| WT gD | SEQ ID | MGGAAARLGAVILFVVIVGLHGVRGKYALADASLKMADPN |
| Protein | NO: 33 | RFRGKDLPVLDQLTDPPGVRRVYHIQAGLPDPFQPPSLPITVY |
| | | YAVLERACRSVLLNAPSEAPQIVRGASEDVRKQPYNLTIAWF |
| | | RMGGNCAIPITVMEYTECSYNKSLGACPIRTQPRWNYYDSFS |
| | | AVSEDNLGFLMHAPAFETAGTYLRLVKINDWTEITQFILEHR |
| | | AKGSCKYALPLRIPPSACLSPQAYQQGVTVDSIGMLPRFIPEN |
| | | QRTVAVYSLKIAGWHGPKAPYTSTLLPPELSETPNATQPELA |
| | | PEDPEDSALLEDPVGTVAPQIPPNWHIPSIQDAATPYHPPATP |
| | | NNMGLIAGAVGGSLLAALVICGIVYWMHRRTRKAPKRIRLP |
| | | HIREDDQPSSHQPLFY |
| Mutant | SEQ ID | TPTLHEYMLDLQPETTDLYGPAGQAEPDRAHYNIVTFCCKC |
| HPV E7 | NO: 34 | DSTLRLCVQSTHVDIRTLEDLLMGTLGIVCPIC |
| Protein | | |
| Mutant | SEQ ID | acacctacattgcatgaatatatgttagatttgcaaccagagacaactgatctctacggtccagctg |
| HPV E7 | NO: 35 | gacaagcagaaccggacagagcccattacaatattgtaaccttttgttgcaagtgtgactctacgc |
| Nucleotide | | ttcggttgtgcgtacaaagcacacacgtagacattcgtactttggaagacctgttaatgggcacac |
| | | taggaattgtgtgccccatctgt |
| Mutant | SEQ ID | PRKLPQLCTELNTSLQDIEITCVYCYDFAFRAACIVYRDGNPY |
| HPV E6 | NO: 36 | AVCDKCLKFYSKISEYRHYCYSVYGTTL |
| Protein | | |
| Mutant | SEQ ID | cccagaaagttaccacagttatgcacagagctgaacacttcactgcaagacatagaaataacctg |
| HPV E6 | NO: 37 | tgtatattgctatgactttgcttttcgggctgcttgcatagtatatagagatgggaatccatatgctgta |
| Nucleotide | | tgtgataaatgtttaaagttttattctaaaattagtgagtatagacattattgttatagtgtatatggaac |
| | | aacatta |
| Mutant | SEQ ID | SVSTYTSLILLVLLLWITAASAFRCFIVYIVFVYIPLFLIHTHAR |
| HPV E5 | NO: 38 | FLI |
| Protein | | |
| Mutant | SEQ ID | tctgtgtctacatacacatcattaatattgttggtattactattgtggataacagcagcctctgcgttta |
| HPV E5 | NO: 39 | ggtgttttattgtatatattgtatttgtttatataccattatttttaatacatacacatgcacgctttttaatt |
| Nucleotide | | |
| Mutant | SEQ ID | ETLCQRLNVCQDKILTHYENDSTDLRDHIDYWKHMRLECAI |
| HPV E2 | NO: 40 | YYKAREMGFKHINHQVVPTLAVSKNKALQAIELQLTLETIYN |
| Protein | | SQYSNEKWSNTTPIVHLKGDANTLKCLRYRFKKHCKLYTAV |
| | | SSTWHWTGHNVKHKSAIVTLTYDSEW |
| Mutant | SEQ ID | gagactctttgccaacgtttaaatgtgtgtcaggacaaaatactaacacattatgaaaatgatagta |
| HPV E2 | NO: 41 | cagacctacgtgaccatatagactattggaaacacatgcgcctagaatgtgctatttattacaagg |
| Nucleotide | | ccagagaaatgggatttaaacatattaaccaccaggtggtgccaacactggctgtatcaaagaat |
| | | aaagcattacaagcaattgaactgcaactaacgttagaaacaatatataactcacaatatagtaatg |
| | | aaaagtggagtaacactacacccatagtacatttaaaaggtgatgctaatactttaaaatgtttaag |
| | | atatagatttaaaaagcattgtaaattgtatactgcagtgtcgtctacatggcattggacaggacata |
| | | atgtaaaacataaaagtgcaattgttacacttacatatgatagtgaatgg |
| Mutant | SEQ ID | TPTLHEYMLDLQPETTDLYGPAGQAEPDRAHYNIVTFCCKC |
| HPV E7, | NO: 42 | DSTLRLCVQSTHVDIRTLEDLLMGTLGIVCPICAAPRKLPQLC |
| E6, E5, and | | TELNTSLQDIEITCVYCYDFAFRAACIVYRDGNPYAVCDKCL |
| E2 Fusion | | KFYSKISEYRHYCYSVYGTTLAASVSTYTSLILLVLLLWITAA |
| Protein | | SAFRCFIVYIVFVYIPLFLIHTHARFLIAAETLCQRLNVCQDKI |
| (E7652 | | LTHYENDSTDLRDHIDYWKHMRLECAIYYKAREMGFKHINH |
| Protein) | | QVVPTLAVSKNKALQAIELQLTLETIYNSQYSNEKWSNTTPI |
| (without N- | | VHLKGDANTLKCLRYRFKKHCKLYTAVSSTWHWTGHNVK |
| term M) | | HKSAIVTLTYDSEW |
| Mutant | SEQ ID | acacctacattgcatgaatatatgttagatttgcaaccagagacaactgatctctacggtccagctg |
| HPV E7, | NO: 43 | gacaagcagaaccggacagagcccattacaatattgtaaccttttgttgcaagtgtgactctacgc |
| E6, E5, and | | ttcggttgtgcgtacaaagcacacacgtagacattcgtactttggaagacctgttaatgggcacac |
| E2 Fusion | | taggaattgtgtgccccatctgtgctgctcccagaaagttaccacagttatgcacagagctgaaca |
| Nucleotide | | cttcactgcaagacatagaaataacctgtgtatattgctatgactttgcttttcgggctgcttgcatag |
| (E7652 | | tatatagagatgggaatccatatgctgtatgtgataaatgtttaaagttttattctaaaattagtgagta |
| Nucleotide) | | tagacattattgttatagtgtatatggaacaacattagctgcttctgtgtctacatacacatcattaatat |
| (without | | tgttggtattactattgtggataacagcagcctctgcgtttaggtgttttattgtatatattgtatttgttta |
| N-term atg) | | tataccattatttttaatacatacacatgcacgctttttaattgctgctgagactctttgccaacgtttaa |
| | | atgtgtgtcaggacaaaatactaacacattatgaaaatgatagtacagacctacgtgaccatatag |
| | | actattggaaacacatgcgcctagaatgtgctatttattacaaggccagagaaatgggatttaaac |
| | | atattaaccaccaggtggtgccaacactggctgtatcaaagaataaagcattacaagcaattgaa |
| | | ctgcaactaacgttagaaacaatatataactcacaatatagtaatgaaaagtggagtaacactaca |
| | | cccatagtacatttaaaaggtgatgctaatactttaaaatgtttaagatatagatttaaaaagcattgt |
| | | aaattgtatactgcagtgtcgtctacatggcattggacaggacataatgtaaaacataaaagtgca |
| | | attgttacacttacatatgatagtgaatgg |
| Mutant | SEQ ID | MGGAAARLGAVILFVVIVGLHGVRGKYCLADASLKMADPN |
| HPV E7, | NO: 44 | RFRGKDLPVLDQLTDPPGVRRVYHIQAGLPDPFQPPSLPITVC |
| E6, E5, and | | YAVLERACRSVLLNAPSEAPQIVRGASEDVRKQPYNLTIAWF |
| E2 Fusion | | RMGGNCAIPITVMEYTECSYNKSLGACPIRTQPRWNYYDSFS |
| Protein | | AVSEDNLGFLMHAPAFETAGTYLRLVKINDWTEITQFILEHR |
| Fused to | | AKGSCKYALPLRIPPSACLSPQAYQQGVTVDSIGPTPTLHEY |
| gDM5 | | MLDLQPETTDLYGPAGQAEPDRAHYNIVTFCCKCDSTLRLC |
| Protein | | VQSTHVDIRTLEDLLMGTLGIVCPICAAPRKLPQLCTELNTSL |
| (also | | QDIEITCVYCYDFAFRAACIVYRDGNPYAVCDKCLKFYSKIS |
| referred to | | EYRHYCYSVYGTTLAASVSTYTSLILLVLLLWITAASAFRCFI |
| herein as | | VYIVFVYIPLFLIHTHARFLIAAETLCQRLNVCQDKILTHYEN |
| gDM5- | | DSTDLRDHIDYWKHMRLECAIYYKAREMGFKHINHQVVPTL |
| E7652 | | AVSKNKALQAIELQLTLETIYNSQYSNEKWSNTTPIVHLKGD |
| Protein) | | ANTLKCLRYRFKKHCKLYTAVSSTWHWTGHNVKHKSAIVT |
| | | LTYDSEWGPATQPELAPEDPEDSALLEDPVGTVAPQIPPNWH |
| | | IPSIQDAATPYHPPATPNNMGLIAGAVGGSLLAALVICGIVY |
| | | WMHRRTRKAPKRIRLPHIREDDQPSSHQPLFY |
| Mutant | SEQ ID | atggggggggctgccgccaggttgggggccgtgattttgtttgtcgtcatagtgggcctccatgg |
| HPV E7, | NO: 45 | ggtccgcggcaaatattgtttggoggatgcctctctcaagatggccgaccccaatcgctttcgcg |
| E6, E5, and | | gcaaagaccttccggtcctggaccagctgaccgaccctccgggggtccggcgcgtgtaccaca |
| E2 Fusion | | tccagggggcctaccggacccgttccagccccccagcctcccgatcacggtttgctacgccgt |
| Protein | | gttggagcgcgcctgccgcagcgtgctcctaaacgcaccgtcggaggccccccagattgtccg |
| Fused to | | cggggcctccgaagacgtccggaaacaaccctacaacctgaccatcgcttggtttcggatggga |
| gDM5 | | ggcaactgtgctatccccatcacggtcatggagtacaccgaatgctcctacaacaagtctctggg |
| Nucleotide | | ggcctgtcccatccgaacgcagccccgctggaactactatgacagcttcagcgccgtcagcga |
| (also | | ggataacctggggttcctgatgcacgcccccgcgtttgagaccgccggcacgtacctgcggctc |
| referred to | | gtgaagataaacgactggacggagattacacagtttatcctggagcaccgagccaagggctcct |
| herein as | | gtaagtacgccctcccgctgcgcatccccccgtcagcctgcctctccccccaggcctaccagca |
| gDM5- | | gggggtgacggtggacagcatcgggcccacacctacattgcatgaatatatgttagatttgcaac |
| E7652 | | cagagacaactgatctctacggtccagctggacaagcagaaccggacagagcccattacaatat |
| Nucleotide) | | tgtaaccttttgttgcaagtgtgactctacgcttcggttgtgcgtacaaagcacacacgtagacattc |
| | | gtactttggaagacctgttaatgggcacactaggaattgtgtgccccatctgtgctgctcccagaa |
| | | agttaccacagttatgcacagagctgaacacttcactgcaagacatagaaataacctgtgtatattg |
| | | ctatgactttgcttttcgggctgcttgcatagtatatagagatgggaatccatatgctgtatgtgataa |
| | | atgtttaaagttttattctaaaattagtgagtatagacattattgttatagtgtatatggaacaacattag |
| | | ctgcttctgtgtctacatacacatcattaatattgttggtattactattgtggataacagcagcctctgc |
| | | gtttaggtgttttattgtatatattgtatttgtttatataccattatttttaatacatacacatgcacgctt |
| | | tttaattgctgctgagactctttgccaacgtttaaatgtgtgtcaggacaaaatactaacacattatgaaa |
| | | atgatagtacagacctacgtgaccatatagactattggaaacacatgcgcctagaatgtgctattta |
| | | ttacaaggccagagaaatgggatttaaacatattaaccaccaggtggtgccaacactggctgtat |
| | | caaagaataaagcattacaagcaattgaactgcaactaacgttagaaacaatatataactcacaat |
| | | atagtaatgaaaagtggagtaacactacacccatagtacatttaaaaggtgatgctaatactttaaa |
| | | atgtttaagatatagatttaaaaagcattgtaaattgtatactgcagtgtcgtctacatggcattggac |
| | | aggacataatgtaaaacataaaagtgcaattgttacacttacatatgatagtgaatgggggcccgc |
| | | cacgcagccagaactcgccccggaagaccccgaggattcggccctcttggaggaccccgtgg |
| | | ggacggtggcgccgcaaatcccaccaaactggcacatcccgtcgatccaggacgccgcgacg |
| | | ccttaccatcccccggccaccccgaacaacatgggcctgatcgccggcgcggtgggcggcag |
| | | tctcctggcagccctggtcatttgcggaattgtgtactggatgcaccgccgcactcggaaagccc |
| | | caaagcgcatacgcctcccccacatccgggaagacgaccagccgtcctcgcaccagcccttgt |
| | | tttac* |
| Mutant | SEQ ID | HGVRGKYCLADASLKMADPNRFRGKDLPVLDQLTDPPGVR |
| HPV E7, | NO: 46 | RVYHIQAGLPDPFQPPSLPITVCYAVLERACRSVLLNAPSEAP |
| E6, E5, and | | QIVRGASEDVRKQPYNLTIAWFRMGGNCAIPITVMEYTECSY |
| E2 Fusion | | NKSLGACPIRTQPRWNYYDSFSAVSEDNLGFLMHAPAFETA |
| Protein | | GTYLRLVKINDWTEITQFILEHRAKGSCKYALPLRIPPSACLS |
| Fused to | | PQAYQQGVTVDSIGPTPTLHEYMLDLQPETTDLYGPAGQAE |
| gDM5 No | | PDRAHYNIVTFCCKCDSTLRLCVQSTHVDIRTLEDLLMGTLG |
| Signal Seq | | IVCPICAAPRKLPQLCTELNTSLQDIEITCVYCYDFAFRAACIV |
| Protein | | YRDGNPYAVCDKCLKFYSKISEYRHYCYSVYGTTLAASVST |
| | | YTSLILLVLLLWITAASAFRCFIVYIVFVYIPLFLIHTHARFLIA |
| | | AETLCQRLNVCQDKILTHYENDSTDLRDHIDYWKHMRLECA |
| | | IYYKAREMGFKHINHQVVPTLAVSKNKALQAIELQLTLETIY |
| | | NSQYSNEKWSNTTPIVHLKGDANTLKCLRYRFKKHCKLYTA |
| | | VSSTWHWTGHNVKHKSAIVTLTYDSEWGPATQPELAPEDPE |
| | | DSALLEDPVGTVAPQIPPNWHIPSIQDAATPYHPPATPNNMG |
| | | LIAGAVGGSLLAALVICGIVYWMHRRTRKAPKRIRLPHIRED |
| | | DQPSSHQPLFY |
| Mutant | SEQ ID | catggggtccgcggcaaatattgtttggcggatgcctctctcaagatggccgaccccaatcgcttt |
| HPV E7, | NO: 47 | cgcggcaaagaccttccggtcctggaccagctgaccgaccctccgggggtccggcgcgtgta |
| E6, E5, and | | ccacatccaggcgggcctaccggacccgttccagccccccagcctcccgatcacggtttgctac |
| E2 Fusion | | gccgtgttggagcgcgcctgccgcagcgtgctcctaaacgcaccgtcggaggccccccagatt |
| Protein | | gtccgcggggcctccgaagacgtccggaaacaaccctacaacctgaccatcgcttggtttcgga |
| Fused to | | tgggaggcaactgtgctatccccatcacggtcatggagtacaccgaatgctcctacaacaagtct |
| gDM5 No | | ctgggggcctgtcccatccgaacgcagccccgctggaactactatgacagcttcagcgccgtca |
| Signal Seq | | gcgaggataacctggggttcctgatgcacgcccccgcgtttgagaccgccggcacgtacctgc |
| Nucleotide | | ggctcgtgaagataaacgactggacggagattacacagtttatcctggagcaccgagccaagg |
| | | gctcctgtaagtacgccctcccgctgcgcatccccccgtcagcctgcctctccccccaggcctac |
| | | cagcagggggtgacggtggacagcatcgggcccacacctacattgcatgaatatatgttagattt |
| | | gcaaccagagacaactgatctctacggtccagctggacaagcagaaccggacagagcccatta |
| | | caatattgtaaccttttgttgcaagtgtgactctacgcttcggttgtgcgtacaaagcacacacgtag |
| | | acattcgtactttggaagacctgttaatgggcacactaggaattgtgtgccccatctgtgctgctcc |
| | | cagaaagttaccacagttatgcacagagctgaacacttcactgcaagacatagaaataacctgtg |
| | | tatattgctatgactttgcttttcgggctgcttgcatagtatatagagatgggaatccatatgctgtatg |
| | | tgataaatgtttaaagttttattctaaaattagtgagtatagacattattgttatagtgtatatggaacaa |
| | | cattagctgcttctgtgtctacatacacatcattaatattgttggtattactattgtggataacagcagc |
| | | ctctgcgtttaggtgttttattgtatatattgtatttgtttatataccattatttttaatacatacacatgcac |
| | | gctttttaattgctgctgagactctttgccaacgtttaaatgtgtgtcaggacaaaatactaacacatt |
| | | atgaaaatgatagtacagacctacgtgaccatatagactattggaaacacatgcgcctagaatgtg |
| | | ctatttattacaaggccagagaaatgggatttaaacatattaaccaccaggtggtgccaacactgg |
| | | ctgtatcaaagaataaagcattacaagcaattgaactgcaactaacgttagaaacaatatataactc |
| | | acaatatagtaatgaaaagtggagtaacactacacccatagtacatttaaaaggtgatgctaatact |
| | | ttaaaatgtttaagatatagatttaaaaagcattgtaaattgtatactgcagtgtcgtctacatggcatt |
| | | ggacaggacataatgtaaaacataaaagtgcaattgttacacttacatatgatagtgaatgggggc |
| | | ccgccacgcagccagaactcgccccggaagaccccgaggattcggccctcttggaggacccc |
| | | gtggggacggtggcgccgcaaatcccaccaaactggcacatcccgtcgatccaggacgccgc |
| | | gacgccttaccatcccccggccaccccgaacaacatgggcctgatcgccggcgcggtgggcg |
| | | gcagtctcctggcagccctggtcatttgcggaattgtgtactggatgcaccgccgcactcggaaa |
| | | gccccaaagcgcatacgcctcccccacatccgggaagacgaccagccgtcctcgcaccagcc |
| | | cttgttttac* |
| WT gD | SEQ ID | atggggggggctgccgccaggttgggggccgtgattttgtttgtcgtcatagtgggcctccatgg |
| Nucleotide | NO: 48 | ggtccgcggcaaatatgccttggcggatgcctctctcaagatggccgaccccaatcgctttcgcg |
| | | gcaaagaccttccggtcctggaccagctgaccgaccctccgggggtccggcgcgtgtaccaca |
| | | tccaggcgggcctaccggacccgttccagccccccagcctcccgatcacggtttactacgccgt |
| | | gttggagcgcgcctgccgcagcgtgctcctaaacgcaccgtcggaggccccccagattgtccg |
| | | cggggcctccgaagacgtccggaaacaaccctacaacctgaccatcgcttggtttcggatggga |
| | | ggcaactgtgctatccccatcacggtcatggagtacaccgaatgctcctacaacaagtctctggg |
| | | ggcctgtcccatccgaacgcagccccgctggaactactatgacagcttcagcgccgtcagcga |
| | | ggataacctggggttcctgatgcacgcccccgcgtttgagaccgccggcacgtacctgcggctc |
| | | gtgaagataaacgactggacggagattacacagtttatcctggagcaccgagccaagggctcct |
| | | gtaagtacgccctcccgctgcgcatccccccgtcagcctgcctctccccccaggcctaccagca |
| | | gggggtgacggtggacagcatcgggatgctgccccgcttcatccccgagaaccagcgcaccg |
| | | tcgccgtatacagcttgaagatcgccgggtggcacgggcccaaggccccatacacgagcacc |
| | | ctgctgcccccggagctgtccgagacccccaacgccacgcagccagaactcgccccggaaga |
| | | ccccgaggattcggccctcttggaggaccccgtggggacggtggcgccgcaaatcccaccaa |
| | | actggcacatcccgtcgatccaggacgccgcgacgccttaccatcccccggccaccccgaaca |
| | | acatgggcctgatcgccggcgcggtgggcggcagtctcctggcagccctggtcatttgcggaat |
| | | tgtgtactggatgcaccgccgcactcggaaagccccaaagcgcatacgcctcccccacatccg |
| | | ggaagacgaccagccgtcctcgcaccagcccttgttttac |
| gDM1 | SEQ ID | MGGAAARLGAVILFVVIVGLHGVRGKYCLADASLKMADPN |
| Protein #2 | NO: 49 | RFRGKDLPVLDQLTDPPGVRRVYHIQAGLPDPFQPPSLPITVC |
| | | YAVLERACRSVLLNAPSEAPQIVRGASEDVRKQPYNLTIAWF |
| | | RMGGNCAIPITVMEYTECSYNKSLGACPIRTQPRWNYYDSFS |
| | | AVSEDNLGFLMHAPAFETAGTYLRLVKINDWTEITQFILEHR |
| | | AKGSCKYALPLRIPPSACLSPQAYQQGVTVDSIGMLPRFIPEN |
| | | QRTVAVYSLKIAGWHGPKAPYTSTLLPPELSETPNATQPELA |
| | | PEDPEDSALLEDPVGTVAPQIPPNWHIPSIQDAATPYHPPATP |
| | | NNMGLIAGAVGGSLLAALVICGIVYWMHRRTRKAPKRIRLP |
| | | HIREDDQPSSHQPLFYHIREDDQPSSHQPLFY |
| gD without | SEQ ID | MGGAAARLGAVILFVVIVGLHGVRGKYALADASLKMADPN |
| trans- | NO: 50 | RFRGKDLPVLDQLTDPPGVRRVYHIQAGLPDPFQPPSLPITVY |
| membrane | | YAVLERACRSVLLNAPSEAPQIVRGASEDVRKQPYNLTIAWF |
| domain | | RMGGNCAIPITVMEYTECSYNKSLGACPIRTQPRWNYYDSFS |
| (TM) | | AVSEDNLGFLMHAPAFETAGTYLRLVKINDWTEITQFILEHR |
| Protein | | AKGSCKYALPLRIPPSACLSPQAYQQGVTVDSIGMLPRFIPEN |
| | | QRTVAVYSLKIAGWHGPNRWPRIRYHAAAVYMTSTLLPPEL |
| | | SETPNATQPELAPEDPEDSALLEDPVGTVAPQIPPNWHIPSIQ |
| | | DAATPYHPPATPNNMGLITRKAPKRIRLPHIREDDQPSSHQPL |
| | | FY |
| gD without | SEQ ID | atggggggggctgccgccaggttgggggccgtgattttgtttgtcgtcatagtgggcctccatgg |
| trans- | NO: 51 | ggtccgcggcaaatatgccttggcggatgcctctctcaagatggccgaccccaatcgctttcgcg |
| membrane | | gcaaagaccttccggtcctggaccagctgaccgaccctccgggggtccggcgcgtgtaccaca |
| domain | | tccagggggcctaccggacccgttccagccccccagcctcccgatcacggtttactacgccgt |
| (TM) | | gttggagcgcgcctgccgcagcgtgctcctaaacgcaccgtcggaggccccccagattgtccg |
| Nucleotide | | cggggcctccgaagacgtccggaaacaaccctacaacctgaccatcgcttggtttcggatggga |
| | | ggcaactgtgctatccccatcacggtcatggagtacaccgaatgctcctacaacaagtctctggg |
| | | ggcctgtcccatccgaacgcagccccgctggaactactatgacagcttcagcgccgtcagcga |
| | | ggataacctggggttcctgatgcacgcccccgcgtttgagaccgccggcacgtacctgcggctc |
| | | gtgaagataaacgactggacggagattacacagtttatcctggagcaccgagccaagggctcct |
| | | gtaagtacgccctcccgctgcgcatccccccgtcagcctgcctctccccccaggcctaccagca |
| | | gggggtgacggtggacagcatcgggatgctgccccgcttcatccccgagaaccagcgcaccg |
| | | tcgccgtatacagcttgaagatcgccgggtggcacgggcccaaccggtggcctaggatccgat |
| | | atcacgcggccgcagtgtacatgacgagcaccctgctgcccccggagctgtccgagaccccca |
| | | acgccacgcagccagaactcgccccggaagaccccgaggattcggccctcttggaggacccc |
| | | gtggggacggtggcgccgcaaatcccaccaaactggcacatcccgtcgatccaggacgccgc |
| | | gacgccttaccatcccccggccaccccgaacaacatgggcctgatcactoggaaagccccaaa |
| | | gcgcatacgcctcccccacatccgggaagacgaccagccgtcctcgcaccagcccttgttttac |
| Super gD | SEQ ID | MGGAAARLGAVILFVVIVGLHGVRGKYALADASLKMADPN |
| with P2A | NO: 52 | RFRGKDLPVLDQLTDPPGVRRVYHIQAGLPDPFQPPSLPITVY |
| sequence | | YAVLERACRSVLLNAPSEAPQIVRGASEDVRKQPYNLTIAWF |
| Protein | | RMGGNCAIPITVMEYTECSYNKSLGACPIRTQPRWNYYDSFS |
| | | AVSEDNLGFLMHAPAFETAGTYLRLVKINDWTEITQFILEHR |
| | | AKGSCKYALPLRIPPSACLSPQAYQQGVTVDSIGMLPRFIPEN |
| | | QRTVAVYSLKIAGWHGPKAPYTSTLLPPELSETPNATQPELA |
| | | PEDPEDSALLEDPVGTVAPQIPPNAHIPSIQDAATPYHPPATPN |
| | | NMGLIAGAVGGSLLAALVICGIVYWMHRRTRKAPKRIRLPHI |
| | | REDDQPSSHQPLFYLSTGSGATNFSLLKQAGDVEENPGP |
| Super gD | SEQ ID | atggggggggctgccgccaggttgggggccgtgattttgtttgtcgtcatagtgggcctccatgg |
| with P2A | NO: 53 | ggtccgcggcaaatatgccttggcggatgcctctctcaagatggccgaccccaatcgctttcgcg |
| sequence | | gcaaagaccttccggtcctggaccagctgaccgaccctccgggggtccggcgcgtgtaccaca |
| Nucleotide | | tccaggcgggcctaccggacccgttccagccccccagcctcccgatcacggtttactacgccgt |
| | | gttggagcgcgcctgccgcagcgtgctcctaaacgcaccgtcggaggccccccagattgtccg |
| | | cggggcctccgaagacgtccggaaacaaccctacaacctgaccatcgcttggtttcggatggga |
| | | ggcaactgtgctatccccatcacggtcatggagtacaccgaatgctcctacaacaagtctctggg |
| | | ggcctgtcccatccgaacgcagccccgctggaactactatgacagcttcagcgccgtcagcga |
| | | ggataacctggggttcctgatgcacgcccccgcgtttgagaccgccggcacgtacctgcggctc |
| | | gtgaagataaacgactggacggagattacacagtttatcctggagcaccgagccaagggctcct |
| | | gtaagtacgccctcccgctgcgcatccccccgtcagcctgcctgtccccccaggcctaccagca |
| | | gggggtgacggtggacagcatcgggatgctgccccgcttcatccccgagaaccagcgcaccg |
| | | tcgccgtatacagcttgaagatcgccgggtggcacgggcccaaggccccatacacgagcacc |
| | | ctgctgcccccggagctgtccgagacccctaacgccacgcagccagaactcgccccggaaga |
| | | ccccgaggattcggccctcttggaggaccccgtggggacggtggcgccgcaaatcccaccaa |
| | | acgcgcacataccgtcgatccaggacgccgcgacgccttaccatcccccggccaccccgaac |
| | | aacatgggcctgatcgccggcgcggtgggcggcagtctcctggcagccctggtcatttgcgga |
| | | attgtgtactggatgcaccgccgcactcggaaagccccaaagcgcatacgcctcccccacatcc |
| | | gggaagacgaccagccgtcctcgcaccagcccttgttttacttgtcgacaggaagcggagctac |
| | | taacttcagcctgctgaagcaggctggagacgtggaggagaaccctggacct |
| E765-wt | SEQ ID | HGDTPTLHEYMLDLQPETTDLYCYEQLNDSSEEEDEIDGPAG |
| Protein | NO: 54 | QAEPDRAHYNIVTFCCKCDSTLRLCVQSTHVDIRTLEDLLMG |
| | | TLGIVCPICSQKPGAHQKRTAMFQDPQERPRKLPQLCTELQT |
| | | TIHDIILECVYCKQQLLRREVYDFAFRDLCIVYRDGNPYAVC |
| | | DKCLKFYSKISEYRHYCYSLYGTTLEQQYNKPLCDLLIRCINC |
| | | QKPLCPEEKQRHLDKKQRFHNIRGRWTGRCMSCCRSSRTRR |
| | | ETQLAAACIHNITGVLFALLCVLLCVCLLIRPLLLSVSTYTSLII |
| | | LVLLLWITAASAFRCFIVYIIFVYIPLFLIHTHARFLITG |
| E765-wt | SEQ ID | catggagatacacctacattgcatgaatatatgttagatttgcaaccagagacaactgatctctact |
| Nucleotide | NO: 55 | gttatgagcaattaaatgacagctcagaggaggaggatgaaatagatggtccagctggacaagc |
| | | agaaccggacagagcccattacaatattgtaaccttttgttgcaagtgtgactctacgcttcggttgt |
| | | gcgtacaaagcacacacgtagacattcgtactttggaagacctgttaatgggcacactaggaatt |
| | | gtgtgccccatctgttctcagaaaccaggcgcccaccaaaagagaactgcaatgtttcaggacc |
| | | cacaggagcgacccagaaagttaccacagttatgcacagagctgcaaacaactatacatgatat |
| | | aatattagaatgtgtgtactgcaagcaacagttactgcgacgtgaggtatatgactttgcttttcggg |
| | | atttatgcatagtatatagagatgggaatccatatgctgtatgtgataaatgtttaaagttttattctaaa |
| | | attagtgagtatagacattattgttatagtttgtatggaacaacattagaacagcaatacaacaaacc |
| | | gttgtgtgatttgttaattaggtgtattaactgtcaaaagccactgtgtcctgaagaaaagcaaagac |
| | | atctggacaaaaagcaaagattccataatataaggggtcggtggaccggtcgatgtatgtcttgtt |
| | | gcagatcatcaagaacacgtagagaaacccagctggcggccgcctgcatccacaacattactg |
| | | gcgtgctttttgctttgctttgtgtgcttttgtgtgtctgcctattaatacgtccgctgcttttgtctgtgtc |
| | | tacatacacatcattaataatattggtattactattgtggataacagcagcctctgcgtttaggtgtttt |
| | | attgtatatattatatttgtttatataccattatttttaatacatacacatgcacgctttttaattacagggc |
| | | c |
| C-terminal | SEQ ID | SNTTPIVHLKGDANTLKCLRYRFKKHCKLYTAVSSTWHWTG |
| HPV E2 | NO: 56 | HNVKHKSAIVTLTYDSEW |
| protein | | |
| C-terminal | SEQ ID | agtaacactacacccatagtacatttaaaaggtgatgctaatactttaaaatgtttaagatatagattt |
| HPV | NO: 57 | aaaaagcattgtaaattgtatactgcagtgtcgtctacatggcattggacaggacataatgtaaaac |
| Nucleotide | | ataaaagtgcaattgttacacttacatatgatagtgaatgg |
| N-terminal | SEQ ID | ETLCQRLNVCQDKILTHYENDSTDLRDHIDYWKHMRLECAI |
| E2 protein | NO: 58 | YYKAREMGFKHINHQVVPTLAVSKNKALQAIELQLTLETIYN |
| | | SQYSNEKW |
| N-terminal | SEQ ID | gagactctttgccaacgtttaaatgtgtgtcaggacaaaatactaacacattatgaaaatgatagta |
| E2 | NO: 59 | cagacctacgtgaccatatagactattggaaacacatgcgcctagaatgtgctatttattacaagg |
| Nucleotide | | ccagagaaatgggatttaaacatattaaccaccaggtggtgccaacactggctgtatcaaagaat |
| | | aaagcattacaagcaattgaactgcaactaacgttagaaacaatatataactcacaatatagtaatg |
| | | aaaagtgg |
| Mutant | SEQ ID | TPTLHEYMLDLQPETTDLYGPAGQAEPDRAHYNIVTFCCKC |
| HPV E7, | NO: 60 | DSTLRLCVQSTHVDIRTLEDLLMGTLGIVCPICAAPRKLPQLC |
| E6, E5 and | | TELNTSLQDIEITCVYCYDFAFRAACIVYRDGNPYAVCDKCL |
| C-terminus | | KFYSKISEYRHYCYSVYGTTLAASVSTYTSLILLVLLLWITAA |
| E2 Fusion | | SAFRCFIVYIVFVYIPLFLIHTHARFLIAASNTTPIVHLKGDAN |
| Protein | | TLKCLRYRFKKHCKLYTAVSSTWHWTGHNVKHKSAIVTLT |
| | | YDSEW |
| Mutant | SEQ ID | acacctacattgcatgaatatatgttagatttgcaaccagagacaactgatctctacggtccagctg |
| HPV E7, | NO: 61 | gacaagcagaaccggacagagcccattacaatattgtaaccttttgttgcaagtgtgactctacgc |
| E6, E5 and | | ttcggttgtgcgtacaaagcacacacgtagacattcgtactttggaagacctgttaatgggcacac |
| C-terminus | | taggaattgtgtgccccatctgtgctgctcccagaaagttaccacagttatgcacagagctgaaca |
| E2 Fusion | | cttcactgcaagacatagaaataacctgtgtatattgctatgactttgcttttcgggctgcttgcatag |
| Nucleotide | | tatatagagatgggaatccatatgctgtatgtgataaatgtttaaagttttattctaaaattagtgagta |
| | | tagacattattgttatagtgtatatggaacaacattagctgcttctgtgtctacatacacatcattaatat |
| | | tgttggtattactattgtggataacagcagcctctgcgtttaggtgttttattgtatatattgtatttgttta |
| | | tataccattatttttaatacatacacatgcacgctttttaattgctgctagtaacactacacccatagta |
| | | catttaaaaggtgatgctaatactttaaaatgtttaagatatagatttaaaaagcattgtaaattgtata |
| | | ctgcagtgtcgtctacatggcattggacaggacataatgtaaaacataaaagtgcaattgttacact |
| | | tacatatgatagtgaatgg |
| Mutant | SEQ ID | TPTLHEYMLDLQPETTDLYGPAGQAEPDRAHYNIVTFCCKC |
| HPV E7, | NO: 62 | DSTLRLCVQSTHVDIRTLEDLLMGTLGIVCPICAAPRKLPQLC |
| E6, E5 and | | TELNTSLQDIEITCVYCYDFAFRAACIVYRDGNPYAVCDKCL |
| N-terminus | | KFYSKISEYRHYCYSVYGTTLAASVSTYTSLILLVLLLWITAA |
| E2 Fusion | | SAFRCFIVYIVFVYIPLFLIHTHARFLIAAETLCQRLNVCQDKI |
| Protein | | LTHYENDSTDLRDHIDYWKHMRLECAIYYKAREMGFKHINH |
| | | QVVPTLAVSKNKALQAIELQLTLETIYNSQYSNEKW |
| Mutant | SEQ ID | acacctacattgcatgaatatatgttagatttgcaaccagagacaactgatctctacggtccagctg |
| HPV E7, | NO: 63 | gacaagcagaaccggacagagcccattacaatattgtaaccttttgttgcaagtgtgactctacgc |
| E6, E5 and | | ttcggttgtgcgtacaaagcacacacgtagacattcgtactttggaagacctgttaatgggcacac |
| N-terminus | | taggaattgtgtgccccatctgtgctgctcccagaaagttaccacagttatgcacagagctgaaca |
| E2 Fusion | | cttcactgcaagacatagaaataacctgtgtatattgctatgactttgcttttcgggctgcttgcatag |
| Nucleotide | | tatatagagatgggaatccatatgctgtatgtgataaatgtttaaagttttattctaaaattagtgagta |
| | | tagacattattgttatagtgtatatggaacaacattagctgcttctgtgtctacatacacatcattaatat |
| | | tgttggtattactattgtggataacagcagcctctgcgtttaggtgttttattgtatatattgtatttgttta |
| | | tataccattatttttaatacatacacatgcacgctttttaattgctgctgagactctttgccaacgtttaa |
| | | atgtgtgtcaggacaaaatactaacacattatgaaaatgatagtacagacctacgtgaccatatag |
| | | actattggaaacacatgcgcctagaatgtgctatttattacaaggccagagaaatgggatttaaac |
| | | atattaaccaccaggtggtgccaacactggctgtatcaaagaataaagcattacaagcaattgaa |
| | | ctgcaactaacgttagaaacaatatataactcacaatatagtaatgaaaagtgg |
| Mutant | SEQ ID | TPTLHEYMLDLQPETTAAAAAAQLNDSSEEEDEIDGPAGQA |
| HPV E7 | NO: 64 | EPDRAHYNIVTFCCKCDSTLRLCVQSTHVDIRTLEDLLMGTL |
| Protein | | GIVCPICSQKP |
| Version 2 | | |
| Mutant | SEQ ID | acccccaccctgcacgagtacatgctggacctgcagcccgagaccaccgccgccgccgccgc |
| HPV E7 | NO: 65 | cgcccagctgaacgacagcagcgaggaggaggacgagatcgacggccccgccggccaggc |
| Nucleotide | | cgagcccgacagggctcactacaacatcgtgaccttctgctgcaagtgcgacagcaccctgag |
| Version 2 | | gctgtgcgtgcagagcacccacgtggacatcaggaccctggaggacctgctgatgggcaccct |
| | | gggcatcgtgtgccccatctgcagccagaagccc |
| Mutant | SEQ ID | HQKRTAMFQDPQERPRKLPQLCTELQTTIHDIILECVYCKQQ |
| HPV E6 | NO: 66 | LLRREVYDAAAAALCIVYRDGNPYAVCDKCLKFYSKISEYR |
| Protein | | HYCYSLYGTTLEQQYNKPLCDLLIRCINCQKPQCPEEKQRHL |
| Version 2 | | DKKQRFHNIRGRWTGRCMSCCRSSRTRRETQL |
| Mutant | SEQ ID | caccagaagaggaccgccatgttccaggacccccaggagaggcccaggaagctgccccagc |
| HPV E6 | NO: 67 | tgtgcaccgagctgcagaccaccatccacgacatcatcctggagtgcgtgtactgcaagcagca |
| Nucleotide | | gctgctgaggagggaggtgtacgacgccgccgccgccgccctgtgcatcgtgtacagggacg |
| Version 2 | | gcaacccctacgccgtgtgcgacaagtgcctgaagttctacagcaagatcagcgagtacaggc |
| | | actactgctacagcctgtacggcaccaccctggagcagcagtacaacaagcccctgtgcgacct |
| | | gctgatcaggtgcatcaactgccagaagccccagtgccccgaggagaagcagaggcacctgg |
| | | acaagaagcagaggttccacaacatcaggggcaggtggaccggcaggtgcatgagctgctgc |
| | | aggagcagcaggaccaggagggagacccagctg |
| Mutant | SEQ ID | CVLLCVCLLIRPLLLSVSTYTSLIILVLLLWITAASAFRCFIVYI |
| HPV E5 | NO: 68 | AFVYIPLFLIHTHA |
| Protein | | |
| Version 2 | | |
| Mutant | SEQ ID | tgcgtgctgctgtgcgtgtgcctgctgatcaggcccctgctgctgagcgtgagcacctacaccag |
| HPV E5 | NO: 69 | cctgatcatcctggtgctgctgctgtggatcaccgccgccagcgccttcaggtgcttcatcgtgta |
| Nucleotide | | catcgccttcgtgtacatccccctgttcctgatccacacccacgcc |
| Version 2 | | |
| Mutant E7, | SEQ ID | TPTLHEYMLDLQPETTAAAAAAQLNDSSEEEDEIDGPAGQA |
| E6 and E5 | NO: 70 | EPDRAHYNIVTFCCKCDSTLRLCVQSTHVDIRTLEDLLMGTL |
| fusion | | GIVCPICSQKPGAHQKRTAMFQDPQERPRKLPQLCTELQTTIH |
| Protein | | DIILECVYCKQQLLRREVYDAAAAALCIVYRDGNPYAVCDK |
| | | CLKFYSKISEYRHYCYSLYGTTLEQQYNKPLCDLLIRCINCQK |
| | | PQCPEEKQRHLDKKQRFHNIRGRWTGRCMSCCRSSRTRRET |
| | | QLGACVLLCVCLLIRPLLLSVSTYTSLIILVLLLWITAASAFRC |
| | | FIVYIAFVYIPLFLIHTHA |
| Mutant E7, | SEQ ID | acccccaccctgcacgagtacatgctggacctgcagcccgagaccaccgccgccgccgccgc |
| E6 and E5 | NO: 71 | cgcccagctgaacgacagcagcgaggaggaggacgagatcgacggccccgccggccaggc |
| fusion | | cgagcccgacagggctcactacaacatcgtgaccttctgctgcaagtgcgacagcaccctgag |
| Nucleotide | | gctgtgcgtgcagagcacccacgtggacatcaggaccctggaggacctgctgatgggcaccct |
| | | gggcatcgtgtgccccatctgcagccagaagcccggcgcccaccagaagaggaccgccatgt |
| | | tccaggacccccaggagaggcccaggaagctgccccagctgtgcaccgagctgcagaccac |
| | | catccacgacatcatcctggagtgcgtgtactgcaagcagcagctgctgaggagggaggtgtac |
| | | gacgccgccgccgccgccctgtgcatcgtgtacagggacggcaacccctacgccgtgtgcga |
| | | caagtgcctgaagttctacagcaagatcagcgagtacaggcactactgctacagcctgtacggc |
| | | accaccctggagcagcagtacaacaagcccctgtgcgacctgctgatcaggtgcatcaactgcc |
| | | agaagccccagtgccccgaggagaagcagaggcacctggacaagaagcagaggttccacaa |
| | | catcaggggcaggtggaccggcaggtgcatgagctgctgcaggagcagcaggaccaggagg |
| | | gagacccagctgggcgcctgcgtgctgctgtgcgtgtgcctgctgatcaggcccctgctgctga |
| | | gcgtgagcacctacaccagcctgatcatcctggtgctgctgctgtggatcaccgccgccagcgc |
| | | cttcaggtgcttcatcgtgtacatcgccttcgtgtacatccccctgttcctgatccacacccacgcc |
| Melapoly | SEQ ID | AAARKFFHRTCKCTGNFAAAANESFALPYWNFATGRNECD |
| Protein #2 | NO: 72 | VAAADQLGYSYAIDLPVSVAAAAAASVYDFFVWLAAAAAA |
| | | STFSFRNALAASQVMNLHNLAATAPDNLGYMAAAIAVVNA |
| | | LLLYAYDYEELYAKVPRNQDWLAAAAFGLANEKSIAAAAP |
| | | R |
| Melapoly | SEQ ID | gccgctgcaaggaagttctttcacagaacctgcaaatgtacaggaaacttcgccgctgcagcca |
| Nucleotide | NO: 73 | atgagagcttcgccctgccctattggaactttgctaccggccggaatgaatgcgacgtggctgca |
| #2 | | gccgatcagctcggatactcttatgccatcgacctgcctgtgagtgtcgctgcagccgctgcagc |
| | | ctcagtgtacgatttctttgtctggctggctgcagccgctgcagccagcactttctcctttcgcaacg |
| | | ccctggctgcatctcaggtcatgaacctccataatctggcagctaccgcaccagacaacctogga |
| | | tatatggcagccgctattgccgtggtcaatgctctgctcctgtacgcctatgactacgaggaactgt |
| | | acgctaaagtccccaggaatcaggattggctcgcagccgctgcatttggtctggccaacgaaaa |
| | | gagcattgcagcagcagcacctagg |
| Melanoma | SEQ ID | AKFVAAWTLKAAAAAARKFFHRTCKCTGNFAAAANESFAL |
| antigens | NO: 74 | PYWNFATGRNECDVAAADQLGYSYAIDLPVSVAAAAAASV |
| with | | YDFFVWLAAAAAASTFSFRNALAASQVMNLHNLAATAPDN |
| universal | | LGYMAAAIAVVNALLLYAYDYEELYAKVPRNQDWLAAAA |
| helper | | FGLANEKSIAAAAPR |
| epitope | | |
| Protein | | |
| Melanoma | SEQ ID | gctaagtttgtggccgcttggacactgaaggccgctgcagccgctgcaaggaagttctttcacag |
| antigens | NO: 75 | aacctgcaaatgtacaggaaacttcgccgctgcagccaatgagagcttcgccctgccctattgga |
| with | | actttgctaccggccggaatgaatgcgacgtggctgcagccgatcagctcggatactcttatgcc |
| universal | | atcgacctgcctgtgagtgtcgctgcagccgctgcagcctcagtgtacgatttctttgtctggctgg |
| helper | | ctgcagccgctgcagccagcactttctcctttcgcaacgccctggctgcatctcaggtcatgaacc |
| epitope | | tccataatctggcagctaccgcaccagacaacctcggatatatggcagccgctattgccgtggtc |
| Nucleotide | | aatgctctgctcctgtacgcctatgactacgaggaactgtacgctaaagtccccaggaatcagga |
| | | ttggctcgcagccgctgcatttggtctggccaacgaaaagagcattgcagcagcagcacctagg |
| Melanoma | SEQ ID | AKFVAAWTLKAAAAAARKFFHRTCKCTGNFAAAANESFAL |
| antigens | NO: 76 | PYWNFATGRNECDVAAADQLGYSYAIDLPVSVAAAAAASV |
| with | | YDFFVWLAAAAAASTFSFRNALAASQVMNLHNLAATAPDN |
| universal | | LGYMAAAIAVVNALLLYAYDYEELYAKVPRNQDWLAAAA |
| helper | | FGLANEKSIAAAAPRETLCQRLNVCQDKILTHYENDSTDLRD |
| epitope and | | HIDYWKHMRLECAIYYKAREMGFKHINHQVVPTLAVSKNK |
| E2 Protein | | ALQAIELQLTLETIYNSQYSNEKWSNTTPIVHLKGDANTLKC |
| | | LRYRFKKHCKLYTAVSSTWHWTGHNVKHKSAIVTLTYDSE |
| | | W |
| Melanoma | SEQ ID | gctaagtttgtggccgcttggacactgaaggccgctgcagccgctgcaaggaagttctttcacag |
| antigens | NO: 77 | aacctgcaaatgtacaggaaacttcgccgctgcagccaatgagagcttcgccctgccctattgga |
| with | | actttgctaccggccggaatgaatgcgacgtggctgcagccgatcagctcggatactcttatgcc |
| universal | | atcgacctgcctgtgagtgtcgctgcagccgctgcagcctcagtgtacgatttctttgtctggctgg |
| helper | | ctgcagccgctgcagccagcactttctcctttcgcaacgccctggctgcatctcaggtcatgaacc |
| epitope and | | tccataatctggcagctaccgcaccagacaacctcggatatatggcagccgctattgccgtggtc |
| E2 | | aatgctctgctcctgtacgcctatgactacgaggaactgtacgctaaagtccccaggaatcagga |
| Nucleotide | | ttggctcgcagccgctgcatttggtctggccaacgaaaagagcattgcagcagcagcacctagg |
| | | gagactctttgccaacgtttaaatgtgtgtcaggacaaaatactaacacattatgaaaatgatagta |
| | | cagacctacgtgaccatatagactattggaaacacatgcgcctagaatgtgctatttattacaagg |
| | | ccagagaaatgggatttaaacatattaaccaccaggtggtgccaacactggctgtatcaaagaat |
| | | aaagcattacaagcaattgaactgcaactaacgttagaaacaatatataactcacaatatagtaatg |
| | | aaaagtggagtaacactacacccatagtacatttaaaaggtgatgctaatactttaaaatgtttaag |
| | | atatagatttaaaaagcattgtaaattgtatactgcagtgtcgtctacatggcattggacaggacata |
| | | atgtaaaacataaaagtgcaattgttacacttacatatgatagtgaatgg |
| Melanoma | SEQ ID | AKFVAAWTLKAAAAAARKFFHRTCKCTGNFAAAANESFAL |
| antigens | NO: 78 | PYWNFATGRNECDVAAADQLGYSYAIDLPVSVAAAAAASV |
| with | | YDFFVWLAAAAAASTFSFRNALAASQVMNLHNLAATAPDN |
| universal | | LGYMAAAIAVVNALLLYAYDYEELYAKVPRNQDWLAAAA |
| helper | | FGLANEKSIAAAAPRSNTTPIVHLKGDANTLKCLRYRFKKHC |
| epitope and | | KLYTAVSSTWHWTGHNVKHKSAIVTLTYDSEW |
| C-terminus | | |
| E2 Protein | | |
| Melanoma | SEQ ID | gctaagtttgtggccgcttggacactgaaggccgctgcagccgctgcaaggaagttctttcacag |
| antigens | NO: 79 | aacctgcaaatgtacaggaaacttcgccgctgcagccaatgagagcttcgccctgccctattgga |
| with | | actttgctaccggccggaatgaatgcgacgtggctgcagccgatcagctcggatactcttatgcc |
| universal | | atcgacctgcctgtgagtgtcgctgcagccgctgcagcctcagtgtacgatttctttgtctggctgg |
| helper | | ctgcagccgctgcagccagcactttctcctttcgcaacgccctggctgcatctcaggtcatgaacc |
| epitope and | | tccataatctggcagctaccgcaccagacaacctoggatatatggcagccgctattgccgtggtc |
| C-terminus | | aatgctctgctcctgtacgcctatgactacgaggaactgtacgctaaagtccccaggaatcagga |
| E2 | | ttggctcgcagccgctgcatttggtctggccaacgaaaagagcattgcagcagcagcacctagg |
| Nucleotide | | agtaacactacacccatagtacatttaaaaggtgatgctaatactttaaaatgtttaagatatagattt |
| | | aaaaagcattgtaaattgtatactgcagtgtcgtctacatggcattggacaggacataatgtaaaac |
| | | ataaaagtgcaattgttacacttacatatgatagtgaatgg |
| Melanoma | SEQ ID | AKFVAAWTLKAAAAAARKFFHRTCKCTGNFAAAANESFAL |
| antigens | NO: 80 | PYWNFATGRNECDVAAADQLGYSYAIDLPVSVAAAAAASV |
| with | | YDFFVWLAAAAAASTFSFRNALAASQVMNLHNLAATAPDN |
| universal | | LGYMAAAIAVVNALLLYAYDYEELYAKVPRNQDWLAAAA |
| helper | | FGLANEKSIAAAAPRETLCQRLNVCQDKILTHYENDSTDLRD |
| epitope and | | HIDYWKHMRLECAIYYKAREMGFKHINHQVVPTLAVSKNK |
| N-terminus | | ALQAIELQLTLETIYNSQYSNEKW |
| E2 Protein | | |
| Melanoma | SEQ ID | gctaagtttgtggccgcttggacactgaaggccgctgcagccgctgcaaggaagttctttcacag |
| antigens | NO: 81 | aacctgcaaatgtacaggaaacttcgccgctgcagccaatgagagcttcgccctgccctattgga |
| with | | actttgctaccggccggaatgaatgcgacgtggctgcagccgatcagctcggatactcttatgcc |
| universal | | atcgacctgcctgtgagtgtcgctgcagccgctgcagcctcagtgtacgatttctttgtctggctgg |
| helper | | ctgcagccgctgcagccagcactttctcctttcgcaacgccctggctgcatctcaggtcatgaacc |
| epitope and | | tccataatctggcagctaccgcaccagacaacctcggatatatggcagccgctattgccgtggtc |
| N-terminus | | aatgctctgctcctgtacgcctatgactacgaggaactgtacgctaaagtccccaggaatcagga |
| E2 | | ttggctcgcagccgctgcatttggtctggccaacgaaaagagcattgcagcagcagcacctagg |
| Nucleotide | | gagactctttgccaacgtttaaatgtgtgtcaggacaaaatactaacacattatgaaaatgatagta |
| | | cagacctacgtgaccatatagactattggaaacacatgcgcctagaatgtgctatttattacaagg |
| | | ccagagaaatgggatttaaacatattaaccaccaggtggtgccaacactggctgtatcaaagaat |
| | | aaagcattacaagcaattgaactgcaactaacgttagaaacaatatataactcacaatatagtaatg |
| | | aaaagtgg |
| gD- | SEQ ID | MGGAAARLGAVILFVVIVGLHGVRGKYALADASLKMADPN |
| E765dt1 | NO: 82 | RFRGKDLPVLDQLTDPPGVRRVYHIQAGLPDPFQPPSLPITVY |
| Protein | | YAVLERACRSVLLNAPSEAPQIVRGASEDVRKQPYNLTIAWF |
| | | RMGGNCAIPITVMEYTECSYNKSLGACPIRTQPRWNYYDSFS |
| | | AVSEDNLGFLMHAPAFETAGTYLRLVKINDWTEITQFILEHR |
| | | AKGSCKYALPLRIPPSACLSPQAYQQGVTVDSIGMLPRFIPEN |
| | | QRTVAVYSLKIAGWHGPHGDTPTLHEYMLDLQPETTGLYGY |
| | | GQLNDSSEEEDEIDGPAGQAEPDRAHYNIVTFCCKCDSTLRL |
| | | CVQSTHVDIRTLEDLLMGTLGIVCPICSQKPGAHQKRTAMFQ |
| | | DPQERPRKLPQLCTELQTTIHDIILECVYCKQQLLRREVYDYA |
| | | YRHLCIVYRDGNPYAVCDKCLKFYSKISEYRHYCYSLYGTTL |
| | | EQQYNKPLCDLLIRCINCQKPQCPEEKQRHLDKKQRFHNIRG |
| | | RWTGRCMSCCRSSRTRRETQLAAATNLDTASTTLLACFLLCF |
| | | CVLLCVCLLIRPLLLSVSTYTSLIILVLLLWITAASAFRCFIVYI |
| | | VFVYIPLFLIHTHAGPKAPYTSTLLPPELSETPNATQPELAPED |
| | | PEDSALLEDPVGTVAPQIPPNWHIPSIQDAATPYHPPATPNNM |
| | | GLIAGAVGGSLLAALVICGIVYWMHRRTRKAPKRIRLPHIRE |
| | | DDQPSSHQPLFY |
| gD- | SEQ ID | atggggggggctgccgccaggttgggggccgtgattttgtttgtcgtcatagtgggcctccatgg |
| E765dt1 | NO: 83 | ggtccgcggcaaatatgccttggcggatgcctctctcaagatggccgaccccaatcgctttcgcg |
| Nucleotide | | gcaaagaccttccggtcctggaccagctgaccgaccctccgggggtccggcgcgtgtaccaca |
| | | tccaggcgggcctaccggacccgttccagccccccagcctcccgatcacggtttactacgccgt |
| | | gttggagcgcgcctgccgcagcgtgctcctaaacgcaccgtcggaggccccccagattgtccg |
| | | cggggcctccgaagacgtccggaaacaaccctacaacctgaccatcgcttggtttcggatggga |
| | | ggcaactgtgctatccccatcacggtcatggagtacaccgaatgctcctacaacaagtctctggg |
| | | ggcctgtcccatccgaacgcagccccgctggaactactatgacagcttcagcgccgtcagcga |
| | | ggataacctggggttcctgatgcacgcccccgcgtttgagaccgccggcacgtacctgcggctc |
| | | gtgaagataaacgactggacggagattacacagtttatcctggagcaccgagccaagggctcct |
| | | gtaagtacgccctcccgctgcgcatccccccgtcagcctgcctgtccccccaggcctaccagca |
| | | gggggtgacggtggacagcatcgggatgctgccccgcttcatccccgagaaccagcgcaccg |
| | | tcgccgtatacagcttgaagatcgccgggtggcacgggccccacggggacactcccaccctgc |
| | | atgaatatatgctggatctgcagcctgagaccacaggcctgtacggctatggacagctgaacga |
| | | cagctccgaggaagaggacgaaatcgatgggccagcaggacaggcagagccagacagagc |
| | | ccactacaatattgtgaccttctgctgtaagtgcgatagtacactgcgactgtgcgtgcagtcaaca |
| | | catgtcgacatccgcactctggaggatctgctgatgggaaccctggggatcgtgtgccctatttgt |
| | | tctcagaagccaggcgctcaccagaaaaggactgcaatgtttcaggatccacaggaacgaccc |
| | | aggaagctgcctcagctgtgcaccgagctgcagactaccatccacgacatcattctggaatgcgt |
| | | gtattgtaaacagcagctgctgaggagagaggtctacgattatgcatacaggcatctgtgcatcgt |
| | | gtatagagacggaaacccatacgccgtctgcgataagtgtctgaaattctattccaagatctctga |
| | | gtacaggcactattgttacagcctgtatggcacaactctggaacagcagtacaacaaacccctgt |
| | | gcgacctgctgatcagatgcattaattgtcagaagccccagtgtcctgaagagaaacagcggca |
| | | cctggataagaaacagaggttccataacatcagaggccggtggacaggaagatgcatgtcttgc |
| | | tgtcgctctagtcgaacccgacgagagacacagctggcagctgcaactaatctggacaccgcc |
| | | agtaccacactgctggcttgtttcctgctgtgcttttgcgtgctgctgtgcgtctgtctgctgatccgg |
| | | cctctgctgctgtcagtgagcacatacactagcctgatcattctggtcctgctgctgtggattacag |
| | | ccgcttccgctttccgctgttttatcgtgtatatcgtgttcgtgtacatccccctgtttctgattcacact |
| | | catgccgggcccaaggccccatacacgagcaccctgctgcccccggagctgtccgagacccc |
| | | taacgccacgcagccagaactcgccccggaagaccccgaggattcggccctcttggaggacc |
| | | ccgtggggacggtggcgccgcaaatcccaccaaactggcacatcccgtcgatccaggacgcc |
| | | gcgacgccttaccatcccccggccaccccgaacaacatgggcctgatcgccggcgcggtggg |
| | | cggcagtctcctggcagccctggtcatttgcggaattgtgtactggatgcaccgccgcactcgga |
| | | aagccccaaagcgcatacgcctcccccacatccgggaagacgaccagccgtcctcgcaccag |
| | | cccttgttttac |
| gDM1- | SEQ ID | MGGAAARLGAVILFVVIVGLHGVRGKYCLADASLKMADPN |
| E765dt1 | NO: 84 | RFRGKDLPVLDQLTDPPGVRRVYHIQAGLPDPFQPPSLPITVC |
| Protein | | YAVLERACRSVLLNAPSEAPQIVRGASEDVRKQPYNLTIAWF |
| | | RMGGNCAIPITVMEYTECSYNKSLGACPIRTQPRWNYYDSFS |
| | | AVSEDNLGFLMHAPAFETAGTYLRLVKINDWTEITQFILEHR |
| | | AKGSCKYALPLRIPPSACLSPQAYQQGVTVDSIGMLPRFIPEN |
| | | QRTVAVYSLKIAGWHGPHGDTPTLHEYMLDLQPETTGLYGY |
| | | GQLNDSSEEEDEIDGPAGQAEPDRAHYNIVTFCCKCDSTLRL |
| | | CVQSTHVDIRTLEDLLMGTLGIVCPICSQKPGAHQKRTAMFQ |
| | | DPQERPRKLPQLCTELQTTIHDIILECVYCKQQLLRREVYDYA |
| | | YRHLCIVYRDGNPYAVCDKCLKFYSKISEYRHYCYSLYGTTL |
| | | EQQYNKPLCDLLIRCINCQKPQCPEEKQRHLDKKQRFHNIRG |
| | | RWTGRCMSCCRSSRTRRETQLAAATNLDTASTTLLACFLLCF |
| | | CVLLCVCLLIRPLLLSVSTYTSLIILVLLLWITAASAFRCFIVYI |
| | | VFVYIPLFLIHTHAGPKAPYTSTLLPPELSETPNATQPELAPED |
| | | PEDSALLEDPVGTVAPQIPPNWHIPSIQDAATPYHPPATPNNM |
| | | GLIAGAVGGSLLAALVICGIVYWMHRRTRKAPKRIRLPHIRE |
| | | DDQPSSHQPLFY |
| gDM1- | SEQ ID | atggggggggctgccgccaggttgggggccgtgattttgtttgtcgtcatagtgggcctccatgg |
| E765dt1 | NO: 85 | ggtccgcggcaaatattgtttggcggatgcctctctcaagatggccgaccccaatcgctttcgcg |
| Nucleotide | | gcaaagaccttccggtcctggaccagctgaccgaccctccgggggtccggcgcgtgtaccaca |
| | | tccaggcgggcctaccggacccgttccagccccccagcctcccgatcacggtttgctacgccgt |
| | | gttggagcgcgcctgccgcagcgtgctcctaaacgcaccgtcggaggccccccagattgtccg |
| | | cggggcctccgaagacgtccggaaacaaccctacaacctgaccatcgcttggtttcggatggga |
| | | ggcaactgtgctatccccatcacggtcatggagtacaccgaatgctcctacaacaagtctctggg |
| | | ggcctgtcccatccgaacgcagccccgctggaactactatgacagcttcagcgccgtcagcga |
| | | ggataacctggggttcctgatgcacgcccccgcgtttgagaccgccggcacgtacctgcggctc |
| | | gtgaagataaacgactggacggagattacacagtttatcctggagcaccgagccaagggctcct |
| | | gtaagtacgccctcccgctgcgcatccccccgtcagcctgcctctccccccaggcctaccagca |
| | | gggggtgacggtggacagcatcgggatgctgccccgcttcatccccgagaaccagcgcaccg |
| | | tcgccgtatacagcttgaagatcgccgggtggcacgggccccacggggacactcccaccctgc |
| | | atgaatatatgctggatctgcagcctgagaccacaggcctgtacggctatggacagctgaacga |
| | | cagctccgaggaagaggacgaaatcgatgggccagcaggacaggcagagccagacagagc |
| | | ccactacaatattgtgaccttctgctgtaagtgcgatagtacactgcgactgtgcgtgcagtcaaca |
| | | catgtcgacatccgcactctggaggatctgctgatgggaaccctggggatcgtgtgccctatttgt |
| | | tctcagaagccaggcgctcaccagaaaaggactgcaatgtttcaggatccacaggaacgaccc |
| | | aggaagctgcctcagctgtgcaccgagctgcagactaccatccacgacatcattctggaatgcgt |
| | | gtattgtaaacagcagctgctgaggagagaggtctacgattatgcatacaggcatctgtgcatcgt |
| | | gtatagagacggaaacccatacgccgtctgcgataagtgtctgaaattctattccaagatctctga |
| | | gtacaggcactattgttacagcctgtatggcacaactctggaacagcagtacaacaaacccctgt |
| | | gcgacctgctgatcagatgcattaattgtcagaagccccagtgtcctgaagagaaacagcggca |
| | | cctggataagaaacagaggttccataacatcagaggccggtggacaggaagatgcatgtcttgc |
| | | tgtcgctctagtcgaacccgacgagagacacagctggcagctgcaactaatctggacaccgcc |
| | | agtaccacactgctggcttgtttcctgctgtgcttttgcgtgctgctgtgcgtctgtctgctgatccgg |
| | | cctctgctgctgtcagtgagcacatacactagcctgatcattctggtcctgctgctgtggattacag |
| | | ccgcttccgctttccgctgttttatcgtgtatatcgtgttcgtgtacatccccctgtttctgattcacact |
| | | catgccgggcccaaggccccatacacgagcaccctgctgcccccggagctgtccgagacccc |
| | | caacgccacgcagccagaactcgccccggaagaccccgaggattcggccctcttggaggacc |
| | | ccgtggggacggtggcgccgcaaatcccaccaaactggcacatcccgtcgatccaggacgcc |
| | | gcgacgccttaccatcccccggccaccccgaacaacatgggcctgatcgccggcgcggtggg |
| | | cggcagtctcctggcagccctggtcatttgcggaattgtgtactggatgcaccgccgcactcgga |
| | | aagccccaaagcgcatacgcctcccccacatccgggaagacgaccagccgtcctcgcaccag |
| | | cccttgttttac |
| gDM2- | SEQ ID | MGGAAARLGAVILFVVIVGLHGVRGKYALADASLKMADPN |
| E765dt1 | NO: 86 | RFRGKDLPVLDQLTDPPGVRRVYHIQAGLPDPFQPPSLPITVY |
| Protein | | YAVLERACRSVLLNAPSEAPQIVRGASEDVRKQPYNLTIAWF |
| | | RMGGNCAIPITVMEYTECSYNKSLGACPIRTQPRWNYYDSFS |
| | | AVSEDNLGFLMHAPAFETAGTYLRLVKINDWTEITQFILEHR |
| | | AKGSCKYALPLRIPPSACLSPQAYQQGVTVDSIGMLPRAIPEN |
| | | QRTVAVYSLKIAGWHGPHGDTPTLHEYMLDLQPETTGLYGY |
| | | GQLNDSSEEEDEIDGPAGQAEPDRAHYNIVTFCCKCDSTLRL |
| | | CVQSTHVDIRTLEDLLMGTLGIVCPICSQKPGAHQKRTAMFQ |
| | | DPQERPRKLPQLCTELQTTIHDIILECVYCKQQLLRREVYDYA |
| | | YRHLCIVYRDGNPYAVCDKCLKFYSKISEYRHYCYSLYGTTL |
| | | EQQYNKPLCDLLIRCINCQKPQCPEEKQRHLDKKQRFHNIRG |
| | | RWTGRCMSCCRSSRTRRETQLAAATNLDTASTTLLACFLLCF |
| | | CVLLCVCLLIRPLLLSVSTYTSLIILVLLLWITAASAFRCFIVYI |
| | | VFVYIPLFLIHTHAGPKAPYTSTLLPPELSETPNATQPELAPED |
| | | PEDSALLEDPVGTVAPQIPPNWHIPSIQDAATPYHPPATPNNM |
| | | GLIAGAVGGSLLAALVICGIVYWMHRRTRKAPKRIRLPHIRE |
| | | DDQPSSHQPLFY |
| gDM2- | SEQ ID | atggggggggctgccgccaggttgggggccgtgattttgtttgtcgtcatagtgggcctccatgg |
| E765dt1 | NO: 87 | ggtccgcggcaaatatgccttggcggatgcctctctcaagatggccgaccccaatcgctttcgcg |
| Nucleotide | | gcaaagaccttccggtcctggaccagctgaccgaccctccgggggtccggcgcgtgtaccaca |
| | | tccaggcgggcctaccggacccgttccagccccccagcctcccgatcacggtttactacgccgt |
| | | gttggagcgcgcctgccgcagcgtgctcctaaacgcaccgtcggaggccccccagattgtccg |
| | | cggggcctccgaagacgtccggaaacaaccctacaacctgaccatcgcttggtttcggatggga |
| | | ggcaactgtgctatccccatcacggtcatggagtacaccgaatgctcctacaacaagtctctggg |
| | | ggcctgtcccatccgaacgcagccccgctggaactactatgacagcttcagcgccgtcagcga |
| | | ggataacctggggttcctgatgcacgcccccgcgtttgagaccgccggcacgtacctgcggctc |
| | | gtgaagataaacgactggacggagattacacagtttatcctggagcaccgagccaagggctcct |
| | | gtaagtacgccctcccgctgcgaatccccccgtcagcctgcctctccccccaggcctaccagca |
| | | gggggtgacggtggacagcatcgggatgctgccccgcgctatccccgagaaccagcgcacc |
| | | gtcgccgtatacagcttgaagatcgccgggtggcacgggccccacggggacactcccaccctg |
| | | catgaatatatgctggatctgcagcctgagaccacaggcctgtacggctatggacagctgaacg |
| | | acagctccgaggaagaggacgaaatcgatgggccagcaggacaggcagagccagacagag |
| | | cccactacaatattgtgaccttctgctgtaagtgcgatagtacactgcgactgtgcgtgcagtcaac |
| | | acatgtcgacatccgcactctggaggatctgctgatgggaaccctggggatcgtgtgccctatttg |
| | | ttctcagaagccaggcgctcaccagaaaaggactgcaatgtttcaggatccacaggaacgaccc |
| | | aggaagctgcctcagctgtgcaccgagctgcagactaccatccacgacatcattctggaatgcgt |
| | | gtattgtaaacagcagctgctgaggagagaggtctacgattatgcatacaggcatctgtgcatcgt |
| | | gtatagagacggaaacccatacgccgtctgcgataagtgtctgaaattctattccaagatctctga |
| | | gtacaggcactattgttacagcctgtatggcacaactctggaacagcagtacaacaaacccctgt |
| | | gcgacctgctgatcagatgcattaattgtcagaagccccagtgtcctgaagagaaacagcggca |
| | | cctggataagaaacagaggttccataacatcagaggccggtggacaggaagatgcatgtcttgc |
| | | tgtcgctctagtcgaacccgacgagagacacagctggcagctgcaactaatctggacaccgcc |
| | | agtaccacactgctggcttgtttcctgctgtgcttttgcgtgctgctgtgcgtctgtctgctgatccgg |
| | | cctctgctgctgtcagtgagcacatacactagcctgatcattctggtcctgctgctgtggattacag |
| | | ccgcttccgctttccgctgttttatcgtgtatatcgtgttcgtgtacatccccctgtttctgattcacact |
| | | catgccgggcccaaggccccatacacgagcaccctgctgcccccggagctgtccgagacccc |
| | | caacgccacgcagccagaactcgccccggaagaccccgaggattcggccctcttggaggacc |
| | | ccgtggggacggtggcgccgcaaatcccaccaaactggcacatcccgtcgatccaggacgcc |
| | | gcgacgccttaccatcccccggccaccccgaacaacatgggcctgatcgccggcgcggtggg |
| | | cggcagtctcctggcagccctggtcatttgcggaattgtgtactggatgcaccgccgcactcgga |
| | | aagccccaaagcgcatacgcctcccccacatccgggaagacgaccagccgtcctcgcaccag |
| | | cccttgttttac |
| gDM3- | SEQ ID | MGGAAARLGAVILFVVIVGLHGVRGKYALADASLKMADPN |
| E765dt1 | NO: 88 | RFRGKDLPVLDQLTDPPGVRRVYHIQAGLPDPFQPPSLPITVY |
| Protein | | YAVLERACRSVLLNAPSEAPQIVRGASEDVRKQPYNLTIAWF |
| | | RMGGNCAIPITVMEYTECSYNKSLGACPIRTQPRWNYYDSFS |
| | | AVSEDNLGFLMHAPAFETAGTYLRLVKINDWTEITQFILEHR |
| | | AKGSCKYALPLRIPPSACLSPQAYQQGVTVDSIGMLPRFIPEN |
| | | QRTVAVYSLKIAGWHGPHGDTPTLHEYMLDLQPETTGLYGY |
| | | GQLNDSSEEEDEIDGPAGQAEPDRAHYNIVTFCCKCDSTLRL |
| | | CVQSTHVDIRTLEDLLMGTLGIVCPICSQKPGAHQKRTAMFQ |
| | | DPQERPRKLPQLCTELQTTIHDIILECVYCKQQLLRREVYDYA |
| | | YRHLCIVYRDGNPYAVCDKCLKFYSKISEYRHYCYSLYGTTL |
| | | EQQYNKPLCDLLIRCINCQKPQCPEEKQRHLDKKQRFHNIRG |
| | | RWTGRCMSCCRSSRTRRETQLAAATNLDTASTTLLACFLLCF |
| | | CVLLCVCLLIRPLLLSVSTYTSLIILVLLLWITAASAFRCFIVYI |
| | | VFVYIPLFLIHTHAGPGLIAGAVGGSLLAALVICGIVYWMHR |
| | | RTRKA |
| gDM3- | SEQ ID | atggggggggctgccgccaggttgggggccgtgattttgtttgtcgtcatagtgggcctccatgg |
| E765dt1 | NO: 89 | ggtccgcggcaaatatgccttggcggatgcctctctcaagatggccgaccccaatcgctttcgcg |
| Nucleotide | | gcaaagaccttccggtcctggaccagctgaccgaccctccgggggtccggcgcgtgtaccaca |
| | | tccaggcgggcctaccggacccgttccagccccccagcctcccgatcacggtttactacgccgt |
| | | gttggagcgcgcctgccgcagcgtgctcctaaacgcaccgtcggaggccccccagattgtccg |
| | | cggggcctccgaagacgtccggaaacaaccctacaacctgaccatcgcttggtttcggatggga |
| | | ggcaactgtgctatccccatcacggtcatggagtacaccgaatgctcctacaacaagtctctggg |
| | | ggcctgtcccatccgaacgcagccccgctggaactactatgacagcttcagcgccgtcagcga |
| | | ggataacctggggttcctgatgcacgcccccgcgtttgagaccgccggcacgtacctgcggctc |
| | | gtgaagataaacgactggacggagattacacagtttatcctggagcaccgagccaagggctcct |
| | | gtaagtacgccctcccgctgcgcatccccccgtcagcctgcctctccccccaggcctaccagca |
| | | gggggtgacggtggacagcatcgggatgctgccccgcttcatccccgagaaccagcgcaccg |
| | | tcgccgtatacagcttgaagatcgccgggtggcacgggccccacggggacactcccaccctgc |
| | | atgaatatatgctggatctgcagcctgagaccacaggcctgtacggctatggacagctgaacga |
| | | cagctccgaggaagaggacgaaatcgatgggccagcaggacaggcagagccagacagagc |
| | | ccactacaatattgtgaccttctgctgtaagtgcgatagtacactgcgactgtgcgtgcagtcaaca |
| | | catgtcgacatccgcactctggaggatctgctgatgggaaccctggggatcgtgtgccctatttgt |
| | | tctcagaagccaggcgctcaccagaaaaggactgcaatgtttcaggatccacaggaacgaccc |
| | | aggaagctgcctcagctgtgcaccgagctgcagactaccatccacgacatcattctggaatgcgt |
| | | gtattgtaaacagcagctgctgaggagagaggtctacgattatgcatacaggcatctgtgcatcgt |
| | | gtatagagacggaaacccatacgccgtctgcgataagtgtctgaaattctattccaagatctctga |
| | | gtacaggcactattgttacagcctgtatggcacaactctggaacagcagtacaacaaacccctgt |
| | | gcgacctgctgatcagatgcattaattgtcagaagccccagtgtcctgaagagaaacagcggca |
| | | cctggataagaaacagaggttccataacatcagaggccggtggacaggaagatgcatgtcttgc |
| | | tgtcgctctagtcgaacccgacgagagacacagctggcagctgcaactaatctggacaccgcc |
| | | agtaccacactgctggcttgtttcctgctgtgcttttgcgtgctgctgtgcgtctgtctgctgatccgg |
| | | cctctgctgctgtcagtgagcacatacactagcctgatcattctggtcctgctgctgtggattacag |
| | | ccgcttccgctttccgctgttttatcgtgtatatcgtgttcgtgtacatccccctgtttctgattcacact |
| | | catgccgggcccggcctgatcgccggcgcggtgggcggcagtctcctggcagccctggtcatt |
| | | tgcggaattgtgtactggatgcaccgccgcactcggaaagcc |
| gD-E765 | SEQ ID | MGGAAARLGAVILFVVIVGLHGVRGKYALADASLKMADPN |
| Protein | NO: 90 | RFRGKDLPVLDQLTDPPGVRRVYHIQAGLPDPFQPPSLPITVY |
| | | YAVLERACRSVLLNAPSEAPQIVRGASEDVRKQPYNLTIAWF |
| | | RMGGNCAIPITVMEYTECSYNKSLGACPIRTQPRWNYYDSFS |
| | | AVSEDNLGFLMHAPAFETAGTYLRLVKINDWTEITQFILEHR |
| | | AKGSCKYALPLRIPPSACLSPQAYQQGVTVDSIGMLPRFIPEN |
| | | QRTVAVYSLKIAGWHGPHGDTPTLHEYMLDLQPETTDLYCY |
| | | EQLNDSSEEEDEIDGPAGQAEPDRAHYNIVTFCCKCDSTLRL |
| | | CVQSTHVDIRTLEDLLMGTLGIVCPICSQKPGAHQKRTAMFQ |
| | | DPQERPRKLPQLCTELQTTIHDIILECVYCKQQLLRREVYDFA |
| | | FRDLCIVYRDGNPYAVCDKCLKFYSKISEYRHYCYSLYGTTL |
| | | EQQYNKPLCDLLIRCINCQKPLCPEEKQRHLDKKQRFHNIRG |
| | | RWTGRCMSCCRSSRTRRETQLAAACIHNITGVLFALLCVLLC |
| | | VCLLIRPLLLSVSTYTSLIILVLLLWITAASAFRCFIVYIIFVYIP |
| | | LFLIHTHARFLITGPNRWPRIRYHAAAVYMTSTLLPPELSETP |
| | | NATQPELAPEDPEDSALLEDPVGTVAPQIPPNWHIPSIQDAAT |
| | | PYHPPATPNNMGLIAGAVGGSLLAALVICGIVYWMHRRTRK |
| | | APKRIRLPHIREDDQPSSHQPLFYDYKDDDDKT |
| gD-E765 | SEQ ID | atggggggggctgccgccaggttgggggccgtgattttgtttgtcgtcatagtgggcctccatgg |
| Nucleotide | NO: 91 | ggtccgcggcaaatatgccttggcggatgcctctctcaagatggccgaccccaatcgctttcgcg |
| | | gcaaagaccttccggtcctggaccagctgaccgaccctccgggggtccggcgcgtgtaccaca |
| | | tccaggcgggcctaccggacccgttccagccccccagcctcccgatcacggtttactacgccgt |
| | | gttggagcgcgcctgccgcagcgtgctcctaaacgcaccgtcggaggccccccagattgtccg |
| | | cggggcctccgaagacgtccggaaacaaccctacaacctgaccatcgcttggtttcggatggga |
| | | ggcaactgtgctatccccatcacggtcatggagtacaccgaatgctcctacaacaagtctctggg |
| | | ggcctgtcccatccgaacgcagccccgctggaactactatgacagcttcagcgccgtcagcga |
| | | ggataacctggggttcctgatgcacgcccccgcgtttgagaccgccggcacgtacctgcggctc |
| | | gtgaagataaacgactggacggagattacacagtttatcctggagcaccgagccaagggctcct |
| | | gtaagtacgccctcccgctgcgcatccccccgtcagcctgcctctccccccaggcctaccagca |
| | | gggggtgacggtggacagcatcgggatgctgccccgcttcatccccgagaaccagcgcaccg |
| | | tcgccgtatacagcttgaagatcgccgggtggcacgggccccatggagatacacctacattgca |
| | | tgaatatatgttagatttgcaaccagagacaactgatctctactgttatgagcaattaaatgacagct |
| | | cagaggaggaggatgaaatagatggtccagctggacaagcagaaccggacagagcccattac |
| | | aatattgtaaccttttgttgcaagtgtgactctacgcttcggttgtgcgtacaaagcacacacgtaga |
| | | cattcgtactttggaagacctgttaatgggcacactaggaattgtgtgccccatctgttctcagaaa |
| | | ccaggcgcccaccaaaagagaactgcaatgtttcaggacccacaggagcgacccagaaagtt |
| | | accacagttatgcacagagctgcaaacaactatacatgatataatattagaatgtgtgtactgcaag |
| | | caacagttactgcgacgtgaggtatatgactttgcttttcgggatttatgcatagtatatagagatgg |
| | | gaatccatatgctgtatgtgataaatgtttaaagttttattctaaaattagtgagtatagacattattgtt |
| | | atagtttgtatggaacaacattagaacagcaatacaacaaaccgttgtgtgatttgttaattaggtgt |
| | | attaactgtcaaaagccactgtgtcctgaagaaaagcaaagacatctggacaaaaagcaaagatt |
| | | ccataatataaggggtcggtggaccggtcgatgtatgtcttgttgcagatcatcaagaacacgtag |
| | | agaaacccagctggcggccgcctgcatccacaacattactggcgtgctttttgctttgctttgtgtg |
| | | cttttgtgtgtctgcctattaatacgtccgctgcttttgtctgtgtctacatacacatcattaataatattg |
| | | gtattactattgtggataacagcagcctctgcgtttaggtgttttattgtatatattatatttgtttatatac |
| | | cattatttttaatacatacacatgcacgctttttaattacagggcccaaccggtggcctaggatccga |
| | | tatcacgcggccgcagtgtacatgacgagcaccctgctgcccccggagctgtccgagaccccc |
| | | aacgccacgcagccagaactcgccccggaagaccccgaggattcggccctcttggaggaccc |
| | | cgtggggacggtggcgccgcaaatcccaccaaactggcacatcccgtcgatccaggacgccg |
| | | cgacgccttaccatcccccggccaccccgaacaacatgggcctgatcgccggcgcggtgggc |
| | | ggcagtctcctggcagccctggtcatttgcggaattgtgtactggatgcaccgccgcactcggaa |
| | | agccccaaagcgcatacgcctcccccacatccgggaagacgaccagccgtcctcgcaccagc |
| | | ccttgttttacgactacaaagacgatgacgacaagact |
| gDM5- | SEQ ID | MGGAAARLGAVILFVVIVGLHGVRGKYCLADASLKMADPN |
| E765 | NO: 92 | RFRGKDLPVLDQLTDPPGVRRVYHIQAGLPDPFQPPSLPITVC |
| Protein | | YAVLERACRSVLLNAPSEAPQIVRGASEDVRKQPYNLTIAWF |
| | | RMGGNCAIPITVMEYTECSYNKSLGACPIRTQPRWNYYDSFS |
| | | AVSEDNLGFLMHAPAFETAGTYLRLVKINDWTEITQFILEHR |
| | | AKGSCKYALPLRIPPSACLSPQAYQQGVTVDSIGPTPTLHEY |
| | | MLDLQPETTGLYGYGQLNDSSEEEDEIDGPAGQAEPDRAHY |
| | | NIVTFCCKCDSTLRLCVQSTHVDIRTLEDLLMGTLGIVCPICS |
| | | QKPGAHQKRTAMFQDPQERPRKLPQLCTELQTTIHDIILECV |
| | | YCKQQLLRREVYDYAYRHLCIVYRDGNPYAVCDKCLKFYS |
| | | KISEYRHYCYSLYGTTLEQQYNKPLCDLLIRCINCQKPQCPEE |
| | | KQRHLDKKQRFHNIRGRWTGRCMSCCRSSRTRRETQLAAAT |
| | | NLDTASTTLLACFLLCFCVLLCVCLLIRPLLLSVSTYTSLIILV |
| | | LLLWITAASAFRCFIVYIVFVYIPLFLIGPATQPELAPEDPEDS |
| | | ALLEDPVGTVAPQIPPNWHIPSIQDAATPYHPPATPNNMGLIA |
| | | GAVGGSLLAALVICGIVYWMHRRTRKAPKRIRLPHIREDDQP |
| | | SSHQPLFY |
| gDM5- | SEQ ID | atggggggggctgccgccaggttgggggccgtgattttgtttgtcgtcatagtgggcctccatgg |
| E765 | NO: 93 | ggtccgcggcaaatattgtttggcggatgcctctctcaagatggccgaccccaatcgctttcgcg |
| Nucleotide | | gcaaagaccttccggtcctggaccagctgaccgaccctccgggggtccggcgcgtgtaccaca |
| | | tccaggcgggcctaccggacccgttccagccccccagcctcccgatcacggtttgctacgccgt |
| | | gttggagcgcgcctgccgcagcgtgctcctaaacgcaccgtcggaggccccccagattgtccg |
| | | cggggcctccgaagacgtccggaaacaaccctacaacctgaccatcgcttggtttcggatggga |
| | | ggcaactgtgctatccccatcacggtcatggagtacaccgaatgctcctacaacaagtctctggg |
| | | ggcctgtcccatccgaacgcagccccgctggaactactatgacagcttcagcgccgtcagcga |
| | | ggataacctggggttcctgatgcacgcccccgcgtttgagaccgccggcacgtacctgcggctc |
| | | gtgaagataaacgactggacggagattacacagtttatcctggagcaccgagccaagggctcct |
| | | gtaagtacgccctcccgctgcgcatccccccgtcagcctgcctctccccccaggcctaccagca |
| | | gggggtgacggtggacagcatcgggccCactcccaccctgcatgaatatatgctggatctgca |
| | | gcctgagaccacaggcctgtacggctatggacagctgaacgacagctccgaggaagaggacg |
| | | aaatcgatgggccagcaggacaggcagagccagacagagcccactacaatattgtgaccttct |
| | | gctgtaagtgcgatagtacactgcgactgtgcgtgcagtcaacacatgtcgacatccgcactctg |
| | | gaggatctgctgatgggaaccctggggatcgtgtgccctatttgttctcagaagccaggcgctca |
| | | ccagaaaaggactgcaatgtttcaggatccacaggaacgacccaggaagctgcctcagctgtg |
| | | caccgagctgcagactaccatccacgacatcattctggaatgcgtgtattgtaaacagcagctgct |
| | | gaggagagaggtctacgattatgcatacaggcatctgtgcatcgtgtatagagacggaaacccat |
| | | acgccgtctgcgataagtgtctgaaattctattccaagatctctgagtacaggcactattgttacag |
| | | cctgtatggcacaactctggaacagcagtacaacaaacccctgtgcgacctgctgatcagatgc |
| | | attaattgtcagaagccccagtgtcctgaagagaaacagcggcacctggataagaaacagaggt |
| | | tccataacatcagaggccggtggacaggaagatgcatgtcttgctgtcgctctagtcgaacccga |
| | | cgagagacacagctggcagctgcaactaatctggacaccgccagtaccacactgctggcttgttt |
| | | cctgctgtgcttttgcgtgctgctgtgcgtctgtctgctgatccggcctctgctgctgtcagtgagca |
| | | catacactagcctgatcattctggtcctgctgctgtggattacagccgcttccgctttccgctgtttta |
| | | tcgtgtatatcgtgttcgtgtacatccccctgtttctgattGGGCCcgccacgcagccagaact |
| | | cgccccggaagaccccgaggattcggccctcttggaggaccccgtggggacggtggcgccg |
| | | caaatcccaccaaactggcacatcccgtcgatccaggacgccgcgacgccttaccatcccccg |
| | | gccaccccgaacaacatgggcctgatcgccggcgcggtgggcggcagtctcctggcagccct |
| | | ggtcatttgcggaattgtgtactggatgcaccgccgcactcggaaagccccaaagcgcatacgc |
| | | ctcccccacatccgggaagacgaccagccgtcctcgcaccagcccttgttttac |
| gD-E7652 | SEQ ID | MGGAAARLGAVILFVVIVGLHGVRGKYALADASLKMADPN |
| Protein | NO: 94 | RFRGKDLPVLDQLTDPPGVRRVYHIQAGLPDPFQPPSLPITVY |
| | | YAVLERACRSVLLNAPSEAPQIVRGASEDVRKQPYNLTIAWF |
| | | RMGGNCAIPITVMEYTECSYNKSLGACPIRTQPRWNYYDSFS |
| | | AVSEDNLGFLMHAPAFETAGTYLRLVKINDWTEITQFILEHR |
| | | AKGSCKYALPLRIPPSACLSPQAYQQGVTVDSIGMLPRFIPEN |
| | | QRTVAVYSLKIAGWHGPTPTLHEYMLDLQPETTDLYGPAGQ |
| | | AEPDRAHYNIVTFCCKCDSTLRLCVQSTHVDIRTLEDLLMGT |
| | | LGIVCPICAAPRKLPQLCTELNTSLQDIEITCVYCYDFAFRAA |
| | | CIVYRDGNPYAVCDKCLKFYSKISEYRHYCYSVYGTTLAAS |
| | | VSTYTSLILLVLLLWITAASAFRCFIVYIVFVYIPLFLIHTHARF |
| | | LIAAETLCQRLNVCQDKILTHYENDSTDLRDHIDYWKHMRL |
| | | ECAIYYKAREMGFKHINHQVVPTLAVSKNKALQAIELQLTLE |
| | | TIYNSQYSNEKWSNTTPIVHLKGDANTLKCLRYRFKKHCKL |
| | | YTAVSSTWHWTGHNVKHKSAIVTLTYDSEWGPKAPYTSTLL |
| | | PPELSETPNATQPELAPEDPEDSALLEDPVGTVAPQIPPNWHI |
| | | PSIQDAATPYHPPATPNNMGLIAGAVGGSLLAALVICGIVYW |
| | | MHRRTRKAPKRIRLPHIREDDQPSSHQPLFY |
| gD-E7652 | SEQ ID | atggggggggctgccgccaggttgggggccgtgattttgtttgtcgtcatagtgggcctccatgg |
| Nucleotide | NO: 95 | ggtccgcggcaaatatgccttggcggatgcctctctcaagatggccgaccccaatcgctttcgcg |
| | | gcaaagaccttccggtcctggaccagctgaccgaccctccgggggtccggcgcgtgtaccaca |
| | | tccagggggcctaccggacccgttccagccccccagcctcccgatcacggtttactacgccgt |
| | | gttggagcgcgcctgccgcagcgtgctcctaaacgcaccgtcggaggccccccagattgtccg |
| | | cggggcctccgaagacgtccggaaacaaccctacaacctgaccatcgcttggtttcggatggga |
| | | ggcaactgtgctatccccatcacggtcatggagtacaccgaatgctcctacaacaagtctctggg |
| | | ggcctgtcccatccgaacgcagccccgctggaactactatgacagcttcagcgccgtcagcga |
| | | ggataacctggggttcctgatgcacgcccccgcgtttgagaccgccggcacgtacctgcggctc |
| | | gtgaagataaacgactggacggagattacacagtttatcctggagcaccgagccaagggctcct |
| | | gtaagtacgccctcccgctgcgcatccccccgtcagcctgcctctccccccaggcctaccagca |
| | | gggggtgacggtggacagcatcgggatgctgccccgcttcatccccgagaaccagcgcaccg |
| | | tcgccgtatacagcttgaagatcgccgggtggcacgggcccacacctacattgcatgaatatatg |
| | | ttagatttgcaaccagagacaactgatctctacggtccagctggacaagcagaaccggacagag |
| | | cccattacaatattgtaaccttttgttgcaagtgtgactctacgcttcggttgtgcgtacaaagcaca |
| | | cacgtagacattcgtactttggaagacctgttaatgggcacactaggaattgtgtgccccatctgtg |
| | | ctgctcccagaaagttaccacagttatgcacagagctgaacacttcactgcaagacatagaaata |
| | | acctgtgtatattgctatgactttgcttttcgggctgcttgcatagtatatagagatgggaatccatat |
| | | gctgtatgtgataaatgtttaaagttttattctaaaattagtgagtatagacattattgttatagtgtatat |
| | | ggaacaacattagctgcttctgtgtctacatacacatcattaatattgttggtattactattgtggataa |
| | | cagcagcctctgcgtttaggtgttttattgtatatattgtatttgtttatataccattatttttaatacataca |
| | | catgcacgctttttaattgctgctgagactctttgccaacgtttaaatgtgtgtcaggacaaaatacta |
| | | acacattatgaaaatgatagtacagacctacgtgaccatatagactattggaaacacatgcgccta |
| | | gaatgtgctatttattacaaggccagagaaatgggatttaaacatattaaccaccaggtggtgcca |
| | | acactggctgtatcaaagaataaagcattacaagcaattgaactgcaactaacgttagaaacaata |
| | | tataactcacaatatagtaatgaaaagtggagtaacactacacccatagtacatttaaaaggtgatg |
| | | ctaatactttaaaatgtttaagatatagatttaaaaagcattgtaaattgtatactgcagtgtcgtctac |
| | | atggcattggacaggacataatgtaaaacataaaagtgcaattgttacacttacatatgatagtgaa |
| | | tgggggcccaaggccccatacacgagcaccctgctgcccccggagctgtccgagacccccaa |
| | | cgccacgcagccagaactcgccccggaagaccccgaggattcggccctcttggaggaccccg |
| | | tggggacggtggcgccgcaaatcccaccaaactggcacatcccgtcgatccaggacgccgcg |
| | | acgccttaccatcccccggccaccccgaacaacatgggcctgatcgccggcgcggtggggg |
| | | cagtctcctggcagccctggtcatttgcggaattgtgtactggatgcaccgccgcactcggaaag |
| | | ccccaaagcgcatacgcctcccccacatccgggaagacgaccagccgtcctcgcaccagccc |
| | | ttgttttac |
| gD(-TM)- | SEQ ID | MGGAAARLGAVILFVVIVGLHGVRGKYALADASLKMADPN |
| E765 | NO: 96 | RFRGKDLPVLDQLTDPPGVRRVYHIQAGLPDPFQPPSLPITVY |
| Protein | | YAVLERACRSVLLNAPSEAPQIVRGASEDVRKQPYNLTIAWF |
| | | RMGGNCAIPITVMEYTECSYNKSLGACPIRTQPRWNYYDSFS |
| | | AVSEDNLGFLMHAPAFETAGTYLRLVKINDWTEITQFILEHR |
| | | AKGSCKYALPLRIPPSACLSPQAYQQGVTVDSIGMLPRFIPEN |
| | | QRTVAVYSLKIAGWHGPHGDTPTLHEYMLDLQPETTDLYCY |
| | | EQLNDSSEEEDEIDGPAGQAEPDRAHYNIVTFCCKCDSTLRL |
| | | CVQSTHVDIRTLEDLLMGTLGIVCPICSQKPGAHQKRTAMFQ |
| | | DPQERPRKLPQLCTELQTTIHDIILECVYCKQQLLRREVYDFA |
| | | FRDLCIVYRDGNPYAVCDKCLKFYSKISEYRHYCYSLYGTTL |
| | | EQQYNKPLCDLLIRCINCQKPLCPEEKQRHLDKKQRFHNIRG |
| | | RWTGRCMSCCRSSRTRRETQLAAACIHNITGVLFALLCVLLC |
| | | VCLLIRPLLLSVSTYTSLIILVLLLWITAASAFRCFIVYIIFVYIP |
| | | LFLIHTHARFLITGPNRWPRIRYHAAAVYMTSTLLPPELSETP |
| | | NATQPELAPEDPEDSALLEDPVGTVAPQIPPNWHIPSIQDAAT |
| | | PYHPPATPNNMGLITRKAPKRIRLPHIREDDQPSSHQPLFY |
| gD(-TM)- | SEQ ID | atggggggggctgccgccaggttgggggccgtgattttgtttgtcgtcatagtgggcctccatgg |
| E765 | NO: 97 | ggtccgcggcaaatatgccttggcggatgcctctctcaagatggccgaccccaatcgctttcgcg |
| Nucleotide | | gcaaagaccttccggtcctggaccagctgaccgaccctccgggggtccggcgcgtgtaccaca |
| | | tccaggcgggcctaccggacccgttccagccccccagcctcccgatcacggtttactacgccgt |
| | | gttggagcgcgcctgccgcagcgtgctcctaaacgcaccgtcggaggccccccagattgtccg |
| | | cggggcctccgaagacgtccggaaacaaccctacaacctgaccatcgcttggtttcggatggga |
| | | ggcaactgtgctatccccatcacggtcatggagtacaccgaatgctcctacaacaagtctctggg |
| | | ggcctgtcccatccgaacgcagccccgctggaactactatgacagcttcagcgccgtcagcga |
| | | ggataacctggggttcctgatgcacgcccccgcgtttgagaccgccggcacgtacctgcggctc |
| | | gtgaagataaacgactggacggagattacacagtttatcctggagcaccgagccaagggctcct |
| | | gtaagtacgccctcccgctgcgcatccccccgtcagcctgcctctccccccaggcctaccagca |
| | | gggggtgacggtggacagcatcgggatgctgccccgcttcatccccgagaaccagcgcaccg |
| | | tcgccgtatacagcttgaagatcgccgggtggcacgggccccatggagatacacctacattgca |
| | | tgaatatatgttagatttgcaaccagagacaactgatctctactgttatgagcaattaaatgacagct |
| | | cagaggaggaggatgaaatagatggtccagctggacaagcagaaccggacagagcccattac |
| | | aatattgtaaccttttgttgcaagtgtgactctacgcttcggttgtgcgtacaaagcacacacgtaga |
| | | cattcgtactttggaagacctgttaatgggcacactaggaattgtgtgccccatctgttctcagaaa |
| | | ccaggcgcccaccaaaagagaactgcaatgtttcaggacccacaggagcgacccagaaagtt |
| | | accacagttatgcacagagctgcaaacaactatacatgatataatattagaatgtgtgtactgcaag |
| | | caacagttactgcgacgtgaggtatatgactttgcttttcgggatttatgcatagtatatagagatgg |
| | | gaatccatatgctgtatgtgataaatgtttaaagttttattctaaaattagtgagtatagacattattgtt |
| | | atagtttgtatggaacaacattagaacagcaatacaacaaaccgttgtgtgatttgttaattaggtgt |
| | | attaactgtcaaaagccactgtgtcctgaagaaaagcaaagacatctggacaaaaagcaaagatt |
| | | ccataatataaggggtcggtggaccggtcgatgtatgtcttgttgcagatcatcaagaacacgtag |
| | | agaaacccagctggcggccgcctgcatccacaacattactggcgtgctttttgctttgctttgtgtg |
| | | cttttgtgtgtctgcctattaatacgtccgctgcttttgtctgtgtctacatacacatcattaataatattg |
| | | gtattactattgtggataacagcagcctctgcgtttaggtgttttattgtatatattatatttgtttatatac |
| | | cattatttttaatacatacacatgcacgctttttaattacagggcccaaccggtggcctaggatccga |
| | | tatcacgcggccgcagtgtacatgacgagcaccctgctgcccccggagctgtccgagaccccc |
| | | aacgccacgcagccagaactcgccccggaagaccccgaggattcggccctcttggaggaccc |
| | | cgtggggacggtggcgccgcaaatcccaccaaactggcacatcccgtcgatccaggacgccg |
| | | cgacgccttaccatcccccggccaccccgaacaacatgggcctgatcactoggaaagccccaa |
| | | agcgcatacgcctcccccacatccgggaagacgaccagccgtcctcgcaccagcccttgtttta |
| | | c |
| SgD-PA2- | SEQ ID | MGGAAARLGAVILFVVIVGLHGVRGKYALADASLKMADPN |
| E7652 | NO: 98 | RFRGKDLPVLDQLTDPPGVRRVYHIQAGLPDPFQPPSLPITVY |
| Protein | | YAVLERACRSVLLNAPSEAPQIVRGASEDVRKQPYNLTIAWF |
| | | RMGGNCAIPITVMEYTECSYNKSLGACPIRTQPRWNYYDSFS |
| | | AVSEDNLGFLMHAPAFETAGTYLRLVKINDWTEITQFILEHR |
| | | AKGSCKYALPLRIPPSACLSPQAYQQGVTVDSIGMLPRFIPEN |
| | | QRTVAVYSLKIAGWHGPKAPYTSTLLPPELSETPNATQPELA |
| | | PEDPEDSALLEDPVGTVAPQIPPNAHIPSIQDAATPYHPPATPN |
| | | NMGLIAGAVGGSLLAALVICGIVYWMHRRTRKAPKRIRLPHI |
| | | REDDQPSSHQPLFYLSTGSGATNFSLLKQAGDVEENPGPDYK |
| | | DDDDKTPTLHEYMLDLQPETTDLYGPAGQAEPDRAHYNIVT |
| | | FCCKCDSTLRLCVQSTHVDIRTLEDLLMGTLGIVCPICAAPRK |
| | | LPQLCTELNTSLQDIEITCVYCYDFAFRAACIVYRDGNPYAV |
| | | CDKCLKFYSKISEYRHYCYSVYGTTLAASVSTYTSLILLVLLL |
| | | WITAASAFRCFIVYIVFVYIPLFLIHTHARFLIAAETLCQRLNV |
| | | CQDKILTHYENDSTDLRDHIDYWKHMRLECAIYYKAREMGF |
| | | KHINHQVVPTLAVSKNKALQAIELQLTLETIYNSQYSNEKWS |
| | | NTTPIVHLKGDANTLKCLRYRFKKHCKLYTAVSSTWHWTG |
| | | HNVKHKSAIVTLTYDSEW |
| SgD-PA2- | SEQ ID | atggggggggctgccgccaggttgggggccgtgattttgtttgtcgtcatagtgggcctccatgg |
| E7652 | NO: 99 | ggtccgcggcaaatatgccttggcggatgcctctctcaagatggccgaccccaatcgctttcgcg |
| Nucleotide | | gcaaagaccttccggtcctggaccagctgaccgaccctccgggggtccggcgcgtgtaccaca |
| | | tccaggcgggcctaccggacccgttccagccccccagcctcccgatcacggtttactacgccgt |
| | | gttggagcgcgcctgccgcagcgtgctcctaaacgcaccgtcggaggccccccagattgtccg |
| | | cggggcctccgaagacgtccggaaacaaccctacaacctgaccatcgcttggtttcggatggga |
| | | ggcaactgtgctatccccatcacggtcatggagtacaccgaatgctcctacaacaagtctctggg |
| | | ggcctgtcccatccgaacgcagccccgctggaactactatgacagcttcagcgccgtcagcga |
| | | ggataacctggggttcctgatgcacgcccccgcgtttgagaccgccggcacgtacctgcggctc |
| | | gtgaagataaacgactggacggagattacacagtttatcctggagcaccgagccaagggctcct |
| | | gtaagtacgccctcccgctgcgcatccccccgtcagcctgcctgtccccccaggcctaccagca |
| | | gggggtgacggtggacagcatcgggatgctgccccgcttcatccccgagaaccagcgcaccg |
| | | tcgccgtatacagcttgaagatcgccgggtggcacgggcccaaggccccatacacgagcacc |
| | | ctgctgcccccggagctgtccgagacccctaacgccacgcagccagaactcgccccggaaga |
| | | ccccgaggattcggccctcttggaggaccccgtggggacggtggcgccgcaaatcccaccaa |
| | | acgcgcacataccgtcgatccaggacgccgcgacgccttaccatcccccggccaccccgaac |
| | | aacatgggcctgatcgccggcgcgggggcggcagtctcctggcagccctggtcatttgcgga |
| | | attgtgtactggatgcaccgccgcactcggaaagccccaaagcgcatacgcctcccccacatcc |
| | | gggaagacgaccagccgtcctcgcaccagcccttgttttacttgtcgacaggaagcggagctac |
| | | taacttcagcctgctgaagcaggctggagacgtggaggagaaccctggacctgactacaaaga |
| | | cgatgacgacaagacacctacattgcatgaatatatgttagatttgcaaccagagacaactgatct |
| | | ctacggtccagctggacaagcagaaccggacagagcccattacaatattgtaaccttttgttgcaa |
| | | gtgtgactctacgcttcggttgtgcgtacaaagcacacacgtagacattcgtactttggaagacct |
| | | gttaatgggcacactaggaattgtgtgccccatctgtgctgctcccagaaagttaccacagttatg |
| | | cacagagctgaacacttcactgcaagacatagaaataacctgtgtatattgctatgactttgcttttc |
| | | gggctgcttgcatagtatatagagatgggaatccatatgctgtatgtgataaatgtttaaagttttatt |
| | | ctaaaattagtgagtatagacattattgttatagtgtatatggaacaacattagctgcttctgtgtctac |
| | | atacacatcattaatattgttggtattactattgtggataacagcagcctctgcgtttaggtgttttattg |
| | | tatatattgtatttgtttatataccattatttttaatacatacacatgcacgctttttaattgctgctgagac |
| | | tctttgccaacgtttaaatgtgtgtcaggacaaaatactaacacattatgaaaatgatagtacagac |
| | | ctacgtgaccatatagactattggaaacacatgcgcctagaatgtgctatttattacaaggccaga |
| | | gaaatgggatttaaacatattaaccaccaggtggtgccaacactggctgtatcaaagaataaagc |
| | | attacaagcaattgaactgcaactaacgttagaaacaatatataactcacaatatagtaatgaaaag |
| | | tggagtaacactacacccatagtacatttaaaaggtgatgctaatactttaaaatgtttaagatatag |
| | | atttaaaaagcattgtaaattgtatactgcagtgtcgtctacatggcattggacaggacataatgtaa |
| | | aacataaaagtgcaattgttacacttacatatgatagtgaatgg |
| Mutant | SEQ ID | MGGAAARLGAVILFVVIVGLHGVRGKYALADASLKMADPN |
| HPV E7, | NO: 100 | RFRGKDLPVLDQLTDPPGVRRVYHIQAGLPDPFQPPSLPITVY |
| E6, E5, and | | YAVLERACRSVLLNAPSEAPQIVRGASEDVRKQPYNLTIAWF |
| E2 Fusion | | RMGGNCAIPITVMEYTECSYNKSLGACPIRTQPRWNYYDSFS |
| Protein | | AVSEDNLGFLMHAPAFETAGTYLRLVKINDWTEITQFILEHR |
| Fused to | | AKGSCKYALPLRIPPSACLSPQAYQQGVTVDSIGMLPRFIPEN |
| gD Protein | | QRTVAVYSLKIAGWHGPTPTLHEYMLDLQPETTDLYGPAGQ |
| | | AEPDRAHYNIVTFCCKCDSTLRLCVQSTHVDIRTLEDLLMGT |
| | | LGIVCPICAAPRKLPQLCTELNTSLQDIEITCVYCYDFAFRAA |
| | | CIVYRDGNPYAVCDKCLKFYSKISEYRHYCYSVYGTTLAAS |
| | | VSTYTSLILLVLLLWITAASAFRCFIVYIVFVYIPLFLIHTHARF |
| | | LIAAETLCQRLNVCQDKILTHYENDSTDLRDHIDYWKHMRL |
| | | ECAIYYKAREMGFKHINHQVVPTLAVSKNKALQAIELQLTLE |
| | | TIYNSQYSNEKWSNTTPIVHLKGDANTLKCLRYRFKKHCKL |
| | | YTAVSSTWHWTGHNVKHKSAIVTLTYDSEWGPKAPYTSTLL |
| | | PPELSETPNATQPELAPEDPEDSALLEDPVGTVAPQIPPNWHI |
| | | PSIQDAATPYHPPATPNNMGLIAGAVGGSLLAALVICGIVYW |
| | | MHRRTRKAPKRIRLPHIREDDQPSSHQPLFY |
| Mutant | SEQ ID | atggggggggctgccgccaggttgggggccgtgattttgtttgtcgtcatagtgggcctccatgg |
| HPV E7, | NO: 101 | ggtccgcggcaaatatgccttggcggatgcctctctcaagatggccgaccccaatcgctttcgcg |
| E6, E5, and | | gcaaagaccttccggtcctggaccagctgaccgaccctccgggggtccggcgcgtgtaccaca |
| E2 Fusion | | tccaggcgggcctaccggacccgttccagccccccagcctcccgatcacggtttactacgccgt |
| Protein | | gttggagcgcgcctgccgcagcgtgctcctaaacgcaccgtcggaggccccccagattgtccg |
| Fused to | | cggggcctccgaagacgtccggaaacaaccctacaacctgaccatcgcttggtttcggatggga |
| gD | | ggcaactgtgctatccccatcacggtcatggagtacaccgaatgctcctacaacaagtctctggg |
| Nucleotide | | ggcctgtcccatccgaacgcagccccgctggaactactatgacagcttcagcgccgtcagcga |
| | | ggataacctggggttcctgatgcacgcccccgcgtttgagaccgccggcacgtacctgcggctc |
| | | gtgaagataaacgactggacggagattacacagtttatcctggagcaccgagccaagggctcct |
| | | gtaagtacgccctcccgctgcgcatccccccgtcagcctgcctctccccccaggcctaccagca |
| | | gggggtgacggtggacagcatcgggatgctgccccgcttcatccccgagaaccagcgcaccg |
| | | tcgccgtatacagcttgaagatcgccgggggcacgggcccacacctacattgcatgaatatatg |
| | | ttagatttgcaaccagagacaactgatctctacggtccagctggacaagcagaaccggacagag |
| | | cccattacaatattgtaaccttttgttgcaagtgtgactctacgcttcggttgtgcgtacaaagcaca |
| | | cacgtagacattcgtactttggaagacctgttaatgggcacactaggaattgtgtgccccatctgtg |
| | | ctgctcccagaaagttaccacagttatgcacagagctgaacacttcactgcaagacatagaaata |
| | | acctgtgtatattgctatgactttgcttttcgggctgcttgcatagtatatagagatgggaatccatat |
| | | gctgtatgtgataaatgtttaaagttttattctaaaattagtgagtatagacattattgttatagtgtatat |
| | | ggaacaacattagctgcttctgtgtctacatacacatcattaatattgttggtattactattgtggataa |
| | | cagcagcctctgcgtttaggtgttttattgtatatattgtatttgtttatataccattatttttaatacataca |
| | | catgcacgctttttaattgctgctgagactctttgccaacgtttaaatgtgtgtcaggacaaaatacta |
| | | acacattatgaaaatgatagtacagacctacgtgaccatatagactattggaaacacatgcgccta |
| | | gaatgtgctatttattacaaggccagagaaatgggatttaaacatattaaccaccaggtggtgcca |
| | | acactggctgtatcaaagaataaagcattacaagcaattgaactgcaactaacgttagaaacaata |
| | | tataactcacaatatagtaatgaaaagtggagtaacactacacccatagtacatttaaaaggtgatg |
| | | ctaatactttaaaatgtttaagatatagatttaaaaagcattgtaaattgtatactgcagtgtcgtctac |
| | | atggcattggacaggacataatgtaaaacataaaagtgcaattgttacacttacatatgatagtgaa |
| | | tgggggcccaaggccccatacacgagcaccctgctgcccccggagctgtccgagacccccaa |
| | | cgccacgcagccagaactcgccccggaagaccccgaggattcggccctcttggaggaccccg |
| | | tggggacggtggcgccgcaaatcccaccaaactggcacatcccgtcgatccaggacgccgcg |
| | | acgccttaccatcccccggccaccccgaacaacatgggcctgatcgccggcgcggtgggcgg |
| | | cagtctcctggcagccctggtcatttgcggaattgtgtactggatgcaccgccgcactcggaaag |
| | | ccccaaagcgcatacgcctcccccacatccgggaagacgaccagccgtcctcgcaccagccc |
| | | ttgttttac |
| Mutant | SEQ ID | MGGAAARLGAVILFVVIVGLHGVRGKYALADASLKMADPN |
| HPV E7, | NO: 102 | RFRGKDLPVLDQLTDPPGVRRVYHIQAGLPDPFQPPSLPITVY |
| E6, E5, and | | YAVLERACRSVLLNAPSEAPQIVRGASEDVRKQPYNLTIAWF |
| C-terminus | | RMGGNCAIPITVMEYTECSYNKSLGACPIRTQPRWNYYDSFS |
| E2 Fusion | | AVSEDNLGFLMHAPAFETAGTYLRLVKINDWTEITQFILEHR |
| Protein | | AKGSCKYALPLRIPPSACLSPQAYQQGVTVDSIGMLPRFIPEN |
| Fused to | | QRTVAVYSLKIAGWHGPHGDTPTLHEYMLDLQPETTAAAA |
| gD Protein | | AAQLNDSSEEEDEIDGPAGQAEPDRAHYNIVTFCCKCDSTLR |
| | | LCVQSTHVDIRTLEDLLMGTLGIVCPICSQKPGAHQKRTAMF |
| | | QDPQERPRKLPQLCTELQTTIHDIILECVYCKQQLLRREVYDA |
| | | AAAALCIVYRDGNPYAVCDKCLKFYSKISEYRHYCYSLYGT |
| | | TLEQQYNKPLCDLLIRCINCQKPQCPEEKQRHLDKKQRFHNI |
| | | RGRWTGRCMSCCRSSRTRRETQLGACVLLCVCLLIRPLLLSV |
| | | STYTSLIILVLLLWITAASAFRCFIVYIAFVYIPLFLIHTHASNT |
| | | TPIVHLKGDANTLKCLRYRFKKHCKLYTAVSSTWHWTGHN |
| | | VKHKSAIVTLTYDSEWGPKAPYTSTLLPPELSETPNATQPELA |
| | | PEDPEDSALLEDPVGTVAPQIPPNWHIPSIQDAATPYHPPATP |
| | | NNMGLIAGAVGGSLLAALVICGIVYWMHRRTRKAPKRIRLP |
| | | HIREDDQPSSHQPLFY |
| Mutant | SEQ ID | atggggggggctgccgccaggttgggggccgtgattttgtttgtcgtcatagtgggcctccatgg |
| HPV E7, | NO: 103 | ggtccgcggcaaatatgccttggcggatgcctctctcaagatggccgaccccaatcgctttcgcg |
| E6, E5, and | | gcaaagaccttccggtcctggaccagctgaccgaccctccgggggtccggcgcgtgtaccaca |
| C-terminus | | tccaggcgggcctaccggacccgttccagccccccagcctcccgatcacggtttactacgccgt |
| E2 Fusion | | gttggagcgcgcctgccgcagcgtgctcctaaacgcaccgtcggaggccccccagattgtccg |
| Protein | | cggggcctccgaagacgtccggaaacaaccctacaacctgaccatcgcttggtttcggatggga |
| Fused to | | ggcaactgtgctatccccatcacggtcatggagtacaccgaatgctcctacaacaagtctctggg |
| gD | | ggcctgtcccatccgaacgcagccccgctggaactactatgacagcttcagcgccgtcagcga |
| Nucleotide | | ggataacctggggttcctgatgcacgcccccgcgtttgagaccgccggcacgtacctgcggctc |
| | | gtgaagataaacgactggacggagattacacagtttatcctggagcaccgagccaagggctcct |
| | | gtaagtacgccctcccgctgcgcatccccccgtcagcctgcctgtccccccaggcctaccagca |
| | | gggggtgacggtggacagcatcgggatgctgccccgcttcatccccgagaaccagcgcaccg |
| | | tcgccgtatacagcttgaagatcgccgggtggcacgggccccacggcgacacccccaccctg |
| | | cacgagtacatgctggacctgcagcccgagaccaccgccgccgccgccgccgcccagctga |
| | | acgacagcagcgaggaggaggacgagatcgacggccccgccggccaggccgagcccgac |
| | | agggctcactacaacatcgtgaccttctgctgcaagtgcgacagcaccctgaggctgtgcgtgc |
| | | agagcacccacgtggacatcaggaccctggaggacctgctgatgggcaccctgggcatcgtgt |
| | | gccccatctgcagccagaagcccggcgcccaccagaagaggaccgccatgttccaggacccc |
| | | caggagaggcccaggaagctgccccagctgtgcaccgagctgcagaccaccatccacgacat |
| | | catcctggagtgcgtgtactgcaagcagcagctgctgaggagggaggtgtacgacgccgccg |
| | | ccgccgccctgtgcatcgtgtacagggacggcaacccctacgccgtgtgcgacaagtgcctga |
| | | agttctacagcaagatcagcgagtacaggcactactgctacagcctgtacggcaccaccctgga |
| | | gcagcagtacaacaagcccctgtgcgacctgctgatcaggtgcatcaactgccagaagcccca |
| | | gtgccccgaggagaagcagaggcacctggacaagaagcagaggttccacaacatcaggggc |
| | | aggtggaccggcaggtgcatgagctgctgcaggagcagcaggaccaggagggagacccag |
| | | ctgggcgcctgcgtgctgctgtgcgtgtgcctgctgatcaggcccctgctgctgagcgtgagca |
| | | cctacaccagcctgatcatcctggtgctgctgctgtggatcaccgccgccagcgccttcaggtgc |
| | | ttcatcgtgtacatcgccttcgtgtacatccccctgttcctgatccacacccacgccagtaacacta |
| | | cacccatagtacatttaaaaggtgatgctaatactttaaaatgtttaagatatagatttaaaaagcatt |
| | | gtaaattgtatactgcagtgtcgtctacatggcattggacaggacataatgtaaaacataaaagtgc |
| | | aattgttacacttacatatgatagtgaatgggggcccaaggccccatacacgagcaccctgctgc |
| | | ccccggagctgtccgagacccccaacgccacgcagccagaactcgccccggaagaccccga |
| | | ggattcggccctcttggaggaccccgtggggacggtggcgccgcaaatcccaccaaactggc |
| | | acatcccgtcgatccaggacgccgcgacgccttaccatcccccggccaccccgaacaacatgg |
| | | gcctgatcgccggcgcggtgggcggcagtctcctggcagccctggtcatttgcggaattgtgta |
| | | ctggatgcaccgccgcactcggaaagccccaaagcgcatacgcctcccccacatccgggaag |
| | | acgaccagccgtcctcgcaccagcccttgttttac |
| Mutant | SEQ ID | MGGAAARLGAVILFVVIVGLHGVRGKYALADASLKMADPN |
| HPV E7, | NO: 104 | RFRGKDLPVLDQLTDPPGVRRVYHIQAGLPDPFQPPSLPITVY |
| E6, E5, and | | YAVLERACRSVLLNAPSEAPQIVRGASEDVRKQPYNLTIAWF |
| N-terminus | | RMGGNCAIPITVMEYTECSYNKSLGACPIRTQPRWNYYDSFS |
| E2 Fusion | | AVSEDNLGFLMHAPAFETAGTYLRLVKINDWTEITQFILEHR |
| Protein | | AKGSCKYALPLRIPPSACLSPQAYQQGVTVDSIGMLPRFIPEN |
| Fused to | | QRTVAVYSLKIAGWHGPHGDTPTLHEYMLDLQPETTAAAA |
| gD Protein | | AAQLNDSSEEEDEIDGPAGQAEPDRAHYNIVTFCCKCDSTLR |
| | | LCVQSTHVDIRTLEDLLMGTLGIVCPICSQKPGAHQKRTAMF |
| | | QDPQERPRKLPQLCTELQTTIHDIILECVYCKQQLLRREVYDA |
| | | AAAALCIVYRDGNPYAVCDKCLKFYSKISEYRHYCYSLYGT |
| | | TLEQQYNKPLCDLLIRCINCQKPQCPEEKQRHLDKKQRFHNI |
| | | RGRWTGRCMSCCRSSRTRRETQLGACVLLCVCLLIRPLLLSV |
| | | STYTSLIILVLLLWITAASAFRCFIVYIAFVYIPLFLIHTHAETL |
| | | CQRLNVCQDKILTHYENDSTDLRDHIDYWKHMRLECAIYYK |
| | | AREMGFKHINHQVVPTLAVSKNKALQAIELQLTLETIYNSQY |
| | | SNEKWGPKAPYTSTLLPPELSETPNATQPELAPEDPEDSALLE |
| | | DPVGTVAPQIPPNWHIPSIQDAATPYHPPATPNNMGLIAGAV |
| | | GGSLLAALVICGIVYWMHRRTRKAPKRIRLPHIREDDQPSSH |
| | | QPLFY |
| Mutant | SEQ ID | atggggggggctgccgccaggttgggggccgtgattttgtttgtcgtcatagtgggcctccatgg |
| HPV E7, | NO: 105 | ggtccgcggcaaatatgccttggcggatgcctctctcaagatggccgaccccaatcgctttcgcg |
| E6, E5, and | | gcaaagaccttccggtcctggaccagctgaccgaccctccgggggtccggcgcgtgtaccaca |
| N-terminus | | tccaggcgggcctaccggacccgttccagccccccagcctcccgatcacggtttactacgccgt |
| E2 Fusion | | gttggagcgcgcctgccgcagcgtgctcctaaacgcaccgtcggaggccccccagattgtccg |
| Protein | | cggggcctccgaagacgtccggaaacaaccctacaacctgaccatcgcttggtttcggatggga |
| Fused to | | ggcaactgtgctatccccatcacggtcatggagtacaccgaatgctcctacaacaagtctctggg |
| gD | | ggcctgtcccatccgaacgcagccccgctggaactactatgacagcttcagcgccgtcagcga |
| Nucleotide | | ggataacctggggttcctgatgcacgcccccgcgtttgagaccgccggcacgtacctgcggctc |
| | | gtgaagataaacgactggacggagattacacagtttatcctggagcaccgagccaagggctcct |
| | | gtaagtacgccctcccgctgcgcatccccccgtcagcctgcctgtccccccaggcctaccagca |
| | | gggggtgacggtggacagcatcgggatgctgccccgcttcatccccgagaaccagcgcaccg |
| | | tcgccgtatacagcttgaagatcgccgggtggcacgggccccacggcgacacccccaccctg |
| | | cacgagtacatgctggacctgcagcccgagaccaccgccgccgccgccgccgcccagctga |
| | | acgacagcagcgaggaggaggacgagatcgacggccccgccggccaggccgagcccgac |
| | | agggctcactacaacatcgtgaccttctgctgcaagtgcgacagcaccctgaggctgtgcgtgc |
| | | agagcacccacgtggacatcaggaccctggaggacctgctgatgggcaccctgggcatcgtgt |
| | | gccccatctgcagccagaagcccggcgcccaccagaagaggaccgccatgttccaggacccc |
| | | caggagaggcccaggaagctgccccagctgtgcaccgagctgcagaccaccatccacgacat |
| | | catcctggagtgcgtgtactgcaagcagcagctgctgaggagggaggtgtacgacgccgccg |
| | | ccgccgccctgtgcatcgtgtacagggacggcaacccctacgccgtgtgcgacaagtgcctga |
| | | agttctacagcaagatcagcgagtacaggcactactgctacagcctgtacggcaccaccctgga |
| | | gcagcagtacaacaagcccctgtgcgacctgctgatcaggtgcatcaactgccagaagcccca |
| | | gtgccccgaggagaagcagaggcacctggacaagaagcagaggttccacaacatcaggggc |
| | | aggtggaccggcaggtgcatgagctgctgcaggagcagcaggaccaggagggagacccag |
| | | ctgggcgcctgcgtgctgctgtgcgtgtgcctgctgatcaggcccctgctgctgagcgtgagca |
| | | cctacaccagcctgatcatcctggtgctgctgctgtggatcaccgccgccagcgccttcaggtgc |
| | | ttcatcgtgtacatcgccttcgtgtacatccccctgttcctgatccacacccacgccgagactctttg |
| | | ccaacgtttaaatgtgtgtcaggacaaaatactaacacattatgaaaatgatagtacagacctacgt |
| | | gaccatatagactattggaaacacatgcgcctagaatgtgctatttattacaaggccagagaaatg |
| | | ggatttaaacatattaaccaccaggtggtgccaacactggctgtatcaaagaataaagcattacaa |
| | | gcaattgaactgcaactaacgttagaaacaatatataactcacaatatagtaatgaaaagtggggg |
| | | cccaaggccccatacacgagcaccctgctgcccccggagctgtccgagacccccaacgccac |
| | | gcagccagaactcgccccggaagaccccgaggattcggccctcttggaggaccccgtgggga |
| | | cggtggcgccgcaaatcccaccaaactggcacatcccgtcgatccaggacgccgcgacgcctt |
| | | accatcccccggccaccccgaacaacatgggcctgatcgccggcgcggtgggcggcagtctc |
| | | ctggcagccctggtcatttgcggaattgtgtactggatgcaccgccgcactcggaaagccccaa |
| | | agcgcatacgcctcccccacatccgggaagacgaccagccgtcctcgcaccagcccttgtttta |
| | | c |
| Mutant | SEQ ID | MGGAAARLGAVILFVVIVGLHGVRGKYCLADASLKMADPN |
| HPV E7, | NO: 106 | RFRGKDLPVLDQLTDPPGVRRVYHIQAGLPDPFQPPSLPITVC |
| E6, E5, and | | YAVLERACRSVLLNAPSEAPQIVRGASEDVRKQPYNLTIAWF |
| C-terminus | | RMGGNCAIPITVMEYTECSYNKSLGACPIRTQPRWNYYDSFS |
| E2 Fusion | | AVSEDNLGFLMHAPAFETAGTYLRLVKINDWTEITQFILEHR |
| Protein | | AKGSCKYALPLRIPPSACLSPQAYQQGVTVDSIGPHGDTPTL |
| Fused to | | HEYMLDLQPETTAAAAAAQLNDSSEEEDEIDGPAGQAEPDR |
| gDM5 | | AHYNIVTFCCKCDSTLRLCVQSTHVDIRTLEDLLMGTLGIVC |
| Protein | | PICSQKPGAHQKRTAMFQDPQERPRKLPQLCTELQTTIHDIIL |
| | | ECVYCKQQLLRREVYDAAAAALCIVYRDGNPYAVCDKCLK |
| | | FYSKISEYRHYCYSLYGTTLEQQYNKPLCDLLIRCINCQKPQC |
| | | PEEKQRHLDKKQRFHNIRGRWTGRCMSCCRSSRTRRETQLG |
| | | ACVLLCVCLLIRPLLLSVSTYTSLIILVLLLWITAASAFRCFIV |
| | | YIAFVYIPLFLIHTHASNTTPIVHLKGDANTLKCLRYRFKKHC |
| | | KLYTAVSSTWHWTGHNVKHKSAIVTLTYDSEWGPATQPEL |
| | | APEDPEDSALLEDPVGTVAPQIPPNWHIPSIQDAATPYHPPAT |
| | | PNNMGLIAGAVGGSLLAALVICGIVYWMHRRTRKAPKRIRL |
| | | PHIREDDQPSSHQPLFY |
| Mutant | SEQ ID | atggggggggctgccgccaggttgggggccgtgattttgtttgtcgtcatagtgggcctccatgg |
| HPV E7, | NO: 107 | ggtccgcggcaaatattgtttggcggatgcctctctcaagatggccgaccccaatcgctttcgcg |
| E6, E5, and | | gcaaagaccttccggtcctggaccagctgaccgaccctccgggggtccggcgcgtgtaccaca |
| C-terminus | | tccaggcgggcctaccggacccgttccagccccccagcctcccgatcacggtttgctacgccgt |
| E2 Fusion | | gttggagcgcgcctgccgcagcgtgctcctaaacgcaccgtcggaggccccccagattgtccg |
| Protein | | cggggcctccgaagacgtccggaaacaaccctacaacctgaccatcgcttggtttcggatggga |
| Fused to | | ggcaactgtgctatccccatcacggtcatggagtacaccgaatgctcctacaacaagtctctggg |
| gDM5 | | ggcctgtcccatccgaacgcagccccgctggaactactatgacagcttcagcgccgtcagcga |
| Nucleotide | | ggataacctggggttcctgatgcacgcccccgcgtttgagaccgccggcacgtacctgcggctc |
| | | gtgaagataaacgactggacggagattacacagtttatcctggagcaccgagccaagggctcct |
| | | gtaagtacgccctcccgctgcgcatccccccgtcagcctgcctctccccccaggcctaccagca |
| | | gggggtgacggtggacagcatcgggccccacggcgacacccccaccctgcacgagtacatg |
| | | ctggacctgcagcccgagaccaccgccgccgccgccgccgcccagctgaacgacagcagcg |
| | | aggaggaggacgagatcgacggccccgccggccaggccgagcccgacagggctcactaca |
| | | acatcgtgaccttctgctgcaagtgcgacagcaccctgaggctgtgcgtgcagagcacccacgt |
| | | ggacatcaggaccctggaggacctgctgatgggcaccctgggcatcgtgtgccccatctgcag |
| | | ccagaagcccggcgcccaccagaagaggaccgccatgttccaggacccccaggagaggccc |
| | | aggaagctgccccagctgtgcaccgagctgcagaccaccatccacgacatcatcctggagtgc |
| | | gtgtactgcaagcagcagctgctgaggagggaggtgtacgacgccgccgccgccgccctgtg |
| | | catcgtgtacagggacggcaacccctacgccgtgtgcgacaagtgcctgaagttctacagcaag |
| | | atcagcgagtacaggcactactgctacagcctgtacggcaccaccctggagcagcagtacaac |
| | | aagcccctgtgcgacctgctgatcaggtgcatcaactgccagaagccccagtgccccgaggag |
| | | aagcagaggcacctggacaagaagcagaggttccacaacatcaggggcaggtggaccggca |
| | | ggtgcatgagctgctgcaggagcagcaggaccaggagggagacccagctgggcgcctgcgt |
| | | gctgctgtgcgtgtgcctgctgatcaggcccctgctgctgagcgtgagcacctacaccagcctg |
| | | atcatcctggtgctgctgctgtggatcaccgccgccagcgccttcaggtgcttcatcgtgtacatc |
| | | gccttcgtgtacatccccctgttcctgatccacacccacgccagtaacactacacccatagtacatt |
| | | taaaaggtgatgctaatactttaaaatgtttaagatatagatttaaaaagcattgtaaattgtatactgc |
| | | agtgtcgtctacatggcattggacaggacataatgtaaaacataaaagtgcaattgttacacttaca |
| | | tatgatagtgaatgggggcccgccacgcagccagaactcgccccggaagaccccgaggattc |
| | | ggccctcttggaggaccccgtggggacggtggcgccgcaaatcccaccaaactggcacatcc |
| | | cgtcgatccaggacgccgcgacgccttaccatcccccggccaccccgaacaacatgggcctg |
| | | atcgccggcgcggtgggcggcagtctcctggcagccctggtcatttgcggaattgtgtactggat |
| | | gcaccgccgcactcggaaagccccaaagcgcatacgcctcccccacatccgggaagacgac |
| | | cagccgtcctcgcaccagcccttgttttac |
| Mutant | SEQ ID | MGGAAARLGAVILFVVIVGLHGVRGKYCLADASLKMADPN |
| HPV E7, | NO: 108 | RFRGKDLPVLDQLTDPPGVRRVYHIQAGLPDPFQPPSLPITVC |
| E6, E5, and | | YAVLERACRSVLLNAPSEAPQIVRGASEDVRKQPYNLTIAWF |
| N-terminus | | RMGGNCAIPITVMEYTECSYNKSLGACPIRTQPRWNYYDSFS |
| E2 Fusion | | AVSEDNLGFLMHAPAFETAGTYLRLVKINDWTEITQFILEHR |
| Protein | | AKGSCKYALPLRIPPSACLSPQAYQQGVTVDSIGPHGDTPTL |
| Fused to | | HEYMLDLQPETTAAAAAAQLNDSSEEEDEIDGPAGQAEPDR |
| gDM5 | | AHYNIVTFCCKCDSTLRLCVQSTHVDIRTLEDLLMGTLGIVC |
| Protein | | PICSQKPGAHQKRTAMFQDPQERPRKLPQLCTELQTTIHDIIL |
| | | ECVYCKQQLLRREVYDAAAAALCIVYRDGNPYAVCDKCLK |
| | | FYSKISEYRHYCYSLYGTTLEQQYNKPLCDLLIRCINCQKPQC |
| | | PEEKQRHLDKKQRFHNIRGRWTGRCMSCCRSSRTRRETQLG |
| | | ACVLLCVCLLIRPLLLSVSTYTSLIILVLLLWITAASAFRCFIV |
| | | YIAFVYIPLFLIHTHAETLCQRLNVCQDKILTHYENDSTDLRD |
| | | HIDYWKHMRLECAIYYKAREMGFKHINHQVVPTLAVSKNK |
| | | ALQAIELQLTLETIYNSQYSNEKWGPATQPELAPEDPEDSALL |
| | | EDPVGTVAPQIPPNWHIPSIQDAATPYHPPATPNNMGLIAGA |
| | | VGGSLLAALVICGIVYWMHRRTRKAPKRIRLPHIREDDQPSS |
| | | HQPLFY |
| Mutant | SEQ ID | atggggggggctgccgccaggttgggggccgtgattttgtttgtcgtcatagtgggcctccatgg |
| HPV E7, | NO: 109 | ggtccgcggcaaatattgtttggcggatgcctctctcaagatggccgaccccaatcgctttcgcg |
| E6, E5, and | | gcaaagaccttccggtcctggaccagctgaccgaccctccgggggtccggcgcgtgtaccaca |
| N-terminus | | tccaggcgggcctaccggacccgttccagccccccagcctcccgatcacggtttgctacgccgt |
| E2 Fusion | | gttggagcgcgcctgccgcagcgtgctcctaaacgcaccgtcggaggccccccagattgtccg |
| Protein | | cggggcctccgaagacgtccggaaacaaccctacaacctgaccatcgcttggtttcggatggga |
| Fused to | | ggcaactgtgctatccccatcacggtcatggagtacaccgaatgctcctacaacaagtctctggg |
| gDM5 | | ggcctgtcccatccgaacgcagccccgctggaactactatgacagcttcagcgccgtcagcga |
| Nucleotide | | ggataacctggggttcctgatgcacgcccccgcgtttgagaccgccggcacgtacctgcggctc |
| | | gtgaagataaacgactggacggagattacacagtttatcctggagcaccgagccaagggctcct |
| | | gtaagtacgccctcccgctgcgcatccccccgtcagcctgcctctccccccaggcctaccagca |
| | | gggggtgacggtggacagcatcgggccccacggcgacacccccaccctgcacgagtacatg |
| | | ctggacctgcagcccgagaccaccgccgccgccgccgccgcccagctgaacgacagcagcg |
| | | aggaggaggacgagatcgacggccccgccggccaggccgagcccgacagggctcactaca |
| | | acatcgtgaccttctgctgcaagtgcgacagcaccctgaggctgtgcgtgcagagcacccacgt |
| | | ggacatcaggaccctggaggacctgctgatgggcaccctgggcatcgtgtgccccatctgcag |
| | | ccagaagcccggcgcccaccagaagaggaccgccatgttccaggacccccaggagaggccc |
| | | aggaagctgccccagctgtgcaccgagctgcagaccaccatccacgacatcatcctggagtgc |
| | | gtgtactgcaagcagcagctgctgaggagggaggtgtacgacgccgccgccgccgccctgtg |
| | | catcgtgtacagggacggcaacccctacgccgtgtgcgacaagtgcctgaagttctacagcaag |
| | | atcagcgagtacaggcactactgctacagcctgtacggcaccaccctggagcagcagtacaac |
| | | aagcccctgtgcgacctgctgatcaggtgcatcaactgccagaagccccagtgccccgaggag |
| | | aagcagaggcacctggacaagaagcagaggttccacaacatcaggggcaggtggaccggca |
| | | ggtgcatgagctgctgcaggagcagcaggaccaggagggagacccagctgggcgcctgcgt |
| | | gctgctgtgcgtgtgcctgctgatcaggcccctgctgctgagcgtgagcacctacaccagcctg |
| | | atcatcctggtgctgctgctgtggatcaccgccgccagcgccttcaggtgcttcatcgtgtacatc |
| | | gccttcgtgtacatccccctgttcctgatccacacccacgccgagactctttgccaacgtttaaatg |
| | | tgtgtcaggacaaaatactaacacattatgaaaatgatagtacagacctacgtgaccatatagact |
| | | attggaaacacatgcgcctagaatgtgctatttattacaaggccagagaaatgggatttaaacatat |
| | | taaccaccaggtggtgccaacactggctgtatcaaagaataaagcattacaagcaattgaactgc |
| | | aactaacgttagaaacaatatataactcacaatatagtaatgaaaagtgggggcccgccacgcag |
| | | ccagaactcgccccggaagaccccgaggattcggccctcttggaggaccccgtggggacggt |
| | | ggcgccgcaaatcccaccaaactggcacatcccgtcgatccaggacgccgcgacgccttacca |
| | | tcccccggccaccccgaacaacatgggcctgatcgccggcgcggtgggcggcagtctcctgg |
| | | cagccctggtcatttgcggaattgtgtactggatgcaccgccgcactcggaaagccccaaagcg |
| | | catacgcctcccccacatccgggaagacgaccagccgtcctcgcaccagcccttgttttac |
| Mutant E7, | SEQ ID | MGGAAARLGAVILFVVIVGLHGVRGKYCLADASLKMADPN |
| E6, and E5 | NO: 110 | RFRGKDLPVLDQLTDPPGVRRVYHIQAGLPDPFQPPSLPITVC |
| fused into | | YAVLERACRSVLLNAPSEAPQIVRGASEDVRKQPYNLTIAWF |
| gDM5 | | RMGGNCAIPITVMEYTECSYNKSLGACPIRTQPRWNYYDSFS |
| Protein | | AVSEDNLGFLMHAPAFETAGTYLRLVKINDWTEITQFILEHR |
| | | AKGSCKYALPLRIPPSACLSPQAYQQGVTVDSIGPHGDTPTL |
| | | HEYMLDLQPETTAAAAAAQLNDSSEEEDEIDGPAGQAEPDR |
| | | AHYNIVTFCCKCDSTLRLCVQSTHVDIRTLEDLLMGTLGIVC |
| | | PICSQKPGAHQKRTAMFQDPQERPRKLPQLCTELQTTIHDIIL |
| | | ECVYCKQQLLRREVYDAAAAALCIVYRDGNPYAVCDKCLK |
| | | FYSKISEYRHYCYSLYGTTLEQQYNKPLCDLLIRCINCQKPQC |
| | | PEEKQRHLDKKQRFHNIRGRWTGRCMSCCRSSRTRRETQLG |
| | | ACVLLCVCLLIRPLLLSVSTYTSLIILVLLLWITAASAFRCFIV |
| | | YIAFVYIPLFLIHTHAGPATQPELAPEDPEDSALLEDPVGTVA |
| | | PQIPPNWHIPSIQDAATPYHPPATPNNMGLIAGAVGGSLLAAL |
| | | VICGIVYWMHRRTRKAPKRIRLPHIREDDQPSSHQPLFY |
| Mutant E7, | SEQ ID | atggggggggctgccgccaggttgggggccgtgattttgtttgtcgtcatagtgggcctccatgg |
| E6, and E5 | NO: 111 | ggtccgcggcaaatattgtttggcggatgcctctctcaagatggccgaccccaatcgctttcgcg |
| fused into | | gcaaagaccttccggtcctggaccagctgaccgaccctccgggggtccggcgcgtgtaccaca |
| gDM5 | | tccaggcgggcctaccggacccgttccagccccccagcctcccgatcacggtttgctacgccgt |
| Nucleotide | | gttggagcgcgcctgccgcagcgtgctcctaaacgcaccgtcggaggccccccagattgtccg |
| | | cggggcctccgaagacgtccggaaacaaccctacaacctgaccatcgcttggtttcggatggga |
| | | ggcaactgtgctatccccatcacggtcatggagtacaccgaatgctcctacaacaagtctctggg |
| | | ggcctgtcccatccgaacgcagccccgctggaactactatgacagcttcagcgccgtcagcga |
| | | ggataacctggggttcctgatgcacgcccccgcgtttgagaccgccggcacgtacctgcggctc |
| | | gtgaagataaacgactggacggagattacacagtttatcctggagcaccgagccaagggctcct |
| | | gtaagtacgccctcccgctgcgcatccccccgtcagcctgcctctccccccaggcctaccagca |
| | | gggggtgacggtggacagcatcgggccccacggcgacacccccaccctgcacgagtacatg |
| | | ctggacctgcagcccgagaccaccgccgccgccgccgccgcccagctgaacgacagcagcg |
| | | aggaggaggacgagatcgacggccccgccggccaggccgagcccgacagggctcactaca |
| | | acatcgtgaccttctgctgcaagtgcgacagcaccctgaggctgtgcgtgcagagcacccacgt |
| | | ggacatcaggaccctggaggacctgctgatgggcaccctgggcatcgtgtgccccatctgcag |
| | | ccagaagcccggcgcccaccagaagaggaccgccatgttccaggacccccaggagaggccc |
| | | aggaagctgccccagctgtgcaccgagctgcagaccaccatccacgacatcatcctggagtgc |
| | | gtgtactgcaagcagcagctgctgaggagggaggtgtacgacgccgccgccgccgccctgtg |
| | | catcgtgtacagggacggcaacccctacgccgtgtgcgacaagtgcctgaagttctacagcaag |
| | | atcagcgagtacaggcactactgctacagcctgtacggcaccaccctggagcagcagtacaac |
| | | aagcccctgtgcgacctgctgatcaggtgcatcaactgccagaagccccagtgccccgaggag |
| | | aagcagaggcacctggacaagaagcagaggttccacaacatcaggggcaggtggaccggca |
| | | ggtgcatgagctgctgcaggagcagcaggaccaggagggagacccagctgggcgcctgcgt |
| | | gctgctgtgcgtgtgcctgctgatcaggcccctgctgctgagcgtgagcacctacaccagcctg |
| | | atcatcctggtgctgctgctgtggatcaccgccgccagcgccttcaggtgcttcatcgtgtacatc |
| | | gccttcgtgtacatccccctgttcctgatccacacccacgccgggcccgccacgcagccagaac |
| | | tcgccccggaagaccccgaggattcggccctcttggaggaccccgtggggacggtggcgccg |
| | | caaatcccaccaaactggcacatcccgtcgatccaggacgccgcgacgccttaccatcccccg |
| | | gccaccccgaacaacatgggcctgatcgccggcgcggtgggcggcagtctcctggcagccct |
| | | ggtcatttgcggaattgtgtactggatgcaccgccgcactcggaaagccccaaagcgcatacgc |
| | | ctcccccacatccgggaagacgaccagccgtcctcgcaccagcccttgttttac |
| gD-PolN | SEQ ID | MGGAAARLGAVILFVVIVGLHGVRGKYALADASLKMADPN |
| Protein | NO: 112 | RFRGKDLPVLDQLTDPPGVRRVYHIQAGLPDPFQPPSLPITVY |
| | | YAVLERACRSVLLNAPSEAPQIVRGASEDVRKQPYNLTIAWF |
| | | RMGGNCAIPITVMEYTECSYNKSLGACPIRTQPRWNYYDSFS |
| | | AVSEDNLGFLMHAPAFETAGTYLRLVKINDWTEITQFILEHR |
| | | AKGSCKYALPLRIPPSACLSPQAYQQGVTVDSIGMLPRFIPEN |
| | | QRTVAVYSLKIAGWHGPPLSYQHFRKLLLLDEEAGPLEEELP |
| | | RLADEGLNRRVAEDLNLGNLNVSIPWTHKVGNFTGLYSSTV |
| | | PVFNPEWQTPSFPKIHLQEDIVDRCKQFVGPLTVNEKRRLKLI |
| | | MPARFYPNVTKYLPLDKGIKPYYPEHAVNHYFQTRHYLHTL |
| | | WKAGILYKRETTRSASFCGSPYSWEQELQHGSCWWLQFRNS |
| | | KPCSEYCLTHLVNLLEDWGPCDEHGEHHIRIPRTPARVTGGV |
| | | FLVDKNPHNTAESRLVVDFSQFSRGITRVSWPKFAVPNLQSL |
| | | TNLLSSNLSWLSLDVSAAFYHIPLHPAAMPGPKAPYTSTLLPP |
| | | ELSETPNATQPELAPEDPEDSALLEDPVGTVAPQIPPNWHIPSI |
| | | QDAATPYHPPATPNNMGLIAGAVGGSLLAALVICGIVYWMH |
| | | RRTRKAPKRIRLPHIREDDQPSSHQPLFY |
| gD-PolN | SEQ ID | atggggggggctgccgccaggttgggggccgtgattttgtttgtcgtcatagtgggcctccatgg |
| Nucleotide | NO: 113 | ggtccgcggcaaatatgccttggcggatgcctctctcaagatggccgaccccaatcgctttcgcg |
| | | gcaaagaccttccggtcctggaccagctgaccgaccctccgggggtccggcgcgtgtaccaca |
| | | tccaggcgggcctaccggacccgttccagccccccagcctcccgatcacggtttactacgccgt |
| | | gttggagcgcgcctgccgcagcgtgctcctaaacgcaccgtcggaggccccccagattgtccg |
| | | cggggcctccgaagacgtccggaaacaaccctacaacctgaccatcgcttggtttcggatggga |
| | | ggcaactgtgctatccccatcacggtcatggagtacaccgaatgctcctacaacaagtctctggg |
| | | ggcctgtcccatccgaacgcagccccgctggaactactatgacagcttcagcgccgtcagcga |
| | | ggataacctggggttcctgatgcacgcccccgcgtttgagaccgccggcacgtacctgcggctc |
| | | gtgaagataaacgactggacggagattacacagtttatcctggagcaccgagccaagggctcct |
| | | gtaagtacgccctcccgctgcgcatccccccgtcagcctgcctctccccccaggcctaccagca |
| | | gggggtgacggtggacagcatcgggatgctgccccgcttcatccccgagaaccagcgcaccg |
| | | tcgccgtatacagcttgaagatcgccgggtggcacgggccccccctgagctaccagcacttcag |
| | | gaagctgctgctgctggacgaggaggccggccccctggaggaggagctgcccaggctggcc |
| | | gacgagggcctgaacaggagggggccgaggacctgaacctgggcaacctgaacgtgagca |
| | | tcccctggacccacaaggtgggcaacttcaccggcctgtacagcagcaccgtgcccgtgttcaa |
| | | ccccgagtggcagacccccagcttccccaagatccacctgcaggaggacatcgtggacaggt |
| | | gcaagcagttcgtgggtcccctgaccgtgaacgagaagaggaggctgaagctgatcatgcccg |
| | | ccaggttctaccccaacgtgaccaagtacctgcccctggacaagggcatcaagccctactaccc |
| | | cgagcacgccgtgaaccactacttccagaccaggcactacctgcacaccctgtggaaggccgg |
| | | catcctgtacaagagggagaccaccaggagcgccagcttctgcggcagcccctacagctggg |
| | | agcaggagctgcagcacggcagctgctggtggctgcagttcaggaacagcaagccctgcagc |
| | | gagtactgcctgacccacctggtgaacctgctggaggactggggtccctgcgacgagcacggc |
| | | gagcaccacatcaggatccccaggacccccgccagggtgaccggcggcgtgttcctggtgga |
| | | caagaacccccacaacaccgccgagagcaggctggtggtggacttcagccagttcagcaggg |
| | | gcatcaccagggtgagctggcccaagttcgccgtgcccaacctgcagagcctgaccaacctgc |
| | | tgagcagcaacctgagctggctgagcctggacgtgagcgccgccttctaccacatccccctgca |
| | | ccccgccgccatgcccgggcccaaggccccatacacgagcaccctgctgcccccggagctgt |
| | | ccgagacccccaacgccacgcagccagaactcgccccggaagaccccgaggattcggccct |
| | | cttggaggaccccgtggggacggtggcgccgcaaatcccaccaaactggcacatcccgtcgat |
| | | ccaggacgccgcgacgccttaccatcccccggccaccccgaacaacatgggcctgatcgccg |
| | | gcgcggtgggcggcagtctcctggcagccctggtcatttgcggaattgtgtactggatgcaccg |
| | | ccgcactcggaaagccccaaagcgcatacgcctcccccacatccgggaagacgaccagccgt |
| | | cctcgcaccagcccttgttttac |
| gDM5- | SEQ ID | MGGAAARLGAVILFVVIVGLHGVRGKYCLADASLKMADPN |
| PolN | NO: 114 | RFRGKDLPVLDQLTDPPGVRRVYHIQAGLPDPFQPPSLPITVC |
| Protein | | YAVLERACRSVLLNAPSEAPQIVRGASEDVRKQPYNLTIAWF |
| | | RMGGNCAIPITVMEYTECSYNKSLGACPIRTQPRWNYYDSFS |
| | | AVSEDNLGFLMHAPAFETAGTYLRLVKINDWTEITQFILEHR |
| | | AKGSCKYALPLRIPPSACLSPQAYQQGVTVDSIGPPLSYQHFR |
| | | KLLLLDEEAGPLEEELPRLADEGLNRRVAEDLNLGNLNVSIP |
| | | WTHKVGNFTGLYSSTVPVFNPEWQTPSFPKIHLQEDIVDRCK |
| | | QFVGPLTVNEKRRLKLIMPARFYPNVTKYLPLDKGIKPYYPE |
| | | HAVNHYFQTRHYLHTLWKAGILYKRETTRSASFCGSPYSWE |
| | | QELQHGSCWWLQFRNSKPCSEYCLTHLVNLLEDWGPCDEH |
| | | GEHHIRIPRTPARVTGGVFLVDKNPHNTAESRLVVDFSQFSR |
| | | GITRVSWPKFAVPNLQSLTNLLSSNLSWLSLDVSAAFYHIPLH |
| | | PAAMPGPATQPELAPEDPEDSALLEDPVGTVAPQIPPNWHIPS |
| | | IQDAATPYHPPATPNNMGLIAGAVGGSLLAALVICGIVYWM |
| | | HRRTRKAPKRIRLPHIREDDQPSSHQPLFY |
| gDM5- | SEQ ID | atggggggggctgccgccaggttgggggccgtgattttgtttgtcgtcatagtgggcctccatgg |
| PolN | NO: 115 | ggtccgcggcaaatattgtttggcggatgcctctctcaagatggccgaccccaatcgctttcgcg |
| Nucleotide | | gcaaagaccttccggtcctggaccagctgaccgaccctccgggggtccggcgcgtgtaccaca |
| | | tccaggcgggcctaccggacccgttccagccccccagcctcccgatcacggtttgctacgccgt |
| | | gttggagcgcgcctgccgcagcgtgctcctaaacgcaccgtcggaggccccccagattgtccg |
| | | cggggcctccgaagacgtccggaaacaaccctacaacctgaccatcgcttggtttcggatggga |
| | | ggcaactgtgctatccccatcacggtcatggagtacaccgaatgctcctacaacaagtctctggg |
| | | ggcctgtcccatccgaacgcagccccgctggaactactatgacagcttcagcgccgtcagcga |
| | | ggataacctggggttcctgatgcacgcccccgcgtttgagaccgccggcacgtacctgcggctc |
| | | gtgaagataaacgactggacggagattacacagtttatcctggagcaccgagccaagggctcct |
| | | gtaagtacgccctcccgctgcgcatccccccgtcagcctgcctctccccccaggcctaccagca |
| | | gggggtgacggtggacagcatcgggccccccctgagctaccagcacttcaggaagctgctgct |
| | | gctggacgaggaggccggccccctggaggaggagctgcccaggctggccgacgagggcct |
| | | gaacaggagggtggccgaggacctgaacctgggcaacctgaacgtgagcatcccctggaccc |
| | | acaaggtgggcaacttcaccggcctgtacagcagcaccgtgcccgtgttcaaccccgagtggc |
| | | agacccccagcttccccaagatccacctgcaggaggacatcgtggacaggtgcaagcagttcg |
| | | tgggtcccctgaccgtgaacgagaagaggaggctgaagctgatcatgcccgccaggttctacc |
| | | ccaacgtgaccaagtacctgcccctggacaagggcatcaagccctactaccccgagcacgccg |
| | | tgaaccactacttccagaccaggcactacctgcacaccctgtggaaggccggcatcctgtacaa |
| | | gagggagaccaccaggagcgccagcttctgcggcagcccctacagctgggagcaggagctg |
| | | cagcacggcagctgctggtggctgcagttcaggaacagcaagccctgcagcgagtactgcctg |
| | | acccacctggtgaacctgctggaggactggggtccctgcgacgagcacggcgagcaccacat |
| | | caggatccccaggacccccgccagggtgaccggcggcgtgttcctggtggacaagaaccccc |
| | | acaacaccgccgagagcaggctggtggtggacttcagccagttcagcaggggcatcaccagg |
| | | gtgagctggcccaagttcgccgtgcccaacctgcagagcctgaccaacctgctgagcagcaac |
| | | ctgagctggctgagcctggacgtgagcgccgccttctaccacatccccctgcaccccgccgcc |
| | | atgcccgggcccgccacgcagccagaactcgccccggaagaccccgaggattcggccctctt |
| | | ggaggaccccgtggggacggtggcgccgcaaatcccaccaaactggcacatcccgtcgatcc |
| | | aggacgccgcgacgccttaccatcccccggccaccccgaacaacatgggcctgatcgccggc |
| | | gcggtgggcggcagtctcctggcagccctggtcatttgcggaattgtgtactggatgcaccgcc |
| | | gcactcggaaagccccaaagcgcatacgcctcccccacatccgggaagacgaccagccgtcc |
| | | tcgcaccagcccttgttttac |
| gD- | SEQ ID | PPGVRRVYHIQAGLPDPFQPPSLPITVYYAVLERACRSVLLNA |
| Melapoly | NO: 116 | PSEAPQIVRGASEDVRKQPYNLTIAWFRMGGNCAIPITVMEY |
| #2 Protein | | TECSYNKSLGACPIRTQPRWNYYDSFSAVSEDNLGFLMHAP |
| | | AFETAGTYLRLVKINDWTEITQFILEHRAKGSCKYALPLRIPP |
| | | SACLSPQAYQQGVTVDSIGMLPRFIPENQRTVAVYSLKIAGW |
| | | HGPNRLAKFVAAWTLKAAAAAARKFFHRTCKCTGNFAAAA |
| | | NESFALPYWNFATGRNECDVAAADQLGYSYAIDLPVSVAAA |
| | | AAASVYDFFVWLAAAAAASTFSFRNALAASQVMNLHNLAA |
| | | TAPDNLGYMAAAIAVVNALLLYAYDYEELYAKVPRNQDWL |
| | | AAAAFGLANEKSIAAAAPRIRYHAAAVYMTSTLLPPELSETP |
| | | NATQPELAPEDPEDSALLEDPVGTVAPQIPPNWHIPSIQDAAT |
| | | PYHPPATPNNMGLIAGAVGGSLLAALVICGIVYWMHRRTRK |
| | | APKRIRLPHIREDDQPSSHQPLFYDYKDDDDKT |
| gD- | SEQ ID | atggggggggctgccgccaggttgggggccgtgattttgtttgtcgtcatagtgggcctccatgg |
| Melapoly | NO: 117 | ggtccgcggcaaatatgccttggcggatgcctctctcaagatggccgaccccaatcgctttcgcg |
| #2 | | gcaaagaccttccggtcctggaccagctgaccgaccctccgggggtccggcgcgtgtaccaca |
| Nucleotide | | tccaggcgggcctaccggacccgttccagccccccagcctcccgatcacggtttactacgccgt |
| | | gttggagcgcgcctgccgcagcgtgctcctaaacgcaccgtcggaggccccccagattgtccg |
| | | cggggcctccgaagacgtccggaaacaaccctacaacctgaccatcgcttggtttcggatggga |
| | | ggcaactgtgctatccccatcacggtcatggagtacaccgaatgctcctacaacaagtctctggg |
| | | ggcctgtcccatccgaacgcagccccgctggaactactatgacagcttcagcgccgtcagcga |
| | | ggataacctggggttcctgatgcacgcccccgcgtttgagaccgccggcacgtacctgcggctc |
| | | gtgaagataaacgactggacggagattacacagtttatcctggagcaccgagccaagggctcct |
| | | gtaagtacgccctcccgctgcgcatccccccgtcagcctgcctgtccccccaggcctaccagca |
| | | gggggtgacggtggacagcatcgggatgctgccccgcttcatccccgagaaccagcgcaccg |
| | | tcgccgtatacagcttgaagatcgccgggtggcacgggcccaaccggttagctaagtttgtggc |
| | | cgcttggacactgaaggccgctgcagccgctgcaaggaagttctttcacagaacctgcaaatgt |
| | | acaggaaacttcgccgctgcagccaatgagagcttcgccctgccctattggaactttgctaccgg |
| | | ccggaatgaatgcgacgtggctgcagccgatcagctcggatactcttatgccatcgacctgcctg |
| | | tgagtgtcgctgcagccgctgcagcctcagtgtacgatttctttgtctggctggctgcagccgctg |
| | | cagccagcactttctcctttcgcaacgccctggctgcatctcaggtcatgaacctccataatctggc |
| | | agctaccgcaccagacaacctcggatatatggcagccgctattgccgtggtcaatgctctgctcc |
| | | tgtacgcctatgactacgaggaactgtacgctaaagtccccaggaatcaggattggctcgcagc |
| | | cgctgcatttggtctggccaacgaaaagagcattgcagcagcagcacctaggatccgatatcac |
| | | gcggccgcagtgtacatgacgagcaccctgctgcccccggagctgtccgagacccccaacgc |
| | | cacgcagccagaactcgccccggaagaccccgaggattcggccctcttggaggaccccgtgg |
| | | ggacggtggcgccgcaaatcccaccaaactggcacatcccgtcgatccaggacgccgcgacg |
| | | ccttaccatcccccggccaccccgaacaacatgggcctgatcgccggcgcgggggcggcag |
| | | tctcctggcagccctggtcatttgcggaattgtgtactggatgcaccgccgcactcggaaagccc |
| | | caaagcgcatacgcctcccccacatccgggaagacgaccagccgtcctcgcaccagcccttgt |
| | | tttacgactacaaagacgatgacgacaagact |
| gDM5- | SEQ ID | MGGAAARLGAVILFVVIVGLHGVRGKYCLADASLKMADPN |
| Melapoly | NO: 118 | RFRGKDLPVLDQLTDPPGVRRVYHIQAGLPDPFQPPSLPITVC |
| #2 Protein | | YAVLERACRSVLLNAPSEAPQIVRGASEDVRKQPYNLTIAWF |
| | | RMGGNCAIPITVMEYTECSYNKSLGACPIRTQPRWNYYDSFS |
| | | AVSEDNLGFLMHAPAFETAGTYLRLVKINDWTEITQFILEHR |
| | | AKGSCKYALPLRIPPSACLSPQAYQQGVTVDSIGPNRLAKFV |
| | | AAWTLKAAAAAARKFFHRTCKCTGNFAAAANESFALPYWN |
| | | FATGRNECDVAAADQLGYSYAIDLPVSVAAAAAASVYDFFV |
| | | WLAAAAAASTFSFRNALAASQVMNLHNLAATAPDNLGYM |
| | | AAAIAVVNALLLYAYDYEELYAKVPRNQDWLAAAAFGLAN |
| | | EKSIAAAAPRGPATQPELAPEDPEDSALLEDPVGTVAPQIPPN |
| | | WHIPSIQDAATPYHPPATPNNMGLIAGAVGGSLLAALVICGI |
| | | VYWMHRRTRKAPKRIRLPHIREDDQPSSHQPLFY |
| gDM5- | SEQ ID | atggggggggctgccgccaggttgggggccgtgattttgtttgtcgtcatagtgggcctccatgg |
| Melapoly | NO: 119 | ggtccgcggcaaatattgtttggcggatgcctctctcaagatggccgaccccaatcgctttcgcg |
| #2 | | gcaaagaccttccggtcctggaccagctgaccgaccctccgggggtccggcgcgtgtaccaca |
| Nucleotide | | tccaggcgggcctaccggacccgttccagccccccagcctcccgatcacggtttgctacgccgt |
| | | gttggagcgcgcctgccgcagcgtgctcctaaacgcaccgtcggaggccccccagattgtccg |
| | | cggggcctccgaagacgtccggaaacaaccctacaacctgaccatcgcttggtttcggatggga |
| | | ggcaactgtgctatccccatcacggtcatggagtacaccgaatgctcctacaacaagtctctggg |
| | | ggcctgtcccatccgaacgcagccccgctggaactactatgacagcttcagcgccgtcagcga |
| | | ggataacctggggttcctgatgcacgcccccgcgtttgagaccgccggcacgtacctgcggctc |
| | | gtgaagataaacgactggacggagattacacagtttatcctggagcaccgagccaagggctcct |
| | | gtaagtacgccctcccgctgcgcatccccccgtcagcctgcctctccccccaggcctaccagca |
| | | gggggtgacggtggacagcatcgggcccaaccggttagctaagtttgtggccgcttggacact |
| | | gaaggccgctgcagccgctgcaaggaagttctttcacagaacctgcaaatgtacaggaaacttc |
| | | gccgctgcagccaatgagagcttcgccctgccctattggaactttgctaccggccggaatgaatg |
| | | cgacgtggctgcagccgatcagctcggatactcttatgccatcgacctgcctgtgagtgtcgctg |
| | | cagccgctgcagcctcagtgtacgatttctttgtctggctggctgcagccgctgcagccagcactt |
| | | tctcctttcgcaacgccctggctgcatctcaggtcatgaacctccataatctggcagctaccgcac |
| | | cagacaacctcggatatatggcagccgctattgccgtggtcaatgctctgctcctgtacgcctatg |
| | | actacgaggaactgtacgctaaagtccccaggaatcaggattggctcgcagccgctgcatttggt |
| | | ctggccaacgaaaagagcattgcagcagcagcacctagggggcccgccacgcagccagaac |
| | | tcgccccggaagaccccgaggattcggccctcttggaggaccccgtggggacggtggcgccg |
| | | caaatcccaccaaactggcacatcccgtcgatccaggacgccgcgacgccttaccatcccccg |
| | | gccaccccgaacaacatgggcctgatcgccggcgcggtgggcggcagtctcctggcagccct |
| | | ggtcatttgcggaattgtgtactggatgcaccgccgcactcggaaagccccaaagcgcatacgc |
| | | ctcccccacatccgggaagacgaccagccgtcctcgcaccagcccttgttttac |
| Melanoma | SEQ ID | MGGAAARLGAVILFVVIVGLHGVRGKYALADASLKMADPN |
| antigens | NO: 120 | RFRGKDLPVLDQLTDPPGVRRVYHIQAGLPDPFQPPSLPITVY |
| with | | YAVLERACRSVLLNAPSEAPQIVRGASEDVRKQPYNLTIAWF |
| universal | | RMGGNCAIPITVMEYTECSYNKSLGACPIRTQPRWNYYDSFS |
| helper | | AVSEDNLGFLMHAPAFETAGTYLRLVKINDWTEITQFILEHR |
| epitope and | | AKGSCKYALPLRIPPSACLSPQAYQQGVTVDSIGMLPRFIPEN |
| E2 fused | | QRTVAVYSLKIAGWHGPNRLAKFVAAWTLKAAAAAARKFF |
| into gD | | HRTCKCTGNFAAAANESFALPYWNFATGRNECDVAAADQL |
| Protein | | GYSYAIDLPVSVAAAAAASVYDFFVWLAAAAAASTFSFRNA |
| | | LAASQVMNLHNLAATAPDNLGYMAAAIAVVNALLLYAYD |
| | | YEELYAKVPRNQDWLAAAAFGLANEKSIAAAAPRETLCQRL |
| | | NVCQDKILTHYENDSTDLRDHIDYWKHMRLECAIYYKARE |
| | | MGFKHINHQVVPTLAVSKNKALQAIELQLTLETIYNSQYSNE |
| | | KWSNTTPIVHLKGDANTLKCLRYRFKKHCKLYTAVSSTWH |
| | | WTGHNVKHKSAIVTLTYDSEWPRIRYHAAAVYMTSTLLPPE |
| | | LSETPNATQPELAPEDPEDSALLEDPVGTVAPQIPPNWHIPSIQ |
| | | DAATPYHPPATPNNMGLIAGAVGGSLLAALVICGIVYWMHR |
| | | RTRKAPKRIRLPHIREDDQPSSHQPLFY |
| Melanoma | SEQ ID | atggggggggctgccgccaggttgggggccgtgattttgtttgtcgtcatagtgggcctccatgg |
| antigens | NO: 121 | ggtccgcggcaaatatgccttggcggatgcctctctcaagatggccgaccccaatcgctttcgcg |
| with | | gcaaagaccttccggtcctggaccagctgaccgaccctccgggggtccggcgcgtgtaccaca |
| universal | | tccaggcgggcctaccggacccgttccagccccccagcctcccgatcacggtttactacgccgt |
| helper | | gttggagcgcgcctgccgcagcgtgctcctaaacgcaccgtcggaggccccccagattgtccg |
| epitope and | | cggggcctccgaagacgtccggaaacaaccctacaacctgaccatcgcttggtttcggatggga |
| E2 fused | | ggcaactgtgctatccccatcacggtcatggagtacaccgaatgctcctacaacaagtctctggg |
| into gD | | ggcctgtcccatccgaacgcagccccgctggaactactatgacagcttcagcgccgtcagcga |
| Nucleotide | | ggataacctggggttcctgatgcacgcccccgcgtttgagaccgccggcacgtacctgcggctc |
| | | gtgaagataaacgactggacggagattacacagtttatcctggagcaccgagccaagggctcct |
| | | gtaagtacgccctcccgctgcgcatccccccgtcagcctgcctgtccccccaggcctaccagca |
| | | gggggtgacggtggacagcatcgggatgctgccccgcttcatccccgagaaccagcgcaccg |
| | | tcgccgtatacagcttgaagatcgccgggtggcacgggcccaaccggttagctaagtttgtggc |
| | | cgcttggacactgaaggccgctgcagccgctgcaaggaagttctttcacagaacctgcaaatgt |
| | | acaggaaacttcgccgctgcagccaatgagagcttcgccctgccctattggaactttgctaccgg |
| | | ccggaatgaatgcgacgtggctgcagccgatcagctcggatactcttatgccatcgacctgcctg |
| | | tgagtgtcgctgcagccgctgcagcctcagtgtacgatttctttgtctggctggctgcagccgctg |
| | | cagccagcactttctcctttcgcaacgccctggctgcatctcaggtcatgaacctccataatctggc |
| | | agctaccgcaccagacaacctoggatatatggcagccgctattgccgtggtcaatgctctgctcc |
| | | tgtacgcctatgactacgaggaactgtacgctaaagtccccaggaatcaggattggctcgcagc |
| | | cgctgcatttggtctggccaacgaaaagagcattgcagcagcagcacctagggagactctttgc |
| | | caacgtttaaatgtgtgtcaggacaaaatactaacacattatgaaaatgatagtacagacctacgtg |
| | | accatatagactattggaaacacatgcgcctagaatgtgctatttattacaaggccagagaaatgg |
| | | gatttaaacatattaaccaccaggtggtgccaacactggctgtatcaaagaataaagcattacaag |
| | | caattgaactgcaactaacgttagaaacaatatataactcacaatatagtaatgaaaagtggagta |
| | | acactacacccatagtacatttaaaaggtgatgctaatactttaaaatgtttaagatatagatttaaaa |
| | | agcattgtaaattgtatactgcagtgtcgtctacatggcattggacaggacataatgtaaaacataa |
| | | aagtgcaattgttacacttacatatgatagtgaatggcctaggatccgatatcacgcggccgcagt |
| | | gtacatgacgagcaccctgctgcccccggagctgtccgagacccccaacgccacgcagccag |
| | | aactcgccccggaagaccccgaggattcggccctcttggaggaccccgtggggacggtggcg |
| | | ccgcaaatcccaccaaactggcacatcccgtcgatccaggacgccgcgacgccttaccatccc |
| | | ccggccaccccgaacaacatgggcctgatcgccggcgcggtgggcggcagtctcctggcagc |
| | | cctggtcatttgcggaattgtgtactggatgcaccgccgcactcggaaagccccaaagcgcata |
| | | cgcctcccccacatccgggaagacgaccagccgtcctcgcaccagcccttgttttac |
| Melanoma | SEQ ID | MGGAAARLGAVILFVVIVGLHGVRGKYALADASLKMADPN |
| antigens | NO: 122 | RFRGKDLPVLDQLTDPPGVRRVYHIQAGLPDPFQPPSLPITVY |
| with | | YAVLERACRSVLLNAPSEAPQIVRGASEDVRKQPYNLTIAWF |
| universal | | RMGGNCAIPITVMEYTECSYNKSLGACPIRTQPRWNYYDSFS |
| helper | | AVSEDNLGFLMHAPAFETAGTYLRLVKINDWTEITQFILEHR |
| epitope and | | AKGSCKYALPLRIPPSACLSPQAYQQGVTVDSIGMLPRFIPEN |
| C-terminus | | QRTVAVYSLKIAGWHGPNRLAKFVAAWTLKAAAAAARKFF |
| E2 fused | | HRTCKCTGNFAAAANESFALPYWNFATGRNECDVAAADQL |
| into gD | | GYSYAIDLPVSVAAAAAASVYDFFVWLAAAAAASTFSFRNA |
| Protein | | LAASQVMNLHNLAATAPDNLGYMAAAIAVVNALLLYAYD |
| | | YEELYAKVPRNQDWLAAAAFGLANEKSIAAAAPRSNTTPIV |
| | | HLKGDANTLKCLRYRFKKHCKLYTAVSSTWHWTGHNVKH |
| | | KSAIVTLTYDSEWPRIRYHAAAVYMTSTLLPPELSETPNATQ |
| | | PELAPEDPEDSALLEDPVGTVAPQIPPNWHIPSIQDAATPYHP |
| | | PATPNNMGLIAGAVGGSLLAALVICGIVYWMHRRTRKAPKR |
| | | IRLPHIREDDQPSSHQPLFY |
| Melanoma | SEQ ID | atggggggggctgccgccaggttgggggccgtgattttgtttgtcgtcatagtgggcctccatgg |
| antigens | NO: 123 | ggtccgcggcaaatatgccttggcggatgcctctctcaagatggccgaccccaatcgctttcgcg |
| with | | gcaaagaccttccggtcctggaccagctgaccgaccctccgggggtccggcgcgtgtaccaca |
| universal | | tccaggcgggcctaccggacccgttccagccccccagcctcccgatcacggtttactacgccgt |
| helper | | gttggagcgcgcctgccgcagcgtgctcctaaacgcaccgtcggaggccccccagattgtccg |
| epitope and | | cggggcctccgaagacgtccggaaacaaccctacaacctgaccatcgcttggtttcggatggga |
| C-terminus | | ggcaactgtgctatccccatcacggtcatggagtacaccgaatgctcctacaacaagtctctggg |
| E2 fused | | ggcctgtcccatccgaacgcagccccgctggaactactatgacagcttcagcgccgtcagcga |
| into gD | | ggataacctggggttcctgatgcacgcccccgcgtttgagaccgccggcacgtacctgcggctc |
| Nucleotide | | gtgaagataaacgactggacggagattacacagtttatcctggagcaccgagccaagggctcct |
| | | gtaagtacgccctcccgctgcgcatccccccgtcagcctgcctgtccccccaggcctaccagca |
| | | gggggtgacggtggacagcatcgggatgctgccccgcttcatccccgagaaccagcgcaccg |
| | | tcgccgtatacagcttgaagatcgccgggtggcacgggcccaaccggttagctaagtttgtggc |
| | | cgcttggacactgaaggccgctgcagccgctgcaaggaagttctttcacagaacctgcaaatgt |
| | | acaggaaacttcgccgctgcagccaatgagagcttcgccctgccctattggaactttgctaccgg |
| | | ccggaatgaatgcgacgtggctgcagccgatcagctcggatactcttatgccatcgacctgcctg |
| | | tgagtgtcgctgcagccgctgcagcctcagtgtacgatttctttgtctggctggctgcagccgctg |
| | | cagccagcactttctcctttcgcaacgccctggctgcatctcaggtcatgaacctccataatctggc |
| | | agctaccgcaccagacaacctoggatatatggcagccgctattgccgtggtcaatgctctgctcc |
| | | tgtacgcctatgactacgaggaactgtacgctaaagtccccaggaatcaggattggctcgcagc |
| | | cgctgcatttggtctggccaacgaaaagagcattgcagcagcagcacctaggagtaacactaca |
| | | cccatagtacatttaaaaggtgatgctaatactttaaaatgtttaagatatagatttaaaaagcattgt |
| | | aaattgtatactgcagtgtcgtctacatggcattggacaggacataatgtaaaacataaaagtgca |
| | | attgttacacttacatatgatagtgaatggcctaggatccgatatcacgcggccgcagtgtacatg |
| | | acgagcaccctgctgcccccggagctgtccgagacccccaacgccacgcagccagaactcgc |
| | | cccggaagaccccgaggattcggccctcttggaggaccccgtggggacggtggcgccgcaa |
| | | atcccaccaaactggcacatcccgtcgatccaggacgccgcgacgccttaccatcccccggcc |
| | | accccgaacaacatgggcctgatcgccggcgcggtgggcggcagtctcctggcagccctggt |
| | | catttgcggaattgtgtactggatgcaccgccgcactcggaaagccccaaagcgcatacgcctc |
| | | ccccacatccgggaagacgaccagccgtcctcgcaccagcccttgttttac |
| Melanoma | SEQ ID | MGGAAARLGAVILFVVIVGLHGVRGKYALADASLKMADPN |
| antigens | NO: 124 | RFRGKDLPVLDQLTDPPGVRRVYHIQAGLPDPFQPPSLPITVY |
| with | | YAVLERACRSVLLNAPSEAPQIVRGASEDVRKQPYNLTIAWF |
| universal | | RMGGNCAIPITVMEYTECSYNKSLGACPIRTQPRWNYYDSFS |
| helper | | AVSEDNLGFLMHAPAFETAGTYLRLVKINDWTEITQFILEHR |
| epitope and | | AKGSCKYALPLRIPPSACLSPQAYQQGVTVDSIGMLPRFIPEN |
| N-terminus | | QRTVAVYSLKIAGWHGPNRLAKFVAAWTLKAAAAAARKFF |
| E2 fused | | HRTCKCTGNFAAAANESFALPYWNFATGRNECDVAAADQL |
| into gD | | GYSYAIDLPVSVAAAAAASVYDFFVWLAAAAAASTFSFRNA |
| Protein | | LAASQVMNLHNLAATAPDNLGYMAAAIAVVNALLLYAYD |
| | | YEELYAKVPRNQDWLAAAAFGLANEKSIAAAAPRETLCQRL |
| | | NVCQDKILTHYENDSTDLRDHIDYWKHMRLECAIYYKARE |
| | | MGFKHINHQVVPTLAVSKNKALQAIELQLTLETIYNSQYSNE |
| | | KWPRIRYHAAAVYMTSTLLPPELSETPNATQPELAPEDPEDS |
| | | ALLEDPVGTVAPQIPPNWHIPSIQDAATPYHPPATPNNMGLIA |
| | | GAVGGSLLAALVICGIVYWMHRRTRKAPKRIRLPHIREDDQP |
| | | SSHQPLFY |
| Melanoma | SEQ ID | atggggggggctgccgccaggttgggggccgtgattttgtttgtcgtcatagtgggcctccatgg |
| antigens | NO: 125 | ggtccgcggcaaatatgccttggcggatgcctctctcaagatggccgaccccaatcgctttcgcg |
| with | | gcaaagaccttccggtcctggaccagctgaccgaccctccgggggtccggcgcgtgtaccaca |
| universal | | tccaggcgggcctaccggacccgttccagccccccagcctcccgatcacggtttactacgccgt |
| helper | | gttggagcgcgcctgccgcagcgtgctcctaaacgcaccgtcggaggccccccagattgtccg |
| epitope and | | cggggcctccgaagacgtccggaaacaaccctacaacctgaccatcgcttggtttcggatggga |
| N-terminus | | ggcaactgtgctatccccatcacggtcatggagtacaccgaatgctcctacaacaagtctctggg |
| E2 fused | | ggcctgtcccatccgaacgcagccccgctggaactactatgacagcttcagcgccgtcagcga |
| into gD | | ggataacctggggttcctgatgcacgcccccgcgtttgagaccgccggcacgtacctgcggctc |
| Nucleotide | | gtgaagataaacgactggacggagattacacagtttatcctggagcaccgagccaagggctcct |
| | | gtaagtacgccctcccgctgcgcatccccccgtcagcctgcctgtccccccaggcctaccagca |
| | | gggggtgacggtggacagcatcgggatgctgccccgcttcatccccgagaaccagcgcaccg |
| | | tcgccgtatacagcttgaagatcgccgggtggcacgggcccaaccggttagctaagtttgtggc |
| | | cgcttggacactgaaggccgctgcagccgctgcaaggaagttctttcacagaacctgcaaatgt |
| | | acaggaaacttcgccgctgcagccaatgagagcttcgccctgccctattggaactttgctaccgg |
| | | ccggaatgaatgcgacgtggctgcagccgatcagctcggatactcttatgccatcgacctgcctg |
| | | tgagtgtcgctgcagccgctgcagcctcagtgtacgatttctttgtctggctggctgcagccgctg |
| | | cagccagcactttctcctttcgcaacgccctggctgcatctcaggtcatgaacctccataatctggc |
| | | agctaccgcaccagacaacctoggatatatggcagccgctattgccgtggtcaatgctctgctcc |
| | | tgtacgcctatgactacgaggaactgtacgctaaagtccccaggaatcaggattggctcgcagc |
| | | cgctgcatttggtctggccaacgaaaagagcattgcagcagcagcacctagggagactctttgc |
| | | caacgtttaaatgtgtgtcaggacaaaatactaacacattatgaaaatgatagtacagacctacgtg |
| | | accatatagactattggaaacacatgcgcctagaatgtgctatttattacaaggccagagaaatgg |
| | | gatttaaacatattaaccaccaggtggtgccaacactggctgtatcaaagaataaagcattacaag |
| | | caattgaactgcaactaacgttagaaacaatatataactcacaatatagtaatgaaaagtggccta |
| | | ggatccgatatcacgcggccgcagtgtacatgacgagcaccctgctgcccccggagctgtccg |
| | | agacccccaacgccacgcagccagaactcgccccggaagaccccgaggattcggccctcttg |
| | | gaggaccccgtggggacggtggcgccgcaaatcccaccaaactggcacatcccgtcgatcca |
| | | ggacgccgcgacgccttaccatcccccggccaccccgaacaacatgggcctgatcgccggcg |
| | | cggtgggcggcagtctcctggcagccctggtcatttgcggaattgtgtactggatgcaccgccg |
| | | cactcggaaagccccaaagcgcatacgcctcccccacatccgggaagacgaccagccgtcct |
| | | cgcaccagcccttgttttac |
| Melanoma | SEQ ID | MGGAAARLGAVILFVVIVGLHGVRGKYALADASLKMADPN |
| antigens | NO: 126 | RFRGKDLPVLDQLTDPPGVRRVYHIQAGLPDPFQPPSLPITVY |
| with | | YAVLERACRSVLLNAPSEAPQIVRGASEDVRKQPYNLTIAWF |
| universal | | RMGGNCAIPITVMEYTECSYNKSLGACPIRTQPRWNYYDSFS |
| helper | | AVSEDNLGFLMHAPAFETAGTYLRLVKINDWTEITQFILEHR |
| epitope and | | AKGSCKYALPLRIPPSACLSPQAYQQGVTVDSIGMLPRFIPEN |
| E2 fused | | QRTVAVYSLKIAGWHGPNRLAKFVAAWTLKAAAAAARKFF |
| into gD | | HRTCKCTGNFAAAANESFALPYWNFATGRNECDVAAADQL |
| with C- | | GYSYAIDLPVSVAAAAAASVYDFFVWLAAAAAASTFSFRNA |
| terminal | | LAASQVMNLHNLAATAPDNLGYMAAAIAVVNALLLYAYD |
| Flag | | YEELYAKVPRNQDWLAAAAFGLANEKSIAAAAPRETLCQRL |
| Protein | | NVCQDKILTHYENDSTDLRDHIDYWKHMRLECAIYYKARE |
| | | MGFKHINHQVVPTLAVSKNKALQAIELQLTLETIYNSQYSNE |
| | | KWSNTTPIVHLKGDANTLKCLRYRFKKHCKLYTAVSSTWH |
| | | WTGHNVKHKSAIVTLTYDSEWPRIRYHAAAVYMTSTLLPPE |
| | | LSETPNATQPELAPEDPEDSALLEDPVGTVAPQIPPNWHIPSIQ |
| | | DAATPYHPPATPNNMGLIAGAVGGSLLAALVICGIVYWMHR |
| | | RTRKAPKRIRLPHIREDDQPSSHQPLFYDYKDDDDKT |
| Melanoma | SEQ ID | atggggggggctgccgccaggttgggggccgtgattttgtttgtcgtcatagtgggcctccatgg |
| antigens | NO: 127 | ggtccgcggcaaatatgccttggcggatgcctctctcaagatggccgaccccaatcgctttcgcg |
| with | | gcaaagaccttccggtcctggaccagctgaccgaccctccgggggtccggcgcgtgtaccaca |
| universal | | tccaggcgggcctaccggacccgttccagccccccagcctcccgatcacggtttactacgccgt |
| helper | | gttggagcgcgcctgccgcagcgtgctcctaaacgcaccgtcggaggccccccagattgtccg |
| epitope and | | cggggcctccgaagacgtccggaaacaaccctacaacctgaccatcgcttggtttcggatggga |
| E2 fused | | ggcaactgtgctatccccatcacggtcatggagtacaccgaatgctcctacaacaagtctctggg |
| into gD | | ggcctgtcccatccgaacgcagccccgctggaactactatgacagcttcagcgccgtcagcga |
| with C- | | ggataacctggggttcctgatgcacgcccccgcgtttgagaccgccggcacgtacctgcggctc |
| terminal | | gtgaagataaacgactggacggagattacacagtttatcctggagcaccgagccaagggctcct |
| Flag | | gtaagtacgccctcccgctgcgcatccccccgtcagcctgcctgtccccccaggcctaccagca |
| Nucleotide | | gggggtgacggtggacagcatcgggatgctgccccgcttcatccccgagaaccagcgcaccg |
| | | tcgccgtatacagcttgaagatcgccgggtggcacgggcccaaccggttagctaagtttgtggc |
| | | cgcttggacactgaaggccgctgcagccgctgcaaggaagttctttcacagaacctgcaaatgt |
| | | acaggaaacttcgccgctgcagccaatgagagcttcgccctgccctattggaactttgctaccgg |
| | | ccggaatgaatgcgacgtggctgcagccgatcagctcggatactcttatgccatcgacctgcctg |
| | | tgagtgtcgctgcagccgctgcagcctcagtgtacgatttctttgtctggctggctgcagccgctg |
| | | cagccagcactttctcctttcgcaacgccctggctgcatctcaggtcatgaacctccataatctggc |
| | | agctaccgcaccagacaacctoggatatatggcagccgctattgccgtggtcaatgctctgctcc |
| | | tgtacgcctatgactacgaggaactgtacgctaaagtccccaggaatcaggattggctcgcagc |
| | | cgctgcatttggtctggccaacgaaaagagcattgcagcagcagcacctagggagactctttgc |
| | | caacgtttaaatgtgtgtcaggacaaaatactaacacattatgaaaatgatagtacagacctacgtg |
| | | accatatagactattggaaacacatgcgcctagaatgtgctatttattacaaggccagagaaatgg |
| | | gatttaaacatattaaccaccaggtggtgccaacactggctgtatcaaagaataaagcattacaag |
| | | caattgaactgcaactaacgttagaaacaatatataactcacaatatagtaatgaaaagtggagta |
| | | acactacacccatagtacatttaaaaggtgatgctaatactttaaaatgtttaagatatagatttaaaa |
| | | agcattgtaaattgtatactgcagtgtcgtctacatggcattggacaggacataatgtaaaacataa |
| | | aagtgcaattgttacacttacatatgatagtgaatggcctaggatccgatatcacgcggccgcagt |
| | | gtacatgacgagcaccctgctgcccccggagctgtccgagacccccaacgccacgcagccag |
| | | aactcgccccggaagaccccgaggattcggccctcttggaggaccccgtggggacggtggcg |
| | | ccgcaaatcccaccaaactggcacatcccgtcgatccaggacgccgcgacgccttaccatccc |
| | | ccggccaccccgaacaacatgggcctgatcgccggcgcggtgggcggcagtctcctggcagc |
| | | cctggtcatttgcggaattgtgtactggatgcaccgccgcactcggaaagccccaaagcgcata |
| | | cgcctcccccacatccgggaagacgaccagccgtcctcgcaccagcccttgttttacgactaca |
| | | aagacgatgacgacaagact |
| Melanoma | SEQ ID | MGGAAARLGAVILFVVIVGLHGVRGKYALADASLKMADPN |
| antigens | NO: 128 | RFRGKDLPVLDQLTDPPGVRRVYHIQAGLPDPFQPPSLPITVY |
| with | | YAVLERACRSVLLNAPSEAPQIVRGASEDVRKQPYNLTIAWF |
| universal | | RMGGNCAIPITVMEYTECSYNKSLGACPIRTQPRWNYYDSFS |
| helper | | AVSEDNLGFLMHAPAFETAGTYLRLVKINDWTEITQFILEHR |
| epitope and | | AKGSCKYALPLRIPPSACLSPQAYQQGVTVDSIGMLPRFIPEN |
| the C- | | QRTVAVYSLKIAGWHGPNRLAKFVAAWTLKAAAAAARKFF |
| terminus of | | HRTCKCTGNFAAAANESFALPYWNFATGRNECDVAAADQL |
| E2 fused | | GYSYAIDLPVSVAAAAAASVYDFFVWLAAAAAASTFSFRNA |
| into gD | | LAASQVMNLHNLAATAPDNLGYMAAAIAVVNALLLYAYD |
| with C- | | YEELYAKVPRNQDWLAAAAFGLANEKSIAAAAPRSNTTPIV |
| terminal | | HLKGDANTLKCLRYRFKKHCKLYTAVSSTWHWTGHNVKH |
| Flag | | KSAIVTLTYDSEWPRIRYHAAAVYMTSTLLPPELSETPNATQ |
| Protein | | PELAPEDPEDSALLEDPVGTVAPQIPPNWHIPSIQDAATPYHP |
| | | PATPNNMGLIAGAVGGSLLAALVICGIVYWMHRRTRKAPKR |
| | | IRLPHIREDDQPSSHQPLFYDYKDDDDKT |
| Melanoma | SEQ ID | atggggggggctgccgccaggttgggggccgtgattttgtttgtcgtcatagtgggcctccatgg |
| antigens | NO: 129 | ggtccgcggcaaatatgccttggcggatgcctctctcaagatggccgaccccaatcgctttcgcg |
| with | | gcaaagaccttccggtcctggaccagctgaccgaccctccgggggtccggcgcgtgtaccaca |
| universal | | tccaggcgggcctaccggacccgttccagccccccagcctcccgatcacggtttactacgccgt |
| helper | | gttggagcgcgcctgccgcagcgtgctcctaaacgcaccgtcggaggccccccagattgtccg |
| epitope and | | cggggcctccgaagacgtccggaaacaaccctacaacctgaccatcgcttggtttcggatggga |
| the C- | | ggcaactgtgctatccccatcacggtcatggagtacaccgaatgctcctacaacaagtctctggg |
| terminus of | | ggcctgtcccatccgaacgcagccccgctggaactactatgacagcttcagcgccgtcagcga |
| E2 fused | | ggataacctggggttcctgatgcacgcccccgcgtttgagaccgccggcacgtacctgcggctc |
| into gD | | gtgaagataaacgactggacggagattacacagtttatcctggagcaccgagccaagggctcct |
| with C- | | gtaagtacgccctcccgctgcgcatccccccgtcagcctgcctgtccccccaggcctaccagca |
| terminal | | gggggtgacggtggacagcatcgggatgctgccccgcttcatccccgagaaccagcgcaccg |
| Flag | | tcgccgtatacagcttgaagatcgccgggtggcacgggcccaaccggttagctaagtttgtggc |
| Nucleotide | | cgcttggacactgaaggccgctgcagccgctgcaaggaagttctttcacagaacctgcaaatgt |
| | | acaggaaacttcgccgctgcagccaatgagagcttcgccctgccctattggaactttgctaccgg |
| | | ccggaatgaatgcgacgtggctgcagccgatcagctcggatactcttatgccatcgacctgcctg |
| | | tgagtgtcgctgcagccgctgcagcctcagtgtacgatttctttgtctggctggctgcagccgctg |
| | | cagccagcactttctcctttcgcaacgccctggctgcatctcaggtcatgaacctccataatctggc |
| | | agctaccgcaccagacaacctcggatatatggcagccgctattgccgtggtcaatgctctgctcc |
| | | tgtacgcctatgactacgaggaactgtacgctaaagtccccaggaatcaggattggctcgcagc |
| | | cgctgcatttggtctggccaacgaaaagagcattgcagcagcagcacctaggagtaacactaca |
| | | cccatagtacatttaaaaggtgatgctaatactttaaaatgtttaagatatagatttaaaaagcattgt |
| | | aaattgtatactgcagtgtcgtctacatggcattggacaggacataatgtaaaacataaaagtgca |
| | | attgttacacttacatatgatagtgaatggcctaggatccgatatcacgcggccgcagtgtacatg |
| | | acgagcaccctgctgcccccggagctgtccgagacccccaacgccacgcagccagaactcgc |
| | | cccggaagaccccgaggattcggccctcttggaggaccccgtggggacggtggcgccgcaa |
| | | atcccaccaaactggcacatcccgtcgatccaggacgccgcgacgccttaccatcccccggcc |
| | | accccgaacaacatgggcctgatcgccggcgcggtgggcggcagtctcctggcagccctggt |
| | | catttgcggaattgtgtactggatgcaccgccgcactcggaaagccccaaagcgcatacgcctc |
| | | ccccacatccgggaagacgaccagccgtcctcgcaccagcccttgttttacgactacaaagacg |
| | | atgacgacaagact |
| Melanoma | SEQ ID | MGGAAARLGAVILFVVIVGLHGVRGKYALADASLKMADPN |
| antigens | NO: 130 | RFRGKDLPVLDQLTDPPGVRRVYHIQAGLPDPFQPPSLPITVY |
| with | | YAVLERACRSVLLNAPSEAPQIVRGASEDVRKQPYNLTIAWF |
| universal | | RMGGNCAIPITVMEYTECSYNKSLGACPIRTQPRWNYYDSFS |
| helper | | AVSEDNLGFLMHAPAFETAGTYLRLVKINDWTEITQFILEHR |
| epitope and | | AKGSCKYALPLRIPPSACLSPQAYQQGVTVDSIGMLPRFIPEN |
| the N- | | QRTVAVYSLKIAGWHGPNRLAKFVAAWTLKAAAAAARKFF |
| terminus of | | HRTCKCTGNFAAAANESFALPYWNFATGRNECDVAAADQL |
| E2 fused | | GYSYAIDLPVSVAAAAAASVYDFFVWLAAAAAASTFSFRNA |
| into gD | | LAASQVMNLHNLAATAPDNLGYMAAAIAVVNALLLYAYD |
| with C- | | YEELYAKVPRNQDWLAAAAFGLANEKSIAAAAPRETLCQRL |
| terminal | | NVCQDKILTHYENDSTDLRDHIDYWKHMRLECAIYYKARE |
| Flag | | MGFKHINHQVVPTLAVSKNKALQAIELQLTLETIYNSQYSNE |
| Protein | | KWPRIRYHAAAVYMTSTLLPPELSETPNATQPELAPEDPEDS |
| | | ALLEDPVGTVAPQIPPNWHIPSIQDAATPYHPPATPNNMGLIA |
| | | GAVGGSLLAALVICGIVYWMHRRTRKAPKRIRLPHIREDDQP |
| | | SSHQPLFYDYKDDDDKT |
| Melanoma | SEQ ID | atggggggggctgccgccaggttgggggccgtgattttgtttgtcgtcatagtgggcctccatgg |
| antigens | NO: 131 | ggtccgcggcaaatatgccttggcggatgcctctctcaagatggccgaccccaatcgctttcgcg |
| with | | gcaaagaccttccggtcctggaccagctgaccgaccctccgggggtccggcgcgtgtaccaca |
| universal | | tccaggcgggcctaccggacccgttccagccccccagcctcccgatcacggtttactacgccgt |
| helper | | gttggagcgcgcctgccgcagcgtgctcctaaacgcaccgtcggaggccccccagattgtccg |
| epitope and | | cggggcctccgaagacgtccggaaacaaccctacaacctgaccatcgcttggtttcggatggga |
| the N- | | ggcaactgtgctatccccatcacggtcatggagtacaccgaatgctcctacaacaagtctctggg |
| terminus of | | ggcctgtcccatccgaacgcagccccgctggaactactatgacagcttcagcgccgtcagcga |
| E2 fused | | ggataacctggggttcctgatgcacgcccccgcgtttgagaccgccggcacgtacctgcggctc |
| into gD | | gtgaagataaacgactggacggagattacacagtttatcctggagcaccgagccaagggctcct |
| with C- | | gtaagtacgccctcccgctgcgcatccccccgtcagcctgcctgtccccccaggcctaccagca |
| terminal | | gggggtgacggtggacagcatcgggatgctgccccgcttcatccccgagaaccagcgcaccg |
| Flag | | tcgccgtatacagcttgaagatcgccgggtggcacgggcccaaccggttagctaagtttgtggc |
| Nucleotide | | cgcttggacactgaaggccgctgcagccgctgcaaggaagttctttcacagaacctgcaaatgt |
| | | acaggaaacttcgccgctgcagccaatgagagcttcgccctgccctattggaactttgctaccgg |
| | | ccggaatgaatgcgacgtggctgcagccgatcagctcggatactcttatgccatcgacctgcctg |
| | | tgagtgtcgctgcagccgctgcagcctcagtgtacgatttctttgtctggctggctgcagccgctg |
| | | cagccagcactttctcctttcgcaacgccctggctgcatctcaggtcatgaacctccataatctggc |
| | | agctaccgcaccagacaacctcggatatatggcagccgctattgccgtggtcaatgctctgctcc |
| | | tgtacgcctatgactacgaggaactgtacgctaaagtccccaggaatcaggattggctcgcagc |
| | | cgctgcatttggtctggccaacgaaaagagcattgcagcagcagcacctagggagactctttgc |
| | | caacgtttaaatgtgtgtcaggacaaaatactaacacattatgaaaatgatagtacagacctacgtg |
| | | accatatagactattggaaacacatgcgcctagaatgtgctatttattacaaggccagagaaatgg |
| | | gatttaaacatattaaccaccaggtggtgccaacactggctgtatcaaagaataaagcattacaag |
| | | caattgaactgcaactaacgttagaaacaatatataactcacaatatagtaatgaaaagtggccta |
| | | ggatccgatatcacgcggccgcagtgtacatgacgagcaccctgctgcccccggagctgtccg |
| | | agacccccaacgccacgcagccagaactcgccccggaagaccccgaggattcggccctcttg |
| | | gaggaccccgtggggacggtggcgccgcaaatcccaccaaactggcacatcccgtcgatcca |
| | | ggacgccgcgacgccttaccatcccccggccaccccgaacaacatgggcctgatcgccggcg |
| | | cggtgggcggcagtctcctggcagccctggtcatttgcggaattgtgtactggatgcaccgccg |
| | | cactcggaaagccccaaagcgcatacgcctcccccacatccgggaagacgaccagccgtcct |
| | | cgcaccagcccttgttttacgactacaaagacgatgacgacaagact |
| Melanoma | SEQ ID | MGGAAARLGAVILFVVIVGLHGVRGKYCLADASLKMADPN |
| antigens | NO: 132 | RFRGKDLPVLDQLTDPPGVRRVYHIQAGLPDPFQPPSLPITVC |
| with | | YAVLERACRSVLLNAPSEAPQIVRGASEDVRKQPYNLTIAWF |
| universal | | RMGGNCAIPITVMEYTECSYNKSLGACPIRTQPRWNYYDSFS |
| helper | | AVSEDNLGFLMHAPAFETAGTYLRLVKINDWTEITQFILEHR |
| epitope and | | AKGSCKYALPLRIPPSACLSPQAYQQGVTVDSIGPAKFVAAW |
| E2 fused | | TLKAAAAAARKFFHRTCKCTGNFAAAANESFALPYWNFAT |
| into gDM5 | | GRNECDVAAADQLGYSYAIDLPVSVAAAAAASVYDFFVWL |
| Protein | | AAAAAASTFSFRNALAASQVMNLHNLAATAPDNLGYMAAA |
| | | IAVVNALLLYAYDYEELYAKVPRNQDWLAAAAFGLANEKSI |
| | | AAAAPRETLCQRLNVCQDKILTHYENDSTDLRDHIDYWKH |
| | | MRLECAIYYKAREMGFKHINHQVVPTLAVSKNKALQAIELQ |
| | | LTLETIYNSQYSNEKWSNTTPIVHLKGDANTLKCLRYRFKKH |
| | | CKLYTAVSSTWHWTGHNVKHKSAIVTLTYDSEWGPATQPE |
| | | LAPEDPEDSALLEDPVGTVAPQIPPNWHIPSIQDAATPYHPPA |
| | | TPNNMGLIAGAVGGSLLAALVICGIVYWMHRRTRKAPKRIR |
| | | LPHIREDDQPSSHQPLFY |
| Melanoma | SEQ ID | atggggggggctgccgccaggttgggggccgtgattttgtttgtcgtcatagtgggcctccatgg |
| antigens | NO: 133 | ggtccgcggcaaatattgtttggcggatgcctctctcaagatggccgaccccaatcgctttcgcg |
| with | | gcaaagaccttccggtcctggaccagctgaccgaccctccgggggtccggcgcgtgtaccaca |
| universal | | tccaggcgggcctaccggacccgttccagccccccagcctcccgatcacggtttgctacgccgt |
| helper | | gttggagcgcgcctgccgcagcgtgctcctaaacgcaccgtcggaggccccccagattgtccg |
| epitope and | | cggggcctccgaagacgtccggaaacaaccctacaacctgaccatcgcttggtttcggatggga |
| E2 fused | | ggcaactgtgctatccccatcacggtcatggagtacaccgaatgctcctacaacaagtctctggg |
| into gDM5 | | ggcctgtcccatccgaacgcagccccgctggaactactatgacagcttcagcgccgtcagcga |
| Nucleotide | | ggataacctggggttcctgatgcacgcccccgcgtttgagaccgccggcacgtacctgcggctc |
| | | gtgaagataaacgactggacggagattacacagtttatcctggagcaccgagccaagggctcct |
| | | gtaagtacgccctcccgctgcgcatccccccgtcagcctgcctctccccccaggcctaccagca |
| | | gggggtgacggtggacagcatcgggcccgctaagtttgtggccgcttggacactgaaggccgc |
| | | tgcagccgctgcaaggaagttctttcacagaacctgcaaatgtacaggaaacttcgccgctgcag |
| | | ccaatgagagcttcgccctgccctattggaactttgctaccggccggaatgaatgcgacgtggct |
| | | gcagccgatcagctcggatactcttatgccatcgacctgcctgtgagtgtcgctgcagccgctgc |
| | | agcctcagtgtacgatttctttgtctggctggctgcagccgctgcagccagcactttctcctttcgca |
| | | acgccctggctgcatctcaggtcatgaacctccataatctggcagctaccgcaccagacaacctc |
| | | ggatatatggcagccgctattgccgtggtcaatgctctgctcctgtacgcctatgactacgaggaa |
| | | ctgtacgctaaagtccccaggaatcaggattggctcgcagccgctgcatttggtctggccaacga |
| | | aaagagcattgcagcagcagcacctagggagactctttgccaacgtttaaatgtgtgtcaggaca |
| | | aaatactaacacattatgaaaatgatagtacagacctacgtgaccatatagactattggaaacacat |
| | | gcgcctagaatgtgctatttattacaaggccagagaaatgggatttaaacatattaaccaccaggt |
| | | ggtgccaacactggctgtatcaaagaataaagcattacaagcaattgaactgcaactaacgttag |
| | | aaacaatatataactcacaatatagtaatgaaaagtggagtaacactacacccatagtacatttaaa |
| | | aggtgatgctaatactttaaaatgtttaagatatagatttaaaaagcattgtaaattgtatactgcagt |
| | | gtcgtctacatggcattggacaggacataatgtaaaacataaaagtgcaattgttacacttacatat |
| | | gatagtgaatgggggcccgccacgcagccagaactcgccccggaagaccccgaggattcgg |
| | | ccctcttggaggaccccgtggggacggtggcgccgcaaatcccaccaaactggcacatcccgt |
| | | cgatccaggacgccgcgacgccttaccatcccccggccaccccgaacaacatgggcctgatc |
| | | gccggcgcggtgggcggcagtctcctggcagccctggtcatttgcggaattgtgtactggatgc |
| | | accgccgcactcggaaagccccaaagcgcatacgcctcccccacatccgggaagacgacca |
| | | gccgtcctcgcaccagcccttgttttac |
| Melanoma | SEQ ID | MGGAAARLGAVILFVVIVGLHGVRGKYCLADASLKMADPN |
| antigens | NO: 134 | RFRGKDLPVLDQLTDPPGVRRVYHIQAGLPDPFQPPSLPITVC |
| with | | YAVLERACRSVLLNAPSEAPQIVRGASEDVRKQPYNLTIAWF |
| universal | | RMGGNCAIPITVMEYTECSYNKSLGACPIRTQPRWNYYDSFS |
| helper | | AVSEDNLGFLMHAPAFETAGTYLRLVKINDWTEITQFILEHR |
| epitope and | | AKGSCKYALPLRIPPSACLSPQAYQQGVTVDSIGPAKFVAAW |
| C-terminus | | TLKAAAAAARKFFHRTCKCTGNFAAAANESFALPYWNFAT |
| E2 fused | | GRNECDVAAADQLGYSYAIDLPVSVAAAAAASVYDFFVWL |
| into gDM5 | | AAAAAASTFSFRNALAASQVMNLHNLAATAPDNLGYMAAA |
| Protein | | IAVVNALLLYAYDYEELYAKVPRNQDWLAAAAFGLANEKSI |
| | | AAAAPRSNTTPIVHLKGDANTLKCLRYRFKKHCKLYTAVSS |
| | | TWHWTGHNVKHKSAIVTLTYDSEWGPATQPELAPEDPEDSA |
| | | LLEDPVGTVAPQIPPNWHIPSIQDAATPYHPPATPNNMGLIAG |
| | | AVGGSLLAALVICGIVYWMHRRTRKAPKRIRLPHIREDDQPS |
| | | SHQPLFY |
| Melanoma | SEQ ID | atggggggggctgccgccaggttgggggccgtgattttgtttgtcgtcatagtgggcctccatgg |
| antigens | NO: 135 | ggtccgcggcaaatattgtttggcggatgcctctctcaagatggccgaccccaatcgctttcgcg |
| with | | gcaaagaccttccggtcctggaccagctgaccgaccctccgggggtccggcgcgtgtaccaca |
| universal | | tccaggcgggcctaccggacccgttccagccccccagcctcccgatcacggtttgctacgccgt |
| helper | | gttggagcgcgcctgccgcagcgtgctcctaaacgcaccgtcggaggccccccagattgtccg |
| epitope and | | cggggcctccgaagacgtccggaaacaaccctacaacctgaccatcgcttggtttcggatggga |
| C-terminus | | ggcaactgtgctatccccatcacggtcatggagtacaccgaatgctcctacaacaagtctctggg |
| E2 fused | | ggcctgtcccatccgaacgcagccccgctggaactactatgacagcttcagcgccgtcagcga |
| into gDM5 | | ggataacctggggttcctgatgcacgcccccgcgtttgagaccgccggcacgtacctgcggctc |
| Nucleotide | | gtgaagataaacgactggacggagattacacagtttatcctggagcaccgagccaagggctcct |
| | | gtaagtacgccctcccgctgcgcatccccccgtcagcctgcctctccccccaggcctaccagca |
| | | gggggtgacggtggacagcatcgggcccgctaagtttgtggccgcttggacactgaaggccgc |
| | | tgcagccgctgcaaggaagttctttcacagaacctgcaaatgtacaggaaacttcgccgctgcag |
| | | ccaatgagagcttcgccctgccctattggaactttgctaccggccggaatgaatgcgacgtggct |
| | | gcagccgatcagctcggatactcttatgccatcgacctgcctgtgagtgtcgctgcagccgctgc |
| | | agcctcagtgtacgatttctttgtctggctggctgcagccgctgcagccagcactttctcctttcgca |
| | | acgccctggctgcatctcaggtcatgaacctccataatctggcagctaccgcaccagacaacctc |
| | | ggatatatggcagccgctattgccgtggtcaatgctctgctcctgtacgcctatgactacgaggaa |
| | | ctgtacgctaaagtccccaggaatcaggattggctcgcagccgctgcatttggtctggccaacga |
| | | aaagagcattgcagcagcagcacctaggagtaacactacacccatagtacatttaaaaggtgat |
| | | gctaatactttaaaatgtttaagatatagatttaaaaagcattgtaaattgtatactgcagtgtcgtcta |
| | | catggcattggacaggacataatgtaaaacataaaagtgcaattgttacacttacatatgatagtga |
| | | atgggggcccgccacgcagccagaactcgccccggaagaccccgaggattcggccctcttgg |
| | | aggaccccgtggggacggtggcgccgcaaatcccaccaaactggcacatcccgtcgatccag |
| | | gacgccgcgacgccttaccatcccccggccaccccgaacaacatgggcctgatcgccggcgc |
| | | ggtgggcggcagtctcctggcagccctggtcatttgcggaattgtgtactggatgcaccgccgc |
| | | actcggaaagccccaaagcgcatacgcctcccccacatccgggaagacgaccagccgtcctc |
| | | gcaccagcccttgttttac |
| Melanoma | SEQ ID | MGGAAARLGAVILFVVIVGLHGVRGKYCLADASLKMADPN |
| antigens | NO: 136 | RFRGKDLPVLDQLTDPPGVRRVYHIQAGLPDPFQPPSLPITVC |
| with | | YAVLERACRSVLLNAPSEAPQIVRGASEDVRKQPYNLTIAWF |
| universal | | RMGGNCAIPITVMEYTECSYNKSLGACPIRTQPRWNYYDSFS |
| helper | | AVSEDNLGFLMHAPAFETAGTYLRLVKINDWTEITQFILEHR |
| epitope and | | AKGSCKYALPLRIPPSACLSPQAYQQGVTVDSIGPAKFVAAW |
| N-terminus | | TLKAAAAAARKFFHRTCKCTGNFAAAANESFALPYWNFAT |
| E2 fused | | GRNECDVAAADQLGYSYAIDLPVSVAAAAAASVYDFFVWL |
| into gDM5 | | AAAAAASTFSFRNALAASQVMNLHNLAATAPDNLGYMAAA |
| Protein | | IAVVNALLLYAYDYEELYAKVPRNQDWLAAAAFGLANEKSI |
| | | AAAAPRETLCQRLNVCQDKILTHYENDSTDLRDHIDYWKH |
| | | MRLECAIYYKAREMGFKHINHQVVPTLAVSKNKALQAIELQ |
| | | LTLETIYNSQYSNEKWGPATQPELAPEDPEDSALLEDPVGTV |
| | | APQIPPNWHIPSIQDAATPYHPPATPNNMGLIAGAVGGSLLA |
| | | ALVICGIVYWMHRRTRKAPKRIRLPHIREDDQPSSHQPLFY |
| Melanoma | SEQ ID | atggggggggctgccgccaggttgggggccgtgattttgtttgtcgtcatagtgggcctccatgg |
| antigens | NO: 137 | ggtccgcggcaaatattgtttggcggatgcctctctcaagatggccgaccccaatcgctttcgcg |
| with | | gcaaagaccttccggtcctggaccagctgaccgaccctccgggggtccggcgcgtgtaccaca |
| universal | | tccaggcgggcctaccggacccgttccagccccccagcctcccgatcacggtttgctacgccgt |
| helper | | gttggagcgcgcctgccgcagcgtgctcctaaacgcaccgtcggaggccccccagattgtccg |
| epitope and | | cggggcctccgaagacgtccggaaacaaccctacaacctgaccatcgcttggtttcggatggga |
| N-terminus | | ggcaactgtgctatccccatcacggtcatggagtacaccgaatgctcctacaacaagtctctggg |
| E2 fused | | ggcctgtcccatccgaacgcagccccgctggaactactatgacagcttcagcgccgtcagcga |
| into gDM5 | | ggataacctggggttcctgatgcacgcccccgcgtttgagaccgccggcacgtacctgcggctc |
| Nucleotide | | gtgaagataaacgactggacggagattacacagtttatcctggagcaccgagccaagggctcct |
| | | gtaagtacgccctcccgctgcgcatccccccgtcagcctgcctctccccccaggcctaccagca |
| | | gggggtgacggtggacagcatcgggcccgctaagtttgtggccgcttggacactgaaggccgc |
| | | tgcagccgctgcaaggaagttctttcacagaacctgcaaatgtacaggaaacttcgccgctgcag |
| | | ccaatgagagcttcgccctgccctattggaactttgctaccggccggaatgaatgcgacgtggct |
| | | gcagccgatcagctcggatactcttatgccatcgacctgcctgtgagtgtcgctgcagccgctgc |
| | | agcctcagtgtacgatttctttgtctggctggctgcagccgctgcagccagcactttctcctttcgca |
| | | acgccctggctgcatctcaggtcatgaacctccataatctggcagctaccgcaccagacaacctc |
| | | ggatatatggcagccgctattgccgtggtcaatgctctgctcctgtacgcctatgactacgaggaa |
| | | ctgtacgctaaagtccccaggaatcaggattggctcgcagccgctgcatttggtctggccaacga |
| | | aaagagcattgcagcagcagcacctagggagactctttgccaacgtttaaatgtgtgtcaggaca |
| | | aaatactaacacattatgaaaatgatagtacagacctacgtgaccatatagactattggaaacacat |
| | | gcgcctagaatgtgctatttattacaaggccagagaaatgggatttaaacatattaaccaccaggt |
| | | ggtgccaacactggctgtatcaaagaataaagcattacaagcaattgaactgcaactaacgttag |
| | | aaacaatatataactcacaatatagtaatgaaaagtgggggcccgccacgcagccagaactcgc |
| | | cccggaagaccccgaggattcggccctcttggaggaccccgtggggacggtggcgccgcaa |
| | | atcccaccaaactggcacatcccgtcgatccaggacgccgcgacgccttaccatcccccggcc |
| | | accccgaacaacatgggcctgatcgccggcgcggtgggcggcagtctcctggcagccctggt |
| | | catttgcggaattgtgtactggatgcaccgccgcactcggaaagccccaaagcgcatacgcctc |
| | | ccccacatccgggaagacgaccagccgtcctcgcaccagcccttgttttac |
| Melanoma | SEQ ID | MGGAAARLGAVILFVVIVGLHGVRGKYCLADASLKMADPN |
| antigens | NO: 138 | RFRGKDLPVLDQLTDPPGVRRVYHIQAGLPDPFQPPSLPITVC |
| with | | YAVLERACRSVLLNAPSEAPQIVRGASEDVRKQPYNLTIAWF |
| universal | | RMGGNCAIPITVMEYTECSYNKSLGACPIRTQPRWNYYDSFS |
| helper | | AVSEDNLGFLMHAPAFETAGTYLRLVKINDWTEITQFILEHR |
| epitope and | | AKGSCKYALPLRIPPSACLSPQAYQQGVTVDSIGPAKFVAAW |
| E2 fused | | TLKAAAAAARKFFHRTCKCTGNFAAAANESFALPYWNFAT |
| into gDM5 | | GRNECDVAAADQLGYSYAIDLPVSVAAAAAASVYDFFVWL |
| with C- | | AAAAAASTFSFRNALAASQVMNLHNLAATAPDNLGYMAAA |
| terminal | | IAVVNALLLYAYDYEELYAKVPRNQDWLAAAAFGLANEKSI |
| Flag | | AAAAPRETLCQRLNVCQDKILTHYENDSTDLRDHIDYWKH |
| Protein | | MRLECAIYYKAREMGFKHINHQVVPTLAVSKNKALQAIELQ |
| | | LTLETIYNSQYSNEKWSNTTPIVHLKGDANTLKCLRYRFKKH |
| | | CKLYTAVSSTWHWTGHNVKHKSAIVTLTYDSEWGPATQPE |
| | | LAPEDPEDSALLEDPVGTVAPQIPPNWHIPSIQDAATPYHPPA |
| | | TPNNMGLIAGAVGGSLLAALVICGIVYWMHRRTRKAPKRIR |
| | | LPHIREDDQPSSHQPLFYDYKDDDDKT |
| Melanoma | SEQ ID | atggggggggctgccgccaggttgggggccgtgattttgtttgtcgtcatagtgggcctccatgg |
| antigens | NO: 139 | ggtccgcggcaaatattgtttggcggatgcctctctcaagatggccgaccccaatcgctttcgcg |
| with | | gcaaagaccttccggtcctggaccagctgaccgaccctccgggggtccggcgcgtgtaccaca |
| universal | | tccaggcgggcctaccggacccgttccagccccccagcctcccgatcacggtttgctacgccgt |
| helper | | gttggagcgcgcctgccgcagcgtgctcctaaacgcaccgtcggaggccccccagattgtccg |
| epitope and | | cggggcctccgaagacgtccggaaacaaccctacaacctgaccatcgcttggtttcggatggga |
| E2 fused | | ggcaactgtgctatccccatcacggtcatggagtacaccgaatgctcctacaacaagtctctggg |
| into gDM5 | | ggcctgtcccatccgaacgcagccccgctggaactactatgacagcttcagcgccgtcagcga |
| with C- | | ggataacctggggttcctgatgcacgcccccgcgtttgagaccgccggcacgtacctgcggctc |
| terminal | | gtgaagataaacgactggacggagattacacagtttatcctggagcaccgagccaagggctcct |
| Flag | | gtaagtacgccctcccgctgcgcatccccccgtcagcctgcctctccccccaggcctaccagca |
| Nucleotide | | gggggtgacggtggacagcatcgggcccgctaagtttgtggccgcttggacactgaaggccgc |
| | | tgcagccgctgcaaggaagttctttcacagaacctgcaaatgtacaggaaacttcgccgctgcag |
| | | ccaatgagagcttcgccctgccctattggaactttgctaccggccggaatgaatgcgacgtggct |
| | | gcagccgatcagctcggatactcttatgccatcgacctgcctgtgagtgtcgctgcagccgctgc |
| | | agcctcagtgtacgattttttgtctggctggctgcagccgctgcagccagcactttctcctttcgca |
| | | acgccctggctgcatctcaggtcatgaacctccataatctggcagctaccgcaccagacaacctc |
| | | ggatatatggcagccgctattgccgtggtcaatgctctgctcctgtacgcctatgactacgaggaa |
| | | ctgtacgctaaagtccccaggaatcaggattggctcgcagccgctgcatttggtctggccaacga |
| | | aaagagcattgcagcagcagcacctagggagactctttgccaacgtttaaatgtgtgtcaggaca |
| | | aaatactaacacattatgaaaatgatagtacagacctacgtgaccatatagactattggaaacacat |
| | | gcgcctagaatgtgctatttattacaaggccagagaaatgggatttaaacatattaaccaccaggt |
| | | ggtgccaacactggctgtatcaaagaataaagcattacaagcaattgaactgcaactaacgttag |
| | | aaacaatatataactcacaatatagtaatgaaaagtggagtaacactacacccatagtacatttaaa |
| | | aggtgatgctaatactttaaaatgtttaagatatagatttaaaaagcattgtaaattgtatactgcagt |
| | | gtcgtctacatggcattggacaggacataatgtaaaacataaaagtgcaattgttacacttacatat |
| | | gatagtgaatgggggcccgccacgcagccagaactcgccccggaagaccccgaggattcgg |
| | | ccctcttggaggaccccgtggggacggtggcgccgcaaatcccaccaaactggcacatcccgt |
| | | cgatccaggacgccgcgacgccttaccatcccccggccaccccgaacaacatgggcctgatc |
| | | gccggcgcggtgggcggcagtctcctggcagccctggtcatttgcggaattgtgtactggatgc |
| | | accgccgcactcggaaagccccaaagcgcatacgcctcccccacatccgggaagacgacca |
| | | gccgtcctcgcaccagcccttgttttacgactacaaagacgatgacgacaagact |
| Melanoma | SEQ ID | MGGAAARLGAVILFVVIVGLHGVRGKYCLADASLKMADPN |
| antigens | NO: 140 | RFRGKDLPVLDQLTDPPGVRRVYHIQAGLPDPFQPPSLPITVC |
| with | | YAVLERACRSVLLNAPSEAPQIVRGASEDVRKQPYNLTIAWF |
| universal | | RMGGNCAIPITVMEYTECSYNKSLGACPIRTQPRWNYYDSFS |
| helper | | AVSEDNLGFLMHAPAFETAGTYLRLVKINDWTEITQFILEHR |
| epitope and | | AKGSCKYALPLRIPPSACLSPQAYQQGVTVDSIGPQDIEITCV |
| the C- | | YCYDFAFRAACIVYRDGNPYAVCDKCLKFYSKISEYRHYCY |
| terminus of | | SVYGTTLAASVSTYTSLILLVLLLWITAASAFRCFIVYIVFVYI |
| E2 fused | | PLFLIHTHARFLIAASNTTPIVHLKGDANTLKCLRYRFKKHCK |
| into gDM5 | | LYTAVSSTWHWTGHNVKHKSAIVTLTYDSEWGPATQPELAP |
| with C- | | EDPEDSALLEDPVGTVAPQIPPNWHIPSIQDAATPYHPPATPN |
| terminal | | NMGLIAGAVGGSLLAALVICGIVYMHRRTRKAPKRIRLPHIR |
| Flag | | EDDQPSSHQPLFYDYKDDDDKT |
| Protein | | |
| Melanoma | SEQ ID | atggggggggctgccgccaggttgggggccgtgattttgtttgtcgtcatagtgggcctccatgg |
| antigens | NO: 141 | ggtccgcggcaaatattgtttggcggatgcctctctcaagatggccgaccccaatcgctttcgcg |
| with | | gcaaagaccttccggtcctggaccagctgaccgaccctccgggggtccggcgcgtgtaccaca |
| universal | | tccaggcgggcctaccggacccgttccagccccccagcctcccgatcacggtttgctacgccgt |
| helper | | gttggagcgcgcctgccgcagcgtgctcctaaacgcaccgtcggaggccccccagattgtccg |
| epitope and | | cggggcctccgaagacgtccggaaacaaccctacaacctgaccatcgcttggtttcggatggga |
| the C- | | ggcaactgtgctatccccatcacggtcatggagtacaccgaatgctcctacaacaagtctctggg |
| terminus of | | ggcctgtcccatccgaacgcagccccgctggaactactatgacagcttcagcgccgtcagcga |
| E2 fused | | ggataacctggggttcctgatgcacgcccccgcgtttgagaccgccggcacgtacctgcggctc |
| into gDM5 | | gtgaagataaacgactggacggagattacacagtttatcctggagcaccgagccaagggctcct |
| with C- | | gtaagtacgccctcccgctgcgcatccccccgtcagcctgcctctccccccaggcctaccagca |
| terminal | | gggggtgacggtggacagcatcgggcccgctaagtttgtggccgcttggacactgaaggccgc |
| Flag | | tgcagccgctgcaaggaagttctttcacagaacctgcaaatgtacaggaaacttcgccgctgcag |
| Nucleotide | | ccaatgagagcttcgccctgccctattggaactttgctaccggccggaatgaatgcgacgtggct |
| | | gcagccgatcagctcggatactcttatgccatcgacctgcctgtgagtgtcgctgcagccgctgc |
| | | agcctcagtgtacgatttctttgtctggctggctgcagccgctgcagccagcactttctcctttcgca |
| | | acgccctggctgcatctcaggtcatgaacctccataatctggcagctaccgcaccagacaacctc |
| | | ggatatatggcagccgctattgccgtggtcaatgctctgctcctgtacgcctatgactacgaggaa |
| | | ctgtacgctaaagtccccaggaatcaggattggctcgcagccgctgcatttggtctggccaacga |
| | | aaagagcattgcagcagcagcacctaggagtaacactacacccatagtacatttaaaaggtgat |
| | | gctaatactttaaaatgtttaagatatagatttaaaaagcattgtaaattgtatactgcagtgtcgtcta |
| | | catggcattggacaggacataatgtaaaacataaaagtgcaattgttacacttacatatgatagtga |
| | | atgggggcccgccacgcagccagaactcgccccggaagaccccgaggattcggccctcttgg |
| | | aggaccccgtggggacggtggcgccgcaaatcccaccaaactggcacatcccgtcgatccag |
| | | gacgccgcgacgccttaccatcccccggccaccccgaacaacatgggcctgatcgccggcgc |
| | | ggtgggcggcagtctcctggcagccctggtcatttgcggaattgtgtactggatgcaccgccgc |
| | | actcggaaagccccaaagcgcatacgcctcccccacatccgggaagacgaccagccgtcctc |
| | | gcaccagcccttgttttacgactacaaagacgatgacgacaagact |
| Melanoma | SEQ ID | MGGAAARLGAVILFVVIVGLHGVRGKYCLADASLKMADPN |
| antigens | NO: 142 | RFRGKDLPVLDQLTDPPGVRRVYHIQAGLPDPFQPPSLPITVC |
| with | | YAVLERACRSVLLNAPSEAPQIVRGASEDVRKQPYNLTIAWF |
| universal | | RMGGNCAIPITVMEYTECSYNKSLGACPIRTQPRWNYYDSFS |
| helper | | AVSEDNLGFLMHAPAFETAGTYLRLVKINDWTEITQFILEHR |
| epitope and | | AKGSCKYALPLRIPPSACLSPQAYQQGVTVDSIGPAKFVAAW |
| the N- | | TLKAAAAAARKFFHRTCKCTGNFAAAANESFALPYWNFAT |
| terminus of | | GRNECDVAAADQLGYSYAIDLPVSVAAAAAASVYDFFVWL |
| E2 fused | | AAAAAASTFSFRNALAASQVMNLHNLAATAPDNLGYMAAA |
| into gDM5 | | IAVVNALLLYAYDYEELYAKVPRNQDWLAAAAFGLANEKSI |
| with C- | | AAAAPRETLCQRLNVCQDKILTHYENDSTDLRDHIDYWKH |
| terminal | | MRLECAIYYKAREMGFKHINHQVVPTLAVSKNKALQAIELQ |
| Flag | | LTLETIYNSQYSNEKWGPATQPELAPEDPEDSALLEDPVGTV |
| Protein | | APQIPPNWHIPSIQDAATPYHPPATPNNMGLIAGAVGGSLLA |
| | | ALVICGIVYWMHRRTRKAPKRIRLPHIREDDQPSSHQPLFYD |
| | | YKDDDDKT |
| Melanoma | SEQ ID | atggggggggctgccgccaggttgggggccgtgattttgtttgtcgtcatagtgggcctccatgg |
| antigens | NO: 143 | ggtccgcggcaaatattgtttggcggatgcctctctcaagatggccgaccccaatcgctttcgcg |
| with | | gcaaagaccttccggtcctggaccagctgaccgaccctccgggggtccggcgcgtgtaccaca |
| universal | | tccaggcgggcctaccggacccgttccagccccccagcctcccgatcacggtttgctacgccgt |
| helper | | gttggagcgcgcctgccgcagcgtgctcctaaacgcaccgtcggaggccccccagattgtccg |
| epitope and | | cggggcctccgaagacgtccggaaacaaccctacaacctgaccatcgcttggtttcggatggga |
| the N- | | ggcaactgtgctatccccatcacggtcatggagtacaccgaatgctcctacaacaagtctctggg |
| terminus of | | ggcctgtcccatccgaacgcagccccgctggaactactatgacagcttcagcgccgtcagcga |
| E2 fused | | ggataacctggggttcctgatgcacgcccccgcgtttgagaccgccggcacgtacctgcggctc |
| into gDM5 | | gtgaagataaacgactggacggagattacacagtttatcctggagcaccgagccaagggctcct |
| with C- | | gtaagtacgccctcccgctgcgcatccccccgtcagcctgcctctccccccaggcctaccagca |
| terminal | | gggggtgacggtggacagcatcgggcccgctaagtttgtggccgcttggacactgaaggccgc |
| Flag | | tgcagccgctgcaaggaagttctttcacagaacctgcaaatgtacaggaaacttcgccgctgcag |
| Nucleotide | | ccaatgagagcttcgccctgccctattggaactttgctaccggccggaatgaatgcgacgtggct |
| | | gcagccgatcagctcggatactcttatgccatcgacctgcctgtgagtgtcgctgcagccgctgc |
| | | agcctcagtgtacgatttctttgtctggctggctgcagccgctgcagccagcactttctcctttcgca |
| | | acgccctggctgcatctcaggtcatgaacctccataatctggcagctaccgcaccagacaacctc |
| | | ggatatatggcagccgctattgccgtggtcaatgctctgctcctgtacgcctatgactacgaggaa |
| | | ctgtacgctaaagtccccaggaatcaggattggctcgcagccgctgcatttggtctggccaacga |
| | | aaagagcattgcagcagcagcacctagggagactctttgccaacgtttaaatgtgtgtcaggaca |
| | | aaatactaacacattatgaaaatgatagtacagacctacgtgaccatatagactattggaaacacat |
| | | gcgcctagaatgtgctatttattacaaggccagagaaatgggatttaaacatattaaccaccaggt |
| | | ggtgccaacactggctgtatcaaagaataaagcattacaagcaattgaactgcaactaacgttag |
| | | aaacaatatataactcacaatatagtaatgaaaagtgggggcccgccacgcagccagaactcgc |
| | | cccggaagaccccgaggattcggccctcttggaggaccccgtggggacggtggcgccgcaa |
| | | atcccaccaaactggcacatcccgtcgatccaggacgccgcgacgccttaccatcccccggcc |
| | | accccgaacaacatgggcctgatcgccggcgcggtgggcggcagtctcctggcagccctggt |
| | | catttgcggaattgtgtactggatgcaccgccgcactcggaaagccccaaagcgcatacgcctc |
| | | ccccacatccgggaagacgaccagccgtcctcgcaccagcccttgttttacgactacaaagacg |
| | | atgacgacaagact |

### EMBODIMENTS

Disclosed herein are the following embodiments:

Embodiment 1. A nucleic acid molecule encoding a polypeptide comprising the amino acid sequence of SEQ ID NO: 11.

Embodiment 2. The nucleic acid molecule of embodiment 1, wherein the nucleic acid sequence comprises the nucleotide sequence of SEQ ID NO: 12.

Embodiment 3. The nucleic acid molecule of embodiment 1, wherein the nucleic acid molecule encodes a polypeptide comprising the amino acid sequence of SEQ ID NO: 9.

Embodiment 4. The nucleic acid molecule of embodiment 3, wherein the nucleic acid sequence comprises the nucleotide sequence of SEQ ID NO: 10.

Embodiment 5. The nucleic acid molecule of embodiment 1 or 3, comprising:
a nucleotide sequence encoding an N-terminal gDM5 sequence comprising the amino acid sequence of SEQ ID NO: 15;
a nucleotide sequence encoding an antigen; and
a nucleotide sequence encoding a C-terminal gDM5 sequence comprising the amino acid sequence of SEQ ID NO: 17.

Embodiment 6. The nucleic acid molecule of embodiment 5, wherein the nucleotide sequence encoding the N-terminal gDM5 sequence comprises the nucleotide sequence of SEQ ID NO: 16.

Embodiment 7. The nucleic acid molecule of embodiment 5, wherein the nucleotide sequence encodes an N-terminal gDM5 sequence comprising the amino acid sequence of SEQ ID NO: 13.

Embodiment 8. The nucleic acid molecule of embodiment 7, wherein the nucleotide sequence encoding the N-terminal gDM5 sequence comprises the nucleotide sequence of SEQ ID NO: 14.

Embodiment 9. The nucleic acid molecule of any one of embodiments 5-8, wherein the nucleotide sequence encoding a C-terminal gDM5 sequence comprises the nucleotide sequence of SEQ ID NO: 18.

Embodiment 10. The nucleic acid molecule of any one of embodiments 5-9, wherein the antigen is selected from a hepatitis virus antigen, an HIV antigen, a melanoma antigen, and an HPV antigen.

Embodiment 11. The nucleic acid molecule of any one of embodiments 5-10, wherein the antigen is a PolN protein from HBV, a gag protein from HIV, an E protein of HPV, HBV3 Protein, Melapoly Protein, E765-wt Protein, Melapoly Protein #2, or Melanoma antigens with universal helper epitope Protein.

Embodiment 12. A gDM5 protein comprising the amino acid sequence of SEQ ID NO: 11.

Embodiment 13. The gDM5 protein of embodiment 12, comprising the amino acid sequence of SEQ ID NO: 9.

Embodiment 14. A fusion protein comprising:
an N-terminal gDM5 sequence comprising the amino acid sequence of SEQ ID NO: 15;
an antigen; and
a C-terminal gDM5 sequence comprising the amino acid sequence of SEQ ID NO: 17.

Embodiment 15. The fusion protein of embodiment 14, wherein the N-terminal gDM5 sequence comprises the amino acid sequence of SEQ ID NO: 13.

Embodiment 16. The fusion protein of embodiment 14 or 15, wherein the antigen is selected from a hepatitis virus antigen, an HIV antigen, a melanoma antigen, and an HPV antigen.

Embodiment 17. The fusion protein of any one of embodiments 14-16, wherein the antigen is a PolN protein from HBV, a gag protein from HIV, an E protein of HPV, HBV3 Protein, Melapoly Protein, E765-wt Protein, Melapoly Protein #2, or Melanoma antigens with universal helper epitope Protein.

Embodiment 18. A vector comprising the nucleic acid molecule of any one of embodiments 1-11.

Embodiment 19. A host cell comprising the vector of embodiment 18.

Embodiment 20. A virus comprising the nucleic acid molecule of any one of embodiments 1-11 or the vector of embodiment 18.

Embodiment 21. The virus of embodiment 20, wherein the virus is an adenovirus.

Embodiment 22. The virus of embodiment 21, wherein the adenovirus is an AdC6, AdC68, or AdC7.

Embodiment 23. A vaccine comprising the nucleic acid molecule of any one of embodiments 1-11, the vector of embodiment 18, or the virus of any one of embodiments 20-22.

Embodiment 24. A method of inducing an immune response in a subject, the method comprising providing to the subject an effective amount of the nucleic acid molecule of any one of embodiments 1-11, the vector of embodiment 18, the fusion protein of any one of embodiments 14-17, the virus of any one of embodiments 20-22, or the vaccine of embodiment 23 to thereby induce an immune response.

Embodiment 25. A nucleic acid molecule encoding:
a mutant human papilloma virus 16 (HPV 16) E7 protein comprising the amino acid sequence of SEQ ID NO: 34;
a mutant HPV 16 E6 protein comprising the amino acid sequence of SEQ ID NO: 36;
a mutant HPV 16 E5 protein comprising the amino acid sequence of SEQ ID NO: 38;
a mutant HPV 16 E2 protein comprising the amino acid sequence of SEQ ID NO: 40;
a C-terminal mutant HPV 16 E2 protein comprising the amino acid sequence of SEQ ID NO: 56;
a N-terminal mutant HPV 16 E2 protein comprising the amino acid sequence of SEQ ID NO: 58;
a mutant HPV 16 E7 protein v2 comprising the amino acid sequence of SEQ ID NO: 64;
a mutant HPV 16 E6 protein v2 comprising the amino acid sequence of SEQ ID NO: 66; or
a mutant HPV 16 E5 protein v2 comprising the amino acid sequence of SEQ ID NO: 68.

Embodiment 26. The nucleic acid molecule of embodiment 25, wherein the nucleic acid molecule encodes the mutant HPV 16 E2 protein comprising the amino acid sequence of SEQ ID NO: 40.

Embodiment 27. The nucleic acid molecule of embodiment 25, wherein the nucleic acid molecule encodes the C-terminal mutant HPV 16 E2 protein comprising the amino acid sequence of SEQ ID NO: 56.

Embodiment 28. The nucleic acid molecule of embodiment 25, wherein the nucleic acid molecule encodes the N-terminal mutant HPV 16 E2 protein comprising the amino acid sequence of SEQ ID NO: 58.

Embodiment 29. The nucleic acid molecule of any one of embodiments 25-28, comprising the:
mutant HPV 16 E7 comprising the nucleotide sequence of SEQ ID NO: 35;
mutant HPV 16 E6 comprising the nucleotide sequence of SEQ ID NO: 37;
mutant HPV 16 E5 comprising the nucleotide sequence of SEQ ID NO: 39;
mutant HPV 16 E2 comprising the nucleotide sequence of SEQ ID NO: 41; C-terminal mutant HPV 16 E2 comprising the nucleotide sequence of SEQ ID NO: 57;
N-terminal mutant HPV 16 E2 comprising the nucleotide sequence of SEQ ID NO: 59;
mutant HPV 16 E7 v2 comprising the nucleotide sequence of SEQ ID NO: 65;
mutant HPV 16 E6 v2 comprising the nucleotide sequence of SEQ ID NO: 67; or
mutant HPV 16 E5 v2 comprising the nucleotide sequence of SEQ ID NO: 69.

Embodiment 30. The nucleic acid molecule of embodiment 29, comprising the mutant HPV 16 E2 comprising the nucleotide sequence of SEQ ID NO: 41.

Embodiment 31. The nucleic acid molecule of embodiment 29, comprising the C-terminal mutant HPV 16 E2 comprising the nucleotide sequence of SEQ ID NO: 57.

Embodiment 32. The nucleic acid molecule of embodiment 29, comprising the N-terminal mutant HPV 16 E2 comprising the nucleotide sequence of SEQ ID NO: 59.

Embodiment 33. A nucleic acid molecule encoding an HPV 16 fusion protein, wherein the HPV 16 fusion protein comprises:
any one of the HPV 16 E7 proteins provided in Table 10, any one of the HPV 16 E6 proteins provided in Table 10, and any one of the HPV 16 E5 proteins provided in Table 10.

Embodiment 34. The nucleic acid molecule of embodiment 33, wherein the HPV 16 fusion protein further comprises any one of the HPV 16 E2 proteins provided in Table 10.

Embodiment 35. The nucleic acid molecule of embodiment 33, comprising:
any one of the HPV 16 E7 nucleotide sequences provided in Table 11, any one of the HPV 16 E6 nucleotide sequences provided in Table 11, and any one of the HPV 16 E5 nucleotide sequences provided in Table 11.

Embodiment 36. The nucleic acid molecule of embodiment 35, further comprising any of the HPV 16 E2 nucleotide sequences provided in Table 11.

Embodiment 37. The nucleic acid molecule of any one of embodiments 33-36, wherein the nucleic acid molecule encodes any one of the HPV 16 fusion proteins provided in Table 12.

Embodiment 38. The nucleic acid molecule of any one of embodiments 33-37, comprising any one of the nucleotide sequences provided in Table 12.

Embodiment 39. A nucleic acid molecule encoding an HPV 16 E2-antigen fusion protein, wherein the HPV 16 E2-antigen fusion protein comprises:
any one of the HPV 16 E2 proteins provided in Table 14; and
any one of the antigens provided in Table 14.

Embodiment 40. The nucleic acid molecule of embodiment 39, wherein the HPV 16 E2-antigen fusion protein comprises any one of the amino acid sequences provided in Table 16.

Embodiment 41. The nucleic acid molecule of embodiment 40, wherein the nucleic acid molecule encodes an HPV 16 E2-melanoma antigen with a universal T helper cell epitope comprising the amino acid sequence of SEQ ID NO: 76.

Embodiment 42. The nucleic acid molecule of any one of embodiments 39-41, comprising:
any one of the HPV 16 E2 nucleotide sequences provided in Table 15; and
any one of the antigen nucleotide sequences provided in Table 15.

Embodiment 43. The nucleic acid molecule of embodiment 42, comprising any one of the nucleotide sequences provided in Table 16.

Embodiment 44. The nucleic acid molecule of embodiment 43, comprising the HPV 16 E2-melanoma antigen with a universal T helper cell epitope comprising the nucleotide sequence of SEQ ID NO: 77.

Embodiment 45. The nucleic acid molecule of any one of embodiments 39-44, wherein the HPV 16 E2-antigen fusion protein further comprises a gD.

Embodiment 46. The nucleic acid molecule of embodiment 45, wherein the gD comprises any one of the amino acid sequences provided in Table 17.

Embodiment 47. The nucleic acid molecule of embodiment 46, wherein the gD comprises any one of the nucleotide sequences provided in Table 18.

Embodiment 48. The nucleic acid molecule of any one of embodiments 45-47, wherein the nucleic acid molecule encodes any one of the gD-HPV 16 E2-antigen fusion proteins provided in Table 19.

Embodiment 49. The nucleic acid molecule of embodiment 48, comprising the nucleotide sequence of any one of the nucleotide sequences provided in Table 19.

Embodiment 50. The nucleic acid molecule of embodiment 48, wherein the nucleic acid molecule encodes:
a melanoma antigens with universal helper epitope and E2 fused into gD Protein comprising the amino acid sequence of SEQ ID NO: 120;
a melanoma antigens with universal helper epitope and C-terminus E2 fused into gD Protein comprising the amino acid sequence of SEQ ID NO: 122; or
a melanoma antigens with universal helper epitope and N-terminus E2 fused into gD Protein comprising the amino acid sequence of SEQ ID NO: 124.

Embodiment 51. The nucleic acid molecule of embodiment 50, comprising the nucleotide sequence of any one of SEQ ID NOs: 121, 123, or 125.

Embodiment 52. A protein comprising any one of the amino acid sequences provided in Tables 7, 8, 10, 12, 14, 16, 17, or 19.

Embodiment 53. The protein of embodiment 52, comprising:
a mutant human papilloma virus 16 (HPV 16) E7 protein comprising the amino acid sequence of SEQ ID NO: 34;
a mutant HPV 16 E6 protein comprising the amino acid sequence of SEQ ID NO: 36;
a mutant HPV 16 E5 protein comprising the amino acid sequence of SEQ ID NO: 38;
a mutant HPV 16 E2 protein comprising the amino acid sequence of SEQ ID NO: 40;
a C-terminal mutant HPV 16 E2 protein comprising the amino acid sequence of SEQ ID NO: 56;
a N-terminal mutant HPV 16 E2 protein comprising the amino acid sequence of SEQ ID NO: 58;
a mutant HPV 16 E7 protein v2 comprising the amino acid sequence of SEQ ID NO: 64;
a mutant HPV 16 E6 protein v2 comprising the amino acid sequence of SEQ ID NO: 66;
a mutant HPV 16 E5 protein v2 comprising the amino acid sequence of SEQ ID NO: 68;
an HPV 16 E2-melanoma antigen with a universal T helper cell epitope comprising the amino acid sequence of SEQ ID NO: 76;
a melanoma antigens with universal helper epitope and E2 fused into gD Protein comprising the amino acid sequence of SEQ ID NO: 120;
a melanoma antigens with universal helper epitope and C-terminus E2 fused into gD Protein comprising the amino acid sequence of SEQ ID NO: 122: or
a melanoma antigens with universal helper epitope and N-terminus E2 fused into gD Protein comprising the amino acid sequence of SEQ ID NO: 124.

Embodiment 54. A vector comprising the nucleic acid molecule of any one of embodiments 25-50.

Embodiment 55. A host cell comprising the vector of embodiment 52.

Embodiment 56. A virus comprising the nucleic acid molecule of any one of embodiments 25-51 or the vector of embodiment 54.

Embodiment 57. The virus of embodiment 56, wherein the virus is an adenovirus.

Embodiment 58. The virus of embodiment 57, wherein the adenovirus is an AdC6, AdC68, or AdC7.

Embodiment 59. A vaccine comprising the nucleic acid molecule of any one of embodiments 25-51, the vector of embodiment 54, or the virus of any one of embodiments 56-58.

Embodiment 60. A method of inducing an immune response in a subject, the method comprising providing to the subject an effective amount of the nucleic acid molecule of any one of embodiments 25-51, the vector of embodiment 54, the fusion protein of embodiment 52 or 53, the virus of any one of embodiments 56-58, or the vaccine of embodiment 59 to thereby induce an immune response.

Disclosed herein are the following additional embodiments:

Embodiment 1A. A nucleic acid molecule encoding a gD mutant comprising the amino acid sequence of any one of SEQ ID NOs: 1, 3, 5, 7, 49, 50, or 52.

Embodiment 2A. The nucleic acid molecule of embodiment 1A, wherein the nucleic acid sequence comprises the nucleotide sequence of any one of SEQ ID NOs: 2, 4, 6, 8, 51, or 53.

Embodiment 3A. The nucleic acid molecule of embodiment 1A or 2A, further comprising a nucleotide sequence encoding an antigen.

Embodiment 4A. The nucleic acid molecule of embodiment 3A, wherein the antigen comprises any one of the amino acid or nucleotide sequences provided in Table 3.

Embodiment 5A. The nucleic acid molecule of embodiment 3A or 4A, wherein the nucleic acid molecule encodes a protein comprising the amino acid sequence of any one of the amino acid sequences provided in Table 4 or Table 6.

Embodiment 6A. The nucleic acid molecule of embodiment 5A, wherein the nucleic acid molecule comprises the nucleotide sequence of any one of the nucleotide sequences provided in Table 5 or Table 6.

Embodiment 7A. A fusion protein comprising:
a gD mutant comprising the amino acid sequence of any one of SEQ ID NOS: 1, 3, 5, 7, 49, 50, or 52; and
an antigen.

Embodiment 8A. The fusion protein of embodiment 7A, wherein the antigen comprises the amino acid sequence of any one of the amino acid sequences provided in Table 3.

Embodiment 9A. The fusion protein of embodiment 7A or 8A, wherein the fusion provide comprises the amino acid sequence of any one of the amino acid sequences provided in Table 6.

Embodiment 10A. A vector comprising the nucleic acid molecule of any one of embodiments 1A-6A.

Embodiment 11A. A host cell comprising the vector of embodiment 10A.

Embodiment 12A. A virus comprising the nucleic acid molecule of any one of embodiments 1A-6A or the vector of embodiment 10A.

Embodiment 13A. The virus of embodiment 12A, wherein the virus is an adenovirus.

Embodiment 14A. The virus of embodiment 13A, wherein the adenovirus is an AdC6, AdC68, or AdC7.

Embodiment 15A. A vaccine comprising the nucleic acid molecule of any one of embodiments 1A-6A, the embodiment of embodiment 10A, or the virus of any one of embodiments 12A-14A.

Embodiment 16A. A method of inducing an immune response in a subject, the method comprising providing to the subject an effective amount of the nucleic acid molecule of any one of embodiments 1A-6A, the vector of embodiment 10A, the fusion protein of any one of embodiments 7A-9A, the virus of any one of embodiments 12A-14A, or the vaccine of embodiment 15A to thereby induce an immune response.

Disclosed herein are the following additional embodiments:

Embodiment 1B. A nucleic acid molecule comprising:
a nucleotide sequence encoding a mutant human papilloma virus 16 (HPV 16) E7 protein comprising the amino acid sequence of SEQ ID NO: 34;
a nucleotide sequence encoding a mutant HPV 16 E6 protein comprising the amino acid sequence of SEQ ID NO: 36;
a nucleotide sequence encoding a mutant HPV 16 E5 protein comprising the amino acid sequence of SEQ ID NO: 38; and
a nucleotide sequence encoding a mutant HPV 16 E2 protein comprising the amino acid sequence of SEQ ID NO: 40.

Embodiment 2B. The nucleic acid molecule of embodiment 1, wherein the nucleic acid molecule encodes a fusion protein comprising the amino acid sequence of SEQ ID NO: 42.

Embodiment 3B. The nucleic acid molecule of embodiment 1 or 2, wherein the nucleic acid molecule comprises the nucleotide sequence of one or more of SEQ ID NO: 35, SEQ ID NO: 37, SEQ ID NO: 39, and SEQ ID NO: 41.

Embodiment 4B. The nucleic acid molecule of any one of the previous embodiments, wherein the nucleic acid molecule comprises the nucleotide sequence of SEQ ID NO: 43.

Embodiment 5B. The nucleic acid molecule of any one of the previous embodiments, further comprising a nucleotide sequence encoding:
an N-terminal gDM5 sequence;
a C-terminal gDM5 sequence; or
both the N-terminal gDM5 sequence and the C-terminal gDM5 sequence.

Embodiment 6B. The nucleic acid molecule of embodiment 5, comprising:
a nucleotide sequence encoding an N-terminal gDM5 sequence;
a nucleotide sequence encoding a mutant HPV 16 E7 protein comprising the amino acid sequence of SEQ ID NO: 34;
a nucleotide sequence encoding a mutant HPV 16 E6 protein comprising the amino acid sequence of SEQ ID NO: 36;
a nucleotide sequence encoding a mutant HPV 16 E5 protein comprising the amino acid sequence of SEQ ID NO: 38;
a nucleotide sequence encoding a mutant HPV 16 E2 protein comprising the amino acid sequence of SEQ ID NO: 40; and
a nucleotide sequence encoding a C-terminal gDM5 sequence.

Embodiment 7B. The nucleic acid molecule of embodiment 5 or 6, wherein the nucleotide sequence encodes an N-terminal gDM5 sequence comprising the amino sequence of SEQ ID NO: 15.

Embodiment 8B. The nucleic acid molecule of embodiment 7, wherein the nucleotide sequence encoding the N-terminal gDM5 sequence comprises the nucleotide sequence of SEQ ID NO: 16.

Embodiment 9B. The nucleic acid molecule of any one of embodiments 5-7, wherein the nucleotide sequence encodes an N-terminal gDM5 sequence comprising the amino sequence of SEQ ID NO: 13.

Embodiment 10B. The nucleic acid molecule of embodiment 9, wherein the nucleotide sequence encoding the N-terminal gDM5 sequence comprises the nucleotide sequence of SEQ ID NO: 14.

Embodiment 11B. The nucleic acid molecule of any one of embodiments 5-10, wherein the nucleotide sequence encodes a C-terminal gDM5 sequence comprising the amino sequence of SEQ ID NO: 17.

Embodiment 12B. The nucleic acid molecule of embodiment 11, wherein the nucleotide sequence encoding a C-terminal gDM5 sequence comprises the nucleotide sequence of SEQ ID NO: 18.

Embodiment 13B. The nucleic acid molecule of any one of embodiments 5-7, 9, and 11 comprising:
a nucleotide sequence encoding an N-terminal gDM5 sequence comprising the amino sequence of SEQ ID NO: 15;
a nucleotide sequence encoding a mutant HPV 16 E7 protein comprising the amino acid sequence of SEQ ID NO: 34;
a nucleotide sequence encoding a mutant HPV 16 E6 protein comprising the amino acid sequence of SEQ ID NO: 36;
a nucleotide sequence encoding a mutant HPV 16 E5 protein comprising the amino acid sequence of SEQ ID NO: 38;
a nucleotide sequence encoding a mutant HPV 16 E2 protein comprising the amino acid sequence of SEQ ID NO: 40; and
a nucleotide sequence encoding a C-terminal gDM5 sequence comprising the amino sequence of SEQ ID NO: 17.

Embodiment 14B. The nucleic acid molecule of embodiment 13, wherein the nucleotide sequence encodes an N-terminal gDM5 sequence comprising the amino sequence of SEQ ID NO: 13.

Embodiment 15B. The nucleic acid molecule of embodiment 13 or 14, wherein the nucleotide sequence encodes a protein comprising the amino acid sequence of SEQ ID NO: 42.

Embodiment 16B. The nucleic acid molecule of any one of embodiments 13-15, wherein the nucleotide sequence comprises SEQ ID NO: 43.

Embodiment 17B. The nucleic acid molecule of any one of embodiments 13-15, wherein the nucleic acid molecule encodes the amino sequence of SEQ ID NO: 46.

Embodiment 18B. The nucleic acid molecule of embodiment 17, wherein the nucleic acid molecule comprises the nucleotide sequence of SEQ ID NO: 47.

Embodiment 19B. The nucleic acid molecule of any one of embodiments 13-15 and 17, wherein the nucleic acid molecule encodes the amino sequence of SEQ ID NO: 44.

Embodiment 20B. The nucleic acid molecule of embodiment 19, wherein the nucleic acid molecule comprises the nucleotide sequence of SEQ ID NO: 45.

Embodiment 21B. A mutant HPV 16 E7 protein comprising the amino acid sequence of SEQ ID NO: 34.

Embodiment 22B. A mutant HPV 16 E6 protein comprising the amino acid sequence of SEQ ID NO: 36.

Embodiment 23B. A mutant HPV 16 E5 protein comprising the amino acid sequence of SEQ ID NO: 38.

Embodiment 24B. A mutant HPV 16 E2 protein comprising the amino acid sequence of SEQ ID NO: 40.

Embodiment 25B. A fusion protein comprising:
a mutant HPV 16 E7 protein comprising the amino acid sequence of SEQ ID NO: 34;
a mutant HPV 16 E6 protein comprising the amino acid sequence of SEQ ID NO: 36;
a mutant HPV 16 E5 protein comprising the amino acid sequence of SEQ ID NO: 38; and
a mutant HPV 16 E2 protein comprising the amino acid sequence of SEQ ID NO: 40.

Embodiment 26B. The fusion protein of embodiment 25, wherein the fusion protein comprises the amino acid sequence of SEQ ID NO: 42.

Embodiment 27B. The fusion protein of embodiment 25 or 26, further comprising:
an N-terminal gDM5 sequence;
a C-terminal gDM5 sequence; or
both the N-terminal gDM5 sequence and the C-terminal gDM5 sequence.

Embodiment 28B. The fusion protein of embodiment 27, comprising:
an N-terminal gDM5 sequence;
a mutant HPV 16 E7 protein comprising the amino acid sequence of SEQ ID NO: 34;
a mutant HPV 16 E6 protein comprising the amino acid sequence of SEQ ID NO: 36;
a mutant HPV 16 E5 protein comprising the amino acid sequence of SEQ ID NO: 38;
a mutant HPV 16 E2 protein comprising the amino acid sequence of SEQ ID NO: 40; and
a C-terminal gDM5 sequence.

Embodiment 29B. The fusion protein of embodiment 28, wherein the fusion protein comprises the amino acid sequence of SEQ ID NO: 42.

Embodiment 30B. The fusion protein of any one of embodiments 27-29, wherein the N-terminal gDM5 sequence comprises the amino sequence of SEQ ID NO: 15.

Embodiment 31B. The fusion protein of any one of embodiments 27-30, wherein the N-terminal gDM5 sequence comprises the amino sequence of SEQ ID NO: 13.

Embodiment 32B. The fusion protein of any one of embodiments 27-31, wherein the C-terminal gDM5 sequence comprises the amino sequence of SEQ ID NO: 17.

Embodiment 33B. The fusion protein of any one of embodiments 27-32, comprising:
an N-terminal gDM5 sequence comprising the amino sequence of SEQ ID NO: 15;
a mutant HPV 16 E7 protein comprising the amino acid sequence of SEQ ID NO: 34;
a mutant HPV 16 E6 protein comprising the amino acid sequence of SEQ ID NO: 36;
a mutant HPV 16 E5 protein comprising the amino acid sequence of SEQ ID NO: 38;
a mutant HPV 16 E2 protein comprising the amino acid sequence of SEQ ID NO: 40; and
a C-terminal gDM5 sequence comprising the amino sequence of SEQ ID NO: 17.

Embodiment 34B. The fusion protein of embodiment 40, wherein the N-terminal gDM5 sequence comprises the amino sequence of SEQ ID NO: 13.

Embodiment 35B. The fusion protein of any one of embodiments 27-34, wherein the fusion protein sequence comprises the amino acid sequence of SEQ ID NO: 46.

Embodiment 36B. The fusion protein of any one of embodiments 27-35, wherein the fusion protein sequence comprises the amino acid sequence of SEQ ID NO: 44.

Embodiment 37B. A nucleic acid molecule encoding the protein of any one of embodiments 21-24.

Embodiment 38B. The nucleic acid molecule of embodiment 37, wherein the nucleic acid molecule comprises the nucleotide sequence of SEQ ID NO: 35, SEQ ID NO: 37, SEQ ID NO: 39, or SEQ ID NO: 41.

Embodiment 39B. A vector comprising the nucleic acid molecule of any one of embodiments 1-20, 37, or 38.

Embodiment 40B. A host cell comprising the vector of embodiment 39.

Embodiment 41B. A virus comprising the vector of embodiment 39 or the nucleic acid of any one of embodiments 1-20, 37, or 38.

Embodiment 42B. The virus of embodiment 41, wherein the virus is an adenovirus.

Embodiment 43B. The virus of embodiment 42, wherein the adenovirus is an AdC6, AdC68, or AdC7.

Embodiment 44B. A vaccine comprising the vector of embodiment 39, the nucleic acid molecule of any one of embodiments 1-20, 37, or 38, or the virus of any one of embodiments 41-43.

Embodiment 45B. A method of inducing an immune response to HPV in a subject, the method comprising providing to the subject an effective amount of the vector of embodiment 39, the nucleic acid molecule of any one of embodiments 1-20, 37, or 38, the fusion protein of any one of embodiments 25-36, the virus of any one of embodiments 41-43, or the vaccine of embodiment 44 to thereby induce an immune response to HPV.

What is claimed:

1. A nucleic acid molecule encoding a polypeptide comprising the amino acid sequence of SEQ ID NO: 11.

2. The nucleic acid molecule of claim 1, wherein the nucleic acid sequence comprises the nucleotide sequence of SEQ ID NO: 12.

3. The nucleic acid molecule of claim 1, wherein the nucleic acid molecule encodes a polypeptide comprising the amino acid sequence of SEQ ID NO: 9.

4. The nucleic acid molecule of claim 3, wherein the nucleic acid sequence comprises the nucleotide sequence of SEQ ID NO: 10.

5. The nucleic acid molecule of claim 1, comprising:
a nucleotide sequence encoding an N-terminal gDM5 sequence comprising the amino acid sequence of SEQ ID NO: 15;
a nucleotide sequence encoding an antigen; and
a nucleotide sequence encoding a C-terminal gDM5 sequence comprising the amino acid sequence of SEQ ID NO: 17.

6. The nucleic acid molecule of claim 5, wherein the nucleotide sequence encoding the N-terminal gDM5 sequence comprises the nucleotide sequence of SEQ ID NO: 16.

7. The nucleic acid molecule of claim 5, wherein the nucleotide sequence encodes an N-terminal gDM5 sequence comprising the amino acid sequence of SEQ ID NO: 13.

8. The nucleic acid molecule of claim 7, wherein the nucleotide sequence encoding the N-terminal gDM5 sequence comprises the nucleotide sequence of SEQ ID NO: 14.

9. The nucleic acid molecule of claim 5, wherein the nucleotide sequence encoding a C-terminal gDM5 sequence comprises the nucleotide sequence of SEQ ID NO: 18.

10. The nucleic acid molecule of claim 5, wherein the antigen is selected from a hepatitis virus antigen, an HIV antigen, a melanoma antigen, and an HPV antigen.

11. The nucleic acid molecule of claim 5, wherein the antigen is a PolN protein from HBV, a gag protein from HIV, an E protein of HPV, HBV3 Protein, Melapoly Protein, E765-wt Protein, Melapoly Protein #2, or Melanoma antigens with universal helper epitope Protein.

12. A gDM5 protein comprising the amino acid sequence of SEQ ID NO: 11.

13. The gDM5 protein of claim 12, comprising the amino acid sequence of SEQ ID NO: 9.

14. A fusion protein comprising:
an N-terminal gDM5 sequence comprising the amino acid sequence of SEQ ID NO: 15;
an antigen; and
a C-terminal gDM5 sequence comprising the amino acid sequence of SEQ ID NO: 17.

15. The fusion protein of claim 14, wherein the N-terminal gDM5 sequence comprises the amino acid sequence of SEQ ID NO: 13.

16. The fusion protein of claim 14, wherein the antigen is selected from a hepatitis virus antigen, an HIV antigen, a melanoma antigen, and an HPV antigen.

17. The fusion protein of claim 14, wherein the antigen is a PolN protein from HBV, a gag protein from HIV, an E protein of HPV, HBV3 Protein, Melapoly Protein, E765-wt Protein, Melapoly Protein #2, or Melanoma antigens with universal helper epitope Protein.

18. A vector comprising the nucleic acid molecule of claim 1.

19. A host cell comprising the vector of claim 18.

20. A virus comprising the vector of claim 18.

21. The virus of claim 20, wherein the virus is an adenovirus.

22. The virus of claim 21, wherein the adenovirus is an AdC6, AdC68, or AdC7.

23. A vaccine comprising the virus of claim 20.

24. A method of inducing an immune response in a subject, the method comprising providing to the subject an effective amount of the vaccine of claim 23 to thereby induce an immune response.

25. A nucleic acid molecule encoding:
a mutant human papilloma virus 16 (HPV 16) E7 protein comprising the amino acid sequence of SEQ ID NO: 34;
a mutant HPV 16 E6 protein comprising the amino acid sequence of SEQ ID NO: 36;
a mutant HPV 16 E5 protein comprising the amino acid sequence of SEQ ID NO: 38;
a mutant HPV 16 E2 protein comprising the amino acid sequence of SEQ ID NO: 40;
a C-terminal mutant HPV 16 E2 protein comprising the amino acid sequence of SEQ ID NO: 56;
a N-terminal mutant HPV 16 E2 protein comprising the amino acid sequence of SEQ ID NO: 58;
a mutant HPV 16 E7 protein v2 comprising the amino acid sequence of SEQ ID NO: 64;
a mutant HPV 16 E6 protein v2 comprising the amino acid sequence of SEQ ID NO: 66; or
a mutant HPV 16 E5 protein v2 comprising the amino acid sequence of SEQ ID NO: 68.

26. The nucleic acid molecule of claim 25, wherein the nucleic acid molecule encodes the mutant HPV 16 E2 protein comprising the amino acid sequence of SEQ ID NO: 40.

27. The nucleic acid molecule of claim 25, wherein the nucleic acid molecule encodes the C-terminal mutant HPV 16 E2 protein comprising the amino acid sequence of SEQ ID NO: 56.

28. The nucleic acid molecule of claim 25, wherein the nucleic acid molecule encodes the N-terminal mutant HPV 16 E2 protein comprising the amino acid sequence of SEQ ID NO: 58.

29. The nucleic acid molecule of claim 25, comprising the:
mutant HPV 16 E7 comprising the nucleotide sequence of SEQ ID NO: 35;
mutant HPV 16 E6 comprising the nucleotide sequence of SEQ ID NO: 37;
mutant HPV 16 E5 comprising the nucleotide sequence of SEQ ID NO: 39;
mutant HPV 16 E2 comprising the nucleotide sequence of SEQ ID NO: 41;
C-terminal mutant HPV 16 E2 comprising the nucleotide sequence of SEQ ID NO: 57;
N-terminal mutant HPV 16 E2 comprising the nucleotide sequence of SEQ ID NO: 59;
mutant HPV 16 E7 v2 comprising the nucleotide sequence of SEQ ID NO: 65;
mutant HPV 16 E6 v2 comprising the nucleotide sequence of SEQ ID NO: 67; or
mutant HPV 16 E5 v2 comprising the nucleotide sequence of SEQ ID NO: 69.

30. The nucleic acid molecule of claim 29, comprising the mutant HPV 16 E2 comprising the nucleotide sequence of SEQ ID NO: 41.

31. The nucleic acid molecule of claim 29, comprising the C-terminal mutant HPV 16 E2 comprising the nucleotide sequence of SEQ ID NO: 57.

32. The nucleic acid molecule of claim 29, comprising the N-terminal mutant HPV 16 E2 comprising the nucleotide sequence of SEQ ID NO: 59.

33. A nucleic acid molecule encoding an HPV 16 fusion protein, wherein the HPV 16 fusion protein comprises:
any one of the HPV 16 E7 proteins provided in Table 10, any one of the HPV 16 E6 proteins provided in Table 10, and any one of the HPV 16 E5 proteins provided in Table 10.

34. The nucleic acid molecule of claim 33, wherein the HPV 16 fusion protein further comprises any one of the HPV 16 E2 proteins provided in Table 10.

35. The nucleic acid molecule of claim 33, comprising:
any one of the HPV 16 E7 nucleotide sequences provided in Table 11, any one of the HPV 16 E6 nucleotide sequences provided in Table 11, and any one of the HPV 16 E5 nucleotide sequences provided in Table 11.

36. The nucleic acid molecule of claim 35, further comprising any of the HPV 16 E2 nucleotide sequences provided in Table 11.

37. The nucleic acid molecule of claim 33, wherein the nucleic acid molecule encodes any one of the HPV 16 fusion proteins provided in Table 12.

38. The nucleic acid molecule of claim 33, comprising any one of the nucleotide sequences provided in Table 12.

39. A nucleic acid molecule encoding an HPV 16 E2-antigen fusion protein, wherein the HPV 16 E2-antigen fusion protein comprises:
any one of the HPV 16 E2 proteins provided in Table 14; and
any one of the antigens provided in Table 14.

40. The nucleic acid molecule of claim 39, wherein the HPV 16 E2-antigen fusion protein comprises any one of the amino acid sequences provided in Table 16.

41. The nucleic acid molecule of claim 40, wherein the nucleic acid molecule encodes an HPV 16 E2-melanoma antigen with a universal T helper cell epitope comprising the amino acid sequence of SEQ ID NO: 76.

42. The nucleic acid molecule of claim 39, comprising:
any one of the HPV 16 E2 nucleotide sequences provided in Table 15; and
any one of the antigen nucleotide sequences provided in Table 15.

43. The nucleic acid molecule of claim 42, comprising any one of the nucleotide sequences provided in Table 16.

44. The nucleic acid molecule of claim 43, comprising the HPV 16 E2-melanoma antigen with a universal T helper cell epitope comprising the nucleotide sequence of SEQ ID NO: 77.

45. The nucleic acid molecule of claim 39, wherein the HPV 16 E2-antigen fusion protein further comprises a gD.

46. The nucleic acid molecule of claim 45, wherein the gD comprises any one of the amino acid sequences provided in Table 17.

47. The nucleic acid molecule of claim 46, wherein the gD comprises any one of the nucleotide sequences provided in Table 18.

48. The nucleic acid molecule of claim 45, wherein the nucleic acid molecule encodes any one of the gD-HPV 16 E2-antigen fusion proteins provided in Table 19.

49. The nucleic acid molecule of claim 48, comprising the nucleotide sequence of any one of the nucleotide sequences provided in Table 19.

50. The nucleic acid molecule of claim 48, wherein the nucleic acid molecule encodes:
a melanoma antigens with universal helper epitope and E2 fused into gD Protein comprising the amino acid sequence of SEQ ID NO: 120;
a melanoma antigens with universal helper epitope and C-terminus E2 fused into gD Protein comprising the amino acid sequence of SEQ ID NO: 122; or
a melanoma antigens with universal helper epitope and N-terminus E2 fused into gD Protein comprising the amino acid sequence of SEQ ID NO: 124.

51. The nucleic acid molecule of claim 50, comprising the nucleotide sequence of any one of SEQ ID NOs: 121, 123, or 125.

52. A protein comprising any one of the amino acid sequences provided in Tables 7, 8, 10, 12, 14, 16, 17, or 19.

53. The protein of claim 52, comprising:
a mutant human papilloma virus 16 (HPV 16) E7 protein comprising the amino acid sequence of SEQ ID NO: 34;
a mutant HPV 16 E6 protein comprising the amino acid sequence of SEQ ID NO: 36;
a mutant HPV 16 E5 protein comprising the amino acid sequence of SEQ ID NO: 38;
a mutant HPV 16 E2 protein comprising the amino acid sequence of SEQ ID NO: 40;
a C-terminal mutant HPV 16 E2 protein comprising the amino acid sequence of SEQ ID NO: 56;
a N-terminal mutant HPV 16 E2 protein comprising the amino acid sequence of SEQ ID NO: 58;
a mutant HPV 16 E7 protein v2 comprising the amino acid sequence of SEQ ID NO: 64;
a mutant HPV 16 E6 protein v2 comprising the amino acid sequence of SEQ ID NO: 66;
a mutant HPV 16 E5 protein v2 comprising the amino acid sequence of SEQ ID NO: 68;
an HPV 16 E2-melanoma antigen with a universal T helper cell epitope comprising the amino acid sequence of SEQ ID NO: 76;
a melanoma antigens with universal helper epitope and E2 fused into gD Protein comprising the amino acid sequence of SEQ ID NO: 120;
a melanoma antigens with universal helper epitope and C-terminus E2 fused into gD Protein comprising the amino acid sequence of SEQ ID NO: 122; or
a melanoma antigens with universal helper epitope and N-terminus E2 fused into gD Protein comprising the amino acid sequence of SEQ ID NO: 124.

54. A vector comprising the nucleic acid molecule of claim 25.

55. A host cell comprising the vector of claim 54.

56. A virus comprising the vector of claim 54.

57. The virus of claim 56, wherein the virus is an adenovirus.

58. The virus of claim 57, wherein the adenovirus is an AdC6, AdC68, or AdC7.

59. A vaccine comprising the virus of claim 56.

60. A method of inducing an immune response in a subject, the method comprising providing to the subject an effective amount of the vaccine of claim 59 to thereby induce an immune response.


SEQUENCE LISTING (143 sequences)

SEQ ID NO: 1 - protein, 394 aa, synthetic construct
MGGAAARLGAVILFVVIVGLHGVRGKYCLADASLKMADPNRFRGKDLPVLDQLTDPPGVRRVYHIQAGLPDPFQPPSLPITVCYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGPKAPYTSTLLPPELSETPNATQPELAPEDPEDSALLEDPVGTVAPQIPPNWHIPSIQDAATPYHPPATPNNMGLIAGAVGGSLLAALVICGIVYWMHRRTRKAPKRIRLPHIREDDQPSSHQPLFY

SEQ ID NO: 2 - DNA, 1182 bp, synthetic construct
  source 1..1182: mol_type = other DNA
atggggggggctgccgccaggttgggggccgtgattttgtttgtcgtcatagtgggcctccatggggtccgcggcaaatattgtttggcggatgcctctctcaagatggccgaccccaatcgctttcgcggcaaagaccttccggtcctggaccagctgaccgaccctccgggggtccggcgcgtgtaccacatccaggcgggcctaccggacccgttccagccccccagcctcccgatcacggtttgctacgccgtgttggagcgcgcctgccgcagcgtgctcctaaacgcaccgtcggaggccccccagattgtccgcggggcctccgaagacgtccggaaacaaccctacaacctgaccatcgcttggtttcggatgggaggcaactgtgctatccccatcacggtcatggagtacaccgaatgctcctacaacaagtctctgggggcctgtcccatccgaacgcagccccgctggaactactatgacagcttcagcgccgtcagcgaggataacctggggttcctgatgcacgcccccgcgtttgagaccgccggcacgtacctgcggctcgtgaagataaacgactggacggagattacacagtttatcctggagcaccgagccaagggctcctgtaagtacgccctcccgctgcgcatccccccgtcagcctgcctctccccccaggcctaccagcagggggtgacggtggacagcatcgggatgctgccccgcttcatccccgagaaccagcgcaccgtcgccgtatacagcttgaagatcgccgggtggcacgggcccaaggccccatacacgagcaccctgctgcccccggagctgtccgagacccccaacgccacgcagccagaactcgccccggaagaccccgaggattcggccctcttggaggaccccgtggggacggtggcgccgcaaatcccaccaaactggcacatcccgtcgatccaggacgccgcgacgccttaccatcccccggccaccccgaacaacatgggcctgatcgccggcgcggtgggcggcagtctcctggcagccctggtcatttgcggaattgtgtactggatgcaccgccgcactcggaaagccccaaagcgcatacgcctcccccacatccgggaagacgaccagccgtcctcgcaccagcccttgttttac

SEQ ID NO: 3 - protein, 394 aa, synthetic construct
MGGAAARLGAVILFVVIVGLHGVRGKYALADASLKMADPNRFRGKDLPVLDQLTDPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRAIPENQRTVAVYSLKIAGWHGPKAPYTSTLLPPELSETPNATQPELAPEDPEDSALLEDPVGTVAPQIPPNWHIPSIQDAATPYHPPATPNNMGLIAGAVGGSLLAALVICGIVYWMHRRTRKAPKRIRLPHIREDDQPSSHQPLFY

SEQ ID NO: 4 - DNA, 1182 bp, synthetic construct
  source 1..1182: mol_type = other DNA
atggggggggctgccgccaggttgggggccgtgattttgtttgtcgtcatagtgggcctccatggggtccgcggcaaatatgccttggcggatgcctctctcaagatggccgaccccaatcgctttcgcggcaaagaccttccggtcctggaccagctgaccgaccctccgggggtccggcgcgtgtaccacatccaggcgggcctaccggacccgttccagccccccagcctcccgatcacggtttactacgccgtgttggagcgcgcctgccgcagcgtgctcctaaacgcaccgtcggaggccccccagattgtccgcggggcctccgaagacgtccggaaacaaccctacaacctgaccatcgcttggtttcggatgggaggcaactgtgctatccccatcacggtcatggagtacaccgaatgctcctacaacaagtctctgggggcctgtcccatccgaacgcagccccgctggaactactatgacagcttcagcgccgtcagcgaggataacctggggttcctgatgcacgcccccgcgtttgagaccgccggcacgtacctgcggctcgtgaagataaacgactggacggagattacacagtttatcctggagcaccgagccaagggctcctgtaagtacgccctcccgctgcgcatccccccgtcagcctgcctctccccccaggcctaccagcagggggtgacggtggacagcatcgggatgctgccccgcgctatccccgagaaccagcgcaccgtcgccgtatacagcttgaagatcgccgggtggcacgggcccaaggccccatacacgagcaccctgctgcccccggagctgtccgagacccccaacgccacgcagccagaactcgccccggaagaccccgaggattcggccctcttggaggaccccgtggggacggtggcgccgcaaatcccaccaaactggcacatcccgtcgatccaggacgccgcgacgccttaccatcccccggccaccccgaacaacatgggcctgatcgccggcgcggtgggcggcagtctcctggcagccctggtcatttgcggaattgtgtactggatgcaccgccgcactcggaaagccccaaagcgcatacgcctcccccacatccgggaagacgaccagccgtcctcgcaccagcccttgttttac

SEQ ID NO: 5 - protein, 300 aa, synthetic construct
MGGAAARLGAVILFVVIVGLHGVRGKYALADASLKMADPNRFRGKDLPVLDQLTDPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGPGLIAGAVGGSLLAALVICGIVYWMHRRTRKA

SEQ ID NO: 6 - DNA, 900 bp, synthetic construct
  source 1..900: mol_type = other DNA
atggggggggctgccgccaggttgggggccgtgattttgtttgtcgtcatagtgggcctccatggggtccgcggcaaatatgccttggcggatgcctctctcaagatggccgaccccaatcgctttcgcggcaaagaccttccggtcctggaccagctgaccgaccctccgggggtccggcgcgtgtaccacatccaggcgggcctaccggacccgttccagccccccagcctcccgatcacggtttactacgccgtgttggagcgcgcctgccgcagcgtgctcctaaacgcaccgtcggaggccccccagattgtccgcggggcctccgaagacgtccggaaacaaccctacaacctgaccatcgcttggtttcggatgggaggcaactgtgctatccccatcacggtcatggagtacaccgaatgctcctacaacaagtctctgggggcctgtcccatccgaacgcagccccgctggaactactatgacagcttcagcgccgtcagcgaggataacctggggttcctgatgcacgcccccgcgtttgagaccgccggcacgtacctgcggctcgtgaagataaacgactggacggagattacacagtttatcctggagcaccgagccaagggctcctgtaagtacgccctcccgctgcgcatccccccgtcagcctgcctctccccccaggcctaccagcagggggtgacggtggacagcatcgggatgctgccccgcttcatccccgagaaccagcgcaccgtcgccgtatacagcttgaagatcgccgggtggcacgggcccggcctgatcgccggcgcggtgggcggcagtctcctggcagccctggtcatttgcggaattgtgtactggatgcaccgccgcactcggaaagcc

SEQ ID NO: 7 - protein, 394 aa, synthetic construct
MGGAAARLGAVILFVVIVGLHGVRGKYCLADASLKMADPNRFRGKDLPVLDQLTDPPGVRRVYHIQAGLPDPFQPPSLPITVCYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRAIPENQRTVAVYSLKIAGWHGPKAPYTSTLLPPELSETPNATQPELAPEDPEDSALLEDPVGTVAPQIPPNWHIPSIQDAATPYHPPATPNNMGLIAGAVGGSLLAALVICGIVYWMHRRTRKAPKRIRLPHIREDDQPSSHQPLFY

SEQ ID NO: 8 - DNA, 1182 bp, synthetic construct
  source 1..1182: mol_type = other DNA
atggggggggctgccgccaggttgggggccgtgattttgtttgtcgtcatagtgggcctccatggggtccgcggcaaatattgtttggcggatgcctctctcaagatggccgaccccaatcgctttcgcggcaaagaccttccggtcctggaccagctgaccgaccctccgggggtccggcgcgtgtaccacatccaggcgggcctaccggacccgttccagccccccagcctcccgatcacggtttgctacgccgtgttggagcgcgcctgccgcagcgtgctcctaaacgcaccgtcggaggccccccagattgtccgcggggcctccgaagacgtccggaaacaaccctacaacctgaccatcgcttggtttcggatgggaggcaactgtgctatccccatcacggtcatggagtacaccgaatgctcctacaacaagtctctgggggcctgtcccatccgaacgcagccccgctggaactactatgacagcttcagcgccgtcagcgaggataacctggggttcctgatgcacgcccccgcgtttgagaccgccggcacgtacctgcggctcgtgaagataaacgactggacggagattacacagtttatcctggagcaccgagccaagggctcctgtaagtacgccctcccgctgcgaatccccccgtcagcctgcctctccccccaggcctaccagcagggggtgacggtggacagcatcgggatgctgccccgcgctatccccgagaaccagcgcaccgtcgccgtatacagcttgaagatcgccgggtggcacgggcccaaggccccatacacgagcaccctgctgcccccggagctgtccgagacccccaacgccacgcagccagaactcgccccggaagaccccgaggattcggccctcttggaggaccccgtggggacggtggcgccgcaaatcccaccaaactggcacatcccgtcgatccaggacgccgcgacgccttaccatcccccggccaccccgaacaacatgggcctgatcgccggcgcggtgggcggcagtctcctggcagccctggtcatttgcggaattgtgtactggatgcaccgccgcactcggaaagccccaaagcgcatacgcctcccccacatccgggaagacgaccagccgtcctcgcaccagcccttgttttac

SEQ ID NO: 9 - protein, 351 aa, synthetic construct
MGGAAARLGAVILFVVIVGLHGVRGKYCLADASLKMADPNRFRGKDLPVLDQLTDPPGVRRVYHIQAGLPDPFQPPSLPITVCYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGPATQPELAPEDPEDSALLEDPVGTVAPQIPPNWHIPSIQDAATPYHPPATPNNMGLIAGAVGGSLLAALVICGIVYWMHRRTRKAPKRIRLPHIREDDQPSSHQPLFY

SEQ ID NO: 10 - DNA, 1053 bp, synthetic construct
  source 1..1053: mol_type = other DNA
atggggggggctgccgccaggttgggggccgtgattttgtttgtcgtcatagtgggcctccatggggtccgcggcaaatattgtttggcggatgcctctctcaagatggccgaccccaatcgctttcgcggcaaagaccttccggtcctggaccagctgaccgaccctccgggggtccggcgcgtgtaccacatccaggcgggcctaccggacccgttccagccccccagcctcccgatcacggtttgctacgccgtgttggagcgcgcctgccgcagcgtgctcctaaacgcaccgtcggaggccccccagattgtccgcggggcctccgaagacgtccggaaacaaccctacaacctgaccatcgcttggtttcggatgggaggcaactgtgctatccccatcacggtcatggagtacaccgaatgctcctacaacaagtctctgggggcctgtcccatccgaacgcagccccgctggaactactatgacagcttcagcgccgtcagcgaggataacctggggttcctgatgcacgcccccgcgtttgagaccgccggcacgtacctgcggctcgtgaagataaacgactggacggagattacacagtttatcctggagcaccgagccaagggctcctgtaagtacgccctcccgctgcgcatccccccgtcagcctgcctctccccccaggcctaccagcagggggtgacggtggacagcatcgggcccgccacgcagccagaactcgccccggaagaccccgaggattcggccctcttggaggaccccgtggggacggtggcgccgcaaatcccaccaaactggcacatcccgtcgatccaggacgccgcgacgccttaccatcccccggccaccccgaacaacatgggcctgatcgccggcgcggtgggcggcagtctcctggcagccctggtcatttgcggaattgtgtactggatgcaccgccgcactcggaaagccccaaagcgcatacgcctcccccacatccgggaagacgaccagccgtcctcgcaccagcccttgttttac

SEQ ID NO: 11 - protein, 331 aa, synthetic construct
HGVRGKYCLADASLKMADPNRFRGKDLPVLDQLTDPPGVRRVYHIQAGLPDPFQPPSLPITVCYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGPATQPELAPEDPEDSALLEDPVGTVAPQIPPNWHIPSIQDAATPYHPPATPNNMGLIAGAVGGSLLAALVICGIVYWMHRRTRKAPKRIRLPHIREDDQPSSHQPLFY

SEQ ID NO: 12 - DNA, 993 bp, synthetic construct
  source 1..993: mol_type = other DNA
catggggtccgcggcaaatattgtttggcggatgcctctctcaagatggccgaccccaatcgctttcgcggcaaagaccttccggtcctggaccagctgaccgaccctccgggggtccggcgcgtgtaccacatccaggcgggcctaccggacccgttccagccccccagcctcccgatcacggtttgctacgccgtgttggagcgcgcctgccgcagcgtgctcctaaacgcaccgtcggaggccccccagattgtccgcggggcctccgaagacgtccggaaacaaccctacaacctgaccatcgcttggtttcggatgggaggcaactgtgctatccccatcacggtcatggagtacaccgaatgctcctacaacaagtctctgggggcctgtcccatccgaacgcagccccgctggaactactatgacagcttcagcgccgtcagcgaggataacctggggttcctgatgcacgcccccgcgtttgagaccgccggcacgtacctgcggctcgtgaagataaacgactggacggagattacacagtttatcctggagcaccgagccaagggctcctgtaagtacgccctcccgctgcgcatccccccgtcagcctgcctctccccccaggcctaccagcagggggtgacggtggacagcatcgggcccgccacgcagccagaactcgccccggaagaccccgaggattcggccctcttggaggaccccgtggggacggtggcgccgcaaatcccaccaaactggcacatcccgtcgatccaggacgccgcgacgccttaccatcccccggccaccccgaacaacatgggcctgatcgccggcgcggtgggcggcagtctcctggcagccctggtcatttgcggaattgtgtactggatgcaccgccgcactcggaaagccccaaagcgcatacgcctcccccacatccgggaagacgaccagccgtcctcgcaccagcccttgttttac

SEQ ID NO: 13 - protein, 244 aa, synthetic construct
MGGAAARLGAVILFVVIVGLHGVRGKYCLADASLKMADPNRFRGKDLPVLDQLTDPPGVRRVYHIQAGLPDPFQPPSLPITVCYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGP

SEQ ID NO: 14 - DNA, 732 bp, synthetic construct
  source 1..732: mol_type = other DNA
atggggggggctgccgccaggttgggggccgtgattttgtttgtcgtcatagtgggcctccatggggtccgcggcaaatattgtttggcggatgcctctctcaagatggccgaccccaatcgctttcgcggcaaagaccttccggtcctggaccagctgaccgaccctccgggggtccggcgcgtgtaccacatccaggcgggcctaccggacccgttccagccccccagcctcccgatcacggtttgctacgccgtgttggagcgcgcctgccgcagcgtgctcctaaacgcaccgtcggaggccccccagattgtccgcggggcctccgaagacgtccggaaacaaccctacaacctgaccatcgcttggtttcggatgggaggcaactgtgctatccccatcacggtcatggagtacaccgaatgctcctacaacaagtctctgggggcctgtcccatccgaacgcagccccgctggaactactatgacagcttcagcgccgtcagcgaggataacctggggttcctgatgcacgcccccgcgtttgagaccgccggcacgtacctgcggctcgtgaagataaacgactggacggagattacacagtttatcctggagcaccgagccaagggctcctgtaagtacgccctcccgctgcgcatccccccgtcagcctgcctctccccccaggcctaccagcagggggtgacggtggacagcatcgggccc

SEQ ID NO: 15 - protein, 224 aa, synthetic construct
HGVRGKYCLADASLKMADPNRFRGKDLPVLDQLTDPPGVRRVYHIQAGLPDPFQPPSLPITVCYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGP

SEQ ID NO: 16 - DNA, 672 bp, synthetic construct
  source 1..672: mol_type = other DNA
catggggtccgcggcaaatattgtttggcggatgcctctctcaagatggccgaccccaatcgctttcgcggcaaagaccttccggtcctggaccagctgaccgaccctccgggggtccggcgcgtgtaccacatccaggcgggcctaccggacccgttccagccccccagcctcccgatcacggtttgctacgccgtgttggagcgcgcctgccgcagcgtgctcctaaacgcaccgtcggaggccccccagattgtccgcggggcctccgaagacgtccggaaacaaccctacaacctgaccatcgcttggtttcggatgggaggcaactgtgctatccccatcacggtcatggagtacaccgaatgctcctacaacaagtctctgggggcctgtcccatccgaacgcagccccgctggaactactatgacagcttcagcgccgtcagcgaggataacctggggttcctgatgcacgcccccgcgtttgagaccgccggcacgtacctgcggctcgtgaagataaacgactggacggagattacacagtttatcctggagcaccgagccaagggctcctgtaagtacgccctcccgctgcgcatccccccgtcagcctgcctctccccccaggcctaccagcagggggtgacggtggacagcatcgggccc

SEQ ID NO: 17 - protein, 107 aa, synthetic construct
ATQPELAPEDPEDSALLEDPVGTVAPQIPPNWHIPSIQDAATPYHPPATPNNMGLIAGAVGGSLLAALVICGIVYWMHRRTRKAPKRIRLPHIREDDQPSSHQPLFY

SEQ ID NO: 18 - DNA, 321 bp, synthetic construct
  source 1..321: mol_type = other DNA
gccacgcagccagaactcgccccggaagaccccgaggattcggccctcttggaggaccccgtggggacggtggcgccgcaaatcccaccaaactggcacatcccgtcgatccaggacgccgcgacgccttaccatcccccggccaccccgaacaacatgggcctgatcgccggcgcggtgggcggcagtctcctggcagccctggtcatttgcggaattgtgtactggatgcaccgccgcactcggaaagccccaaagcgcatacgcctcccccacatccgggaagacgaccagccgtcctcgcaccagcccttgttttac

SEQ ID NO: 19 - protein, 337 aa, synthetic construct
MHGDTPTLHEYMLDLQPETTGLYGYGQLNDSSEEEDEIDGPAGQAEPDRAHYNIVTFCCKCDSTLRLCVQSTHVDIRTLEDLLMGTLGIVCPICSQKPGAHQKRTAMFQDPQERPRKLPQLCTELQTTIHDIILECVYCKQQLLRREVYDYAYRHLCIVYRDGNPYAVCDKCLKFYSKISEYRHYCYSLYGTTLEQQYNKPLCDLLIRCINCQKPQCPEEKQRHLDKKQRFHNIRGRWTGRCMSCCRSSRTRRETQLAAATNLDTASTTLLACFLLCFCVLLCVCLLIRPLLLSVSTYTSLIILVLLLWITAASAFRCFIVYIVFVYIPLFLIHTHA

SEQ ID NO: 20 - DNA, 1011 bp, synthetic construct
  source 1..1011: mol_type = other DNA
atgcacggggacactcccaccctgcatgaatatatgctggatctgcagcctgagaccacaggcctgtacggctatggacagctgaacgacagctccgaggaagaggacgaaatcgatgggccagcaggacaggcagagccagacagagcccactacaatattgtgaccttctgctgtaagtgcgatagtacactgcgactgtgcgtgcagtcaacacatgtcgacatccgcactctggaggatctgctgatgggaaccctggggatcgtgtgccctatttgttctcagaagccaggcgctcaccagaaaaggactgcaatgtttcaggatccacaggaacgacccaggaagctgcctcagctgtgcaccgagctgcagactaccatccacgacatcattctggaatgcgtgtattgtaaacagcagctgctgaggagagaggtctacgattatgcatacaggcatctgtgcatcgtgtatagagacggaaacccatacgccgtctgcgataagtgtctgaaattctattccaagatctctgagtacaggcactattgttacagcctgtatggcacaactctggaacagcagtacaacaaacccctgtgcgacctgctgatcagatgcattaattgtcagaagccccagtgtcctgaagagaaacagcggcacctggataagaaacagaggttccataacatcagaggccggtggacaggaagatgcatgtcttgctgtcgctctagtcgaacccgacgagagacacagctggcagctgcaactaatctggacaccgccagtaccacactgctggcttgtttcctgctgtgcttttgcgtgctgctgtgcgtctgtctgctgatccggcctctgctgctgtcagtgagcacatacactagcctgatcattctggtcctgctgctgtggattacagccgcttccgctttccgctgttttatcgtgtatatcgtgttcgtgtacatccccctgtttctgattcacactcatgcc

SEQ ID NO: 21 - protein, 318 aa, synthetic construct
MHGDTPTLHEYMLDLQPETTAAAAAAQLNDSSEEEDEIDGPAGQAEPDRAHYNIVTFCCKCDSTLRLCVQSTHVDIRTLEDLLMGTLGIVCPICSQKPGAHQKRTAMFQDPQERPRKLPQLCTELQTTIHDIILECVYCKQQLLRREVYDAAAAALCIVYRDGNPYAVCDKCLKFYSKISEYRHYCYSLYGTTLEQQYNKPLCDLLIRCINCQKPQCPEEKQRHLDKKQRFHNIRGRWTGRCMSCCRSSRTRRETQLGACVLLCVCLLIRPLLLSVSTYTSLIILVLLLWITAASAFRCFIVYIAFVYIPLFLIHTHA

SEQ ID NO: 22 - DNA, 954 bp, synthetic construct
  source 1..954: mol_type = other DNA
atgcacggcgacacccccaccctgcacgagtacatgctggacctgcagcccgagaccaccgccgccgccgccgccgcccagctgaacgacagcagcgaggaggaggacgagatcgacggccccgccggccaggccgagcccgacagggctcactacaacatcgtgaccttctgctgcaagtgcgacagcaccctgaggctgtgcgtgcagagcacccacgtggacatcaggaccctggaggacctgctgatgggcaccctgggcatcgtgtgccccatctgcagccagaagcccggcgcccaccagaagaggaccgccatgttccaggacccccaggagaggcccaggaagctgccccagctgtgcaccgagctgcagaccaccatccacgacatcatcctggagtgcgtgtactgcaagcagcagctgctgaggagggaggtgtacgacgccgccgccgccgccctgtgcatcgtgtacagggacggcaacccctacgccgtgtgcgacaagtgcctgaagttctacagcaagatcagcgagtacaggcactactgctacagcctgtacggcaccaccctggagcagcagtacaacaagcccctgtgcgacctgctgatcaggtgcatcaactgccagaagccccagtgccccgaggagaagcagaggcacctggacaagaagcagaggttccacaacatcaggggcaggtggaccggcaggtgcatgagctgctgcaggagcagcaggaccaggagggagacccagctgggcgcctgcgtgctgctgtgcgtgtgcctgctgatcaggcccctgctgctgagcgtgagcacctacaccagcctgatcatcctggtgctgctgctgtggatcaccgccgccagcgccttcaggtgcttcatcgtgtacatcgccttcgtgtacatccccctgttcctgatccacacccacgcc

SEQ ID NO: 23 - protein, 350 aa, synthetic construct
MTPTLHEYMLDLQPETTDLYGPAGQAEPDRAHYNIVTFCCKCDSTLRLCVQSTHVDIRTLEDLLMGTLGIVCPICAAPRKLPQLCTELNTSLQDIEITCVYCYDFAFRAACIVYRDGNPYAVCDKCLKFYSKISEYRHYCYSVYGTTLAASVSTYTSLILLVLLLWITAASAFRCFIVYIVFVYIPLFLIHTHARFLIAAETLCQRLNVCQDKILTHYENDSTDLRDHIDYWKHMRLECAIYYKAREMGFKHINHQVVPTLAVSKNKALQAIELQLTLETIYNSQYSNEKWSNTTPIVHLKGDANTLKCLRYRFKKHCKLYTAVSSTWHWTGHNVKHKSAIVTLTYDSEW

SEQ ID NO: 24 - DNA, 1050 bp, synthetic construct
  source 1..1050: mol_type = other DNA
atgacacctacattgcatgaatatatgttagatttgcaaccagagacaactgatctctacggtccagctggacaagcagaaccggacagagcccattacaatattgtaaccttttgttgcaagtgtgactctacgcttcggttgtgcgtacaaagcacacacgtagacattcgtactttggaagacctgttaatgggcacactaggaattgtgtgccccatctgtgctgctcccagaaagttaccacagttatgcacagagctgaacacttcactgcaagacatagaaataacctgtgtatattgctatgactttgcttttcgggctgcttgcatagtatatagagatgggaatccatatgctgtatgtgataaatgtttaaagttttattctaaaattagtgagtatagacattattgttatagtgtatatggaacaacattagctgcttctgtgtctacatacacatcattaatattgttggtattactattgtggataacagcagcctctgcgtttaggtgttttattgtatatattgtatttgtttatataccattatttttaatacatacacatgcacgctttttaattgctgctgagactctttgccaacgtttaaatgtgtgtcaggacaaaatactaacacattatgaaaatgatagtacagacctacgtgaccatatagactattggaaacacatgcgcctagaatgtgctatttattacaaggccagagaaatgggatttaaacatattaaccaccaggtggtgccaacactggctgtatcaaagaataaagcattacaagcaattgaactgcaactaacgttagaaacaatatataactcacaatatagtaatgaaaagtggagtaacactacacccatagtacatttaaaaggtgatgctaatactttaaaatgtttaagatatagatttaaaaagcattgtaaattgtatactgcagtgtcgtctacatggcattggacaggacataatgtaaaacataaaagtgcaattgttacacttacatatgatagtgaatgg

SEQ ID NO: 25 - protein, 306 aa, synthetic construct
MPLSYQHFRKLLLLDEEAGPLEEELPRLADEGLNRRVAEDLNLGNLNVSIPWTHKVGNFTGLYSSTVPVFNPEWQTPSFPKIHLQEDIVDRCKQFVGPLTVNEKRRLKLIMPARFYPNVTKYLPLDKGIKPYYPEHAVNHYFQTRHYLHTLWKAGILYKRETTRSASFCGSPYSWEQELQHGSCWWLQFRNSKPCSEYCLTHLVNLLEDWGPCDEHGEHHIRIPRTPARVTGGVFLVDKNPHNTAESRLVVDFSQFSRGITRVSWPKFAVPNLQSLTNLLSSNLSWLSLDVSAAFYHIPLHPAAMP

SEQ ID NO: 26 - DNA, 918 bp, synthetic construct
  source 1..918: mol_type = other DNA
atgcccctgagctaccagcacttcaggaagctgctgctgctggacgaggaggccggccccctggaggaggagctgcccaggctggccgacgagggcctgaacaggagggtggccgaggacctgaacctgggcaacctgaacgtgagcatcccctggacccacaaggtgggcaacttcaccggcctgtacagcagcaccgtgcccgtgttcaaccccgagtggcagacccccagcttccccaagatccacctgcaggaggacatcgtggacaggtgcaagcagttcgtgggtcccctgaccgtgaacgagaagaggaggctgaagctgatcatgcccgccaggttctaccccaacgtgaccaagtacctgcccctggacaagggcatcaagccctactaccccgagcacgccgtgaaccactacttccagaccaggcactacctgcacaccctgtggaaggccggcatcctgtacaagagggagaccaccaggagcgccagcttctgcggcagcccctacagctgggagcaggagctgcagcacggcagctgctggtggctgcagttcaggaacagcaagccctgcagcgagtactgcctgacccacctggtgaacctgctggaggactggggtccctgcgacgagcacggcgagcaccacatcaggatccccaggacccccgccagggtgaccggcggcgtgttcctggtggacaagaacccccacaacaccgccgagagcaggctggtggtggacttcagccagttcagcaggggcatcaccagggtgagctggcccaagttcgccgtgcccaacctgcagagcctgaccaacctgctgagcagcaacctgagctggctgagcctggacgtgagcgccgccttctaccacatccccctgcaccccgccgccatgccc

SEQ ID NO: 27 - protein, 411 aa, synthetic construct
MHFRKLLLLDEEAGPLEEELPRLADEGLNRRVAEDLNLGNLPEWQTPSFPKIHLQEDIVDRCKQFVGPLTVNEKRRLKLIMPARFYPNVTKYLPLDKGIKPYYPEHAVNHYFQTRHYLHTLWKAGILYKRETTRSASFCGSPYSWEQELQHGSCWWLQFRNSKPCSEYCLTHLVNLLEDWGPCDEHGEHHIRIPRTPARVTQAFTFSPTYKAFLSKQYLNLYPVARQRPGLCQVFADATPTGWGLAMGHQRMRGTFVAPLPIHTAELLAACFARSRSGAKILGTDNSVVLSRKYTSFPWLLGCAANWILRGTSFVYVPSALNPADDVGSNLEDPASRELVVSYVNVNMGLKIRQLLWFHISCLTFGRETVIEYLVSFGVWIRTPPAYRPPNAPILSTLPETTVVRRRDRGR

SEQ ID NO: 28 - DNA, 1233 bp, synthetic construct
  source 1..1233: mol_type = other DNA
atgcattttcgcaaactgctgctgctggatgaagaagcgggaccgctggaagaagaactgccgcgcctggcggatgaaggcctgaaccgccgcgtggcggaagatctgaacctgggcaacctgccggaatggcagaccccgagctttccgaaaattcatctgcaggaagatattgtggatcgctgcaaacagtttgtgggaccgctgaccgtgaacgaaaaacgccgcctgaaactgattatgccggcgcgcttttatccgaacgtgaccaaatatctgccgctggataaaggcattaaaccgtattatccggaacatgcggtgaaccattattttcagacccgccattatctgcataccctgtggaaagcgggcattctgtataaacgcgaaaccacccgcagcgcgagcttttgcggcagcccgtatagctgggaacaggaactgcagcatggcagctgctggtggctgcagtttcgcaacagcaaaccgtgcagcgaatattgcctgacccatctggtgaacctgctggaagattggggaccgtgcgatgaacatggcgaacatcatattcgcattccgcgcaccccggcgcgcgtgacccaggcgtttacctttagcccgacctataaagcgtttctgagcaaacagtatctgaacctgtatccggtggcgcgccagcgcccgggcctgtgccaggtgtttgcggatgcgaccccgaccggctggggcctggcgatgggccatcagcgcatgcgcggcacctttgtggcgccgctgccgattcataccgcggaactgctggcggcgtgctttgcgcgcagccgcagcggcgcgaaaattctgggcaccgataacagcgtggtgctgagccgcaaatataccagctttccgtggctgctgggctgcgcggcgaactggattctgcgcggcaccagctttgtgtatgtgccgagcgcgctgaacccggcggatgatgtgggcagcaacctggaagatccggcgagccgcgaactggtggtgagctatgtgaacgtgaacatgggcctgaaaattcgccagctgctgtggtttcatattagctgcctgacctttggccgcgaaaccgtgattgaatatctggtgagctttggcgtgtggattcgcaccccgccggcgtatcgcccgccgaacgcgccgattctgagcaccctgccggaaaccaccgtggtgcgccgccgagatcgaggccgc

SEQ ID NO: 29 - protein, 500 aa, synthetic construct
MGARASVLSGGELDRWEKIRLRPGGKKKYKLKHIVWASRELERFAVNPGLLETSEGCRQILGQLQPSLQTGSEELRSLYNTVATLYCVHQRIEVKDTKEALEKIEEEQNKSKKKAQQAAADTGNSSQVSQNYPIVQNLQGQMVHQAISPRTLNAWVKVVEEKAFSPEVIPMFSALSEGATPQDLNTMLNTVGGHQAAMQMLKETINEEAAEWDRLHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGGPGHKARVLAEAMSQVTNSATIMMQRGNFRNQRKTVKCFNCGKEGHIAKNCRAPRKKGCWKCGKEGHQMKDCTERQANFLGKIWPSHKGRPGNFLQSRPEPTAPPEESFRFGEETTTPSQKQEPIDKELYPLASLRSLFGNDPSSQ

SEQ ID NO: 30 - DNA, 1500 bp, synthetic construct
  source 1..1500: mol_type = other DNA
atgggcgccagagccagcgtgctgagcgggggagagctggatcgctgggaaaagattagactgaggcccggagggaagaaaaagtacaagctgaaacacatcgtgtgggccagcagggagctggagcgcttcgccgtgaaccccggactgctggagacctccgaaggctgtcggcagatcctggggcagctgcagccttccctgcagacaggctccgaggagctgcggtctctgtataatacagtggccacactgtactgtgtgcaccagcggattgaggtgaaggatacaaaagaggccctggagaagattgaggaggagcagaacaagagcaagaagaaagcccagcaggccgccgccgacaccggcaatagctcccaggtgagccagaactatccaattgtgcagaatctgcagggccagatggtgcaccaggccatttccccacggacactgaacgcctgggtgaaggtggtggaggagaaggccttcagccccgaagtgatccctatgttttccgccctgtccgaaggcgccacccctcaggacctgaacaccatgctgaacacagtggggggacaccaggccgccatgcagatgctgaaggaaaccatcaacgaggaggccgccgagtgggatcggctgcaccccgtgcacgccgggccaatcgcccctggccagatgagggagcccagaggctctgacatcgccggcaccacatctaccctgcaggaacagatcgggtggatgaccaataacccaccaatcccagtgggcgagatctacaagaggtggattattctgggactgaacaaaattgtgcgcatgtattcccctacatccatcctggacatcaggcagggacctaaggaacctttccgcgactacgtggatcggttctacaaaaccctgcgcgccgagcaggccagccaggaagtgaagaattggatgacagagaccctgctggtgcagaacgccaatcctgactgtaaaaccatcctgaaggccctgggacctgccgccacactggaggaaatgatgaccgcctgccagggcgtgggcggccctggccacaaagccagggtgctggccgaggccatgtctcaggtgaccaactctgccacaattatgatgcagcgggggaactttcggaaccagaggaagaccgtgaagtgcttcaactgtggcaaggagggacacatcgccaagaattgccgcgccccacggaagaaggggtgttggaaatgcgggaaggagggccaccagatgaaagactgcacagagcggcaggccaactttctgggcaagatttggcccagccacaagggccgccccggaaactttctgcagtccaggcctgagcctaccgccccccctgaggaatccttccggttcggcgaggagacaaccaccccaagccagaagcaggaacccatcgacaaagagctgtaccccctggccagcctgagatccctgttcgggaatgaccccagctctcag

SEQ ID NO: 31 - protein, 194 aa, synthetic construct
MGMQVQIQSLFLLLLWVPGSRGAKFVAAWTLKAAAAAARKFFHRTCKCTGNFAAAANESFALPYWNFATGRNECDVAAADQLGYSYAIDLPVSVAAAAAASVYDFFVWLAAAAAASTFSFRNALAASQVMNLHNLAATAPDNLGYMAAAIAVVNALLLYAYDYEELYAKVPRNQDWLAAAAFGLANEKSIAAAA

SEQ ID NO: 32 - DNA, 582 bp, synthetic construct
  source 1..582: mol_type = other DNA
atgggtatgcaggtccagatccagtcactcttcctcctcctcctgtgggtgcccggttcacggggtgctaagtttgtggccgcttggacactgaaggccgctgcagccgctgcaaggaagttctttcacagaacctgcaaatgtacaggaaacttcgccgctgcagccaatgagagcttcgccctgccctattggaactttgctaccggccggaatgaatgcgacgtggctgcagccgatcagctcggatactcttatgccatcgacctgcctgtgagtgtcgctgcagccgctgcagcctcagtgtacgatttctttgtctggctggctgcagccgctgcagccagcactttctcctttcgcaacgccctggctgcatctcaggtcatgaacctccataatctggcagctaccgcaccagacaacctcggatatatggcagccgctattgccgtggtcaatgctctgctcctgtacgcctatgactacgaggaactgtacgctaaagtccccaggaatcaggattggctcgcagccgctgcatttggtctggccaacgaaaagagcattgcagcagcagca

SEQ ID NO: 33 - protein, 394 aa, synthetic construct
MGGAAARLGAVILFVVIVGLHGVRGKYALADASLKMADPNRFRGKDLPVLDQLTDPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGPKAPYTSTLLPPELSETPNATQPELAPEDPEDSALLEDPVGTVAPQIPPNWHIPSIQDAATPYHPPATPNNMGLIAGAVGGSLLAALVICGIVYWMHRRTRKAPKRIRLPHIREDDQPSSHQPLFY

SEQ ID NO: 34 - protein, 74 aa, synthetic construct
TPTLHEYMLDLQPETTDLYGPAGQAEPDRAHYNIVTFCCKCDSTLRLCVQSTHVDIRTLEDLLMGTLGIVCPIC

SEQ ID NO: 35 - DNA, 222 bp, synthetic construct
  source 1..222: mol_type = other DNA
acacctacattgcatgaatatatgttagatttgcaaccagagacaactgatctctacggtccagctggacaagcagaaccggacagagcccattacaatattgtaaccttttgttgcaagtgtgactctacgcttcggttgtgcgtacaaagcacacacgtagacattcgtactttggaagacctgttaatgggcacactaggaattgtgtgccccatctgt

SEQ ID NO: 36 - protein, 71 aa, synthetic construct
PRKLPQLCTELNTSLQDIEITCVYCYDFAFRAACIVYRDGNPYAVCDKCLKFYSKISEYRHYCYSVYGTTL

SEQ ID NO: 37 - DNA, 213 bp, synthetic construct
  source 1..213: mol_type = other DNA
cccagaaagttaccacagttatgcacagagctgaacacttcactgcaagacatagaaataacctgtgtatattgctatgactttgcttttcgggctgcttgcatagtatatagagatgggaatccatatgctgtatgtgataaatgtttaaagttttattctaaaattagtgagtatagacattattgttatagtgtatatggaacaacatta

SEQ ID NO: 38 - protein, 48 aa, synthetic construct
SVSTYTSLILLVLLLWITAASAFRCFIVYIVFVYIPLFLIHTHARFLI

SEQ ID NO: 39 - DNA, 144 bp, synthetic construct
  source 1..144: mol_type = other DNA
tctgtgtctacatacacatcattaatattgttggtattactattgtggataacagcagcctctgcgtttaggtgttttattgtatatattgtatttgtttatataccattatttttaatacatacacatgcacgctttttaatt

SEQ ID NO: 40 - protein, 150 aa, synthetic construct
ETLCQRLNVCQDKILTHYENDSTDLRDHIDYWKHMRLECAIYYKAREMGFKHINHQVVPTLAVSKNKALQAIELQLTLETIYNSQYSNEKWSNTTPIVHLKGDANTLKCLRYRFKKHCKLYTAVSSTWHWTGHNVKHKSAIVTLTYDSEW

SEQ ID NO: 41 - DNA, 450 bp, synthetic construct
  source 1..450: mol_type = other DNA
gagactctttgccaacgtttaaatgtgtgtcaggacaaaatactaacacattatgaaaatgatagtacagacctacgtgaccatatagactattggaaacacatgcgcctagaatgtgctatttattacaaggccagagaaatgggatttaaacatattaaccaccaggtggtgccaacactggctgtatcaaagaataaagcattacaagcaattgaactgcaactaacgttagaaacaatatataactcacaatatagtaatgaaaagtggagtaacactacacccatagtacatttaaaaggtgatgctaatactttaaaatgtttaagatatagatttaaaaagcattgtaaattgtatactgcagtgtcgtctacatggcattggacaggacataatgtaaaacataaaagtgcaattgttacacttacatatgatagtgaatgg

SEQ ID NO: 42 - protein, 349 aa, synthetic construct
TPTLHEYMLDLQPETTDLYGPAGQAEPDRAHYNIVTFCCKCDSTLRLCVQSTHVDIRTLEDLLMGTLGIVCPICAAPRKLPQLCTELNTSLQDIEITCVYCYDFAFRAACIVYRDGNPYAVCDKCLKFYSKISEYRHYCYSVYGTTLAASVSTYTSLILLVLLLWITAASAFRCFIVYIVFVYIPLFLIHTHARFLIAAETLCQRLNVCQDKILTHYENDSTDLRDHIDYWKHMRLECAIYYKAREMGFKHINHQVVPTLAVSKNKALQAIELQLTLETIYNSQYSNEKWSNTTPIVHLKGDANTLKCLRYRFKKHCKLYTAVSSTWHWTGHNVKHKSAIVTLTYDSEW

SEQ ID NO: 43 - DNA, 1047 bp, synthetic construct
  source 1..1047: mol_type = other DNA
acacctacattgcatgaatatatgttagatttgcaaccagagacaactgatctctacggtccagctggacaagcagaaccggacagagcccattacaatattgtaaccttttgttgcaagtgtgactctacgcttcggttgtgcgtacaaagcacacacgtagacattcgtactttggaagacctgttaatgggcacactaggaattgtgtgccccatctgtgctgctcccagaaagttaccacagttatgcacagagctgaacacttcactgcaagacatagaaataacctgtgtatattgctatgactttgcttttcgggctgcttgcatagtatatagagatgggaatccatatgctgtatgtgataaatgtttaaagttttattctaaaattagtgagtatagacattattgttatagtgtatatggaacaacattagctgcttctgtgtctacatacacatcattaatattgttggtattactattgtggataacagcagcctctgcgtttaggtgttttattgtatatattgtatttgtttatataccattatttttaatacatacacatgcacgctttttaattgctgctgagactctttgccaacgtttaaatgtgtgtcaggacaaaatactaacacattatgaaaatgatagtacagacctacgtgaccatatagactattggaaacacatgcgcctagaatgtgctatttattacaaggccagagaaatgggatttaaacatattaaccaccaggtggtgccaacactggctgtatcaaagaataaagcattacaagcaattgaactgcaactaacgttagaaacaatatataactcacaatatagtaatgaaaagtggagtaacactacacccatagtacatttaaaaggtgatgctaatactttaaaatgtttaagatatagatttaaaaagcattgtaaattgtatactgcagtgtcgtctacatggcattggacaggacataatgtaaaacataaaagtgcaattgttacacttacatatgatagtgaatgg

SEQ ID NO: 44 - protein, 702 aa, synthetic construct
MGGAAARLGAVILFVVIVGLHGVRGKYCLADASLKMADPNRFRGKDLPVLDQLTDPPGVRRVYHIQAGLPDPFQPPSLPITVCYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGPTPTLHEYMLDLQPETTDLYGPAGQAEPDRAHYNIVTFCCKCDSTLRLCVQSTHVDIRTLEDLLMGTLGIVCPICAAPRKLPQLCTELNTSLQDIEITCVYCYDFAFRAACIVYRDGNPYAVCDKCLKFYSKISEYRHYCYSVYGTTLAASVSTYTSLILLVLLLWITAASAFRCFIVYIVFVYIPLFLIHTHARFLIAAETLCQRLNVCQDKILTHYENDSTDLRDHIDYWKHMRLECAIYYKAREMGFKHINHQVVPTLAVSKNKALQAIELQLTLETIYNSQYSNEKWSNTTPIVHLKGDANTLKCLRYRFKKHCKLYTAVSSTWHWTGHNVKHKSAIVTLTYDSEWGPATQPELAPEDPEDSALLEDPVGTVAPQIPPNWHIPSIQDAATPYHPPATPNNMGLIAGAVGGSLLAALVICGIVYWMHRRTRKAPKRIRLPHIREDDQPSSHQPLFY

SEQ ID NO: 45 - DNA, 2106 bp, synthetic construct
  source 1..2106: mol_type = other DNA
atggggggggctgccgccaggttgggggccgtgattttgtttgtcgtcatagtgggcctccatggggtccgcggcaaatattgtttggcggatgcctctctcaagatggccgaccccaatcgctttcgcggcaaagaccttccggtcctggaccagctgaccgaccctccgggggtccggcgcgtgtaccacatccaggcgggcctaccggacccgttccagccccccagcctcccgatcacggtttgctacgccgtgttggagcgcgcctgccgcagcgtgctcctaaacgcaccgtcggaggccccccagattgtccgcggggcctccgaagacgtccggaaacaaccctacaacctgaccatcgcttggtttcggatgggaggcaactgtgctatccccatcacggtcatggagtacaccgaatgctcctacaacaagtctctgggggcctgtcccatccgaacgcagccccgctggaactactatgacagcttcagcgccgtcagcgaggataacctggggttcctgatgcacgcccccgcgtttgagaccgccggcacgtacctgcggctcgtgaagataaacgactggacggagattacacagtttatcctggagcaccgagccaagggctcctgtaagtacgccctcccgctgcgcatccccccgtcagcctgcctctccccccaggcctaccagcagggggtgacggtggacagcatcgggcccacacctacattgcatgaatatatgttagatttgcaaccagagacaactgatctctacggtccagctggacaagcagaaccggacagagcccattacaatattgtaaccttttgttgcaagtgtgactctacgcttcggttgtgcgtacaaagcacacacgtagacattcgtactttggaagacctgttaatgggcacactaggaattgtgtgccccatctgtgctgctcccagaaagttaccacagttatgcacagagctgaacacttcactgcaagacatagaaataacctgtgtatattgctatgactttgcttttcgggctgcttgcatagtatatagagatgggaatccatatgctgtatgtgataaatgtttaaagttttattctaaaattagtgagtatagacattattgttatagtgtatatggaacaacattagctgcttctgtgtctacatacacatcattaatattgttggtattactattgtggataacagcagcctctgcgtttaggtgttttattgtatatattgtatttgtttatataccattatttttaatacatacacatgcacgctttttaattgctgctgagactctttgccaacgtttaaatgtgtgtcaggacaaaatactaacacattatgaaaatgatagtacagacctacgtgaccatatagactattggaaacacatgcgcctagaatgtgctatttattacaaggccagagaaatgggatttaaacatattaaccaccaggtggtgccaacactggctgtatcaaagaataaagcattacaagcaattgaactgcaactaacgttagaaacaatatataactcacaatatagtaatgaaaagtggagtaacactacacccatagtacatttaaaaggtgatgctaatactttaaaatgtttaagatatagatttaaaaagcattgtaaattgtatactgcagtgtcgtctacatggcattggacaggacataatgtaaaacataaaagtgcaattgttacacttacatatgatagtgaatgggggcccgccacgcagccagaactcgccccggaagaccccgaggattcggccctcttggaggaccccgtggggacggtggcgccgcaaatcccaccaaactggcacatcccgtcgatccaggacgccgcgacgccttaccatcccccggccaccccgaacaacatgggcctgatcgccggcgcggtgggcggcagtctcctggcagccctggtcatttgcggaattgtgtactggatgcaccgccgcactcggaaagccccaaagcgcatacgcctcccccacatccgggaagacgaccagccgtcctcgcaccagcccttgttttac

SEQ ID NO: 46 - protein, 682 aa, synthetic construct
HGVRGKYCLADASLKMADPNRFRGKDLPVLDQLTDPPGVRRVYHIQAGLPDPFQPPSLPITVCYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGPTPTLHEYMLDLQPETTDLYGPAGQAEPDRAHYNIVTFCCKCDSTLRLCVQSTHVDIRTLEDLLMGTLGIVCPICAAPRKLPQLCTELNTSLQDIEITCVYCYDFAFRAACIVYRDGNPYAVCDKCLKFYSKISEYRHYCYSVYGTTLAASVSTYTSLILLVLLLWITAASAFRCFIVYIVFVYIPLFLIHTHARFLIAAETLCQRLNVCQDKILTHYENDSTDLRDHIDYWKHMRLECAIYYKAREMGFKHINHQVVPTLAVSKNKALQAIELQLTLETIYNSQYSNEKWSNTTPIVHLKGDANTLKCLRYRFKKHCKLYTAVSSTWHWTGHNVKHKSAIVTLTYDSEWGPATQPELAPEDPEDSALLEDPVGTVAPQIPPNWHIPSIQDAATPYHPPATPNNMGLIAGAVGGSLLAALVICGIVYWMHRRTRKAPKRIRLPHIREDDQPSSHQPLFY

SEQ ID NO: 47 - DNA, 2046 bp, synthetic construct
  source 1..2046: mol_type = other DNA
catggggtccgcggcaaatattgtttggcggatgcctctctcaagatggccgaccccaatcgctttcgcggcaaagaccttccggtcctggaccagctgaccgaccctccgggggtccggcgcgtgtaccacatccaggcgggcctaccggacccgttccagccccccagcctcccgatcacggtttgctacgccgtgttggagcgcgcctgccgcagcgtgctcctaaacgcaccgtcggaggccccccagattgtccgcggggcctccgaagacgtccggaaacaaccctacaacctgaccatcgcttggtttcggatgggaggcaactgtgctatccccatcacggtcatggagtacaccgaatgctcctacaacaagtctctgggggcctgtcccatccgaacgcagccccgctggaactactatgacagcttcagcgccgtcagcgaggataacctggggttcctgatgcacgcccccgcgtttgagaccgccggcacgtacctgcggctcgtgaagataaacgactggacggagattacacagtttatcctggagcaccgagccaagggctcctgtaagtacgccctcccgctgcgcatccccccgtcagcctgcctctccccccaggcctaccagcagggggtgacggtggacagcatcgggcccacacctacattgcatgaatatatgttagatttgcaaccagagacaactgatctctacggtccagctggacaagcagaaccggacagagcccattacaatattgtaaccttttgttgcaagtgtgactctacgcttcggttgtgcgtacaaagcacacacgtagacattcgtactttggaagacctgttaatgggcacactaggaattgtgtgccccatctgtgctgctcccagaaagttaccacagttatgcacagagctgaacacttcactgcaagacatagaaataacctgtgtatattgctatgactttgcttttcgggctgcttgcatagtatatagagatgggaatccatatgctgtatgtgataaatgtttaaagttttattctaaaattagtgagtatagacattattgttatagtgtatatggaacaacattagctgcttctgtgtctacatacacatcattaatattgttggtattactattgtggataacagcagcctctgcgtttaggtgttttattgtatatattgtatttgtttatataccattatttttaatacatacacatgcacgctttttaattgctgctgagactctttgccaacgtttaaatgtgtgtcaggacaaaatactaacacattatgaaaatgatagtacagacctacgtgaccatatagactattggaaacacatgcgcctagaatgtgctatttattacaaggccagagaaatgggatttaaacatattaaccaccaggtggtgccaacactggctgtatcaaagaataaagcattacaagcaattgaactgcaactaacgttagaaacaatatataactcacaatatagtaatgaaaagtggagtaacactacacccatagtacatttaaaaggtgatgctaatactttaaaatgtttaagatatagatttaaaaagcattgtaaattgtatactgcagtgtcgtctacatggcattggacaggacataatgtaaaacataaaagtgcaattgttacacttacatatgatagtgaatgggggcccgccacgcagccagaactcgccccggaagaccccgaggattcggccctcttggaggaccccgtggggacggtggcgccgcaaatcccaccaaactggcacatcccgtcgatccaggacgccgcgacgccttaccatcccccggccaccccgaacaacatgggcctgatcgccggcgcggtgggcggcagtctcctggcagccctggtcatttgcggaattgtgtactggatgcaccgccgcactcggaaagccccaaagcgcatacgcctcccccacatccgggaagacgaccagccgtcctcgcaccagcccttgttttac

SEQ ID NO: 48 - DNA, 1182 bp, synthetic construct
  source 1..1182: mol_type = other DNA
atggggggggctgccgccaggttgggggccgtgattttgtttgtcgtcatagtgggcctccatggggtccgcggcaaatatgccttggcggatgcctctctcaagatggccgaccccaatcgctttcgcggcaaagaccttccggtcctggaccagctgaccgaccctccgggggtccggcgcgtgtaccacatccaggcgggcctaccggacccgttccagccccccagcctcccgatcacggtttactacgccgtgttggagcgcgcctgccgcagcgtgctcctaaacgcaccgtcggaggccccccagattgtccgcggggcctccgaagacgtccggaaacaaccctacaacctgaccatcgcttggtttcggatgggaggcaactgtgctatccccatcacggtcatggagtacaccgaatgctcctacaacaagtctctgggggcctgtcccatccgaacgcagccccgctggaactactatgacagcttcagcgccgtcagcgaggataacctggggttcctgatgcacgcccccgcgtttgagaccgccggcacgtacctgcggctcgtgaagataaacgactggacggagattacacagtttatcctggagcaccgagccaagggctcctgtaagtacgccctcccgctgcgcatccccccgtcagcctgcctctccccccaggcctaccagcagggggtgacggtggacagcatcgggatgctgccccgcttcatccccgagaaccagcgcaccgtcgccgtatacagcttgaagatcgccgggtggcacgggcccaaggccccatacacgagcaccctgctgcccccggagctgtccgagacccccaacgccacgcagccagaactcgccccggaagaccccgaggattcggccctcttggaggaccccgtggggacggtggcgccgcaaatcccaccaaactggcacatcccgtcgatccaggacgccgcgacgccttaccatcccccggccaccccgaacaacatgggcctgatcgccggcgcggtgggcggcagtctcctggcagccctggtcatttgcggaattgtgtactggatgcaccgccgcactcggaaagccccaaagcgcatacgcctcccccacatccgggaagacgaccagccgtcctcgcaccagcccttgttttac

SEQ ID NO: 49 - protein, 410 aa, synthetic construct
MGGAAARLGAVILFVVIVGLHGVRGKYCLADASLKMADPNRFRGKDLPVLDQLTDPPGVRRVYHIQAGLPDPFQPPSLPITVCYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGPKAPYTSTLLPPELSETPNATQPELAPEDPEDSALLEDPVGTVAPQIPPNWHIPSIQDAATPYHPPATPNNMGLIAGAVGGSLLAALVICGIVYWMHRRTRKAPKRIRLPHIREDDQPSSHQPLFYHIREDDQPSSHQPLFY

SEQ ID NO: 50 - protein, 381 aa, synthetic construct
MGGAAARLGAVILFVVIVGLHGVRGKYALADASLKMADPNRFRGKDLPVLDQLTDPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGPNRWPRIRYHAAAVYMTSTLLPPELSETPNATQPELAPEDPEDSALLEDPVGTVAPQIPPNWHIPSIQDAATPYHPPATPNNMGLITRKAPKRIRLPHIREDDQPSSHQPLFY

SEQ ID NO: 51 - DNA, 1143 bp, synthetic construct
  source 1..1143: mol_type = other DNA
atggggggggctgccgccaggttgggggccgtgattttgtttgtcgtcatagtgggcctccatggggtccgcggcaaatatgccttggcggatgcctctctcaagatggccgaccccaatcgctttcgcggcaaagaccttccggtcctggaccagctgaccgaccctccgggggtccggcgcgtgtaccacatccaggcgggcctaccggacccgttccagccccccagcctcccgatcacggtttactacgccgtgttggagcgcgcctgccgcagcgtgctcctaaacgcaccgtcggaggccccccagattgtccgcggggcctccgaagacgtccggaaacaaccctacaacctgaccatcgcttggtttcggatgggaggcaactgtgctatccccatcacggtcatggagtacaccgaatgctcctacaacaagtctctgggggcctgtcccatccgaacgcagccccgctggaactactatgacagcttcagcgccgtcagcgaggataacctggggttcctgatgcacgcccccgcgtttgagaccgccggcacgtacctgcggctcgtgaagataaacgactggacggagattacacagtttatcctggagcaccgagccaagggctcctgtaagtacgccctcccgctgcgcatccccccgtcagcctgcctctccccccaggcctaccagcagggggtgacggtggacagcatcgggatgctgccccgcttcatccccgagaaccagcgcaccgtcgccgtatacagcttgaagatcgccgggtggcacgggcccaaccggtggcctaggatccgatatcacgcggccgcagtgtacatgacgagcaccctgctgcccccggagctgtccgagacccccaacgccacgcagccagaactcgccccggaagaccccgaggattcggccctcttggaggaccccgtggggacggtggcgccgcaaatcccaccaaactggcacatcccgtcgatccaggacgccgcgacgccttaccatcccccggccaccccgaacaacatgggcctgatcactcggaaagccccaaagcgcatacgcctcccccacatccgggaagacgaccagccgtcctcgcaccagcccttgttttac

SEQ ID NO: 52 - protein, 419 aa, synthetic construct
MGGAAARLGAVILFVVIVGLHGVRGKYALADASLKMADPNRFRGKDLPVLDQLTDPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGPKAPYTSTLLPPELSETPNATQPELAPEDPEDSALLEDPVGTVAPQIPPNAHIPSIQDAATPYHPPATPNNMGLIAGAVGGSLLAALVICGIVYWMHRRTRKAPKRIRLPHIREDDQPSSHQPLFYLSTGSGATNFSLLKQAGDVEENPGP

SEQ ID NO: 53 - DNA, 1257 bp, synthetic construct
  source 1..1257: mol_type = other DNA
atggggggggctgccgccaggttgggggccgtgattttgtttgtcgtcatagtgggcctccatggggtccgcggcaaatatgccttggcggatgcctctctcaagatggccgaccccaatcgctttcgcggcaaagaccttccggtcctggaccagctgaccgaccctccgggggtccggcgcgtgtaccacatccaggcgggcctaccggacccgttccagccccccagcctcccgatcacggtttactacgccgtgttggagcgcgcctgccgcagcgtgctcctaaacgcaccgtcggaggccccccagattgtccgcggggcctccgaagacgtccggaaacaaccctacaacctgaccatcgcttggtttcggatgggaggcaactgtgctatccccatcacggtcatggagtacaccgaatgctcctacaacaagtctctgggggcctgtcccatccgaacgcagccccgctggaactactatgacagcttcagcgccgtcagcgaggataacctggggttcctgatgcacgcccccgcgtttgagaccgccggcacgtacctgcggctcgtgaagataaacgactggacggagattacacagtttatcctggagcaccgagccaagggctcctgtaagtacgccctcccgctgcgcatccccccgtcagcctgcctgtccccccaggcctaccagcagggggtgacggtggacagcatcgggatgctgccccgcttcatccccgagaaccagcgcaccgtcgccgtatacagcttgaagatcgccgggtggcacgggcccaaggccccatacacgagcaccctgctgcccccggagctgtccgagacccctaacgccacgcagccagaactcgccccggaagaccccgaggattcggccctcttggaggaccccgtggggacggtggcgccgcaaatcccaccaaacgcgcacataccgtcgatccaggacgccgcgacgccttaccatcccccggccaccccgaacaacatgggcctgatcgccggcgcggtgggcggcagtctcctggcagccctggtcatttgcggaattgtgtactggatgcaccgccgcactcggaaagccccaaagcgcatacgcctcccccacatccgggaagacgaccagccgtcctcgcaccagcccttgttttacttgtcgacaggaagcggagctactaacttcagcctgctgaagcaggctggagacgtggaggagaaccctggacct

SEQ ID NO: 54 - protein, 337 aa, synthetic construct
HGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQKPGAHQKRTAMFQDPQERPRKLPQLCTELQTTIHDIILECVYCKQQLLRREVYDFAFRDLCIVYRDGNPYAVCDKCLKFYSKISEYRHYCYSLYGTTLEQQYNKPLCDLLIRCINCQKPLCPEEKQRHLDKKQRFHNIRGRWTGRCMSCCRSSRTRRETQLAAACIHNITGVLFALLCVLLCVCLLIRPLLLSVSTYTSLIILVLLLWITAASAFRCFIVYIIFVYIPLFLIHTHARFLITG

SEQ ID NO: 55 - DNA, 1013 bp, synthetic construct
  source 1..1013: mol_type = other DNA
catggagatacacctacattgcatgaatatatgttagatttgcaaccagagacaactgatctctactgttatgagcaattaaatgacagctcagaggaggaggatgaaatagatggtccagctggacaagcagaaccggacagagcccattacaatattgtaaccttttgttgcaagtgtgactctacgcttcggttgtgcgtacaaagcacacacgtagacattcgtactttggaagacctgttaatgggcacactaggaattgtgtgccccatctgttctcagaaaccaggcgcccaccaaaagagaactgcaatgtttcaggacccacaggagcgacccagaaagttaccacagttatgcacagagctgcaaacaactatacatgatataatattagaatgtgtgtactgcaagcaacagttactgcgacgtgaggtatatgactttgcttttcgggatttatgcatagtatatagagatgggaatccatatgctgtatgtgataaatgtttaaagttttattctaaaattagtgagtatagacattattgttatagtttgtatggaacaacattagaacagcaatacaacaaaccgttgtgtgatttgttaattaggtgtattaactgtcaaaagccactgtgtcctgaagaaaagcaaagacatctggacaaaaagcaaagattccataatataaggggtcggtggaccggtcgatgtatgtcttgttgcagatcatcaagaacacgtagagaaacccagctggcggccgcctgcatccacaacattactggcgtgctttttgctttgctttgtgtgcttttgtgtgtctgcctattaatacgtccgctgcttttgtctgtgtctacatacacatcattaataatattggtattactattgtggataacagcagcctctgcgtttaggtgttttattgtatatattatatttgtttatataccattatttttaatacatacacatgcacgctttttaattacagggcc

SEQ ID NO: 56 - protein, 59 aa, synthetic construct
SNTTPIVHLKGDANTLKCLRYRFKKHCKLYTAVSSTWHWTGHNVKHKSAIVTLTYDSEW

SEQ ID NO: 57 - DNA, 177 bp, synthetic construct
  source 1..177: mol_type = other DNA
agtaacactacacccatagtacatttaaaaggtgatgctaatactttaaaatgtttaagatatagatttaaaaagcattgtaaattgtatactgcagtgtcgtctacatggcattggacaggacataatgtaaaacataaaagtgcaattgttacacttacatatgatagtgaatgg

SEQ ID NO: 58 - protein, 91 aa, synthetic construct
ETLCQRLNVCQDKILTHYENDSTDLRDHIDYWKHMRLECAIYYKAREMGFKHINHQVVPTLAVSKNKALQAIELQLTLETIYNSQYSNEKW

SEQ ID NO: 59 - DNA, 273 bp, synthetic construct
  source 1..273: mol_type = other DNA
gagactctttgccaacgtttaaatgtgtgtcaggacaaaatactaacacattatgaaaatgatagtacagacctacgtgaccatatagactattggaaacacatgcgcctagaatgtgctatttattacaaggccagagaaatgggatttaaacatattaaccaccaggtggtgccaacactggctgtatcaaagaataaagcattacaagcaattgaactgcaactaacgttagaaacaatatataactcacaatatagtaatgaaaagtgg

SEQ ID NO: 60 - protein, 258 aa, synthetic construct
TPTLHEYMLDLQPETTDLYGPAGQAEPDRAHYNIVTFCCKCDSTLRLCVQSTHVDIRTLEDLLMGTLGIVCPICAAPRKLPQLCTELNTSLQDIEITCVYCYDFAFRAACIVYRDGNPYAVCDKCLKFYSKISEYRHYCYSVYGTTLAASVSTYTSLILLVLLLWITAASAFRCFIVYIVFVYIPLFLIHTHARFLIAASNTTPIVHLKGDANTLKCLRYRFKKHCKLYTAVSSTWHWTGHNVKHKSAIVTLTYDSEW

SEQ ID NO: 61 - DNA, 774 bp, synthetic construct
  source 1..774: mol_type = other DNA
acacctacattgcatgaatatatgttagatttgcaaccagagacaactgatctctacggtccagctggacaagcagaaccggacagagcccattacaatattgtaaccttttgttgcaagtgtgactctacgcttcggttgtgcgtacaaagcacacacgtagacattcgtactttggaagacctgttaatgggcacactaggaattgtgtgccccatctgtgctgctcccagaaagttaccacagttatgcacagagctgaacacttcactgcaagacatagaaataacctgtgtatattgctatgactttgcttttcgggctgcttgcatagtatatagagatgggaatccatatgctgtatgtgataaatgtttaaagttttattctaaaattagtgagtatagacattattgttatagtgtatatggaacaacattagctgcttctgtgtctacatacacatcattaatattgttggtattactattgtggataacagcagcctctgcgtttaggtgttttattgtatatattgtatttgtttatataccattatttttaatacatacacatgcacgctttttaattgctgctagtaacactacacccatagtacatttaaaaggtgatgctaatactttaaaatgtttaagatatagatttaaaaagcattgtaaattgtatactgcagtgtcgtctacatggcattggacaggacataatgtaaaacataaaagtgcaattgttacacttacatatgatagtgaatgg

SEQ ID NO: 62 - protein, 290 aa, synthetic construct
TPTLHEYMLDLQPETTDLYGPAGQAEPDRAHYNIVTFCCKCDSTLRLCVQSTHVDIRTLEDLLMGTLGIVCPICAAPRKLPQLCTELNTSLQDIEITCVYCYDFAFRAACIVYRDGNPYAVCDKCLKFYSKISEYRHYCYSVYGTTLAASVSTYTSLILLVLLLWITAASAFRCFIVYIVFVYIPLFLIHTHARFLIAAETLCQRLNVCQDKILTHYENDSTDLRDHIDYWKHMRLECAIYYKAREMGFKHINHQVVPTLAVSKNKALQAIELQLTLETIYNSQYSNEKW

SEQ ID NO: 63 - DNA, 870 bp, synthetic construct
  source 1..870: mol_type = other DNA
acacctacattgcatgaatatatgttagatttgcaaccagagacaactgatctctacggtccagctggacaagcagaaccggacagagcccattacaatattgtaaccttttgttgcaagtgtgactctacgcttcggttgtgcgtacaaagcacacacgtagacattcgtactttggaagacctgttaatgggcacactaggaattgtgtgccccatctgtgctgctcccagaaagttaccacagttatgcacagagctgaacacttcactgcaagacatagaaataacctgtgtatattgctatgactttgcttttcgggctgcttgcatagtatatagagatgggaatccatatgctgtatgtgataaatgtttaaagttttattctaaaattagtgagtatagacattattgttatagtgtatatggaacaacattagctgcttctgtgtctacatacacatcattaatattgttggtattactattgtggataacagcagcctctgcgtttaggtgttttattgtatatattgtatttgtttatataccattatttttaatacatacacatgcacgctttttaattgctgctgagactctttgccaacgtttaaatgtgtgtcaggacaaaatactaacacattatgaaaatgatagtacagacctacgtgaccatatagactattggaaacacatgcgcctagaatgtgctatttattacaaggccagagaaatgggatttaaacatattaaccaccaggtggtgccaacactggctgtatcaaagaataaagcattacaagcaattgaactgcaactaacgttagaaacaatatataactcacaatatagtaatgaaaagtgg

SEQ ID NO: 64 - protein, 94 aa, synthetic construct
TPTLHEYMLDLQPETTAAAAAAQLNDSSEEEDEIDGPAGQAEPDRAHYNIVTFCCKCDSTLRLCVQSTHVDIRTLEDLLMGTLGIVCPICSQKP

SEQ ID NO: 65 - DNA, 282 bp, synthetic construct
  source 1..282: mol_type = other DNA
acccccaccctgcacgagtacatgctggacctgcagcccgagaccaccgccgccgccgccgccgcccagctgaacgacagcagcgaggaggaggacgagatcgacggccccgccggccaggccgagcccgacagggctcactacaacatcgtgaccttctgctgcaagtgcgacagcaccctgaggctgtgcgtgcagagcacccacgtggacatcaggaccctggaggacctgctgatgggcaccctgggcatcgtgtgccccatctgcagccagaagccc

SEQ ID NO: 66 - protein, 157 aa, synthetic construct
HQKRTAMFQDPQERPRKLPQLCTELQTTIHDIILECVYCKQQLLRREVYDAAAAALCIVYRDGNPYAVCDKCLKFYSKISEYRHYCYSLYGTTLEQQYNKPLCDLLIRCINCQKPQCPEEKQRHLDKKQRFHNIRGRWTGRCMSCCRSSRTRRETQL

SEQ ID NO: 67 - DNA, 471 bp, synthetic construct
  source 1..471: mol_type = other DNA
caccagaagaggaccgccatgttccaggacccccaggagaggcccaggaagctgccccagctgtgcaccgagctgcagaccaccatccacgacatcatcctggagtgcgtgtactgcaagcagcagctgctgaggagggaggtgtacgacgccgccgccgccgccctgtgcatcgtgtacagggacggcaacccctacgccgtgtgcgacaagtgcctgaagttctacagcaagatcagcgagtacaggcactactgctacagcctgtacggcaccaccctggagcagcagtacaacaagcccctgtgcgacctgctgatcaggtgcatcaactgccagaagccccagtgccccgaggagaagcagaggcacctggacaagaagcagaggttccacaacatcaggggcaggtggaccggcaggtgcatgagctgctgcaggagcagcaggaccaggagggagacccagctg

SEQ ID NO: 68 - protein, 59 aa, synthetic construct
CVLLCVCLLIRPLLLSVSTYTSLIILVLLLWITAASAFRCFIVYIAFVYIPLFLIHTHA

SEQ ID NO: 69 - DNA, 177 bp, synthetic construct
  source 1..177: mol_type = other DNA
tgcgtgctgctgtgcgtgtgcctgctgatcaggcccctgctgctgagcgtgagcacctacaccagcctgatcatcctggtgctgctgctgtggatcaccgccgccagcgccttcaggtgcttcatcgtgtacatcgccttcgtgtacatccccctgttcctgatccacacccacgcc

SEQ ID NO: 70 - protein, 314 aa, synthetic construct
TPTLHEYMLDLQPETTAAAAAAQLNDSSEEEDEIDGPAGQAEPDRAHYNIVTFCCKCDSTLRLCVQSTHVDIRTLEDLLMGTLGIVCPICSQKPGAHQKRTAMFQDPQERPRKLPQLCTELQTTIHDIILECVYCKQQLLRREVYDAAAAALCIVYRDGNPYAVCDKCLKFYSKISEYRHYCYSLYGTTLEQQYNKPLCDLLIRCINCQKPQCPEEKQRHLDKKQRFHNIRGRWTGRCMSCCRSSRTRRETQLGACVLLCVCLLIRPLLLSVSTYTSLIILVLLLWITAASAFRCFIVYIAFVYIPLFLIHTHA

SEQ ID NO: 71 - DNA, 942 bp, synthetic construct
  source 1..942: mol_type = other DNA
acccccaccctgcacgagtacatgctggacctgcagcccgagaccaccgccgccgccgccgccgcccagctgaacgacagcagcgaggaggaggacgagatcgacggccccgccggccaggccgagcccgacagggctcactacaacatcgtgaccttctgctgcaagtgcgacagcaccctgaggctgtgcgtgcagagcacccacgtggacatcaggaccctggaggacctgctgatgggcaccctgggcatcgtgtgccccatctgcagccagaagcccggcgcccaccagaagaggaccgccatgttccaggacccccaggagaggcccaggaagctgccccagctgtgcaccgagctgcagaccaccatccacgacatcatcctggagtgcgtgtactgcaagcagcagctgctgaggagggaggtgtacgacgccgccgccgccgccctgtgcatcgtgtacagggacggcaacccctacgccgtgtgcgacaagtgcctgaagttctacagcaagatcagcgagtacaggcactactgctacagcctgtacggcaccaccctggagcagcagtacaacaagcccctgtgcgacctgctgatcaggtgcatcaactgccagaagccccagtgccccgaggagaagcagaggcacctggacaagaagcagaggttccacaacatcaggggcaggtggaccggcaggtgcatgagctgctgcaggagcagcaggaccaggagggagacccagctgggcgcctgcgtgctgctgtgcgtgtgcctgctgatcaggcccctgctgctgagcgtgagcacctacaccagcctgatcatcctggtgctgctgctgtggatcaccgccgccagcgccttcaggtgcttcatcgtgtacatcgccttcgtgtacatccccctgttcctgatccacacccacgcc

SEQ ID NO: 72 - protein, 161 aa, synthetic construct
AAARKFFHRTCKCTGNFAAAANESFALPYWNFATGRNECDVAAADQLGYSYAIDLPVSVAAAAAASVYDFFVWLAAAAAASTFSFRNALAASQVMNLHNLAATAPDNLGYMAAAIAVVNALLLYAYDYEELYAKVPRNQDWLAAAAFGLANEKSIAAAAPR

SEQ ID NO: 73 - DNA, 483 bp, synthetic construct
  source 1..483: mol_type = other DNA
gccgctgcaaggaagttctttcacagaacctgcaaatgtacaggaaacttcgccgctgcagccaatgagagcttcgccctgccctattggaactttgctaccggccggaatgaatgcgacgtggctgcagccgatcagctcggatactcttatgccatcgacctgcctgtgagtgtcgctgcagccgctgcagcctcagtgtacgatttctttgtctggctggctgcagccgctgcagccagcactttctcctttcgcaacgccctggctgcatctcaggtcatgaacctccataatctggcagctaccgcaccagacaacctcggatatatggcagccgctattgccgtggtcaatgctctgctcctgtacgcctatgactacgaggaactgtacgctaaagtccccaggaatcaggattggctcgcagccgctgcatttggtctggccaacgaaaagagcattgcagcagcagcacctagg

SEQ ID NO: 74 - protein, 174 aa, synthetic construct
AKFVAAWTLKAAAAAARKFFHRTCKCTGNFAAAANESFALPYWNFATGRNECDVAAADQLGYSYAIDLPVSVAAAAAASVYDFFVWLAAAAAASTFSFRNALAASQVMNLHNLAATAPDNLGYMAAAIAVVNALLLYAYDYEELYAKVPRNQDWLAAAAFGLANEKSIAAAAPR

SEQ ID NO: 75 - DNA, 522 bp, synthetic construct
  source 1..522: mol_type = other DNA
gctaagtttgtggccgcttggacactgaaggccgctgcagccgctgcaaggaagttctttcacagaacctgcaaatgtacaggaaacttcgccgctgcagccaatgagagcttcgccctgccctattggaactttgctaccggccggaatgaatgcgacgtggctgcagccgatcagctcggatactcttatgccatcgacctgcctgtgagtgtcgctgcagccgctgcagcctcagtgtacgatttctttgtctggctggctgcagccgctgcagccagcactttctcctttcgcaacgccctggctgcatctcaggtcatgaacctccataatctggcagctaccgcaccagacaacctcggatatatggcagccgctattgccgtggtcaatgctctgctcctgtacgcctatgactacgaggaactgtacgctaaagtccccaggaatcaggattggctcgcagccgctgcatttggtctggccaacgaaaagagcattgcagcagcagcacctagg

SEQ ID NO: 76 - protein, 324 aa, synthetic construct
AKFVAAWTLKAAAAAARKFFHRTCKCTGNFAAAANESFALPYWNFATGRNECDVAAADQLGYSYAIDLPVSVAAAAAASVYDFFVWLAAAAAASTFSFRNALAASQVMNLHNLAATAPDNLGYMAAAIAVVNALLLYAYDYEELYAKVPRNQDWLAAAAFGLANEKSIAAAAPRETLCQRLNVCQDKILTHYENDSTDLRDHIDYWKHMRLECAIYYKAREMGFKHINHQVVPTLAVSKNKALQAIELQLTLETIYNSQYSNEKWSNTTPIVHLKGDANTLKCLRYRFKKHCKLYTAVSSTWHWTGHNVKHKSAIVTLTYDSEW

SEQ ID NO: 77 - DNA, 972 bp, synthetic construct
  source 1..972: mol_type = other DNA
gctaagtttgtggccgcttggacactgaaggccgctgcagccgctgcaaggaagttctttcacagaacctgcaaatgtacaggaaacttcgccgctgcagccaatgagagcttcgccctgccctattggaactttgctaccggccggaatgaatgcgacgtggctgcagccgatcagctcggatactcttatgccatcgacctgcctgtgagtgtcgctgcagccgctgcagcctcagtgtacgatttctttgtctggctggctgcagccgctgcagccagcactttctcctttcgcaacgccctggctgcatctcaggtcatgaacctccataatctggcagctaccgcaccagacaacctcggatatatggcagccgctattgccgtggtcaatgctctgctcctgtacgcctatgactacgaggaactgtacgctaaagtccccaggaatcaggattggctcgcagccgctgcatttggtctggccaacgaaaagagcattgcagcagcagcacctagggagactctttgccaacgtttaaatgtgtgtcaggacaaaatactaacacattatgaaaatgatagtacagacctacgtgaccatatagactattggaaacacatgcgcctagaatgtgctatttattacaaggccagagaaatgggatttaaacatattaaccaccaggtggtgccaacactggctgtatcaaagaataaagcattacaagcaattgaactgcaactaacgttagaaacaatatataactcacaatatagtaatgaaaagtggagtaacactacacccatagtacatttaaaaggtgatgctaatactttaaaatgtttaagatatagatttaaaaagcattgtaaattgtatactgcagtgtcgtctacatggcattggacaggacataatgtaaaacataaaagtgcaattgttacacttacatatgatagtgaatgg

SEQ ID NO: 78 - protein, 233 aa, synthetic construct
AKFVAAWTLKAAAAAARKFFHRTCKCTGNFAAAANESFALPYWNFATGRNECDVAAADQLGYSYAIDLPVSVAAAAAASVYDFFVWLAAAAAASTFSFRNALAASQVMNLHNLAATAPDNLGYMAAAIAVVNALLLYAYDYEELYAKVPRNQDWLAAAAFGLANEKSIAAAAPRSNTTPIVHLKGDANTLKCLRYRFKKHCKLYTAVSSTWHWTGHNVKHKSAIVTLTYDSEW

SEQ ID NO: 79 - DNA, 699 bp, synthetic construct
  source 1..699: mol_type = other DNA
gctaagtttgtggccgcttggacactgaaggccgctgcagccgctgcaaggaagttctttcacagaacctgcaaatgtacaggaaacttcgccgctgcagccaatgagagcttcgccctgccctattggaactttgctaccggccggaatgaatgcgacgtggctgcagccgatcagctcggatactcttatgccatcgacctgcctgtgagtgtcgctgcagccgctgcagcctcagtgtacgatttctttgtctggctggctgcagccgctgcagccagcactttctcctttcgcaacgccctggctgcatctcaggtcatgaacctccataatctggcagctaccgcaccagacaacctcggatatatggcagccgctattgccgtggtcaatgctctgctcctgtacgcctatgactacgaggaactgtacgctaaagtccccaggaatcaggattggctcgcagccgctgcatttggtctggccaacgaaaagagcattgcagcagcagcacctaggagtaacactacacccatagtacatttaaaaggtgatgctaatactttaaaatgtttaagatatagatttaaaaagcattgtaaattgtatactgcagtgtcgtctacatggcattggacaggacataatgtaaaacataaaagtgcaattgttacacttacatatgatagtgaatgg

SEQ ID NO: 80 - protein, 265 aa, synthetic construct
AKFVAAWTLKAAAAAARKFFHRTCKCTGNFAAAANESFALPYWNFATGRNECDVAAADQLGYSYAIDLPVSVAAAAAASVYDFFVWLAAAAAASTFSFRNALAASQVMNLHNLAATAPDNLGYMAAAIAVVNALLLYAYDYEELYAKVPRNQDWLAAAAFGLANEKSIAAAAPRETLCQRLNVCQDKILTHYENDSTDLRDHIDYWKHMRLECAIYYKAREMGFKHINHQVVPTLAVSKNKALQAIELQLTLETIYNSQYSNEKW

SEQ ID NO: 81 - DNA, 795 bp, synthetic construct
  source 1..795: mol_type = other DNA
gctaagtttgtggccgcttggacactgaaggccgctgcagccgctgcaaggaagttctttcacagaacctgcaaatgtacaggaaacttcgccgctgcagccaatgagagcttcgccctgccctattggaactttgctaccggccggaatgaatgcgacgtggctgcagccgatcagctcggatactcttatgccatcgacctgcctgtgagtgtcgctgcagccgctgcagcctcagtgtacgatttctttgtctggctggctgcagccgctgcagccagcactttctcctttcgcaacgccctggctgcatctcaggtcatgaacctccataatctggcagctaccgcaccagacaacctcggatatatggcagccgctattgccgtggtcaatgctctgctcctgtacgcctatgactacgaggaactgtacgctaaagtccccaggaatcaggattggctcgcagccgctgcatttggtctggccaacgaaaagagcattgcagcagcagcacctagggagactctttgccaacgtttaaatgtgtgtcaggacaaaatactaacacattatgaaaatgatagtacagacctacgtgaccatatagactattggaaacacatgcgcctagaatgtgctatttattacaaggccagagaaatgggatttaaacatattaaccaccaggtggtgccaacactggctgtatcaaagaataaagcattacaagcaattgaactgcaactaacgttagaaacaatatataactcacaatatagtaatgaaaagtgg

SEQ ID NO: 82 - protein, 732 aa, synthetic construct
MGGAAARLGAVILFVVIVGLHGVRGKYALADASLKMADPNRFRGKDLPVLDQLTDPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGPHGDTPTLHEYMLDLQPETTGLYGYGQLNDSSEEEDEIDGPAGQAEPDRAHYNIVTFCCKCDSTLRLCVQSTHVDIRTLEDLLMGTLGIVCPICSQKPGAHQKRTAMFQDPQERPRKLPQLCTELQTTIHDIILECVYCKQQLLRREVYDYAYRHLCIVYRDGNPYAVCDKCLKFYSKISEYRHYCYSLYGTTLEQQYNKPLCDLLIRCINCQKPQCPEEKQRHLDKKQRFHNIRGRWTGRCMSCCRSSRTRRETQLAAATNLDTASTTLLACFLLCFCVLLCVCLLIRPLLLSVSTYTSLIILVLLLWITAASAFRCFIVYIVFVYIPLFLIHTHAGPKAPYTSTLLPPELSETPNATQPELAPEDPEDSALLEDPVGTVAPQIPPNWHIPSIQDAATPYHPPATPNNMGLIAGAVGGSLLAALVICGIVYWMHRRTRKAPKRIRLPHIREDDQPSSHQPLFY

SEQ ID NO: 83 - DNA, 2196 bp, synthetic construct
  source 1..2196: mol_type = other DNA
atggggggggctgccgccaggttgggggccgtgattttgtttgtcgtcatagtgggcctccatggggtccgcggcaaatatgccttggcggatgcctctctcaagatggccgaccccaatcgctttcgcggcaaagaccttccggtcctggaccagctgaccgaccctccgggggtccggcgcgtgtaccacatccaggcgggcctaccggacccgttccagccccccagcctcccgatcacggtttactacgccgtgttggagcgcgcctgccgcagcgtgctcctaaacgcaccgtcggaggccccccagattgtccgcggggcctccgaagacgtccggaaacaaccctacaacctgaccatcgcttggtttcggatgggaggcaactgtgctatccccatcacggtcatggagtacaccgaatgctcctacaacaagtctctgggggcctgtcccatccgaacgcagccccgctggaactactatgacagcttcagcgccgtcagcgaggataacctggggttcctgatgcacgcccccgcgtttgagaccgccggcacgtacctgcggctcgtgaagataaacgactggacggagattacacagtttatcctggagcaccgagccaagggctcctgtaagtacgccctcccgctgcgcatccccccgtcagcctgcctgtccccccaggcctaccagcagggggtgacggtggacagcatcgggatgctgccccgcttcatccccgagaaccagcgcaccgtcgccgtatacagcttgaagatcgccgggtggcacgggccccacggggacactcccaccctgcatgaatatatgctggatctgcagcctgagaccacaggcctgtacggctatggacagctgaacgacagctccgaggaagaggacgaaatcgatgggccagcaggacaggcagagccagacagagcccactacaatattgtgaccttctgctgtaagtgcgatagtacactgcgactgtgcgtgcagtcaacacatgtcgacatccgcactctggaggatctgctgatgggaaccctggggatcgtgtgccctatttgttctcagaagccaggcgctcaccagaaaaggactgcaatgtttcaggatccacaggaacgacccaggaagctgcctcagctgtgcaccgagctgcagactaccatccacgacatcattctggaatgcgtgtattgtaaacagcagctgctgaggagagaggtctacgattatgcatacaggcatctgtgcatcgtgtatagagacggaaacccatacgccgtctgcgataagtgtctgaaattctattccaagatctctgagtacaggcactattgttacagcctgtatggcacaactctggaacagcagtacaacaaacccctgtgcgacctgctgatcagatgcattaattgtcagaagccccagtgtcctgaagagaaacagcggcacctggataagaaacagaggttccataacatcagaggccggtggacaggaagatgcatgtcttgctgtcgctctagtcgaacccgacgagagacacagctggcagctgcaactaatctggacaccgccagtaccacactgctggcttgtttcctgctgtgcttttgcgtgctgctgtgcgtctgtctgctgatccggcctctgctgctgtcagtgagcacatacactagcctgatcattctggtcctgctgctgtggattacagccgcttccgctttccgctgttttatcgtgtatatcgtgttcgtgtacatccccctgtttctgattcacactcatgccgggcccaaggccccatacacgagcaccctgctgcccccggagctgtccgagacccctaacgccacgcagccagaactcgccccggaagaccccgaggattcggccctcttggaggaccccgtggggacggtggcgccgcaaatcccaccaaactggcacatcccgtcgatccaggacgccgcgacgccttaccatcccccggccaccccgaacaacatgggcctgatcgccggcgcggtgggcggcagtctcctggcagccctggtcatttgcggaattgtgtactggatgcaccgccgcactcggaaagccccaaagcgcatacgcctcccccacatccgggaagacgaccagccgtcctcgcaccagcccttgttttac

SEQ ID NO: 84 - protein, 732 aa, synthetic construct
MGGAAARLGAVILFVVIVGLHGVRGKYCLADASLKMADPNRFRGKDLPVLDQLTDPPGVRRVYHIQAGLPDPFQPPSLPITVCYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGPHGDTPTLHEYMLDLQPETTGLYGYGQLNDSSEEEDEIDGPAGQAEPDRAHYNIVTFCCKCDSTLRLCVQSTHVDIRTLEDLLMGTLGIVCPICSQKPGAHQKRTAMFQDPQERPRKLPQLCTELQTTIHDIILECVYCKQQLLRREVYDYAYRHLCIVYRDGNPYAVCDKCLKFYSKISEYRHYCYSLYGTTLEQQYNKPLCDLLIRCINCQKPQCPEEKQRHLDKKQRFHNIRGRWTGRCMSCCRSSRTRRETQLAAATNLDTASTTLLACFLLCFCVLLCVCLLIRPLLLSVSTYTSLIILVLLLWITAASAFRCFIVYIVFVYIPLFLIHTHAGPKAPYTSTLLPPELSETPNATQPELAPEDPEDSALLEDPVGTVAPQIPPNWHIPSIQDAATPYHPPATPNNMGLIAGAVGGSLLAALVICGIVYWMHRRTRKAPKRIRLPHIREDDQPSSHQPLFY

SEQ ID NO: 85 - DNA, 2196 bp, synthetic construct
  source 1..2196: mol_type = other DNA
atggggggggctgccgccaggttgggggccgtgattttgtttgtcgtcatagtgggcctccatggggtccgcggcaaatattgtttggcggatgcctctctcaagatggccgaccccaatcgctttcgcggcaaagaccttccggtcctggaccagctgaccgaccctccgggggtccggcgcgtgtaccacatccaggcgggcctaccggacccgttccagccccccagcctcccgatcacggtttgctacgccgtgttggagcgcgcctgccgcagcgtgctcctaaacgcaccgtcggaggccccccagattgtccgcggggcctccgaagacgtccggaaacaaccctacaacctgaccatcgcttggtttcggatgggaggcaactgtgctatccccatcacggtcatggagtacaccgaatgctcctacaacaagtctctgggggcctgtcccatccgaacgcagccccgctggaactactatgacagcttcagcgccgtcagcgaggataacctggggttcctgatgcacgcccccgcgtttgagaccgccggcacgtacctgcggctcgtgaagataaacgactggacggagattacacagtttatcctggagcaccgagccaagggctcctgtaagtacgccctcccgctgcgcatccccccgtcagcctgcctctccccccaggcctaccagcagggggtgacggtggacagcatcgggatgctgccccgcttcatccccgagaaccagcgcaccgtcgccgtatacagcttgaagatcgccgggtggcacgggccccacggggacactcccaccctgcatgaatatatgctggatctgcagcctgagaccacaggcctgtacggctatggacagctgaacgacagctccgaggaagaggacgaaatcgatgggccagcaggacaggcagagccagacagagcccactacaatattgtgaccttctgctgtaagtgcgatagtacactgcgactgtgcgtgcagtcaacacatgtcgacatccgcactctggaggatctgctgatgggaaccctggggatcgtgtgccctatttgttctcagaagccaggcgctcaccagaaaaggactgcaatgtttcaggatccacaggaacgacccaggaagctgcctcagctgtgcaccgagctgcagactaccatccacgacatcattctggaatgcgtgtattgtaaacagcagctgctgaggagagaggtctacgattatgcatacaggcatctgtgcatcgtgtatagagacggaaacccatacgccgtctgcgataagtgtctgaaattctattccaagatctctgagtacaggcactattgttacagcctgtatggcacaactctggaacagcagtacaacaaacccctgtgcgacctgctgatcagatgcattaattgtcagaagccccagtgtcctgaagagaaacagcggcacctggataagaaacagaggttccataacatcagaggccggtggacaggaagatgcatgtcttgctgtcgctctagtcgaacccgacgagagacacagctggcagctgcaactaatctggacaccgccagtaccacactgctggcttgtttcctgctgtgcttttgcgtgctgctgtgcgtctgtctgctgatccggcctctgctgctgtcagtgagcacatacactagcctgatcattctggtcctgctgctgtggattacagccgcttccgctttccgctgttttatcgtgtatatcgtgttcgtgtacatccccctgtttctgattcacactcatgccgggcccaaggccccatacacgagcaccctgctgcccccggagctgtccgagacccccaacgccacgcagccagaactcgccccggaagaccccgaggattcggccctcttggaggaccccgtggggacggtggcgccgcaaatcccaccaaactggcacatcccgtcgatccaggacgccgcgacgccttaccatcccccggccaccccgaacaacatgggcctgatcgccggcgcggtgggcggcagtctcctggcagccctggtcatttgcggaattgtgtactggatgcaccgccgcactcggaaagccccaaagcgcatacgcctcccccacatccgggaagacgaccagccgtcctcgcaccagcccttgttttac

SEQ ID NO: 86 - protein, 732 aa, synthetic construct
MGGAAARLGAVILFVVIVGLHGVRGKYALADASLKMADPNRFRGKDLPVLDQLTDPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRAIPENQRTVAVYSLKIAGWHGPHGDTPTLHEYMLDLQPETTGLYGYGQLNDSSEEEDEIDGPAGQAEPDRAHYNIVTFCCKCDSTLRLCVQSTHVDIRTLEDLLMGTLGIVCPICSQKPGAHQKRTAMFQDPQERPRKLPQLCTELQTTIHDIILECVYCKQQLLRREVYDYAYRHLCIVYRDGNPYAVCDKCLKFYSKISEYRHYCYSLYGTTLEQQYNKPLCDLLIRCINCQKPQCPEEKQRHLDKKQRFHNIRGRWTGRCMSCCRSSRTRRETQLAAATNLDTASTTLLACFLLCFCVLLCVCLLIRPLLLSVSTYTSLIILVLLLWITAASAFRCFIVYIVFVYIPLFLIHTHAGPKAPYTSTLLPPELSETPNATQPELAPEDPEDSALLEDPVGTVAPQIPPNWHIPSIQDAATPYHPPATPNNMGLIAGAVGGSLLAALVICGIVYWMHRRTRKAPKRIRLPHIREDDQPSSHQPLFY

SEQ ID NO: 87 - DNA, 2196 bp, synthetic construct
  source 1..2196: mol_type = other DNA
atggggggggctgccgccaggttgggggccgtgattttgtttgtcgtcatagtgggcctccatggggtccgcggcaaatatgccttggcggatgcctctctcaagatggccgaccccaatcgctttcgcggcaaagaccttccggtcctggaccagctgaccgaccctccgggggtccggcgcgtgtaccacatccaggcgggcctaccggacccgttccagccccccagcctcccgatcacggtttactacgccgtgttggagcgcgcctgccgcagcgtgctcctaaacgcaccgtcggaggccccccagattgtccgcggggcctccgaagacgtccggaaacaaccctacaacctgaccatcgcttggtttcggatgggaggcaactgtgctatccccatcacggtcatggagtacaccgaatgctcctacaacaagtctctgggggcctgtcccatccgaacgcagccccgctggaactactatgacagcttcagcgccgtcagcgaggataacctggggttcctgatgcacgcccccgcgtttgagaccgccggcacgtacctgcggctcgtgaagataaacgactggacggagattacacagtttatcctggagcaccgagccaagggctcctgtaagtacgccctcccgctgcgaatccccccgtcagcctgcctctccccccaggcctaccagcagggggtgacggtggacagcatcgggatgctgccccgcgctatccccgagaaccagcgcaccgtcgccgtatacagcttgaagatcgccgggtggcacgggccccacggggacactcccaccctgcatgaatatatgctggatctgcagcctgagaccacaggcctgtacggctatggacagctgaacgacagctccgaggaagaggacgaaatcgatgggccagcaggacaggcagagccagacagagcccactacaatattgtgaccttctgctgtaagtgcgatagtacactgcgactgtgcgtgcagtcaacacatgtcgacatccgcactctggaggatctgctgatgggaaccctggggatcgtgtgccctatttgttctcagaagccaggcgctcaccagaaaaggactgcaatgtttcaggatccacaggaacgacccaggaagctgcctcagctgtgcaccgagctgcagactaccatccacgacatcattctggaatgcgtgtattgtaaacagcagctgctgaggagagaggtctacgattatgcatacaggcatctgtgcatcgtgtatagagacggaaacccatacgccgtctgcgataagtgtctgaaattctattccaagatctctgagtacaggcactattgttacagcctgtatggcacaactctggaacagcagtacaacaaacccctgtgcgacctgctgatcagatgcattaattgtcagaagccccagtgtcctgaagagaaacagcggcacctggataagaaacagaggttccataacatcagaggccggtggacaggaagatgcatgtcttgctgtcgctctagtcgaacccgacgagagacacagctggcagctgcaactaatctggacaccgccagtaccacactgctggcttgtttcctgctgtgcttttgcgtgctgctgtgcgtctgtctgctgatccggcctctgctgctgtcagtgagcacatacactagcctgatcattctggtcctgctgctgtggattacagccgcttccgctttccgctgttttatcgtgtatatcgtgttcgtgtacatccccctgtttctgattcacactcatgccgggcccaaggccccatacacgagcaccctgctgcccccggagctgtccgagacccccaacgccacgcagccagaactcgccccggaagaccccgaggattcggccctcttggaggaccccgtggggacggtggcgccgcaaatcccaccaaactggcacatcccgtcgatccaggacgccgcgacgccttaccatcccccggccaccccgaacaacatgggcctgatcgccggcgcggtgggcggcagtctcctggcagccctggtcatttgcggaattgtgtactggatgcaccgccgcactcggaaagccccaaagcgcatacgcctcccccacatccgggaagacgaccagccgtcctcgcaccagcccttgttttac

SEQ ID NO: 88 - protein, 638 aa, synthetic construct
MGGAAARLGAVILFVVIVGLHGVRGKYALADASLKMADPNRFRGKDLPVLDQLTDPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGPHGDTPTLHEYMLDLQPETTGLYGYGQLNDSSEEEDEIDGPAGQAEPDRAHYNIVTFCCKCDSTLRLCVQSTHVDIRTLEDLLMGTLGIVCPICSQKPGAHQKRTAMFQDPQERPRKLPQLCTELQTTIHDIILECVYCKQQLLRREVYDYAYRHLCIVYRDGNPYAVCDKCLKFYSKISEYRHYCYSLYGTTLEQQYNKPLCDLLIRCINCQKPQCPEEKQRHLDKKQRFHNIRGRWTGRCMSCCRSSRTRRETQLAAATNLDTASTTLLACFLLCFCVLLCVCLLIRPLLLSVSTYTSLIILVLLLWITAASAFRCFIVYIVFVYIPLFLIHTHAGPGLIAGAVGGSLLAALVICGIVYWMHRRTRKA

SEQ ID NO: 89 - DNA, 1914 bp, synthetic construct
  source 1..1914: mol_type = other DNA
atggggggggctgccgccaggttgggggccgtgattttgtttgtcgtcatagtgggcctccatggggtccgcggcaaatatgccttggcggatgcctctctcaagatggccgaccccaatcgctttcgcggcaaagaccttccggtcctggaccagctgaccgaccctccgggggtccggcgcgtgtaccacatccaggcgggcctaccggacccgttccagccccccagcctcccgatcacggtttactacgccgtgttggagcgcgcctgccgcagcgtgctcctaaacgcaccgtcggaggccccccagattgtccgcggggcctccgaagacgtccggaaacaaccctacaacctgaccatcgcttggtttcggatgggaggcaactgtgctatccccatcacggtcatggagtacaccgaatgctcctacaacaagtctctgggggcctgtcccatccgaacgcagccccgctggaactactatgacagcttcagcgccgtcagcgaggataacctggggttcctgatgcacgcccccgcgtttgagaccgccggcacgtacctgcggctcgtgaagataaacgactggacggagattacacagtttatcctggagcaccgagccaagggctcctgtaagtacgccctcccgctgcgcatccccccgtcagcctgcctctccccccaggcctaccagcagggggtgacggtggacagcatcgggatgctgccccgcttcatccccgagaaccagcgcaccgtcgccgtatacagcttgaagatcgccgggtggcacgggccccacggggacactcccaccctgcatgaatatatgctggatctgcagcctgagaccacaggcctgtacggctatggacagctgaacgacagctccgaggaagaggacgaaatcgatgggccagcaggacaggcagagccagacagagcccactacaatattgtgaccttctgctgtaagtgcgatagtacactgcgactgtgcgtgcagtcaacacatgtcgacatccgcactctggaggatctgctgatgggaaccctggggatcgtgtgccctatttgttctcagaagccaggcgctcaccagaaaaggactgcaatgtttcaggatccacaggaacgacccaggaagctgcctcagctgtgcaccgagctgcagactaccatccacgacatcattctggaatgcgtgtattgtaaacagcagctgctgaggagagaggtctacgattatgcatacaggcatctgtgcatcgtgtatagagacggaaacccatacgccgtctgcgataagtgtctgaaattctattccaagatctctgagtacaggcactattgttacagcctgtatggcacaactctggaacagcagtacaacaaacccctgtgcgacctgctgatcagatgcattaattgtcagaagccccagtgtcctgaagagaaacagcggcacctggataagaaacagaggttccataacatcagaggccggtggacaggaagatgcatgtcttgctgtcgctctagtcgaacccgacgagagacacagctggcagctgcaactaatctggacaccgccagtaccacactgctggcttgtttcctgctgtgcttttgcgtgctgctgtgcgtctgtctgctgatccggcctctgctgctgtcagtgagcacatacactagcctgatcattctggtcctgctgctgtggattacagccgcttccgctttccgctgttttatcgtgtatatcgtgttcgtgtacatccccctgtttctgattcacactcatgccgggcccggcctgatcgccggcgcggtgggcggcagtctcctggcagccctggtcatttgcggaattgtgtactggatgcaccgccgcactcggaaagcc

SEQ ID NO: 90 - protein, 752 aa, synthetic construct
MGGAAARLGAVILFVVIVGLHGVRGKYALADASLKMADPNRFRGKDLPVLDQLTDPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGPHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQKPGAHQKRTAMFQDPQERPRKLPQLCTELQTTIHDIILECVYCKQQLLRREVYDFAFRDLCIVYRDGNPYAVCDKCLKFYSKISEYRHYCYSLYGTTLEQQYNKPLCDLLIRCINCQKPLCPEEKQRHLDKKQRFHNIRGRWTGRCMSCCRSSRTRRETQLAAACIHNITGVLFALLCVLLCVCLLIRPLLLSVSTYTSLIILVLLLWITAASAFRCFIVYIIFVYIPLFLIHTHARFLITGPNRWPRIRYHAAAVYMTSTLLPPELSETPNATQPELAPEDPEDSALLEDPVGTVAPQIPPNWHIPSIQDAATPYHPPATPNNMGLIAGAVGGSLLAALVICGIVYWMHRRTRKAPKRIRLPHIREDDQPSSHQPLFYDYKDDDDKT

SEQ ID NO: 91 - DNA, 2256 bp, synthetic construct
  source 1..2256: mol_type = other DNA
atggggggggctgccgccaggttgggggccgtgattttgtttgtcgtcatagtgggcctccatggggtccgcggcaaatatgccttggcggatgcctctctcaagatggccgaccccaatcgctttcgcggcaaagaccttccggtcctggaccagctgaccgaccctccgggggtccggcgcgtgtaccacatccaggcgggcctaccggacccgttccagccccccagcctcccgatcacggtttactacgccgtgttggagcgcgcctgccgcagcgtgctcctaaacgcaccgtcggaggccccccagattgtccgcggggcctccgaagacgtccggaaacaaccctacaacctgaccatcgcttggtttcggatgggaggcaactgtgctatccccatcacggtcatggagtacaccgaatgctcctacaacaagtctctgggggcctgtcccatccgaacgcagccccgctggaactactatgacagcttcagcgccgtcagcgaggataacctggggttcctgatgcacgcccccgcgtttgagaccgccggcacgtacctgcggctcgtgaagataaacgactggacggagattacacagtttatcctggagcaccgagccaagggctcctgtaagtacgccctcccgctgcgcatccccccgtcagcctgcctctccccccaggcctaccagcagggggtgacggtggacagcatcgggatgctgccccgcttcatccccgagaaccagcgcaccgtcgccgtatacagcttgaagatcgccgggtggcacgggccccatggagatacacctacattgcatgaatatatgttagatttgcaaccagagacaactgatctctactgttatgagcaattaaatgacagctcagaggaggaggatgaaatagatggtccagctggacaagcagaaccggacagagcccattacaatattgtaaccttttgttgcaagtgtgactctacgcttcggttgtgcgtacaaagcacacacgtagacattcgtactttggaagacctgttaatgggcacactaggaattgtgtgccccatctgttctcagaaaccaggcgcccaccaaaagagaactgcaatgtttcaggacccacaggagcgacccagaaagttaccacagttatgcacagagctgcaaacaactatacatgatataatattagaatgtgtgtactgcaagcaacagttactgcgacgtgaggtatatgactttgcttttcgggatttatgcatagtatatagagatgggaatccatatgctgtatgtgataaatgtttaaagttttattctaaaattagtgagtatagacattattgttatagtttgtatggaacaacattagaacagcaatacaacaaaccgttgtgtgatttgttaattaggtgtattaactgtcaaaagccactgtgtcctgaagaaaagcaaagacatctggacaaaaagcaaagattccataatataaggggtcggtggaccggtcgatgtatgtcttgttgcagatcatcaagaacacgtagagaaacccagctggcggccgcctgcatccacaacattactggcgtgctttttgctttgctttgtgtgcttttgtgtgtctgcctattaatacgtccgctgcttttgtctgtgtctacatacacatcattaataatattggtattactattgtggataacagcagcctctgcgtttaggtgttttattgtatatattatatttgtttatataccattatttttaatacatacacatgcacgctttttaattacagggcccaaccggtggcctaggatccgatatcacgcggccgcagtgtacatgacgagcaccctgctgcccccggagctgtccgagacccccaacgccacgcagccagaactcgccccggaagaccccgaggattcggccctcttggaggaccccgtggggacggtggcgccgcaaatcccaccaaactggcacatcccgtcgatccaggacgccgcgacgccttaccatcccccggccaccccgaacaacatgggcctgatcgccggcgcggtgggcggcagtctcctggcagccctggtcatttgcggaattgtgtactggatgcaccgccgcactcggaaagccccaaagcgcatacgcctcccccacatccgggaagacgaccagccgtcctcgcaccagcccttgttttacgactacaaagacgatgacgacaagact

SEQ ID NO: 92 - protein, 682 aa, synthetic construct
MGGAAARLGAVILFVVIVGLHGVRGKYCLADASLKMADPNRFRGKDLPVLDQLTDPPGVRRVYHIQAGLPDPFQPPSLPITVCYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGPTPTLHEYMLDLQPETTGLYGYGQLNDSSEEEDEIDGPAGQAEPDRAHYNIVTFCCKCDSTLRLCVQSTHVDIRTLEDLLMGTLGIVCPICSQKPGAHQKRTAMFQDPQERPRKLPQLCTELQTTIHDIILECVYCKQQLLRREVYDYAYRHLCIVYRDGNPYAVCDKCLKFYSKISEYRHYCYSLYGTTLEQQYNKPLCDLLIRCINCQKPQCPEEKQRHLDKKQRFHNIRGRWTGRCMSCCRSSRTRRETQLAAATNLDTASTTLLACFLLCFCVLLCVCLLIRPLLLSVSTYTSLIILVLLLWITAASAFRCFIVYIVFVYIPLFLIGPATQPELAPEDPEDSALLEDPVGTVAPQIPPNWHIPSIQDAATPYHPPATPNNMGLIAGAVGGSLLAALVICGIVYWMHRRTRKAPKRIRLPHIREDDQPSSHQPLFY

SEQ ID NO: 93 - DNA, 2046 bp, synthetic construct
  source 1..2046: mol_type = other DNA
atggggggggctgccgccaggttgggggccgtgattttgtttgtcgtcatagtgggcctccatggggtccgcggcaaatattgtttggcggatgcctctctcaagatggccgaccccaatcgctttcgcggcaaagaccttccggtcctggaccagctgaccgaccctccgggggtccggcgcgtgtaccacatccaggcgggcctaccggacccgttccagccccccagcctcccgatcacggtttgctacgccgtgttggagcgcgcctgccgcagcgtgctcctaaacgcaccgtcggaggccccccagattgtccgcggggcctccgaagacgtccggaaacaaccctacaacctgaccatcgcttggtttcggatgggaggcaactgtgctatccccatcacggtcatggagtacaccgaatgctcctacaacaagtctctgggggcctgtcccatccgaacgcagccccgctggaactactatgacagcttcagcgccgtcagcgaggataacctggggttcctgatgcacgcccccgcgtttgagaccgccggcacgtacctgcggctcgtgaagataaacgactggacggagattacacagtttatcctggagcaccgagccaagggctcctgtaagtacgccctcccgctgcgcatccccccgtcagcctgcctctccccccaggcctaccagcagggggtgacggtggacagcatcgggcccactcccaccctgcatgaatatatgctggatctgcagcctgagaccacaggcctgtacggctatggacagctgaacgacagctccgaggaagaggacgaaatcgatgggccagcaggacaggcagagccagacagagcccactacaatattgtgaccttctgctgtaagtgcgatagtacactgcgactgtgcgtgcagtcaacacatgtcgacatccgcactctggaggatctgctgatgggaaccctggggatcgtgtgccctatttgttctcagaagccaggcgctcaccagaaaaggactgcaatgtttcaggatccacaggaacgacccaggaagctgcctcagctgtgcaccgagctgcagactaccatccacgacatcattctggaatgcgtgtattgtaaacagcagctgctgaggagagaggtctacgattatgcatacaggcatctgtgcatcgtgtatagagacggaaacccatacgccgtctgcgataagtgtctgaaattctattccaagatctctgagtacaggcactattgttacagcctgtatggcacaactctggaacagcagtacaacaaacccctgtgcgacctgctgatcagatgcattaattgtcagaagccccagtgtcctgaagagaaacagcggcacctggataagaaacagaggttccataacatcagaggccggtggacaggaagatgcatgtcttgctgtcgctctagtcgaacccgacgagagacacagctggcagctgcaactaatctggacaccgccagtaccacactgctggcttgtttcctgctgtgcttttgcgtgctgctgtgcgtctgtctgctgatccggcctctgctgctgtcagtgagcacatacactagcctgatcattctggtcctgctgctgtggattacagccgcttccgctttccgctgttttatcgtgtatatcgtgttcgtgtacatccccctgtttctgattgggcccgccacgcagccagaactcgccccggaagaccccgaggattcggccctcttggaggaccccgtggggacggtggcgccgcaaatcccaccaaactggcacatcccgtcgatccaggacgccgcgacgccttaccatcccccggccaccccgaacaacatgggcctgatcgccggcgcggtgggcggcagtctcctggcagccctggtcatttgcggaattgtgtactggatgcaccgccgcactcggaaagccccaaagcgcatacgcctcccccacatccgggaagacgaccagccgtcctcgcaccagcccttgttttac

SEQ ID NO: 94 - protein, 745 aa, synthetic construct
MGGAAARLGAVILFVVIVGLHGVRGKYALADASLKMADPNRFRGKDLPVLDQLTDPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGPTPTLHEYMLDLQPETTDLYGPAGQAEPDRAHYNIVTFCCKCDSTLRLCVQSTHVDIRTLEDLLMGTLGIVCPICAAPRKLPQLCTELNTSLQDIEITCVYCYDFAFRAACIVYRDGNPYAVCDKCLKFYSKISEYRHYCYSVYGTTLAASVSTYTSLILLVLLLWITAASAFRCFIVYIVFVYIPLFLIHTHARFLIAAETLCQRLNVCQDKILTHYENDSTDLRDHIDYWKHMRLECAIYYKAREMGFKHINHQVVPTLAVSKNKALQAIELQLTLETIYNSQYSNEKWSNTTPIVHLKGDANTLKCLRYRFKKHCKLYTAVSSTWHWTGHNVKHKSAIVTLTYDSEWGPKAPYTSTLLPPELSETPNATQPELAPEDPEDSALLEDPVGTVAPQIPPNWHIPSIQDAATPYHPPATPNNMGLIAGAVGGSLLAALVICGIVYWMHRRTRKAPKRIRLPHIREDDQPSSHQPLFY

SEQ ID NO: 95 - DNA, 2235 bp, synthetic construct
  source 1..2235: mol_type = other DNA
atggggggggctgccgccaggttgggggccgtgattttgtttgtcgtcatagtgggcctccatggggtccgcggcaaatatgccttggcggatgcctctctcaagatggccgaccccaatcgctttcgcggcaaagaccttccggtcctggaccagctgaccgaccctccgggggtccggcgcgtgtaccacatccaggcgggcctaccggacccgttccagccccccagcctcccgatcacggtttactacgccgtgttggagcgcgcctgccgcagcgtgctcctaaacgcaccgtcggaggccccccagattgtccgcggggcctccgaagacgtccggaaacaaccctacaacctgaccatcgcttggtttcggatgggaggcaactgtgctatccccatcacggtcatggagtacaccgaatgctcctacaacaagtctctgggggcctgtcccatccgaacgcagccccgctggaactactatgacagcttcagcgccgtcagcgaggataacctggggttcctgatgcacgcccccgcgtttgagaccgccggcacgtacctgcggctcgtgaagataaacgactggacggagattacacagtttatcctggagcaccgagccaagggctcctgtaagtacgccctcccgctgcgcatccccccgtcagcctgcctctccccccaggcctaccagcagggggtgacggtggacagcatcgggatgctgccccgcttcatccccgagaaccagcgcaccgtcgccgtatacagcttgaagatcgccgggtggcacgggcccacacctacattgcatgaatatatgttagatttgcaaccagagacaactgatctctacggtccagctggacaagcagaaccggacagagcccattacaatattgtaaccttttgttgcaagtgtgactctacgcttcggttgtgcgtacaaagcacacacgtagacattcgtactttggaagacctgttaatgggcacactaggaattgtgtgccccatctgtgctgctcccagaaagttaccacagttatgcacagagctgaacacttcactgcaagacatagaaataacctgtgtatattgctatgactttgcttttcgggctgcttgcatagtatatagagatgggaatccatatgctgtatgtgataaatgtttaaagttttattctaaaattagtgagtatagacattattgttatagtgtatatggaacaacattagctgcttctgtgtctacatacacatcattaatattgttggtattactattgtggataacagcagcctctgcgtttaggtgttttattgtatatattgtatttgtttatataccattatttttaatacatacacatgcacgctttttaattgctgctgagactctttgccaacgtttaaatgtgtgtcaggacaaaatactaacacattatgaaaatgatagtacagacctacgtgaccatatagactattggaaacacatgcgcctagaatgtgctatttattacaaggccagagaaatgggatttaaacatattaaccaccaggtggtgccaacactggctgtatcaaagaataaagcattacaagcaattgaactgcaactaacgttagaaacaatatataactcacaatatagtaatgaaaagtggagtaacactacacccatagtacatttaaaaggtgatgctaatactttaaaatgtttaagatatagatttaaaaagcattgtaaattgtatactgcagtgtcgtctacatggcattggacaggacataatgtaaaacataaaagtgcaattgttacacttacatatgatagtgaatgggggcccaaggccccatacacgagcaccctgctgcccccggagctgtccgagacccccaacgccacgcagccagaactcgccccggaagaccccgaggattcggccctcttggaggaccccgtggggacggtggcgccgcaaatcccaccaaactggcacatcccgtcgatccaggacgccgcgacgccttaccatcccccggccaccccgaacaacatgggcctgatcgccggcgcggtgggcggcagtctcctggcagccctggtcatttgcggaattgtgtactggatgcaccgccgcactcggaaagccccaaagcgcatacgcctcccccacatccgggaagacgaccagccgtcctcgcaccagcccttgttttac

SEQ ID NO: 96 - protein, 719 aa, synthetic construct
MGGAAARLGAVILFVVIVGLHGVRGKYALADASLKMADPNRFRGKDLPVLDQLTDPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGPHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQKPGAHQKRTAMFQDPQERPRKLPQLCTELQTTIHDIILECVYCKQQLLRREVYDFAFRDLCIVYRDGNPYAVCDKCLKFYSKISEYRHYCYSLYGTTLEQQYNKPLCDLLIRCINCQKPLCPEEKQRHLDKKQRFHNIRGRWTGRCMSCCRSSRTRRETQLAAACIHNITGVLFALLCVLLCVCLLIRPLLLSVSTYTSLIILVLLLWITAASAFRCFIVYIIFVYIPLFLIHTHARFLITGPNRWPRIRYHAAAVYMTSTLLPPELSETPNATQPELAPEDPEDSALLEDPVGTVAPQIPPNWHIPSIQDAATPYHPPATPNNMGLITRKAPKRIRLPHIREDDQPSSHQPLFY

SEQ ID NO: 97 - DNA, 2157 bp, synthetic construct
  source 1..2157: mol_type = other DNA
atggggggggctgccgccaggttgggggccgtgattttgtttgtcgtcatagtgggcctccatggggtccgcggcaaatatgccttggcggatgcctctctcaagatggccgaccccaatcgctttcgcggcaaagaccttccggtcctggaccagctgaccgaccctccgggggtccggcgcgtgtaccacatccaggcgggcctaccggacccgttccagccccccagcctcccgatcacggtttactacgccgtgttggagcgcgcctgccgcagcgtgctcctaaacgcaccgtcggaggccccccagattgtccgcggggcctccgaagacgtccggaaacaaccctacaacctgaccatcgcttggtttcggatgggaggcaactgtgctatccccatcacggtcatggagtacaccgaatgctcctacaacaagtctctgggggcctgtcccatccgaacgcagccccgctggaactactatgacagcttcagcgccgtcagcgaggataacctggggttcctgatgcacgcccccgcgtttgagaccgccggcacgtacctgcggctcgtgaagataaacgactggacggagattacacagtttatcctggagcaccgagccaagggctcctgtaagtacgccctcccgctgcgcatccccccgtcagcctgcctctccccccaggcctaccagcagggggtgacggtggacagcatcgggatgctgccccgcttcatccccgagaaccagcgcaccgtcgccgtatacagcttgaagatcgccgggtggcacgggccccatggagatacacctacattgcatgaatatatgttagatttgcaaccagagacaactgatctctactgttatgagcaattaaatgacagctcagaggaggaggatgaaatagatggtccagctggacaagcagaaccggacagagcccattacaatattgtaaccttttgttgcaagtgtgactctacgcttcggttgtgcgtacaaagcacacacgtagacattcgtactttggaagacctgttaatgggcacactaggaattgtgtgccccatctgttctcagaaaccaggcgcccaccaaaagagaactgcaatgtttcaggacccacaggagcgacccagaaagttaccacagttatgcacagagctgcaaacaactatacatgatataatattagaatgtgtgtactgcaagcaacagttactgcgacgtgaggtatatgactttgcttttcgggatttatgcatagtatatagagatgggaatccatatgctgtatgtgataaatgtttaaagttttattctaaaattagtgagtatagacattattgttatagtttgtatggaacaacattagaacagcaatacaacaaaccgttgtgtgatttgttaattaggtgtattaactgtcaaaagccactgtgtcctgaagaaaagcaaagacatctggacaaaaagcaaagattccataatataaggggtcggtggaccggtcgatgtatgtcttgttgcagatcatcaagaacacgtagagaaacccagctggcggccgcctgcatccacaacattactggcgtgctttttgctttgctttgtgtgcttttgtgtgtctgcctattaatacgtccgctgcttttgtctgtgtctacatacacatcattaataatattggtattactattgtggataacagcagcctctgcgtttaggtgttttattgtatatattatatttgtttatataccattatttttaatacatacacatgcacgctttttaattacagggcccaaccggtggcctaggatccgatatcacgcggccgcagtgtacatgacgagcaccctgctgcccccggagctgtccgagacccccaacgccacgcagccagaactcgccccggaagaccccgaggattcggccctcttggaggaccccgtggggacggtggcgccgcaaatcccaccaaactggcacatcccgtcgatccaggacgccgcgacgccttaccatcccccggccaccccgaacaacatgggcctgatcactcggaaagccccaaagcgcatacgcctcccccacatccgggaagacgaccagccgtcctcgcaccagcccttgttttac

SEQ ID NO: 98 - protein, 776 aa, synthetic construct
MGGAAARLGAVILFVVIVGLHGVRGKYALADASLKMADPNRFRGKDLPVLDQLTDPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGPKAPYTSTLLPPELSETPNATQPELAPEDPEDSALLEDPVGTVAPQIPPNAHIPSIQDAATPYHPPATPNNMGLIAGAVGGSLLAALVICGIVYWMHRRTRKAPKRIRLPHIREDDQPSSHQPLFYLSTGSGATNFSLLKQAGDVEENPGPDYKDDDDKTPTLHEYMLDLQPETTDLYGPAGQAEPDRAHYNIVTFCCKCDSTLRLCVQSTHVDIRTLEDLLMGTLGIVCPICAAPRKLPQLCTELNTSLQDIEITCVYCYDFAFRAACIVYRDGNPYAVCDKCLKFYSKISEYRHYCYSVYGTTLAASVSTYTSLILLVLLLWITAASAFRCFIVYIVFVYIPLFLIHTHARFLIAAETLCQRLNVCQDKILTHYENDSTDLRDHIDYWKHMRLECAIYYKAREMGFKHINHQVVPTLAVSKNKALQAIELQLTLETIYNSQYSNEKWSNTTPIVHLKGDANTLKCLRYRFKKHCKLYTAVSSTWHWTGHNVKHKSAIVTLTYDSEW

SEQ ID NO: 99 - DNA, 2328 bp, synthetic construct
  source 1..2328: mol_type = other DNA
atggggggggctgccgccaggttgggggccgtgattttgtttgtcgtcatagtgggcctccatggggtccgcggcaaatatgccttggcggatgcctctctcaagatggccgaccccaatcgctttcgcggcaaagaccttccggtcctggaccagctgaccgaccctccgggggtccggcgcgtgtaccacatccaggcgggcctaccggacccgttccagccccccagcctcccgatcacggtttactacgccgtgttggagcgcgcctgccgcagcgtgctcctaaacgcaccgtcggaggccccccagattgtccgcggggcctccgaagacgtccggaaacaaccctacaacctgaccatcgcttggtttcggatgggaggcaactgtgctatccccatcacggtcatggagtacaccgaatgctcctacaacaagtctctgggggcctgtcccatccgaacgcagccccgctggaactactatgacagcttcagcgccgtcagcgaggataacctggggttcctgatgcacgcccccgcgtttgagaccgccggcacgtacctgcggctcgtgaagataaacgactggacggagattacacagtttatcctggagcaccgagccaagggctcctgtaagtacgccctcccgctgcgcatccccccgtcagcctgcctgtccccccaggcctaccagcagggggtgacggtggacagcatcgggatgctgccccgcttcatccccgagaaccagcgcaccgtcgccgtatacagcttgaagatcgccgggtggcacgggcccaaggccccatacacgagcaccctgctgcccccggagctgtccgagacccctaacgccacgcagccagaactcgccccggaagaccccgaggattcggccctcttggaggaccccgtggggacggtggcgccgcaaatcccaccaaacgcgcacataccgtcgatccaggacgccgcgacgccttaccatcccccggccaccccgaacaacatgggcctgatcgccggcgcggtgggcggcagtctcctggcagccctggtcatttgcggaattgtgtactggatgcaccgccgcactcggaaagccccaaagcgcatacgcctcccccacatccgggaagacgaccagccgtcctcgcaccagcccttgttttacttgtcgacaggaagcggagctactaacttcagcctgctgaagcaggctggagacgtggaggagaaccctggacctgactacaaagacgatgacgacaagacacctacattgcatgaatatatgttagatttgcaaccagagacaactgatctctacggtccagctggacaagcagaaccggacagagcccattacaatattgtaaccttttgttgcaagtgtgactctacgcttcggttgtgcgtacaaagcacacacgtagacattcgtactttggaagacctgttaatgggcacactaggaattgtgtgccccatctgtgctgctcccagaaagttaccacagttatgcacagagctgaacacttcactgcaagacatagaaataacctgtgtatattgctatgactttgcttttcgggctgcttgcatagtatatagagatgggaatccatatgctgtatgtgataaatgtttaaagttttattctaaaattagtgagtatagacattattgttatagtgtatatggaacaacattagctgcttctgtgtctacatacacatcattaatattgttggtattactattgtggataacagcagcctctgcgtttaggtgttttattgtatatattgtatttgtttatataccattatttttaatacatacacatgcacgctttttaattgctgctgagactctttgccaacgtttaaatgtgtgtcaggacaaaatactaacacattatgaaaatgatagtacagacctacgtgaccatatagactattggaaacacatgcgcctagaatgtgctatttattacaaggccagagaaatgggatttaaacatattaaccaccaggtggtgccaacactggctgtatcaaagaataaagcattacaagcaattgaactgcaactaacgttagaaacaatatataactcacaatatagtaatgaaaagtggagtaacactacacccatagtacatttaaaaggtgatgctaatactttaaaatgtttaagatatagatttaaaaagcattgtaaattgtatactgcagtgtcgtctacatggcattggacaggacataatgtaaaacataaaagtgcaattgttacacttacatatgatagtgaatgg

SEQ ID NO: 100 - protein, 745 aa, synthetic construct; identical to SEQ ID NO: 94

SEQ ID NO: 101 - DNA, 2235 bp, synthetic construct; identical to SEQ ID NO: 95
  source 1..2235: mol_type = other DNA

SEQ ID NO: 102 - protein, 772 aa, synthetic construct
MGGAAARLGAVILFVVIVGLHGVRGKYALADASLKMADPNRFRGKDLPVLDQLTDPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGPHGDTPTLHEYMLDLQPETTAAAAAAQLNDSSEEEDEIDGPAGQAEPDRAHYNIVTFCCKCDSTLRLCVQSTHVDIRTLEDLLMGTLGIVCPICSQKPGAHQKRTAMFQDPQERPRKLPQLCTELQTTIHDIILECVYCKQQLLRREVYDAAAAALCIVYRDGNPYAVCDKCLKFYSKISEYRHYCYSLYGTTLEQQYNKPLCDLLIRCINCQKPQCPEEKQRHLDKKQRFHNIRGRWTGRCMSCCRSSRTRRETQLGACVLLCVCLLIRPLLLSVSTYTSLIILVLLLWITAASAFRCFIVYIAFVYIPLFLIHTHASNTTPIVHLKGDANTLKCLRYRFKKHCKLYTAVSSTWHWTGHNVKHKSAIVTLTYDSEWGPKAPYTSTLLPPELSETPNATQPELAPEDPEDSALLEDPVGTVAPQIPPNWHIPSIQDAATPYHPPATPNNMGLIAGAVGGSLLAALVICGIVYWMHRRTRKAPKRIRLPHIREDDQPSSHQPLFY

SEQ ID NO: 103 - DNA, 2316 bp, synthetic construct
  source 1..2316: mol_type = other DNA
atggggggggctgccgccaggttgggggccgtgattttgtttgtcgtcatagtgggcctccatggggtccgcggcaaatatgccttggcggatgcctctctcaagatggccgaccccaatcgctttcgcggcaaagaccttccggtcctggaccagctgaccgaccctccgggggtccggcgcgtgtaccacatccaggcgggcctaccggacccgttccagccccccagcctcccgatcacggtttactacgccgtgttggagcgcgcctgccgcagcgtgctcctaaacgcaccgtcggaggccccccagattgtccgcggggcctccgaagacgtccggaaacaaccctacaacctgaccatcgcttggtttcggatgggaggcaactgtgctatccccatcacggtcatggagtacaccgaatgctcctacaacaagtctctgggggcctgtcccatccgaacgcagccccgctggaactactatgacagcttcagcgccgtcagcgaggataacctggggttcctgatgcacgcccccgcgtttgagaccgccggcacgtacctgcggctcgtgaagataaacgactggacggagattacacagtttatcctggagcaccgagccaagggctcctgtaagtacgccctcccgctgcgcatccccccgtcagcctgcctgtccccccaggcctaccagcagggggtgacggtggacagcatcgggatgctgccccgcttcatccccgagaaccagcgcaccgtcgccgtatacagcttgaagatcgccgggtggcacgggccccacggcgacacccccaccctgcacgagtacatgctggacctgcagcccgagaccaccgccgccgccgccgccgcccagctgaacgacagcagcgaggaggaggacgagatcgacggccccgccggccaggccgagcccgacagggctcactacaacatcgtgaccttctgctgcaagtgcgacagcaccctgaggctgtgcgtgcagagcacccacgtggacatcaggaccctggaggacctgctgatgggcaccctgggcatcgtgtgccccatctgcagccagaagcccggcgcccaccagaagaggaccgccatgttccaggacccccaggagaggcccaggaagctgccccagctgtgcaccgagctgcagaccaccatccacgacatcatcctggagtgcgtgtactgcaagcagcagctgctgaggagggaggtgtacgacgccgccgccgccgccctgtgcatcgtgtacagggacggcaacccctacgccgtgtgcgacaagtgcctgaagttctacagcaagatcagcgagtacaggcactactgctacagcctgtacggcaccaccctggagcagcagtacaacaagcccctgtgcgacctgctgatcaggtgcatcaactgccagaagccccagtgccccgaggagaagcagaggcacctggacaagaagcagaggttccacaacatcaggggcaggtggaccggcaggtgcatgagctgctgcaggagcagcaggaccaggagggagacccagctgggcgcctgcgtgctgctgtgcgtgtgcctgctgatcaggcccctgctgctgagcgtgagcacctacaccagcctgatcatcctggtgctgctgctgtggatcaccgccgccagcgccttcaggtgcttcatcgtgtacatcgccttcgtgtacatccccctgttcctgatccacacccacgccagtaacactacacccatagtacatttaaaaggtgatgctaatactttaaaatgtttaagatatagatttaaaaagcattgtaaattgtatactgcagtgtcgtctacatggcattggacaggacataatgtaaaacataaaagtgcaattgttacacttacatatgatagtgaatgggggcccaaggccccatacacgagcaccctgctgcccccggagctgtccgagacccccaacgccacgcagccagaactcgccccggaagaccccgaggattcggccctcttggaggaccccgtggggacggtggcgccgcaaatcccaccaaactggcacatcccgtcgatccaggacgccgcgacgccttaccatcccccggccaccccgaacaacatgggcctgatcgccggcgcggtgggcggcagtctcctggcagccctggtcatttgcggaattgtgtactggatgcaccgccgcactcggaaagccccaaagcgcatacgcctcccccacatccgggaagacgaccagccgtcctcgcaccagcccttgttttac

SEQ ID NO: 104 - protein, 804 aa, synthetic construct
MGGAAARLGAVILFVVIVGLHGVRGKYALADASLKMADPNRFRGKDLPVLDQLTDPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGPHGDTPTLHEYMLDLQPETTAAAAAAQLNDSSEEEDEIDGPAGQAEPDRAHYNIVTFCCKCDSTLRLCVQSTHVDIRTLEDLLMGTLGIVCPICSQKPGAHQKRTAMFQDPQERPRKLPQLCTELQTTIHDIILECVYCKQQLLRREVYDAAAAALCIVYRDGNPYAVCDKCLKFYSKISEYRHYCYSLYGTTLEQQYNKPLCDLLIRCINCQKPQCPEEKQRHLDKKQRFHNIRGRWTGRCMSCCRSSRTRRETQLGACVLLCVCLLIRPLLLSVSTYTSLIILVLLLWITAASAFRCFIVYIAFVYIPLFLIHTHAETLCQRLNVCQDKILTHYENDSTDLRDHIDYWKHMRLECAIYYKAREMGFKHINHQVVPTLAVSKNKALQAIELQLTLETIYNSQYSNEKWGPKAPYTSTLLPPELSETPNATQPELAPEDPEDSALLEDPVGTVAPQIPPNWHIPSIQDAATPYHPPATPNNMGLIAGAVGGSLLAALVICGIVYWMHRRTRKAPKRIRLPHIREDDQPSSHQPLFY

SEQ ID NO: 105 - DNA, 2412 bp, synthetic construct
  source 1..2412: mol_type = other DNA
atggggggggctgccgccaggttgggggccgtgattttgtttgtcgtcatagtgggcctccatggggtccgcggcaaatatgccttggcggatgcctctctcaagatggccgaccccaatcgctttcgcggcaaagaccttccggtcctggaccagctgaccgaccctccgggggtccggcgcgtgtaccacatccaggcgggcctaccggacccgttccagccccccagcctcccgatcacggtttactacgccgtgttggagcgcgcctgccgcagcgtgctcctaaacgcaccgtcggaggccccccagattgtccgcggggcctccgaagacgtccggaaacaaccctacaacctgaccatcgcttggtttcggatgggaggcaactgtgctatccccatcacggtcatggagtacaccgaatgctcctacaacaagtctctgggggcctgtcccatccgaacgcagccccgctggaactactatgacagcttcagcgccgtcagcgaggataacctggggttcctgatgcacgcccccgcgtttgagaccgccggcacgtacctgcggctcgtgaagataaacgactggacggagattacacagtttatcctggagcaccgagccaagggctcctgtaagtacgccctcccgctgcgcatccccccgtcagcctgcctgtccccccaggcctaccagcagggggtgacggtggacagcatcgggatgctgccccgcttcatccccgagaaccagcgcaccgtcgccgtatacagcttgaagatcgccgggtggcacgggccccacggcgacacccccaccctgcacgagtacatgctggacctgcagcccgagaccaccgccgccgccgccgccgcccagctgaacgacagcagcgaggaggaggacgagatcgacggccccgccggccaggccgagcccgacagggctcactacaacatcgtgaccttctgctgcaagtgcgacagcaccctgaggctgtgcgtgcagagcacccacgtggacatcaggaccctggaggacctgctgatgggcaccctgggcatcgtgtgccccatctgcagccagaagcccggcgcccaccagaagaggaccgccatgttccaggacccccaggagaggcccaggaagctgccccagctgtgcaccgagctgcagaccaccatccacgacatcatcctggagtgcgtgtactgcaagcagcagctgctgaggagggaggtgtacgacgccgccgccgccgccctgtgcatcgtgtacagggacggcaacccctacgccgtgtgcgacaagtgcctgaagttctacagcaagatcagcgagtacaggcactactgctacagcctgtacggcaccaccctggagcagcagtacaacaagcccctgtgcgacctgctgatcaggtgcatcaactgccagaagccccagtgccccgaggagaagcagaggcacctggacaagaagcagaggttccacaacatcaggggcaggtggaccggcaggtgcatgagctgctgcaggagcagcaggaccaggagggagacccagctgggcgcctgcgtgctgctgtgcgtgtgcctgctgatcaggcccctgctgctgagcgtgagcacctacaccagcctgatcatcctggtgctgctgctgtggatcaccgccgccagcgccttcaggtgcttcatcgtgtacatcgccttcgtgtacatccccctgttcctgatccacacccacgccgagactctttgccaacgtttaaatgtgtgtcaggacaaaatactaacacattatgaaaatgatagtacagacctacgtgaccatatagactattggaaacacatgcgcctagaatgtgctatttattacaaggccagagaaatgggatttaaacatattaaccaccaggtggtgccaacactggctgtatcaaagaataaagcattacaagcaattgaactgcaactaacgttagaaacaatatataactcacaatatagtaatgaaaagtgggggcccaaggccccatacacgagcaccctgctgcccccggagctgtccgagacccccaacgccacgcagccagaactcgccccggaagaccccgaggattcggccctcttggaggaccccgtggggacggtggcgccgcaaatcccaccaaactggcacatcccgtcgatccaggacgccgcgacgccttaccatcccccggccaccccgaacaacatgggcctgatcgccggcgcggtgggcggcagtctcctggcagccctggtcatttgcggaattgtgtactggatgcaccgccgcactcggaaagccccaaagcgcatacgcctcccccacatccgggaagacgaccagccgtcctcgcaccagcccttgttttac

SEQ ID NO: 106 - protein, 729 aa, synthetic construct
MGGAAARLGAVILFVVIVGLHGVRGKYCLADASLKMADPNRFRGKDLPVLDQLTDPPGVRRVYHIQAGLPDPFQPPSLPITVCYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGPHGDTPTLHEYMLDLQPETTAAAAAAQLNDSSEEEDEIDGPAGQAEPDRAHYNIVTFCCKCDSTLRLCVQSTHVDIRTLEDLLMGTLGIVCPICSQKPGAHQKRTAMFQDPQERPRKLPQLCTELQTTIHDIILECVYCKQQLLRREVYDAAAAALCIVYRDGNPYAVCDKCLKFYSKISEYRHYCYSLYGTTLEQQYNKPLCDLLIRCINCQKPQCPEEKQRHLDKKQRFHNIRGRWTGRCMSCCRSSRTRRETQLGACVLLCVCLLIRPLLLSVSTYTSLIILVLLLWITAASAFRCFIVYIAFVYIPLFLIHTHASNTTPIVHLKGDANTLKCLRYRFKKHCKLYTAVSSTWHWTGHNVKHKSAIVTLTYDSEWGPATQPELAPEDPEDSALLEDPVGTVAPQIPPNWHIPSIQDAATPYHPPATPNNMGLIAGAVGGSLLAALVICGIVYWMHRRTRKAPKRIRLPHIREDDQPSSHQPLFY

SEQ ID NO: 107 - DNA, 2187 bp, synthetic construct
  source 1..2187: mol_type = other DNA
atggggggggctgccgccaggttgggggccgtgattttgtttgtcgtcatagtgggcctccatggggtccgcggcaaatattgtttggcggatgcctctctcaagatggccgaccccaatcgctttcgcggcaaagaccttccggtcctggaccagctgaccgaccctccgggggtccggcgcgtgtaccacatccaggcgggcctaccggacccgttccagccccccagcctcccgatcacggtttgctacgccgtgttggagcgcgcctgccgcagcgtgctcctaaacgcaccgtcggaggccccccagattgtccgcggggcctccgaagacgtccggaaacaaccctacaacctgaccatcgcttggtttcggatgggaggcaactgtgctatccccatcacggtcatggagtacaccgaatgctcctacaacaagtctctgggggcctgtcccatccgaacgcagccccgctggaactactatgacagcttcagcgccgtcagcgaggataacctggggttcctgatgcacgcccccgcgtttgagaccgccggcacgtacctgcggctcgtgaagataaacgactggacggagattacacagtttatcctggagcaccgagccaagggctcctgtaagtacgccctcccgctgcgcatccccccgtcagcctgcctctccccccaggcctaccagcagggggtgacggtggacagcatcgggccccacggcgacacccccaccctgcacgagtacatgctggacctgcagcccgagaccaccgccgccgccgccgccgcccagctgaacgacagcagcgaggaggaggacgagatcgacggccccgccggccaggccgagcccgacagggctcactacaacatcgtgaccttctgctgcaagtgcgacagcaccctgaggctgtgcgtgcagagcacccacgtggacatcaggaccctggaggacctgctgatgggcaccctgggcatcgtgtgccccatctgcagccagaagcccggcgcccaccagaagaggaccgccatgttccaggacccccaggagaggcccaggaagctgccccagctgtgcaccgagctgcagaccaccatccacgacatcatcctggagtgcgtgtactgcaagcagcagctgctgaggagggaggtgtacgacgccgccgccgccgccctgtgcatcgtgtacagggacggcaacccctacgccgtgtgcgacaagtgcctgaagttctacagcaagatcagcgagtacaggcactactgctacagcctgtacggcaccaccctggagcagcagtacaacaagcccctgtgcgacctgctgatcaggtgcatcaactgccagaagccccagtgccccgaggagaagcagaggcacctggacaagaagcagaggttccacaacatcaggggcaggtggaccggcaggtgcatgagctgctgcaggagcagcaggaccaggagggagacccagctgggcgcctgcgtgctgctgtgcgtgtgcctgctgatcaggcccctgctgctgagcgtgagcacctacaccagcctgatcatcctggtgctgctgctgtggatcaccgccgccagcgccttcaggtgcttcatcgtgtacatcgccttcgtgtacatccccctgttcctgatccacacccacgccagtaacactacacccatagtacatttaaaaggtgatgctaatactttaaaatgtttaagatatagatttaaaaagcattgtaaattgtatactgcagtgtcgtctacatggcattggacaggacataatgtaaaacataaaagtgcaattgttacacttacatatgatagtgaatgggggcccgccacgcagccagaactcgccccggaagaccccgaggattcggccctcttggaggaccccgtggggacggtggcgccgcaaatcccaccaaactggcacatcccgtcgatccaggacgccgcgacgccttaccatcccccggccaccccgaacaacatgggcctgatcgccggcgcggtgggcggcagtctcctggcagccctggtcatttgcggaattgtgtactggatgcaccgccgcactcggaaagccccaaagcgcatacgcctcccccacatccgggaagacgaccagccgtcctcgcaccagcccttgttttac

SEQ ID NO: 108 - protein, 761 aa, synthetic construct
MGGAAARLGAVILFVVIVGLHGVRGKYCLADASLKMADPNRFRGKDLPVLDQLTDPPGVRRVYHIQAGLPDPFQPPSLPITVCYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGPHGDTPTLHEYMLDLQPETTAAAAAAQLNDSSEEEDEIDGPAGQAEPDRAHYNIVTFCCKCDSTLRLCVQSTHVDIRTLEDLLMGTLGIVCPICSQKPGAHQKRTAMFQDPQERPRKLPQLCTELQTTIHDIILECVYCKQQLLRREVYDAAAAALCIVYRDGNPYAVCDKCLKFYSKISEYRHYCYSLYGTTLEQQYNKPLCDLLIRCINCQKPQCPEEKQRHLDKKQRFHNIRGRWTGRCMSCCRSSRTRRETQLGACVLLCVCLLIRPLLLSVSTYTSLIILVLLLWITAASAFRCFIVYIAFVYIPLFLIHTHAETLCQRLNVCQDKILTHYENDSTDLRDHIDYWKHMRLECAIYYKAREMGFKHINHQVVPTLAVSKNKALQAIELQLTLETIYNSQYSNEKWGPATQPELAPEDPEDSALLEDPVGTVAPQIPPNWHIPSIQDAATPYHPPATPNNMGLIAGAVGGSLLAALVICGIVYWMHRRTRKAPKRIRLPHIREDDQPSSHQPLFY

SEQ ID NO: 109 - DNA, 2283 bp, synthetic construct
  source 1..2283: mol_type = other DNA
atggggggggctgccgccaggttgggggccgtgattttgtttgtcgtcatagtgggcctccatggggtccgcggcaaatattgtttggcggatgcctctctcaagatggccgaccccaatcgctttcgcggcaaagaccttccggtcctggaccagctgaccgaccctccgggggtccggcgcgtgtaccacatccaggcgggcctaccggacccgttccagccccccagcctcccgatcacggtttgctacgccgtgttggagcgcgcctgccgcagcgtgctcctaaacgcaccgtcggaggccccccagattgtccgcggggcctccgaagacgtccggaaacaaccctacaacctgaccatcgcttggtttcggatgggaggcaactgtgctatccccatcacggtcatggagtacaccgaatgctcctacaacaagtctctgggggcctgtcccatccgaacgcagccccgctggaactactatgacagcttcagcgccgtcagcgaggataacctggggttcctgatgcacgcccccgcgtttgagaccgccggcacgtacctgcggctcgtgaagataaacgactggacggagattacacagtttatcctggagcaccgagccaagggctcctgtaagtacgccctcccgctgcgcatccccccgtcagcctgcctctccccccaggcctaccagcagggggtgacggtggacagcatcgggccccacggcgacacccccaccctgcacgagtacatgctggacctgcagcccgagaccaccgccgccgccgccgccgcccagctgaacgacagcagcgaggaggaggacgagatcgacggccccgccggccaggccgagcccgacagggctcactacaacatcgtgaccttctgctgcaagtgcgacagcaccctgaggctgtgcgtgcagagcacccacgtggacatcaggaccctggaggacctgctgatgggcaccctgggcatcgtgtgccccatctgcagccagaagcccggcgcccaccagaagaggaccgccatgttccaggacccccaggagaggcccaggaagctgccccagctgtgcaccgagctgcagaccaccatccacgacatcatcctggagtgcgtgtactgcaagcagcagctgctgaggagggaggtgtacgacgccgccgccgccgccctgtgcatcgtgtacagggacggcaacccctacgccgtgtgcgacaagtgcctgaagttctacagcaagatcagcgagtacaggcactactgctacagcctgtacggcaccaccctggagcagcagtacaacaagcccctgtgcgacctgctgatcaggtgcatcaactgccagaagccccagtgccccgaggagaagcagaggcacctggacaagaagcagaggttccacaacatcaggggcaggtggaccggcaggtgcatgagctgctgcaggagcagcaggaccaggagggagacccagctgggcgcctgcgtgctgctgtgcgtgtgcctgctgatcaggcccctgctgctgagcgtgagcacctacaccagcctgatcatcctggtgctgctgctgtggatcaccgccgccagcgccttcaggtgcttcatcgtgtacatcgccttcgtgtacatccccctgttcctgatccacacccacgccgagactctttgccaacgtttaaatgtgtgtcaggacaaaatactaacacattatgaaaatgatagtacagacctacgtgaccatatagactattggaaacacatgcgcctagaatgtgctatttattacaaggccagagaaatgggatttaaacatattaaccaccaggtggtgccaacactggctgtatcaaagaataaagcattacaagcaattgaactgcaactaacgttagaaacaatatataactcacaatatagtaatgaaaagtgggggcccgccacgcagccagaactcgccccggaagaccccgaggattcggccctcttggaggaccccgtggggacggtggcgccgcaaatcccaccaaactggcacatcccgtcgatccaggacgccgcgacgccttaccatcccccggccaccccgaacaacatgggcctgatcgccggcgcggtgggcggcagtctcctggcagccctggtcatttgcggaattgtgtactggatgcaccgccgcactcggaaagccccaaagcgcatacgcctcccccacatccgggaagacgaccagccgtcctcgcaccagcccttgttttac

SEQ ID NO: 110 - protein, 670 aa, synthetic construct
MGGAAARLGAVILFVVIVGLHGVRGKYCLADASLKMADPNRFRGKDLPVLDQLTDPPGVRRVYHIQAGLPDPFQPPSLPITVCYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGPHGDTPTLHEYMLDLQPETTAAAAAAQLNDSSEEEDEIDGPAGQAEPDRAHYNIVTFCCKCDSTLRLCVQSTHVDIRTLEDLLMGTLGIVCPICSQKPGAHQKRTAMFQDPQERPRKLPQLCTELQTTIHDIILECVYCKQQLLRREVYDAAAAALCIVYRDGNPYAVCDKCLKFYSKISEYRHYCYSLYGTTLEQQYNKPLCDLLIRCINCQKPQCPEEKQRHLDKKQRFHNIRGRWTGRCMSCCRSSRTRRETQLGACVLLCVCLLIRPLLLSVSTYTSLIILVLLLWITAASAFRCFIVYIAFVYIPLFLIHTHAGPATQPELAPEDPEDSALLEDPVGTVAPQIPPNWHIPSIQDAATPYHPPATPNNMGLIAGAVGGSLLAALVICGIVYWMHRRTRKAPKRIRLPHIREDDQPSSHQPLFY

SEQ ID NO: 111 - DNA, 2010 bp, synthetic construct
  source 1..2010: mol_type = other DNA
atggggggggctgccgccaggttgggggccgtgattttgtttgtcgtcatagtgggcctccatggggtccgcggcaaatattgtttggcggatgcctctctcaagatggccgaccccaatcgctttcgcggcaaagaccttccggtcctggaccagctgaccgaccctccgggggtccggcgcgtgtaccacatccaggcgggcctaccggacccgttccagccccccagcctcccgatcacggtttgctacgccgtgttggagcgcgcctgccgcagcgtgctcctaaacgcaccgtcggaggccccccagattgtccgcggggcctccgaagacgtccggaaacaaccctacaacctgaccatcgcttggtttcggatgggaggcaactgtgctatccccatcacggtcatggagtacaccgaatgctcctacaacaagtctctgggggcctgtcccatccgaacgcagccccgctggaactactatgacagcttcagcgccgtcagcgaggataacctggggttcctgatgcacgcccccgcgtttgagaccgccggcacgtacctgcggctcgtgaagataaacgactggacggagattacacagtttatcctggagcaccgagccaagggctcctgtaagtacgccctcccgctgcgcatccccccgtcagcctgcctctccccccaggcctaccagcagggggtgacggtggacagcatcgggccccacggcgacacccccaccctgcacgagtacatgctggacctgcagcccgagaccaccgccgccgccgccgccgcccagctgaacgacagcagcgaggaggaggacgagatcgacggccccgccggccaggccgagcccgacagggctcactacaacatcgtgaccttctgctgcaagtgcgacagcaccctgaggctgtgcgtgcagagcacccacgtggacatcaggaccctggaggacctgctgatgggcaccctgggcatcgtgtgccccatctgcagccagaagcccggcgcccaccagaagaggaccgccatgttccaggacccccaggagaggcccaggaagctgccccagctgtgcaccgagctgcagaccaccatccacgacatcatcctggagtgcgtgtactgcaagcagcagctgctgaggagggaggtgtacgacgccgccgccgccgccctgtgcatcgtgtacagggacggcaacccctacgccgtgtgcgacaagtgcctgaagttctacagcaagatcagcgagtacaggcactactgctacagcctgtacggcaccaccctggagcagcagtacaacaagcccctgtgcgacctgctgatcaggtgcatcaactgccagaagccccagtgccccgaggagaagcagaggcacctggacaagaagcagaggttccacaacatcaggggcaggtggaccggcaggtgcatgagctgctgcaggagcagcaggaccaggagggagacccagctgggcgcctgcgtgctgctgtgcgtgtgcctgctgatcaggcccctgctgctgagcgtgagcacctacaccagcctgatcatcctggtgctgctgctgtggatcaccgccgccagcgccttcaggtgcttcatcgtgtacatcgccttcgtgtacatccccctgttcctgatccacacccacgccgggcccgccacgcagccagaactcgccccggaagaccccgaggattcggccctcttggaggaccccgtggggacggtggcgccgcaaatcccaccaaactggcacatcccgtcgatccaggacgccgcgacgccttaccatcccccggccaccccgaacaacatgggcctgatcgccggcgcggtgggcggcagtctcctggcagccctggtcatttgcggaattgtgtactggatgcaccgccgcactcggaaagccccaaagcgcatacgcctcccccacatccgggaagacgaccagccgtcctcgcaccagcccttgttttac

SEQ ID NO: 112 - protein, 701 aa, synthetic construct
MGGAAARLGAVILFVVIVGLHGVRGKYALADASLKMADPNRFRGKDLPVLDQLTDPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGPPLSYQHFRKLLLLDEEAGPLEEELPRLADEGLNRRVAEDLNLGNLNVSIPWTHKVGNFTGLYSSTVPVFNPEWQTPSFPKIHLQEDIVDRCKQFVGPLTVNEKRRLKLIMPARFYPNVTKYLPLDKGIKPYYPEHAVNHYFQTRHYLHTLWKAGILYKRETTRSASFCGSPYSWEQELQHGSCWWLQFRNSKPCSEYCLTHLVNLLEDWGPCDEHGEHHIRIPRTPARVTGGVFLVDKNPHNTAESRLVVDFSQFSRGITRVSWPKFAVPNLQSLTNLLSSNLSWLSLDVSAAFYHIPLHPAAMPGPKAPYTSTLLPPELSETPNATQPELAPEDPEDSALLEDPVGTVAPQIPPNWHIPSIQDAATPYHPPATPNNMGLIAGAVGGSLLAALVICGIVYWMHRRTRKAPKRIRLPHIREDDQPSSHQPLFY

SEQ ID NO: 113 - DNA, 2103 bp, synthetic construct
  source 1..2103: mol_type = other DNA
atggggggggctgccgccaggttgggggccgtgattttgtttgtcgtcatagtgggcctccatggggtccgcggcaaatatgccttggcggatgcctctctcaagatggccgaccccaatcgctttcgcggcaaagaccttccggtcctggaccagctgaccgaccctccgggggtccggcgcgtgtaccacatccaggcgggcctaccggacccgttccagccccccagcctcccgatcacggtttactacgccgtgttggagcgcgcctgccgcagcgtgctcctaaacgcaccgtcggaggccccccagattgtccgcggggcctccgaagacgtccggaaacaaccctacaacctgaccatcgcttggtttcggatgggaggcaactgtgctatccccatcacggtcatggagtacaccgaatgctcctacaacaagtctctgggggcctgtcccatccgaacgcagccccgctggaactactatgacagcttcagcgccgtcagcgaggataacctggggttcctgatgcacgcccccgcgtttgagaccgccggcacgtacctgcggctcgtgaagataaacgactggacggagattacacagtttatcctggagcaccgagccaagggctcctgtaagtacgccctcccgctgcgcatccccccgtcagcctgcctctccccccaggcctaccagcagggggtgacggtggacagcatcgggatgctgccccgcttcatccccgagaaccagcgcaccgtcgccgtatacagcttgaagatcgccgggtggcacgggccccccctgagctaccagcacttcaggaagctgctgctgctggacgaggaggccggccccctggaggaggagctgcccaggctggccgacgagggcctgaacaggagggtggccgaggacctgaacctgggcaacctgaacgtgagcatcccctggacccacaaggtgggcaacttcaccggcctgtacagcagcaccgtgcccgtgttcaaccccgagtggcagacccccagcttccccaagatccacctgcaggaggacatcgtggacaggtgcaagcagttcgtgggtcccctgaccgtgaacgagaagaggaggctgaagctgatcatgcccgccaggttctaccccaacgtgaccaagtacctgcccctggacaagggcatcaagccctactaccccgagcacgccgtgaaccactacttccagaccaggcactacctgcacaccctgtggaaggccggcatcctgtacaagagggagaccaccaggagcgccagcttctgcggcagcccctacagctgggagcaggagctgcagcacggcagctgctggtggctgcagttcaggaacagcaagccctgcagcgagtactgcctgacccacctggtgaacctgctggaggactggggtccctgcgacgagcacggcgagcaccacatcaggatccccaggacccccgccagggtgaccggcggcgtgttcctggtggacaagaacccccacaacaccgccgagagcaggctggtggtggacttcagccagttcagcaggggcatcaccagggtgagctggcccaagttcgccgtgcccaacctgcagagcctgaccaacctgctgagcagcaacctgagctggctgagcctggacgtgagcgccgccttctaccacatccccctgcaccccgccgccatgcccgggcccaaggccccatacacgagcaccctgctgcccccggagctgtccgagacccccaacgccacgcagccagaactcgccccggaagaccccgaggattcggccctcttggaggaccccgtggggacggtggcgccgcaaatcccaccaaactggcacatcccgtcgatccaggacgccgcgacgccttaccatcccccggccaccccgaacaacatgggcctgatcgccggcgcggtgggcggcagtctcctggcagccctggtcatttgcggaattgtgtactggatgcaccgccgcactcggaaagccccaaagcgcatacgcctcccccacatccgggaagacgaccagccgtcctcgcaccagcccttgttttac

SEQ ID NO: 114 - protein, 658 aa, synthetic construct
MGGAAARLGAVILFVVIVGLHGVRGKYCLADASLKMADPNRFRGKDLPVLDQLTDPPGVRRVYHIQAGLPDPFQPPSLPITVCYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGPPLSYQHFRKLLLLDEEAGPLEEELPRLADEGLNRRVAEDLNLGNLNVSIPWTHKVGNFTGLYSSTVPVFNPEWQTPSFPKIHLQEDIVDRCKQFVGPLTVNEKRRLKLIMPARFYPNVTKYLPLDKGIKPYYPEHAVNHYFQTRHYLHTLWKAGILYKRETTRSASFCGSPYSWEQELQHGSCWWLQFRNSKPCSEYCLTHLVNLLEDWGPCDEHGEHHIRIPRTPARVTGGVFLVDKNPHNTAESRLVVDFSQFSRGITRVSWPKFAVPNLQSLTNLLSSNLSWLSLDVSAAFYHIPLHPAAMPGPATQPELAPEDPEDSALLEDPVGTVAPQIPPNWHIPSIQDAATPYHPPATPNNMGLIAGAVGGSLLAALVICGIVYWMHRRTRKAPKRIRLPHIREDDQPSSHQPLFY

SEQ ID NO: 115 - DNA, 1974 bp, synthetic construct
  source 1..1974: mol_type = other DNA
atggggggggctgccgccaggttgggggccgtgattttgtttgtcgtcatagtgggcctccatggggtccgcggcaaatattgtttggcggatgcctctctcaagatggccgaccccaatcgctttcgcggcaaagaccttccggtcctggaccagctgaccgaccctccgggggtccggcgcgtgtaccacatccaggcgggcctaccggacccgttccagccccccagcctcccgatcacggtttgctacgccgtgttggagcgcgcctgccgcagcgtgctcctaaacgcaccgtcggaggccccccagattgtccgcggggcctccgaagacgtccggaaacaaccctacaacctgaccatcgcttggtttcggatgggaggcaactgtgctatccccatcacggtcatggagtacaccgaatgctcctacaacaagtctctgggggcctgtcccatccgaacgcagccccgctggaactactatgacagcttcagcgccgtcagcgaggataacctggggttcctgatgcacgcccccgcgtttgagaccgccggcacgtacctgcggctcgtgaagataaacgactggacggagattacacagtttatcctggagcaccgagccaagggctcctgtaagtacgccctcccgctgcgcatccccccgtcagcctgcctctccccccaggcctaccagcagggggtgacggtggacagcatcgggccccccctgagctaccagcacttcaggaagctgctgctgctggacgaggaggccggccccctggaggaggagctgcccaggctggccgacgagggcctgaacaggagggtggccgaggacctgaacctgggcaacctgaacgtgagcatcccctggacccacaaggtgggcaacttcaccggcctgtacagcagcaccgtgcccgtgttcaaccccgagtggcagacccccagcttccccaagatccacctgcaggaggacatcgtggacaggtgcaagcagttcgtgggtcccctgaccgtgaacgagaagaggaggctgaagctgatcatgcccgccaggttctaccccaacgtgaccaagtacctgcccctggacaagggcatcaagccctactaccccgagcacgccgtgaaccactacttccagaccaggcactacctgcacaccctgtggaaggccggcatcctgtacaagagggagaccaccaggagcgccagcttctgcggcagcccctacagctgggagcaggagctgcagcacggcagctgctggtggctgcagttcaggaacagcaagccctgcagcgagtactgcctgacccacctggtgaacctgctggaggactggggtccctgcgacgagcacggcgagcaccacatcaggatccccaggacccccgccagggtgaccggcggcgtgttcctggtggacaagaacccccacaacaccgccgagagcaggctggtggtggacttcagccagttcagcaggggcatcaccagggtgagctggcccaagttcgccgtgcccaacctgcagagcctgaccaacctgctgagcagcaacctgagctggctgagcctggacgtgagcgccgccttctaccacatccccctgcaccccgccgccatgcccgggcccgccacgcagccagaactcgccccggaagaccccgaggattcggccctcttggaggaccccgtggggacggtggcgccgcaaatcccaccaaactggcacatcccgtcgatccaggacgccgcgacgccttaccatcccccggccaccccgaacaacatgggcctgatcgccggcgcggtgggcggcagtctcctggcagccctggtcatttgcggaattgtgtactggatgcaccgccgcactcggaaagccccaaagcgcatacgcctcccccacatccgggaagacgaccagccgtcctcgcaccagcccttgttttac

SEQ ID NO: 116 - protein, 531 aa, synthetic construct
PPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGPNRLAKFVAAWTLKAAAAAARKFFHRTCKCTGNFAAAANESFALPYWNFATGRNECDVAAADQLGYSYAIDLPVSVAAAAAASVYDFFVWLAAAAAASTFSFRNALAASQVMNLHNLAATAPDNLGYMAAAIAVVNALLLYAYDYEELYAKVPRNQDWLAAAAFGLANEKSIAAAAPRIRYHAAAVYMTSTLLPPELSETPNATQPELAPEDPEDSALLEDPVGTVAPQIPPNWHIPSIQDAATPYHPPATPNNMGLIAGAVGGSLLAALVICGIVYWMHRRTRKAPKRIRLPHIREDDQPSSHQPLFYDYKDDDDKT

SEQ ID NO: 117 - DNA, 1758 bp, synthetic construct
  source 1..1758: mol_type = other DNA
atggggggggctgccgccaggttgggggccgtgattttgtttgtcgtcatagtgggcctccatggggtccgcggcaaatatgccttggcggatgcctctctcaagatggccgaccccaatcgctttcgcggcaaagaccttccggtcctggaccagctgaccgaccctccgggggtccggcgcgtgtaccacatccaggcgggcctaccggacccgttccagccccccagcctcccgatcacggtttactacgccgtgttggagcgcgcctgccgcagcgtgctcctaaacgcaccgtcggaggccccccagattgtccgcggggcctccgaagacgtccggaaacaaccctacaacctgaccatcgcttggtttcggatgggaggcaactgtgctatccccatcacggtcatggagtacaccgaatgctcctacaacaagtctctgggggcctgtcccatccgaacgcagccccgctggaactactatgacagcttcagcgccgtcagcgaggataacctggggttcctgatgcacgcccccgcgtttgagaccgccggcacgtacctgcggctcgtgaagataaacgactggacggagattacacagtttatcctggagcaccgagccaagggctcctgtaagtacgccctcccgctgcgcatccccccgtcagcctgcctgtccccccaggcctaccagcagggggtgacggtggacagcatcgggatgctgccccgcttcatccccgagaaccagcgcaccgtcgccgtatacagcttgaagatcgccgggtggcacgggcccaaccggttagctaagtttgtggccgcttggacactgaaggccgctgcagccgctgcaaggaagttctttcacagaacctgcaaatgtacaggaaacttcgccgctgcagccaatgagagcttcgccctgccctattggaactttgctaccggccggaatgaatgcgacgtggctgcagccgatcagctcggatactcttatgccatcgacctgcctgtgagtgtcgctgcagccgctgcagcctcagtgtacgatttctttgtctggctggctgcagccgctgcagccagcactttctcctttcgcaacgccctggctgcatctcaggtcatgaacctccataatctggcagctaccgcaccagacaacctcggatatatggcagccgctattgccgtggtcaatgctctgctcctgtacgcctatgactacgaggaactgtacgctaaagtccccaggaatcaggattggctcgcagccgctgcatttggtctggccaacgaaaagagcattgcagcagcagcacctaggatccgatatcacgcggccgcagtgtacatgacgagcaccctgctgcccccggagctgtccgagacccccaacgccacgcagccagaactcgccccggaagaccccgaggattcggccctcttggaggaccccgtggggacggtggcgccgcaaatcccaccaaactggcacatcccgtcgatccaggacgccgcgacgccttaccatcccccggccaccccgaacaacatgggcctgatcgccggcgcggtgggcggcagtctcctggcagccctggtcatttgcggaattgtgtactggatgcaccgccgcactcggaaagccccaaagcgcatacgcctcccccacatccgggaagacgaccagccgtcctcgcaccagcccttgttttacgactacaaagacgatgacgacaagact

SEQ ID NO: 118 - protein, 530 aa, synthetic construct
MGGAAARLGAVILFVVIVGLHGVRGKYCLADASLKMADPNRFRGKDLPVLDQLTDPPGVRRVYHIQAGLPDPFQPPSLPITVCYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGPNRLAKFVAAWTLKAAAAAARKFFHRTCKCTGNFAAAANESFALPYWNFATGRNECDVAAADQLGYSYAIDLPVSVAAAAAASVYDFFVWLAAAAAASTFSFRNALAASQVMNLHNLAATAPDNLGYMAAAIAVVNALLLYAYDYEELYAKVPRNQDWLAAAAFGLANEKSIAAAAPRGPATQPELAPEDPEDSALLEDPVGTVAPQIPPNWHIPSIQDAATPYHPPATPNNMGLIAGAVGGSLLAALVICGIVYWMHRRTRKAPKRIRLPHIREDDQPSSHQPLFY

SEQ ID NO: 119 - DNA, 1590 bp, synthetic construct
  source 1..1590: mol_type = other DNA
atggggggggctgccgccaggttgggggccgtgattttgtttgtcgtcatagtgggcctccatggggtccgcggcaaatattgtttggcggatgcctctctcaagatggccgaccccaatcgctttcgcggcaaagaccttccggtcctggaccagctgaccgaccctccgggggtccggcgcgtgtaccacatccaggcgggcctaccggacccgttccagccccccagcctcccgatcacggtttgctacgccgtgttggagcgcgcctgccgcagcgtgctcctaaacgcaccgtcggaggccccccagattgtccgcggggcctccgaagacgtccggaaacaaccctacaacctgaccatcgcttggtttcggatgggaggcaactgtgctatccccatcacggtcatggagtacaccgaatgctcctacaacaagtctctgggggcctgtcccatccgaacgcagccccgctggaactactatgacagcttcagcgccgtcagcgaggataacctggggttcctgatgcacgcccccgcgtttgagaccgccggcacgtacctgcggctcgtgaagataaacgactggacggagattacacagtttatcctggagcaccgagccaagggctcctgtaagtacgccctcccgctgcgcatccccccgtcagcctgcctctccccccaggcctaccagcagggggtgacggtggacagcatcgggcccaaccggttagctaagtttgtggccgcttggacactgaaggccgctgcagccgctgcaaggaagttctttcacagaacctgcaaatgtacaggaaacttcgccgctgcagccaatgagagcttcgccctgccctattggaactttgctaccggccggaatgaatgcgacgtggctgcagccgatcagctcggatactcttatgccatcgacctgcctgtgagtgtcgctgcagccgctgcagcctcagtgtacgatttctttgtctggctggctgcagccgctgcagccagcactttctcctttcgcaacgccctggctgcatctcaggtcatgaacctccataatctggcagctaccgcaccagacaacctcggatatatggcagccgctattgccgtggtcaatgctctgctcctgtacgcctatgactacgaggaactgtacgctaaagtccccaggaatcaggattggctcgcagccgctgcatttggtctggccaacgaaaagagcattgcagcagcagcacctagggggcccgccacgcagccagaactcgccccggaagaccccgaggattcggccctcttggaggaccccgtggggacggtggcgccgcaaatcccaccaaactggcacatcccgtcgatccaggacgccgcgacgccttaccatcccccggccaccccgaacaacatgggcctgatcgccggcgcggtgggcggcagtctcctggcagccctggtcatttgcggaattgtgtactggatgcaccgccgcactcggaaagccccaaagcgcatacgcctcccccacatccgggaagacgaccagccgtcctcgcaccagcccttgttttac

SEQ ID NO: 120 - protein, 729 aa, synthetic construct
MGGAAARLGAVILFVVIVGLHGVRGKYALADASLKMADPNRFRGKDLPVLDQLTDPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGPNRLAKFVAAWTLKAAAAAARKFFHRTCKCTGNFAAAANESFALPYWNFATGRNECDVAAADQLGYSYAIDLPVSVAAAAAASVYDFFVWLAAAAAASTFSFRNALAASQVMNLHNLAATAPDNLGYMAAAIAVVNALLLYAYDYEELYAKVPRNQDWLAAAAFGLANEKSIAAAAPRETLCQRLNVCQDKILTHYENDSTDLRDHIDYWKHMRLECAIYYKAREMGFKHINHQVVPTLAVSKNKALQAIELQLTLETIYNSQYSNEKWSNTTPIVHLKGDANTLKCLRYRFKKHCKLYTAVSSTWHWTGHNVKHKSAIVTLTYDSEWPRIRYHAAAVYMTSTLLPPELSETPNATQPELAPEDPEDSALLEDPVGTVAPQIPPNWHIPSIQDAATPYHPPATPNNMGLIAGAVGGSLLAALVICGIVYWMHRRTRKAPKRIRLPHIREDDQPSSHQPLFY

SEQ ID NO: 121 - DNA, 2187 bp, synthetic construct
  source 1..2187: mol_type = other DNA
atggggggggctgccgccaggttgggggccgtgattttgtttgtcgtcatagtgggcctccatggggtccgcggcaaatatgccttggcggatgcctctctcaagatggccgaccccaatcgctttcgcggcaaagaccttccggtcctggaccagctgaccgaccctccgggggtccggcgcgtgtaccacatccaggcgggcctaccggacccgttccagccccccagcctcccgatcacggtttactacgccgtgttggagcgcgcctgccgcagcgtgctcctaaacgcaccgtcggaggccccccagattgtccgcggggcctccgaagacgtccggaaacaaccctacaacctgaccatcgcttggtttcggatgggaggcaactgtgctatccccatcacggtcatggagtacaccgaatgctcctacaacaagtctctgggggcctgtcccatccgaacgcagccccgctggaactactatgacagcttcagcgccgtcagcgaggataacctggggttcctgatgcacgcccccgcgtttgagaccgccggcacgtacctgcggctcgtgaagataaacgactggacggagattacacagtttatcctggagcaccgagccaagggctcctgtaagtacgccctcccgctgcgcatccccccgtcagcctgcctgtccccccaggcctaccagcagggggtgacggtggacagcatcgggatgctgccccgcttcatccccgagaaccagcgcaccgtcgccgtatacagcttgaagatcgccgggtggcacgggcccaaccggttagctaagtttgtggccgcttggacactgaaggccgctgcagccgctgcaaggaagttctttcacagaacctgcaaatgtacaggaaacttcgccgctgcagccaatgagagcttcgccctgccctattggaactttgctaccggccggaatgaatgcgacgtggctgcagccgatcagctcggatactcttatgccatcgacctgcctgtgagtgtcgctgcagccgctgcagcctcagtgtacgatttctttgtctggctggctgcagccgctgcagccagcactttctcctttcgcaacgccctggctgcatctcaggtcatgaacctccataatctggcagctaccgcaccagacaacctcggatatatggcagccgctattgccgtggtcaatgctctgctcctgtacgcctatgactacgaggaactgtacgctaaagtccccaggaatcaggattggctcgcagccgctgcatttggtctggccaacgaaaagagcattgcagcagcagcacctagggagactctttgccaacgtttaaatgtgtgtcaggacaaaatactaacacattatgaaaatgatagtacagacctacgtgaccatatagactattggaaacacatgcgcctagaatgtgctatttattacaaggccagagaaatgggatttaaacatattaaccaccaggtggtgccaacactggctgtatcaaagaataaagcattacaagcaattgaactgcaactaacgttagaaacaatatataactcacaatatagtaatgaaaagtggagtaacactacacccatagtacatttaaaaggtgatgctaatactttaaaatgtttaagatatagatttaaaaagcattgtaaattgtatactgcagtgtcgtctacatggcattggacaggacataatgtaaaacataaaagtgcaattgttacacttacatatgatagtgaatggcctaggatccgatatcacgcggccgcagtgtacatgacgagcaccctgctgcccccggagctgtccgagacccccaacgccacgcagccagaactcgccccggaagaccccgaggattcggccctcttggaggaccccgtggggacggtggcgccgcaaatcccaccaaactggcacatcccgtcgatccaggacgccgcgacgccttaccatcccccggccaccccgaacaacatgggcctgatcgccggcgcggtgggcggcagtctcctggcagccctggtcatttgcggaattgtgtactggatgcaccgccgcactcggaaagccccaaagcgcatacgcctcccccacatccgggaagacgaccagccgtcctcgcaccagcccttgttttac

SEQ ID NO: 122 - protein, 638 aa, synthetic construct
MGGAAARLGAVILFVVIVGLHGVRGKYALADASLKMADPNRFRGKDLPVLDQLTDPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGPNRLAKFVAAWTLKAAAAAARKFFHRTCKCTGNFAAAANESFALPYWNFATGRNECDVAAADQLGYSYAIDLPVSVAAAAAASVYDFFVWLAAAAAASTFSFRNALAASQVMNLHNLAATAPDNLGYMAAAIAVVNALLLYAYDYEELYAKVPRNQDWLAAAAFGLANEKSIAAAAPRSNTTPIVHLKGDANTLKCLRYRFKKHCKLYTAVSSTWHWTGHNVKHKSAIVTLTYDSEWPRIRYHAAAVYMTSTLLPPELSETPNATQPELAPEDPEDSALLEDPVGTVAPQIPPNWHIPSIQDAATPYHPPATPNNMGLIAGAVGGSLLAALVICGIVYWMHRRTRKAPKRIRLPHIREDDQPSSHQPLFY

SEQ ID NO: 123 - DNA, 1914 bp, synthetic construct
  source 1..1914: mol_type = other DNA
atggggggggctgccgccaggttgggggccgtgattttgtttgtcgtcatagtgggcctccatggggtccgcggcaaatatgccttggcggatgcctctctcaagatggccgaccccaatcgctttcgcggcaaagaccttccggtcctggaccagctgaccgaccctccgggggtccggcgcgtgtaccacatccaggcgggcctaccggacccgttccagccccccagcctcccgatcacggtttactacgccgtgttggagcgcgcctgccgcagcgtgctcctaaacgcaccgtcggaggccccccagattgtccgcggggcctccgaagacgtccggaaacaaccctacaacctgaccatcgcttggtttcggatgggaggcaactgtgctatccccatcacggtcatggagtacaccgaatgctcctacaacaagtctctgggggcctgtcccatccgaacgcagccccgctggaactactatgacagcttcagcgccgtcagcgaggataacctggggttcctgatgcacgcccccgcgtttgagaccgccggcacgtacctgcggctcgtgaagataaacgactggacggagattacacagtttatcctggagcaccgagccaagggctcctgtaagtacgccctcccgctgcgcatccccccgtcagcctgcctgtccccccaggcctaccagcagggggtgacggtggacagcatcgggatgctgccccgcttcatccccgagaaccagcgcaccgtcgccgtatacagcttgaagatcgccgggtggcacgggcccaaccggttagctaagtttgtggccgcttggacactgaaggccgctgcagccgctgcaaggaagttctttcacagaacctgcaaatgtacaggaaacttcgccgctgcagccaatgagagcttcgccctgccctattggaactttgctaccggccggaatgaatgcgacgtggctgcagccgatcagctcggatactcttatgccatcgacctgcctgtgagtgtcgctgcagccgctgcagcctcagtgtacgatttctttgtctggctggctgcagccgctgcagccagcactttctcctttcgcaacgccctggctgcatctcaggtcatgaacctccataatctggcagctaccgcaccagacaacctcggatatatggcagccgctattgccgtggtcaatgctctgctcctgtacgcctatgactacgaggaactgtacgctaaagtccccaggaatcaggattggctcgcagccgctgcatttggtctggccaacgaaaagagcattgcagcagcagcacctaggagtaacactacacccatagtacatttaaaaggtgatgctaatactttaaaatgtttaagatatagatttaaaaagcattgtaaattgtatactgcagtgtcgtctacatggcattggacaggacataatgtaaaacataaaagtgcaattgttacacttacatatgatagtgaatggcctaggatccgatatcacgcggccgcagtgtacatgacgagcaccctgctgcccccggagctgtccgagacccccaacgccacgcagccagaactcgccccggaagaccccgaggattcggccctcttggaggaccccgtggggacggtggcgccgcaaatcccaccaaactggcacatcccgtcgatccaggacgccgcgacgccttaccatcccccggccaccccgaacaacatgggcctgatcgccggcgcggtgggcggcagtctcctggcagccctggtcatttgcggaattgtgtactggatgcaccgccgcactcggaaagccccaaagcgcatacgcctcccccacatccgggaagacgaccagccgtcctcgcaccagcccttgttttac

SEQ ID NO: 124 - protein, 670 aa, synthetic construct
MGGAAARLGAVILFVVIVGLHGVRGKYALADASLKMADPNRFRGKDLPVLDQLTDPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGPNRLAKFVAAWTLKAAAAAARKFFHRTCKCTGNFAAAANESFALPYWNFATGRNECDVAAADQLGYSYAIDLPVSVAAAAAASVYDFFVWLAAAAAASTFSFRNALAASQVMNLHNLAATAPDNLGYMAAAIAVVNALLLYAYDYEELYAKVPRNQDWLAAAAFGLANEKSIAAAAPRETLCQRLNVCQDKILTHYENDSTDLRDHIDYWKHMRLECAIYYKAREMGFKHINHQVVPTLAVSKNKALQAIELQLTLETIYNSQYSNEKWPRIRYHAAAVYMTSTLLPPELSETPNATQPELAPEDPEDSALLEDPVGTVAPQIPPNWHIPSIQDAATPYHPPATPNNMGLIAGAVGGSLLAALVICGIVYWMHRRTRKAPKRIRLPHIREDDQPSSHQPLFY

SEQ ID NO: 125 - DNA, 2010 bp, synthetic construct
  source 1..2010: mol_type = other DNA
atggggggggctgccgccaggttgggggccgtgattttgtttgtcgtcatagtgggcctccatggggtccgcggcaaatatgccttggcggatgcctctctcaagatggccgaccccaatcgctttcgcggcaaagaccttccggtcctggaccagctgaccgaccctccgggggtccggcgcgtgtaccacatccaggcgggcctaccggacccgttccagccccccagcctcccgatcacggtttactacgccgtgttggagcgcgcctgccgcagcgtgctcctaaacgcaccgtcggaggccccccagattgtccgcggggcctccgaagacgtccggaaacaaccctacaacctgaccatcgcttggtttcggatgggaggcaactgtgctatccccatcacggtcatggagtacaccgaatgctcctacaacaagtctctgggggcctgtcccatccgaacgcagccccgctggaactactatgacagcttcagcgccgtcagcgaggataacctggggttcctgatgcacgcccccgcgtttgagaccgccggcacgtacctgcggctcgtgaagataaacgactggacggagattacacagtttatcctggagcaccgagccaagggctcctgtaagtacgccctcccgctgcgcatccccccgtcagcctgcctgtccccccaggcctaccagcagggggtgacggtggacagcatcgggatgctgccccgcttcatccccgagaaccagcgcaccgtcgccgtatacagcttgaagatcgccgggtggcacgggcccaaccggttagctaagtttgtggccgcttggacactgaaggccgctgcagccgctgcaaggaagttctttcacagaacctgcaaatgtacaggaaacttcgccgctgcagccaatgagagcttcgccctgccctattggaactttgctaccggccggaatgaatgcgacgtggctgcagccgatcagctcggatactcttatgccatcgacctgcctgtgagtgtcgctgcagccgctgcagcctcagtgtacgatttctttgtctggctggctgcagccgctgcagccagcactttctcctttcgcaacgccctggctgcatctcaggtcatgaacctccataatctggcagctaccgcaccagacaacctcggatatatggcagccgctattgccgtggtcaatgctctgctcctgtacgcctatgactacgaggaactgtacgctaaagtccccaggaatcaggattggctcgcagccgctgcatttggtctggccaacgaaaagagcattgcagcagcagcacctagggagactctttgccaacgtttaaatgtgtgtcaggacaaaatactaacacattatgaaaatgatagtacagacctacgtgaccatatagactattggaaacacatgcgcctagaatgtgctatttattacaaggccagagaaatgggatttaaacatattaaccaccaggtggtgccaacactggctgtatcaaagaataaagcattacaagcaattgaactgcaactaacgttagaaacaatatataactcacaatatagtaatgaaaagtggcctaggatccgatatcacgcggccgcagtgtacatgacgagcaccctgctgcccccggagctgtccgagacccccaacgccacgcagccagaactcgccccggaagaccccgaggattcggccctcttggaggaccccgtggggacggtggcgccgcaaatcccaccaaactggcacatcccgtcgatccaggacgccgcgacgccttaccatcccccggccaccccgaacaacatgggcctgatcgccggcgcggtgggcggcagtctcctggcagccctggtcatttgcggaattgtgtactggatgcaccgccgcactcggaaagccccaaagcgcatacgcctcccccacatccgggaagacgaccagccgtcctcgcaccagcccttgttttac

SEQ ID NO: 126 - protein, 738 aa, synthetic construct
MGGAAARLGAVILFVVIVGLHGVRGKYALADASLKMADPNRFRGKDLPVLDQLTDPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGPNRLAKFVAAWTLKAAAAAARKFFHRTCKCTGNFAAAANESFALPYWNFATGRNECDVAAADQLGYSYAIDLPVSVAAAAAASVYDFFVWLAAAAAASTFSFRNALAASQVMNLHNLAATAPDNLGYMAAAIAVVNALLLYAYDYEELYAKVPRNQDWLAAAAFGLANEKSIAAAAPRETLCQRLNVCQDKILTHYENDSTDLRDHIDYWKHMRLECAIYYKAREMGFKHINHQVVPTLAVSKNKALQAIELQLTLETIYNSQYSNEKWSNTTPIVHLKGDANTLKCLRYRFKKHCKLYTAVSSTWHWTGHNVKHKSAIVTLTYDSEWPRIRYHAAAVYMTSTLLPPELSETPNATQPELAPEDPEDSALLEDPVGTVAPQIPPNWHIPSIQDAATPYHPPATPNNMGLIAGAVGGSLLAALVICGIVYWMHRRTRKAPKRIRLPHIREDDQPSSHQPLFYDYKDDDDKT

SEQ ID NO: 127 - DNA, 2214 bp, synthetic construct
  source 1..2214: mol_type = other DNA
atggggggggctgccgccaggttgggggccgtgattttgtttgtcgtcatagtgggcctccatggggtccgcggcaaatatgccttggcggatgcctctctcaagatggccgaccccaatcgctttcgcggcaaagaccttccggtcctggaccagctgaccgaccctccgggggtccggcgcgtgtaccacatccaggcgggcctaccggacccgttccagccccccagcctcccgatcacggtttactacgccgtgttggagcgcgcctgccgcagcgtgctcctaaacgcaccgtcggaggccccccagattgtccgcggggcctccgaagacgtccggaaacaaccctacaacctgaccatcgcttggtttcggatgggaggcaactgtgctatccccatcacggtcatggagtacaccgaatgctcctacaacaagtctctgggggcctgtcccatccgaacgcagccccgctggaactactatgacagcttcagcgccgtcagcgaggataacctggggttcctgatgcacgcccccgcgtttgagaccgccggcacgtacctgcggctcgtgaagataaacgactggacggagattacacagtttatcctggagcaccgagccaagggctcctgtaagtacgccctcccgctgcgcatccccccgtcagcctgcctgtccccccaggcctaccagcagggggtgacggtggacagcatcgggatgctgccccgcttcatccccgagaaccagcgcaccgtcgccgtatacagcttgaagatcgccgggtggcacgggcccaaccggttagctaagtttgtggccgcttggacactgaaggccgctgcagccgctgcaaggaagttctttcacagaacctgcaaatgtacaggaaacttcgccgctgcagccaatgagagcttcgccctgccctattggaactttgctaccggccggaatgaatgcgacgtggctgcagccgatcagctcggatactcttatgccatcgacctgcctgtgagtgtcgctgcagccgctgcagcctcagtgtacgatttctttgtctggctggctgcagccgctgcagccagcactttctcctttcgcaacgccctggctgcatctcaggtcatgaacctccataatctggcagctaccgcaccagacaacctcggatatatggcagccgctattgccgtggtcaatgctctgctcctgtacgcctatgactacgaggaactgtacgctaaagtccccaggaatcaggattggctcgcagccgctgcatttggtctggccaacgaaaagagcattgcagcagcagcacctagggagactctttgccaacgtttaaatgtgtgtcaggacaaaatactaacacattatgaaaatgatagtacagacctacgtgaccatatagactattggaaacacatgcgcctagaatgtgctatttattacaaggccagagaaatgggatttaaacatattaaccaccaggtggtgccaacactggctgtatcaaagaataaagcattacaagcaattgaactgcaactaacgttagaaacaatatataactcacaatatagtaatgaaaagtggagtaacactacacccatagtacatttaaaaggtgatgctaatactttaaaatgtttaagatatagatttaaaaagcattgtaaattgtatactgcagtgtcgtctacatggcattggacaggacataatgtaaaacataaaagtgcaattgttacacttacatatgatagtgaatggcctaggatccgatatcacgcggccgcagtgtacatgacgagcaccctgctgcccccggagctgtccgagacccccaacgccacgcagccagaactcgccccggaagaccccgaggattcggccctcttggaggaccccgtggggacggtggcgccgcaaatcccaccaaactggcacatcccgtcgatccaggacgccgcgacgccttaccatcccccggccaccccgaacaacatgggcctgatcgccggcgcggtgggcggcagtctcctggcagccctggtcatttgcggaattgtgtactggatgcaccgccgcactcggaaagccccaaagcgcatacgcctcccccacatccgggaagacgaccagccgtcctcgcaccagcccttgttttacgactacaaagacgatgacgacaagact

SEQ ID NO: 128 - protein, 647 aa, synthetic construct
MGGAAARLGAVILFVVIVGLHGVRGKYALADASLKMADPNRFRGKDLPVLDQLTDPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGPNRLAKFVAAWTLKAAAAAARKFFHRTCKCTGNFAAAANESFALPYWNFATGRNECDVAAADQLGYSYAIDLPVSVAAAAAASVYDFFVWLAAAAAASTFSFRNALAASQVMNLHNLAATAPDNLGYMAAAIAVVNALLLYAYDYEELYAKVPRNQDWLAAAAFGLANEKSIAAAAPRSNTTPIVHLKGDANTLKCLRYRFKKHCKLYTAVSSTWHWTGHNVKHKSAIVTLTYDSEWPRIRYHAAAVYMTSTLLPPELSETPNATQPELAPEDPEDSALLEDPVGTVAPQIPPNWHIPSIQDAATPYHPPATPNNMGLIAGAVGGSLLAALVICGIVYWMHRRTRKAPKRIRLPHIREDDQPSSHQPLFYDYKDDDDKT

SEQ ID NO: 129 - DNA, 1941 bp, synthetic construct
  source 1..1941: mol_type = other DNA
atggggggggctgccgccaggttgggggccgtgattttgtttgtcgtcatagtgggcctccatggggtccgcggcaaatatgccttggcggatgcctctctcaagatggccgaccccaatcgctttcgcggcaaagaccttccggtcctggaccagctgaccgaccctccgggggtccggcgcgtgtaccacatccaggcgggcctaccggacccgttccagccccccagcctcccgatcacggtttactacgccgtgttggagcgcgcctgccgcagcgtgctcctaaacgcaccgtcggaggccccccagattgtccgcggggcctccgaagacgtccggaaacaaccctacaacctgaccatcgcttggtttcggatgggaggcaactgtgctatccccatcacggtcatggagtacaccgaatgctcctacaacaagtctctgggggcctgtcccatccgaacgcagccccgctggaactactatgacagcttcagcgccgtcagcgaggataacctggggttcctgatgcacgcccccgcgtttgagaccgccggcacgtacctgcggctcgtgaagataaacgactggacggagattacacagtttatcctggagcaccgagccaagggctcctgtaagtacgccctcccgctgcgcatccccccgtcagcctgcctgtccccccaggcctaccagcagggggtgacggtggacagcatcgggatgctgccccgcttcatccccgagaaccagcgcaccgtcgccgtatacagcttgaagatcgccgggtggcacgggcccaaccggttagctaagtttgtggccgcttggacactgaaggccgctgcagccgctgcaaggaagttctttcacagaacctgcaaatgtacaggaaacttcgccgctgcagccaatgagagcttcgccctgccctattggaactttgctaccggccggaatgaatgcgacgtggctgcagccgatcagctcggatactcttatgccatcgacctgcctgtgagtgtcgctgcagccgctgcagcctcagtgtacgatttctttgtctggctggctgcagccgctgcagccagcactttctcctttcgcaacgccctggctgcatctcaggtcatgaacctccataatctggcagctaccgcaccagacaacctcggatatatggcagccgctattgccgtggtcaatgctctgctcctgtacgcctatgactacgaggaactgtacgctaaagtccccaggaatcaggattggctcgcagccgctgcatttggtctggccaacgaaaagagcattgcagcagcagcacctaggagtaacactacacccatagtacatttaaaaggtgatgctaatactttaaaatgtttaagatatagatttaaaaagcattgtaaattgtatactgcagtgtcgtctacatggcattggacaggacataatgtaaaacataaaagtgcaattgttacacttacatatgatagtgaatggcctaggatccgatatcacgcggccgcagtgtacatgacgagcaccctgctgcccccggagctgtccgagacccccaacgccacgcagccagaactcgccccggaagaccccgaggattcggccctcttggaggaccccgtggggacggtggcgccgcaaatcccaccaaactggcacatcccgtcgatccaggacgccgcgacgccttaccatcccccggccaccccgaacaacatgggcctgatcgccggcgcggtgggcggcagtctcctggcagccctggtcatttgcggaattgtgtactggatgcaccgccgcactcggaaagccccaaagcgcatacgcctcccccacatccgggaagacgaccagccgtcctcgcaccagcccttgttttacgactacaaagacgatgacgacaagact

SEQ ID NO: 130 - protein, 679 aa, synthetic construct
MGGAAARLGAVILFVVIVGLHGVRGKYALADASLKMADPNRFRGKDLPVLDQLTDPPGVRRVYHIQAGLPDPFQPPSLPITVYYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGMLPRFIPENQRTVAVYSLKIAGWHGPNRLAKFVAAWTLKAAAAAARKFFHRTCKCTGNFAAAANESFALPYWNFATGRNECDVAAADQLGYSYAIDLPVSVAAAAAASVYDFFVWLAAAAAASTFSFRNALAASQVMNLHNLAATAPDNLGYMAAAIAVVNALLLYAYDYEELYAKVPRNQDWLAAAAFGLANEKSIAAAAPRETLCQRLNVCQDKILTHYENDSTDLRDHIDYWKHMRLECAIYYKAREMGFKHINHQVVPTLAVSKNKALQAIELQLTLETIYNSQYSNEKWPRIRYHAAAVYMTSTLLPPELSETPNATQPELAPEDPEDSALLEDPVGTVAPQIPPNWHIPSIQDAATPYHPPATPNNMGLIAGAVGGSLLAALVICGIVYWMHRRTRKAPKRIRLPHIREDDQPSSHQPLFYDYKDDDDKT

SEQ ID NO: 131 - DNA, 2037 bp, synthetic construct
  source 1..2037: mol_type = other DNA
atggggggggctgccgccaggttgggggccgtgattttgtttgtcgtcatagtgggcctccatggggtccgcggcaaatatgccttggcggatgcctctctcaagatggccgaccccaatcgctttcgcggcaaagaccttccggtcctggaccagctgaccgaccctccgggggtccggcgcgtgtaccacatccaggcgggcctaccggacccgttccagccccccagcctcccgatcacggtttactacgccgtgttggagcgcgcctgccgcagcgtgctcctaaacgcaccgtcggaggccccccagattgtccgcggggcctccgaagacgtccggaaacaaccctacaacctgaccatcgcttggtttcggatgggaggcaactgtgctatccccatcacggtcatggagtacaccgaatgctcctacaacaagtctctgggggcctgtcccatccgaacgcagccccgctggaactactatgacagcttcagcgccgtcagcgaggataacctggggttcctgatgcacgcccccgcgtttgagaccgccggcacgtacctgcggctcgtgaagataaacgactggacggagattacacagtttatcctggagcaccgagccaagggctcctgtaagtacgccctcccgctgcgcatccccccgtcagcctgcctgtccccccaggcctaccagcagggggtgacggtggacagcatcgggatgctgccccgcttcatccccgagaaccagcgcaccgtcgccgtatacagcttgaagatcgccgggtggcacgggcccaaccggttagctaagtttgtggccgcttggacactgaaggccgctgcagccgctgcaaggaagttctttcacagaacctgcaaatgtacaggaaacttcgccgctgcagccaatgagagcttcgccctgccctattggaactttgctaccggccggaatgaatgcgacgtggctgcagccgatcagctcggatactcttatgccatcgacctgcctgtgagtgtcgctgcagccgctgcagcctcagtgtacgatttctttgtctggctggctgcagccgctgcagccagcactttctcctttcgcaacgccctggctgcatctcaggtcatgaacctccataatctggcagctaccgcaccagacaacctcggatatatggcagccgctattgccgtggtcaatgctctgctcctgtacgcctatgactacgaggaactgtacgctaaagtccccaggaatcaggattggctcgcagccgctgcatttggtctggccaacgaaaagagcattgcagcagcagcacctagggagactctttgccaacgtttaaatgtgtgtcaggacaaaatactaacacattatgaaaatgatagtacagacctacgtgaccatatagactattggaaacacatgcgcctagaatgtgctatttattacaaggccagagaaatgggatttaaacatattaaccaccaggtggtgccaacactggctgtatcaaagaataaagcattacaagcaattgaactgcaactaacgttagaaacaatatataactcacaatatagtaatgaaaagtggcctaggatccgatatcacgcggccgcagtgtacatgacgagcaccctgctgcccccggagctgtccgagacccccaacgccacgcagccagaactcgccccggaagaccccgaggattcggccctcttggaggaccccgtggggacggtggcgccgcaaatcccaccaaactggcacatcccgtcgatccaggacgccgcgacgccttaccatcccccggccaccccgaacaacatgggcctgatcgccggcgcggtgggcggcagtctcctggcagccctggtcatttgcggaattgtgtactggatgcaccgccgcactcggaaagccccaaagcgcatacgcctcccccacatccgggaagacgaccagccgtcctcgcaccagcccttgttttacgactacaaagacgatgacgacaagact

SEQ ID NO: 132 - protein, 677 aa, synthetic construct
MGGAAARLGAVILFVVIVGLHGVRGKYCLADASLKMADPNRFRGKDLPVLDQLTDPPGVRRVYHIQAGLPDPFQPPSLPITVCYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGPAKFVAAWTLKAAAAAARKFFHRTCKCTGNFAAAANESFALPYWNFATGRNECDVAAADQLGYSYAIDLPVSVAAAAAASVYDFFVWLAAAAAASTFSFRNALAASQVMNLHNLAATAPDNLGYMAAAIAVVNALLLYAYDYEELYAKVPRNQDWLAAAAFGLANEKSIAAAAPRETLCQRLNVCQDKILTHYENDSTDLRDHIDYWKHMRLECAIYYKAREMGFKHINHQVVPTLAVSKNKALQAIELQLTLETIYNSQYSNEKWSNTTPIVHLKGDANTLKCLRYRFKKHCKLYTAVSSTWHWTGHNVKHKSAIVTLTYDSEWGPATQPELAPEDPEDSALLEDPVGTVAPQIPPNWHIPSIQDAATPYHPPATPNNMGLIAGAVGGSLLAALVICGIVYWMHRRTRKAPKRIRLPHIREDDQPSSHQPLFY

SEQ ID NO: 133 - DNA, 2031 bp, synthetic construct
  source 1..2031: mol_type = other DNA
atggggggggctgccgccaggttgggggccgtgattttgtttgtcgtcatagtgggcctccatggggtccgcggcaaatattgtttggcggatgcctctctcaagatggccgaccccaatcgctttcgcggcaaagaccttccggtcctggaccagctgaccgaccctccgggggtccggcgcgtgtaccacatccaggcgggcctaccggacccgttccagccccccagcctcccgatcacggtttgctacgccgtgttggagcgcgcctgccgcagcgtgctcctaaacgcaccgtcggaggccccccagattgtccgcggggcctccgaagacgtccggaaacaaccctacaacctgaccatcgcttggtttcggatgggaggcaactgtgctatccccatcacggtcatggagtacaccgaatgctcctacaacaagtctctgggggcctgtcccatccgaacgcagccccgctggaactactatgacagcttcagcgccgtcagcgaggataacctggggttcctgatgcacgcccccgcgtttgagaccgccggcacgtacctgcggctcgtgaagataaacgactggacggagattacacagtttatcctggagcaccgagccaagggctcctgtaagtacgccctcccgctgcgcatccccccgtcagcctgcctctccccccaggcctaccagcagggggtgacggtggacagcatcgggcccgctaagtttgtggccgcttggacactgaaggccgctgcagccgctgcaaggaagttctttcacagaacctgcaaatgtacaggaaacttcgccgctgcagccaatgagagcttcgccctgccctattggaactttgctaccggccggaatgaatgcgacgtggctgcagccgatcagctcggatactcttatgccatcgacctgcctgtgagtgtcgctgcagccgctgcagcctcagtgtacgatttctttgtctggctggctgcagccgctgcagccagcactttctcctttcgcaacgccctggctgcatctcaggtcatgaacctccataatctggcagctaccgcaccagacaacctcggatatatggcagccgctattgccgtggtcaatgctctgctcctgtacgcctatgactacgaggaactgtacgctaaagtccccaggaatcaggattggctcgcagccgctgcatttggtctggccaacgaaaagagcattgcagcagcagcacctagggagactctttgccaacgtttaaatgtgtgtcaggacaaaatactaacacattatgaaaatgatagtacagacctacgtgaccatatagactattggaaacacatgcgcctagaatgtgctatttattacaaggccagagaaatgggatttaaacatattaaccaccaggtggtgccaacactggctgtatcaaagaataaagcattacaagcaattgaactgcaactaacgttagaaacaatatataactcacaatatagtaatgaaaagtggagtaacactacacccatagtacatttaaaaggtgatgctaatactttaaaatgtttaagatatagatttaaaaagcattgtaaattgtatactgcagtgtcgtctacatggcattggacaggacataatgtaaaacataaaagtgcaattgttacacttacatatgatagtgaatgggggcccgccacgcagccagaactcgccccggaagaccccgaggattcggccctcttggaggaccccgtggggacggtggcgccgcaaatcccaccaaactggcacatcccgtcgatccaggacgccgcgacgccttaccatcccccggccaccccgaacaacatgggcctgatcgccggcgcggtgggcggcagtctcctggcagccctggtcatttgcggaattgtgtactggatgcaccgccgcactcggaaagccccaaagcgcatacgcctcccccacatccgggaagacgaccagccgtcctcgcaccagcccttgttttac

SEQ ID NO: 134 - protein, 586 aa, synthetic construct
MGGAAARLGAVILFVVIVGLHGVRGKYCLADASLKMADPNRFRGKDLPVLDQLTDPPGVRRVYHIQAGLPDPFQPPSLPITVCYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGPAKFVAAWTLKAAAAAARKFFHRTCKCTGNFAAAANESFALPYWNFATGRNECDVAAADQLGYSYAIDLPVSVAAAAAASVYDFFVWLAAAAAASTFSFRNALAASQVMNLHNLAATAPDNLGYMAAAIAVVNALLLYAYDYEELYAKVPRNQDWLAAAAFGLANEKSIAAAAPRSNTTPIVHLKGDANTLKCLRYRFKKHCKLYTAVSSTWHWTGHNVKHKSAIVTLTYDSEWGPATQPELAPEDPEDSALLEDPVGTVAPQIPPNWHIPSIQDAATPYHPPATPNNMGLIAGAVGGSLLAALVICGIVYWMHRRTRKAPKRIRLPHIREDDQPSSHQPLFY

SEQ ID NO: 135 - DNA, 1758 bp, synthetic construct
  source 1..1758: mol_type = other DNA
atggggggggctgccgccaggttgggggccgtgattttgtttgtcgtcatagtgggcctccatggggtccgcggcaaatattgtttggcggatgcctctctcaagatggccgaccccaatcgctttcgcggcaaagaccttccggtcctggaccagctgaccgaccctccgggggtccggcgcgtgtaccacatccaggcgggcctaccggacccgttccagccccccagcctcccgatcacggtttgctacgccgtgttggagcgcgcctgccgcagcgtgctcctaaacgcaccgtcggaggccccccagattgtccgcggggcctccgaagacgtccggaaacaaccctacaacctgaccatcgcttggtttcggatgggaggcaactgtgctatccccatcacggtcatggagtacaccgaatgctcctacaacaagtctctgggggcctgtcccatccgaacgcagccccgctggaactactatgacagcttcagcgccgtcagcgaggataacctggggttcctgatgcacgcccccgcgtttgagaccgccggcacgtacctgcggctcgtgaagataaacgactggacggagattacacagtttatcctggagcaccgagccaagggctcctgtaagtacgccctcccgctgcgcatccccccgtcagcctgcctctccccccaggcctaccagcagggggtgacggtggacagcatcgggcccgctaagtttgtggccgcttggacactgaaggccgctgcagccgctgcaaggaagttctttcacagaacctgcaaatgtacaggaaacttcgccgctgcagccaatgagagcttcgccctgccctattggaactttgctaccggccggaatgaatgcgacgtggctgcagccgatcagctcggatactcttatgccatcgacctgcctgtgagtgtcgctgcagccgctgcagcctcagtgtacgatttctttgtctggctggctgcagccgctgcagccagcactttctcctttcgcaacgccctggctgcatctcaggtcatgaacctccataatctggcagctaccgcaccagacaacctcggatatatggcagccgctattgccgtggtcaatgctctgctcctgtacgcctatgactacgaggaactgtacgctaaagtccccaggaatcaggattggctcgcagccgctgcatttggtctggccaacgaaaagagcattgcagcagcagcacctaggagtaacactacacccatagtacatttaaaaggtgatgctaatactttaaaatgtttaagatatagatttaaaaagcattgtaaattgtatactgcagtgtcgtctacatggcattggacaggacataatgtaaaacataaaagtgcaattgttacacttacatatgatagtgaatgggggcccgccacgcagccagaactcgccccggaagaccccgaggattcggccctcttggaggaccccgtggggacggtggcgccgcaaatcccaccaaactggcacatcccgtcgatccaggacgccgcgacgccttaccatcccccggccaccccgaacaacatgggcctgatcgccggcgcggtgggcggcagtctcctggcagccctggtcatttgcggaattgtgtactggatgcaccgccgcactcggaaagccccaaagcgcatacgcctcccccacatccgggaagacgaccagccgtcctcgcaccagcccttgttttac

SEQ ID NO: 136 - protein, 618 aa, synthetic construct
MGGAAARLGAVILFVVIVGLHGVRGKYCLADASLKMADPNRFRGKDLPVLDQLTDPPGVRRVYHIQAGLPDPFQPPSLPITVCYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGPAKFVAAWTLKAAAAAARKFFHRTCKCTGNFAAAANESFALPYWNFATGRNECDVAAADQLGYSYAIDLPVSVAAAAAASVYDFFVWLAAAAAASTFSFRNALAASQVMNLHNLAATAPDNLGYMAAAIAVVNALLLYAYDYEELYAKVPRNQDWLAAAAFGLANEKSIAAAAPRETLCQRLNVCQDKILTHYENDSTDLRDHIDYWKHMRLECAIYYKAREMGFKHINHQVVPTLAVSKNKALQAIELQLTLETIYNSQYSNEKWGPATQPELAPEDPEDSALLEDPVGTVAPQIPPNWHIPSIQDAATPYHPPATPNNMGLIAGAVGGSLLAALVICGIVYWMHRRTRKAPKRIRLPHIREDDQPSSHQPLFY

SEQ ID NO: 137 - DNA, 1854 bp, synthetic construct
  source 1..1854: mol_type = other DNA
atggggggggctgccgccaggttgggggccgtgattttgtttgtcgtcatagtgggcctccatggggtccgcggcaaatattgtttggcggatgcctctctcaagatggccgaccccaatcgctttcgcggcaaagaccttccggtcctggaccagctgaccgaccctccgggggtccggcgcgtgtaccacatccaggcgggcctaccggacccgttccagccccccagcctcccgatcacggtttgctacgccgtgttggagcgcgcctgccgcagcgtgctcctaaacgcaccgtcggaggccccccagattgtccgcggggcctccgaagacgtccggaaacaaccctacaacctgaccatcgcttggtttcggatgggaggcaactgtgctatccccatcacggtcatggagtacaccgaatgctcctacaacaagtctctgggggcctgtcccatccgaacgcagccccgctggaactactatgacagcttcagcgccgtcagcgaggataacctggggttcctgatgcacgcccccgcgtttgagaccgccggcacgtacctgcggctcgtgaagataaacgactggacggagattacacagtttatcctggagcaccgagccaagggctcctgtaagtacgccctcccgctgcgcatccccccgtcagcctgcctctccccccaggcctaccagcagggggtgacggtggacagcatcgggcccgctaagtttgtggccgcttggacactgaaggccgctgcagccgctgcaaggaagttctttcacagaacctgcaaatgtacaggaaacttcgccgctgcagccaatgagagcttcgccctgccctattggaactttgctaccggccggaatgaatgcgacgtggctgcagccgatcagctcggatactcttatgccatcgacctgcctgtgagtgtcgctgcagccgctgcagcctcagtgtacgatttctttgtctggctggctgcagccgctgcagccagcactttctcctttcgcaacgccctggctgcatctcaggtcatgaacctccataatctggcagctaccgcaccagacaacctcggatatatggcagccgctattgccgtggtcaatgctctgctcctgtacgcctatgactacgaggaactgtacgctaaagtccccaggaatcaggattggctcgcagccgctgcatttggtctggccaacgaaaagagcattgcagcagcagcacctagggagactctttgccaacgtttaaatgtgtgtcaggacaaaatactaacacattatgaaaatgatagtacagacctacgtgaccatatagactattggaaacacatgcgcctagaatgtgctatttattacaaggccagagaaatgggatttaaacatattaaccaccaggtggtgccaacactggctgtatcaaagaataaagcattacaagcaattgaactgcaactaacgttagaaacaatatataactcacaatatagtaatgaaaagtgggggcccgccacgcagccagaactcgccccggaagaccccgaggattcggccctcttggaggaccccgtggggacggtggcgccgcaaatcccaccaaactggcacatcccgtcgatccaggacgccgcgacgccttaccatcccccggccaccccgaacaacatgggcctgatcgccggcgcggtgggcggcagtctcctggcagccctggtcatttgcggaattgtgtactggatgcaccgccgcactcggaaagccccaaagcgcatacgcctcccccacatccgggaagacgaccagccgtcctcgcaccagcccttgttttac

SEQ ID NO: 138 - protein, 686 aa, synthetic construct
MGGAAARLGAVILFVVIVGLHGVRGKYCLADASLKMADPNRFRGKDLPVLDQLTDPPGVRRVYHIQAGLPDPFQPPSLPITVCYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGPAKFVAAWTLKAAAAAARKFFHRTCKCTGNFAAAANESFALPYWNFATGRNECDVAAADQLGYSYAIDLPVSVAAAAAASVYDFFVWLAAAAAASTFSFRNALAASQVMNLHNLAATAPDNLGYMAAAIAVVNALLLYAYDYEELYAKVPRNQDWLAAAAFGLANEKSIAAAAPRETLCQRLNVCQDKILTHYENDSTDLRDHIDYWKHMRLECAIYYKAREMGFKHINHQVVPTLAVSKNKALQAIELQLTLETIYNSQYSNEKWSNTTPIVHLKGDANTLKCLRYRFKKHCKLYTAVSSTWHWTGHNVKHKSAIVTLTYDSEWGPATQPELAPEDPEDSALLEDPVGTVAPQIPPNWHIPSIQDAATPYHPPATPNNMGLIAGAVGGSLLAALVICGIVYWMHRRTRKAPKRIRLPHIREDDQPSSHQPLFYDYKDDDDKT

SEQ ID NO: 139 - DNA, 2058 bp, synthetic construct
  source 1..2058: mol_type = other DNA
atggggggggctgccgccaggttgggggccgtgattttgtttgtcgtcatagtgggcctccatggggtccgcggcaaatattgtttggcggatgcctctctcaagatggccgaccccaatcgctttcgcggcaaagaccttccggtcctggaccagctgaccgaccctccgggggtccggcgcgtgtaccacatccaggcgggcctaccggacccgttccagccccccagcctcccgatcacggtttgctacgccgtgttggagcgcgcctgccgcagcgtgctcctaaacgcaccgtcggaggccccccagattgtccgcggggcctccgaagacgtccggaaacaaccctacaacctgaccatcgcttggtttcggatgggaggcaactgtgctatccccatcacggtcatggagtacaccgaatgctcctacaacaagtctctgggggcctgtcccatccgaacgcagccccgctggaactactatgacagcttcagcgccgtcagcgaggataacctggggttcctgatgcacgcccccgcgtttgagaccgccggcacgtacctgcggctcgtgaagataaacgactggacggagattacacagtttatcctggagcaccgagccaagggctcctgtaagtacgccctcccgctgcgcatccccccgtcagcctgcctctccccccaggcctaccagcagggggtgacggtggacagcatcgggcccgctaagtttgtggccgcttggacactgaaggccgctgcagccgctgcaaggaagttctttcacagaacctgcaaatgtacaggaaacttcgccgctgcagccaatgagagcttcgccctgccctattggaactttgctaccggccggaatgaatgcgacgtggctgcagccgatcagctcggatactcttatgccatcgacctgcctgtgagtgtcgctgcagccgctgcagcctcagtgtacgatttctttgtctggctggctgcagccgctgcagccagcactttctcctttcgcaacgccctggctgcatctcaggtcatgaacctccataatctggcagctaccgcaccagacaacctcggatatatggcagccgctattgccgtggtcaatgctctgctcctgtacgcctatgactacgaggaactgtacgctaaagtccccaggaatcaggattggctcgcagccgctgcatttggtctggccaacgaaaagagcattgcagcagcagcacctagggagactctttgccaacgtttaaatgtgtgtcaggacaaaatactaacacattatgaaaatgatagtacagacctacgtgaccatatagactattggaaacacatgcgcctagaatgtgctatttattacaaggccagagaaatgggatttaaacatattaaccaccaggtggtgccaacactggctgtatcaaagaataaagcattacaagcaattgaactgcaactaacgttagaaacaatatataactcacaatatagtaatgaaaagtggagtaacactacacccatagtacatttaaaaggtgatgctaatactttaaaatgtttaagatatagatttaaaaagcattgtaaattgtatactgcagtgtcgtctacatggcattggacaggacataatgtaaaacataaaagtgcaattgttacacttacatatgatagtgaatgggggcccgccacgcagccagaactcgccccggaagaccccgaggattcggccctcttggaggaccccgtggggacggtggcgccgcaaatcccaccaaactggcacatcccgtcgatccaggacgccgcgacgccttaccatcccccggccaccccgaacaacatgggcctgatcgccggcgcggtgggcggcagtctcctggcagccctggtcatttgcggaattgtgtactggatgcaccgccgcactcggaaagccccaaagcgcatacgcctcccccacatccgggaagacgaccagccgtcctcgcaccagcccttgttttacgactacaaagacgatgacgacaagact

SEQ ID NO: 140 - protein, 528 aa, synthetic construct
MGGAAARLGAVILFVVIVGLHGVRGKYCLADASLKMADPNRFRGKDLPVLDQLTDPPGVRRVYHIQAGLPDPFQPPSLPITVCYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGPQDIEITCVYCYDFAFRAACIVYRDGNPYAVCDKCLKFYSKISEYRHYCYSVYGTTLAASVSTYTSLILLVLLLWITAASAFRCFIVYIVFVYIPLFLIHTHARFLIAASNTTPIVHLKGDANTLKCLRYRFKKHCKLYTAVSSTWHWTGHNVKHKSAIVTLTYDSEWGPATQPELAPEDPEDSALLEDPVGTVAPQIPPNWHIPSIQDAATPYHPPATPNNMGLIAGAVGGSLLAALVICGIVYMHRRTRKAPKRIRLPHIREDDQPSSHQPLFYDYKDDDDKT

SEQ ID NO: 141 - DNA, 1785 bp, synthetic construct
  source 1..1785: mol_type = other DNA
atggggggggctgccgccaggttgggggccgtgattttgtttgtcgtcatagtgggcctccatggggtccgcggcaaatattgtttggcggatgcctctctcaagatggccgaccccaatcgctttcgcggcaaagaccttccggtcctggaccagctgaccgaccctccgggggtccggcgcgtgtaccacatccaggcgggcctaccggacccgttccagccccccagcctcccgatcacggtttgctacgccgtgttggagcgcgcctgccgcagcgtgctcctaaacgcaccgtcggaggccccccagattgtccgcggggcctccgaagacgtccggaaacaaccctacaacctgaccatcgcttggtttcggatgggaggcaactgtgctatccccatcacggtcatggagtacaccgaatgctcctacaacaagtctctgggggcctgtcccatccgaacgcagccccgctggaactactatgacagcttcagcgccgtcagcgaggataacctggggttcctgatgcacgcccccgcgtttgagaccgccggcacgtacctgcggctcgtgaagataaacgactggacggagattacacagtttatcctggagcaccgagccaagggctcctgtaagtacgccctcccgctgcgcatccccccgtcagcctgcctctccccccaggcctaccagcagggggtgacggtggacagcatcgggcccgctaagtttgtggccgcttggacactgaaggccgctgcagccgctgcaaggaagttctttcacagaacctgcaaatgtacaggaaacttcgccgctgcagccaatgagagcttcgccctgccctattggaactttgctaccggccggaatgaatgcgacgtggctgcagccgatcagctcggatactcttatgccatcgacctgcctgtgagtgtcgctgcagccgctgcagcctcagtgtacgatttctttgtctggctggctgcagccgctgcagccagcactttctcctttcgcaacgccctggctgcatctcaggtcatgaacctccataatctggcagctaccgcaccagacaacctcggatatatggcagccgctattgccgtggtcaatgctctgctcctgtacgcctatgactacgaggaactgtacgctaaagtccccaggaatcaggattggctcgcagccgctgcatttggtctggccaacgaaaagagcattgcagcagcagcacctaggagtaacactacacccatagtacatttaaaaggtgatgctaatactttaaaatgtttaagatatagatttaaaaagcattgtaaattgtatactgcagtgtcgtctacatggcattggacaggacataatgtaaaacataaaagtgcaattgttacacttacatatgatagtgaatgggggcccgccacgcagccagaactcgccccggaagaccccgaggattcggccctcttggaggaccccgtggggacggtggcgccgcaaatcccaccaaactggcacatcccgtcgatccaggacgccgcgacgccttaccatcccccggccaccccgaacaacatgggcctgatcgccggcgcggtgggcggcagtctcctggcagccctggtcatttgcggaattgtgtactggatgcaccgccgcactcggaaagccccaaagcgcatacgcctcccccacatccgggaagacgaccagccgtcctcgcaccagcccttgttttacgactacaaagacgatgacgacaagact

SEQ ID NO: 142 - protein, 627 aa, synthetic construct
MGGAAARLGAVILFVVIVGLHGVRGKYCLADASLKMADPNRFRGKDLPVLDQLTDPPGVRRVYHIQAGLPDPFQPPSLPITVCYAVLERACRSVLLNAPSEAPQIVRGASEDVRKQPYNLTIAWFRMGGNCAIPITVMEYTECSYNKSLGACPIRTQPRWNYYDSFSAVSEDNLGFLMHAPAFETAGTYLRLVKINDWTEITQFILEHRAKGSCKYALPLRIPPSACLSPQAYQQGVTVDSIGPAKFVAAWTLKAAAAAARKFFHRTCKCTGNFAAAANESFALPYWNFATGRNECDVAAADQLGYSYAIDLPVSVAAAAAASVYDFFVWLAAAAAASTFSFRNALAASQVMNLHNLAATAPDNLGYMAAAIAVVNALLLYAYDYEELYAKVPRNQDWLAAAAFGLANEKSIAAAAPRETLCQRLNVCQDKILTHYENDSTDLRDHIDYWKHMRLECAIYYKAREMGFKHINHQVVPTLAVSKNKALQAIELQLTLETIYNSQYSNEKWGPATQPELAPEDPEDSALLEDPVGTVAPQIPPNWHIPSIQDAATPYHPPATPNNMGLIAGAVGGSLLAALVICGIVYWMHRRTRKAPKRIRLPHIREDDQPSSHQPLFYDYKDDDDKT

SEQ ID NO: 143 - DNA, 1881 bp, synthetic construct
  source 1..1881: mol_type = other DNA
atggggggggctgccgccaggttgggggccgtgattttgtttgtcgtcatagtgggcctccatggggtccgcggcaaatattgtttggcggatgcctctctcaagatggccgaccccaatcgctttcgcggcaaagaccttccggtcctggaccagctgaccgaccctccgggggtccggcgcgtgtaccacatccaggcgggcctaccggacccgttccagccccccagcctcccgatcacggtttgctacgccgtgttggagcgcgcctgccgcagcgtgctcctaaacgcaccgtcggaggccccccagattgtccgcggggcctccgaagacgtccggaaacaaccctacaacctgaccatcgcttggtttcggatgggaggcaactgtgctatccccatcacggtcatggagtacaccgaatgctcctacaacaagtctctgggggcctgtcccatccgaacgcagccccgctggaactactatgacagcttcagcgccgtcagcgaggataacctggggttcctgatgcacgcccccgcgtttgagaccgccggcacgtacctgcggctcgtgaagataaacgactggacggagattacacagtttatcctggagcaccgagccaagggctcctgtaagtacgccctcccgctgcgcatccccccgtcagcctgcctctccccccaggcctaccagcagggggtgacggtggacagcatcgggcccgctaagtttgtggccgcttggacactgaaggccgctgcagccgctgcaaggaagttctttcacagaacctgcaaatgtacaggaaacttcgccgctgcagccaatgagagcttcgccctgccctattggaactttgctaccggccggaatgaatgcgacgtggctgcagccgatcagctcggatactcttatgccatcgacctgcctgtgagtgtcgctgcagccgctgcagcctcagtgtacgatttctttgtctggctggctgcagccgctgcagccagcactttctcctttcgcaacgccctggctgcatctcaggtcatgaacctccataatctggcagctaccgcaccagacaacctcggatatatggcagccgctattgccgtggtcaatgctctgctcctgtacgcctatgactacgaggaactgtacgctaaagtccccaggaatcaggattggctcgcagccgctgcatttggtctggccaacgaaaagagcattgcagcagcagcacctagggagactctttgccaacgtttaaatgtgtgtcaggacaaaatactaacacattatgaaaatgatagtacagacctacgtgaccatatagactattggaaacacatgcgcctagaatgtgctatttattacaaggccagagaaatgggatttaaacatattaaccaccaggtggtgccaacactggctgtatcaaagaataaagcattacaagcaattgaactgcaactaacgttagaaacaatatataactcacaatatagtaatgaaaagtgggggcccgccacgcagccagaactcgccccggaagaccccgaggattcggccctcttggaggaccccgtggggacggtggcgccgcaaatcccaccaaactggcacatcccgtcgatccaggacgccgcgacgccttaccatcccccggccaccccgaacaacatgggcctgatcgccggcgcggtgggcggcagtctcctggcagccctggtcatttgcggaattgtgtactggatgcaccgccgcactcggaaagccccaaagcgcatacgcctcccccacatccgggaagacgaccagccgtcctcgcaccagcccttgttttacgactacaaagacgatgacgacaagact